(19) United States
(12) Patent Application Publication
(10) Pub. No.: US 2023/0141403 A1
(43) Pub. Date: May 11, 2023

(54) NOVEL IMIDAZOPYRAZINE DERIVATIVES

(71) Applicant: Hoffmann-La Roche Inc., Little Falls, NJ (US)

(72) Inventors: Zhanling CHENG, Shanghai (CN); Xingchun HAN, Shanghai (CN); Christian KRAMER, Lörrach (DE); Holger KUEHNE, Lörrach (DE); Christian LERNER, Bottmingen (CH); Mingming LI, Shanghai (CN); Matthias NETTEKOVEN, Grenzach-Wyhlen (DE); Theodor STOLL, Binningen (CH); Min WANG, Shanghai (CN); Song YANG, Shanghai (CN)

(73) Assignee: Hoffmann-La Roche Inc., Little Falls, NJ (US)

(21) Appl. No.: 18/076,383

(22) Filed: Dec. 6, 2022

Related U.S. Application Data
(63) Continuation of application No. PCT/EP2021/065085, filed on Jun. 7, 2021.

(30) Foreign Application Priority Data
Jun. 8, 2020 (CN) PCT/CN2020/094920

(51) Int. Cl. C07D 487/04; A61P 31/04

(52) U.S. Cl. CPC C07D 487/04; A61P 31/04

(57) ABSTRACT

The invention provides novel imidazopyrazine derivatives having the general formula (I), or pharmaceutically acceptable salts thereof, wherein X, m, n, and R¹ to R³ are as described herein:

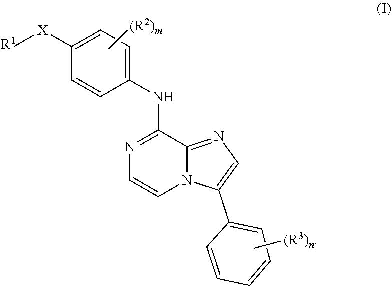

Further provided are pharmaceutical compositions including the compounds, processes of manufacturing the compounds and methods of using the compounds as medicaments, in particular methods of using the compounds as antibiotics for the treatment or prevention of bacterial infections and resulting diseases.

### CROSS-REFERENCE TO RELATED APPLICATIONS

This application is a Continuation Application of International Patent Application No. PCT/EP2021/065085, filed on Jun. 7, 2021, which claims benefit of priority to International Patent Application No. PCT/CN2020/094920, filed on Jun. 8, 2020, each of which is incorporated herein by reference in its entirety.

### BACKGROUND

The present invention relates to novel imidazolepyrazole derivatives which exhibit antibacterial properties. The invention also relates to methods of using the compounds for the treatment or prevention of bacterial infections and resulting diseases, in particular for the treatment or prevention of infections with Acinetobacter baumannii and resulting diseases.

Acinetobacter baumannii is a Gram-negative, aerobic, nonfermenting bacterium recognized over the last decades as an emergining pathogen with very limited treatment options.

A. baumannii is considered to be a serious threat by the US Centers for Disease Control and Prevention and belongs to the so called ‘ESKAPE’ pathogens (Enterococcus faecium, Staphylococcus aureus, Klebsiella pneumoniae, Acinetobacter baumannii, Pseudomonas aeruginosa and Enterobacter species & E. coli) that currently cause the majority of nosocomial infections and effectively “escape” the activity of antimicrobial agents.

A. baumannii is most often encountered in intensive care units and surgical wards, where extensive antibiotic use has enabled selection for resistance against all known antimicrobials and where it causes infections that include bacteremia, pneumonia, meningitis, urinary tract infection, and wound infection.

A. baumannii has an exceptional ability to upregulate and acquire resistance determinants and shows an environmental persistence that allows its survival and spread in the nosocomial setting, making this organism a frequent cause of outbreaks of infection and an endemic, health care-associated pathogen.

Due to increasing antibiotic resistance to most if not all available therapeutic options, Multi-Drug Resistant (MDR) A. baumanniii infections, especially those caused by Carbapenem resistant A. baumannii, are extremely difficult or even impossible to treat with high mortality rate as well as increased morbidity and length of stay in intensive care unit.

Acinetobacter baumannii has been defined and still remains “a prime example of a mismatch between unmet medical needs and the current antimicrobial research and development pipeline” according to the Antimicrobial Availability Task Force (AATF) of the Infectious Diseases Society of America (IDSA). Thus, there is a high demand and need to identify compounds suitable for the treatment of diseases and infections caused by Acinetobacter baumannii.

The present invention provides novel compounds which exhibit activity against drug-susceptible as well as drug-resistant strains of Acinetobacter baumannii.

### SUMMARY OF THE DISCLOSURE

In a first aspect, the present invention provides compounds of formula (I):

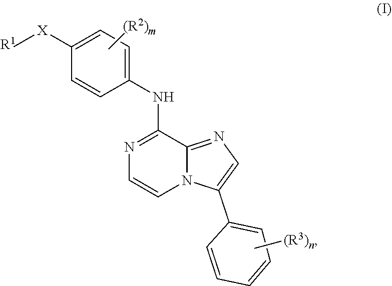

or a pharmaceutically acceptable salts thereof, wherein X, m, n, and R¹ to R³ are as defined herein.

In one aspect, the present invention provides a process of manufacturing the compounds of formula (I) described herein, comprising:
(i) reacting a heteroaryl halide (IV), wherein R¹, R², X and m are as defined herein and Y is bromo or iodo,

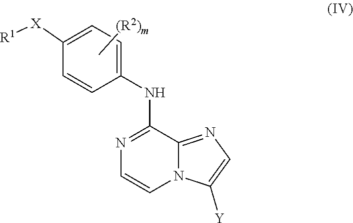

with a compound (V), wherein R³ and n are as defined herein and R is hydrogen or C₁-C₆-alkyl or the two R groups, taken together with the atoms to which they are attached, form a cyclic boronic acid ester,

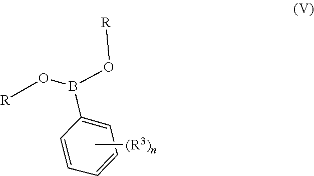

in the presence of a transition metal catalyst, such as 1,1′-bis(diphenylphosphino)ferrocene-palladium(II)dichloride dichloromethane complex, to afford said compound of formula (I); or
(ii) reacting a heteroaryl chloride (VI), wherein R³ and n are as defined herein,

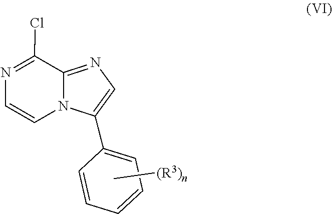

with an aniline derivative (III), wherein R¹, R², X, and m are as defined herein,

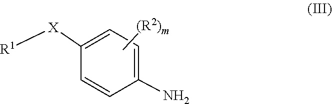

under acidic or basic conditions in the presence or absence of a transition metal catalyst (depending on the nature and hence the reactivity of the chosen aniline derivative III) in a suitable solvent, to afford said compound of formula (I); and
(iii) optionally converting said compound of formula (I) to a pharmaceutically acceptable salt thereof.

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use as antibiotic.

### DETAILED DESCRIPTION OF THE DISCLOSURE

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The following definitions are provided to facilitate understanding of certain terms used frequently herein and are not meant to limit the scope of the present disclosure. All references referred to herein are incorporated by reference in their entirety.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “alkynyl” denotes a monovalent linear or branched hydrocarbon group of 2 to 6 carbon atoms with at least one triple bond (“C₂-C₆-alkynyl”). In particular embodiments, alkynyl has 2 to 4 carbon atoms with at least one triple bond. Examples of alkynyl include ethynyl, propynyl, n-butynyl or isobutynyl. A preferred, yet non-limiting example of alkynyl is prop-2-ynyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains 1 to 4 carbon atoms. In still other embodiments, the alkoxy group contains 1 to 3 carbon atoms.

Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “alkynyloxy” refers to an alkynyl group, as previously defined, attached to the parent molecular moiety via an oxygen atom. A particularly preferred, yet non-limiting example of alkynyloxy is propynoxy (e.g., prop-2-ynoxy).

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃-C₁₀-cycloalkyl”). In some preferred embodiments, the cycloalkyl group is a saturated monocyclic hydrocarbon group of 3 to 8 ring carbon atoms, in particular 3 to 6 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclopentenyl, cyclohexyl, cycloheptyl, and spiro[2.3]hexan-5-yl. A particularly preferred, yet non-limiting example of cycloalkyl includes cyclopentenyl.

The term “aminoalkyl” refers to an alkyl group, wherein at least one of the hydrogen atoms of the alkyl group has been replaced by an amino group. Preferably, “aminoalkyl” refers to an alkyl group wherein 1, 2 or 3 hydrogen atoms of the alkyl group have been replaced by an amino group. Preferred, yet non-limiting examples of aminoalkyl are aminomethyl and 1-aminoethyl.

The term “heterocyclyl” refers to a saturated or partly unsaturated mono- or bicyclic, preferably monocyclic ring system of 3 to 10 ring atoms, preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidin-3-yl, azetidin-2-yl, oxetan-3-yl, oxetan-2-yl, 2-oxopyrrolidin-1-yl, 2-oxopyrrolidin-3-yl, 5-oxopyrrolidin-2-yl, 5-oxopyrrolidin-3-yl, 2-oxo-1-piperidyl, 2-oxo-3-piperidyl, 2-oxo-4-piperidyl, 6-oxo-2-piperidyl, 6-oxo-3-piperidyl, 1-piperidinyl, 2-piperidinyl, 3-piperidinyl, 4-piperidinyl, morpholino, morpholin-2-yl, morpholin-3-yl, pyrrolidinyl (e.g., pyrrolidin-3-yl), piperazinyl (e.g., piperazin-1-yl), 3-azabicyclo[3.1.0]hexan-6-yl, or 2,5-diazabicyclo[2.2.1]heptan-2-yl. Particularly preferred, yet non-limiting examples of heterocyclyl include pyrrolidinyl, imidazolidinyl, piperazinyl, piperidyl, and isothiazolidinyl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. A particularly preferred, yet non-limiting example of aryl is phenyl.

The term “heteroaryl” refers to a mono- or multivalent, monocyclic or bicyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, thiazol-4-yl, and 1,2,4-oxadiazol-3-yl. Most preferably, “heteroaryl” refers to 3-pyridyl, 4-pyridyl, 1H-pyrazol-5-yl, thiazol-4-yl, or 1,2,4-oxadiazol-3-yl.

The term “heteroaryloxy” refers to a heteroaryl group, as previously defined, attached to the parent molecular moiety via an oxygen atom. A particularly preferred, yet non-limiting example of heteroaryloxy is pyridyloxy (e.g., 2-pyridyloxy).

The term “hydroxy” refers to an —OH group.

The term “amino” refers to an —NH₂ group.

The term “cyano” refers to a —CN (nitrile) group.

The term “oxo” refers to a double bonded oxygen (═O).

The term “carbonyl” refers to a group C═O.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl, trifluoroethyl, 2-fluoroethyl, and 2,2-difluoroethyl.

The term “cyanoalkyl” refers to an alkyl group, wherein at least one of the hydrogen atoms of the alkyl group has been replaced by a cyano group. Preferably, “cyanoalkyl” refers to an alkyl group wherein 1, 2 or 3 hydrogen atoms of the alkyl group have been replaced by a cyano group.

Particularly preferred, yet non-limiting examples of cyanoalkyl are cyanomethyl, and cyanoethyl (e.g., 2-cyanoethyl).

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Particularly preferred, yet non-limiting examples of haloalkoxy are difluoromethoxy and trifluoromethoxy.

The term “cyanoalkoxy” refers to an alkoxy group, wherein at least one of the hydrogen atoms of the alkoxy group has been replaced by a cyano group. Preferably, “cyanoalkoxy” refers to an alkoxy group wherein 1, 2 or 3 hydrogen atoms of the alkoxy group have been replaced by a cyano group. Particularly preferred, yet non-limiting examples of cyanoalkoxy are cyanomethoxy, and cyanoethoxy (e.g., 2-cyanoethoxy).

The term “aminoalkoxy” refers to an alkoxy group, wherein at least one of the hydrogen atoms of the alkoxy group has been replaced by an amino group. Preferably, “aminoalkoxy” refers to an alkoxy group wherein 1, 2 or 3 hydrogen atoms of the alkoxy group have been replaced by an amino group. Preferred, yet non-limiting examples of aminoalkoxy are aminomethoxy and aminoethoxy.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl, hydroxyethyl (e.g. 2-hydroxyethyl), and 3-hydroxy-3-methyl-butyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochlorides, fumarates, lactates (in particular derived from L-(+)-lactic acid), tartrates (in particular derived from L-(+)-tartaric acid) and trifluoroacetates.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “mammal” as used herein includes both humans and non-humans and includes but is not limited to humans, non-human primates, canines, felines, murines, bovines, equines, and porcines. In a particularly preferred embodiment, the term “mammal” refers to humans.

The term “nosocomial infection” refers to a hospital-acquired infection (HAI), which is an infection that is acquired in a hospital or other health care facility. To emphasize both hospital and nonhospital settings, it is sometimes instead called a health care-associated infection (HAI or HCAI). Such an infection can be acquired in hospitals, nursing homes, rehabilitation facilities, outpatient clinics, or other clinical settings.

Compounds of the Invention

In a first aspect, the present invention provides a compound of formula (I):

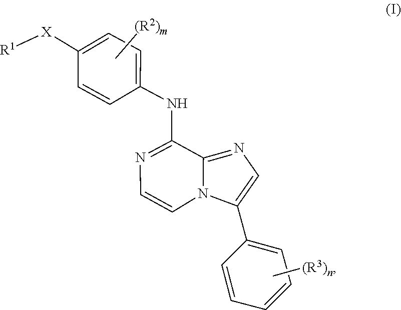

or a pharmaceutically acceptable salt thereof, wherein:
X is selected from a covalent bond, carbonyl, —(CH₂)ₚ—C(O)—NR⁴—, —NR⁴—C(O)—(CH₂)ₛ—, —(CH₂)_{q}SO₂—, —SO₂(CH₂)_{q}—, —NR⁵—S(O)₂—, —S(O)₂—NR⁵—, a group

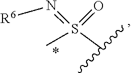

and a group

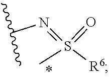

wherein
   the asterisk indicates the point of attachment of R¹ to X; and
   the wavy line indicates the point of attachment of X to the remainder of formula (I);
R¹ is selected from hydrogen, halogen, cyano, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, halo-C₁-C₆-alkyl, cyano-C₁-C₆-alkyl, hydroxy-C₁-C₆-alkyl, amino-C₁-C₆-alkyl, C₁-C₆-alkyl-NH—C₁-C₆-alkyl, (C₁-C₆-alkyl)₂N—C₁-C₆-alkyl, (C₁-C₆-alkyl)₃N⁺—C₁-C₆-alkyl, C₁-C₆-alkyl-NH—, (C₁-C₆-alkyl)₂N—, C₁-C₆-alkoxy, halo-C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)—NH—C₁-C₆-alkoxy, C₁-C₆-alkyl-NH—C(O)—, and a group

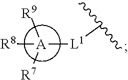

and
R² is, at each occurrence, independently selected from hydrogen, halogen, C₁-C₆-alkyl, hydroxy-C₁-C₆-alkyl and C₁-C₆-alkyl-NH—C(O)—; or
R¹ and one occurrence of R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with 1-2 substituents independently selected from halogen, cyano, hydroxy, amino, C₁-C₆-alkyl, C₁-C₆-alkoxy, halo-C₁-C₆-alkyl, halo-C₁-C₆-alkoxy and amino-C₁-C₆-alkyl-NH—;
R³ is, at each occurrence, independently selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy;
R⁴ is selected from hydrogen and C₁-C₆-alkyl;
R⁵ is selected from hydrogen and C₁-C₆-alkyl; or
R⁶ is selected from C₁-C₆-alkyl, amino-C₁-C₆-alkyl, (3- to 14-membered heterocyclyl)-C(O)—NH—C₁-C₆-alkyl; wherein said 3- to 14-membered heterocyclyl is optionally substituted with 1-2 hydroxy substituents;
R⁷, R⁸, and R⁹ are each independently selected from hydrogen, halogen, cyano, hydroxy, amino, C₁-C₆-alkyl, amino-C₁-C₆-alkyl, halo-C₁-C₆-alkyl, cyano-C₁-C₆-alkyl, hydroxy-C₁-C₆-alkyl, C₁-C₆-alkoxy, halo-C₁-C₆-alkoxy, C₁-C₆-alkyl-NH—C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl and oxo; wherein said 3- to 14-membered heterocyclyl is optionally substituted with 1-3 substituents selected from hydroxy, amino, halogen, cyano, C₁-C₆-alkyl, halo-C₁-C₆-alkyl, C₁-C₆-alkoxy, halo-C₁-C₆-alkoxy, and hydroxy-(3- to 14-membered heterocyclyl)-C(O)—;
A is selected from 3- to 14-membered heterocyclyl, 5- to 14-membered heteroaryl, C₆-C₁₄-aryl, and C₃-C₁₀-cycloalkyl;
L¹ is a covalent bond or C₁-C₆-alkyl;
m is an integer selected from 1, 2, 3 and 4;
n is an integer selected from 1, 2, 3, 4 and 5;
p is an integer selected from 0 and 1;
q is an integer selected from 0 and 1; and
r and s are both an integer selected from 1, 2, 3 and 4;
provided that when X is carbonyl, R¹ is not C₁-C₆-alkyl-NH— or (C₁-C₆-alkyl)₂N—.
In a further aspect, the present the present invention provides a compound of formula (I), or a pharmaceutically acceptable salt thereof, wherein: X is selected from a covalent bond, carbonyl, —(CH₂)ₚ—C(O)—NR⁴—, —NR⁴—C(O)—(CH₂)ₚ—, —(CH₂)_{q}SO₂—, —SO₂(CH₂)_{q}—, —NR⁵—S(O)₂—, —S(O)₂—NR⁵—, a group

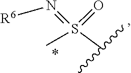

and a group

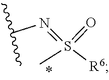

wherein
   the asterisk indicates the point of attachment of R¹ to X; and
   the wavy line indicates the point of attachment of X to the remainder of formula (I);
R¹ is selected from hydrogen, halogen, cyano, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, halo-C₁-C₆-alkyl, cyano-C₁-C₆-alkyl, hydroxy-C₁-C₆-alkyl, amino-C₁-C₆-alkyl, C₁-C₆-alkyl-NH—C₁-C₆-alkyl, (C₁-C₆-alkyl)₂N—C₁-C₆-alkyl, (C₁-C₆-alkyl)₃N⁺—C₁-C₆-alkyl, C₁-C₆-alkyl-NH—, (C₁-C₆-alkyl)₂N—, C₁-C₆-alkoxy, halo-C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)—NH—C₁-C₆-alkoxy, C₁-C₆-alkyl-NH—C(O)—, and a group

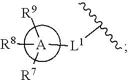

and
R² is, at each occurrence, independently selected from hydrogen, halogen, C₁-C₆-alkyl, hydroxy-C₁-C₆-alkyl and C₁-C₆-alkyl-NH—C(O)—; or
R¹ and one occurrence of R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with 1-2 substituents independently selected from halogen, cyano, hydroxy, amino, C₁-C₆-alkyl, C₁-C₆-alkoxy, halo-C₁-C₆-alkyl, halo-C₁-C₆-alkoxy and amino-C₁-C₆-alkyl-NH—;
R³ is, at each occurrence, independently selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy;
R⁴ is selected from hydrogen and C₁-C₆-alkyl;
R⁵ is selected from hydrogen and C₁-C₆-alkyl; or
R⁶ is selected from C₁-C₆-alkyl, amino-C₁-C₆-alkyl, (3- to 14-membered heterocyclyl)-C(O)—NH—C₁-C₆-alkyl; wherein said 3- to 14-membered heterocyclyl is optionally substituted with 1-2 hydroxy substituents;
R⁷, R⁸, and R⁹ are each independently selected from hydrogen, halogen, cyano, hydroxy, amino, C₁-C₆-alkyl, amino-C₁-C₆-alkyl, halo-C₁-C₆-alkyl, cyano-C₁-C₆-alkyl, hydroxy-C₁-C₆-alkyl, C₁-C₆-alkoxy, halo-C₁-C₆-alkoxy, C₁-C₆-alkyl-NH—C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl and oxo; wherein said 3- to 14-membered heterocyclyl is optionally substituted with 1-3 substituents selected from hydroxy, amino, halogen, cyano, C₁-C₆-alkyl, halo-C₁-C₆-alkyl, C₁-C₆-alkoxy, halo-C₁-C₆-alkoxy, and hydroxy-(3- to 14-membered heterocyclyl)-C(O)—;
A is selected from 3- to 14-membered heterocyclyl, 5- to 14-membered heteroaryl, C₆-C₁₄-aryl, and C₃-C₁₀-cycloalkyl;
L¹ is a covalent bond or C₁-C₆-alkyl;
m is an integer selected from 1, 2, 3 and 4;
n is an integer selected from 1, 2, 3, 4 and 5;
p is an integer selected from 0 and 1;
q is an integer selected from 0 and 1; and
r is an integer selected from 1, 2, 3 and 4.

In one embodiment, the invention provides a compound of formula (I) as defined herein, or a pharmaceutically acceptable salt thereof, wherein the compound of formula (I) is a compound of formula (I-I):

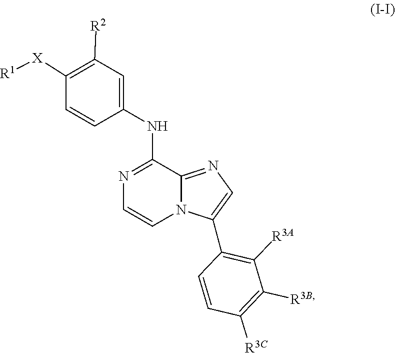

wherein:
X, R¹, and R² are as defined herein; and
R^{3A}, R^{3B}, and R^{3C} are each independently defined like R³ herein.

In one embodiment, the invention provides a compound of formula (I) as defined herein, or a pharmaceutically acceptable salt thereof, wherein X is selected from a covalent bond, carbonyl, —(CH₂)ₚ—C(O)—NR⁴—, —NR⁴—C(O)—(CH₂)ₚ—, —(CH₂)_{q}SO₂—, —NR⁵—S(O)₂—, a group

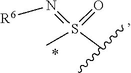

and a group

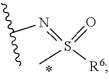

wherein
R⁴ and R⁵ are each independently selected from hydrogen and C₁-C₆-alkyl;
R⁶ is selected from C₁-C₆-alkyl, amino-C₁-C₆-alkyl, and hydroxy-(3- to 14-membered heterocyclyl)-C(O)—NH—C₁-C₆-alkyl;
the asterisk indicates the point of attachment of R¹ to X; and
the wavy line indicates the point of attachment of X to the remainder of formula (I); and
p and q are each independently 0 or 1.

In one embodiment, the invention provides a compound of formula (I) as defined herein, or a pharmaceutically acceptable salt thereof, wherein X is selected from a covalent bond, carbonyl, —(CH₂)ₚ—C(O)—NR⁴—, —(CH₂)_{q}SO₂—, —NR⁵—S(O)₂—, a group

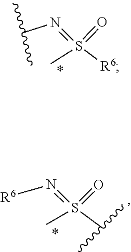

and a group

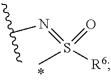

wherein
R⁴ and R⁵ are each independently selected from hydrogen and C₁-C₆-alkyl;
R⁶ is selected from C₁-C₆-alkyl, amino-C₁-C₆-alkyl, and hydroxy-(3- to 14-membered heterocyclyl)-C(O)—NH—C₁-C₆-alkyl;
the asterisk indicates the point of attachment of R¹ to X; and
the wavy line indicates the point of attachment of X to the remainder of formula (I); and
p and q are each independently 0 or 1.

In a preferred embodiment, the invention provides a compound of formula (I) as defined herein, or a pharmaceutically acceptable salt thereof, wherein X is selected from a covalent bond, carbonyl, —(CH₂)ₚ—C(O)—NR⁴—, —NR⁴—C(O)—(CH₂)ₚ—, —(CH₂)_{q}SO₂—, —NR⁵—S(O)₂—, a group

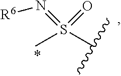

and a group

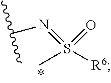

wherein
R⁴ and R⁵ are both hydrogen;
R⁶ is C₁-C₆-alkyl or hydroxy-(3- to 14-membered heterocyclyl)-C(O)—NH—C₁-C₆-alkyl; the asterisk indicates the point of attachment of R¹ to X; and the wavy line indicates the point of attachment of X to the remainder of formula (I);
p is 0 or 1; and
q is 0.

In a preferred embodiment, the invention provides a compound of formula (I) as defined herein, or a pharmaceutically acceptable salt thereof, wherein X is selected from a covalent bond, carbonyl, —(CH₂)ₚ—C(O)—NR⁴—, —(CH₂)_{q}SO₂—, —NR⁵—S(O)₂—, a group

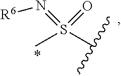

and a group

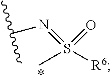

wherein
R⁴ and R⁵ are both hydrogen;
R⁶ is C₁-C₆-alkyl or hydroxy-(3- to 14-membered heterocyclyl)-C(O)—NH—C₁-C₆-alkyl;
the asterisk indicates the point of attachment of R¹ to X; and
the wavy line indicates the point of attachment of X to the remainder of formula (I);
p is 0 or 1; and
q is 0.

In a particularly preferred embodiment, the invention provides a compound of formula (I) as defined herein, or a pharmaceutically acceptable salt thereof, wherein X is selected from a covalent bond, carbonyl, —(CH₂)ₚ—C(O)—NR⁴—, —NR⁴—C(O)—(CH₂)ₚ—, —(CH₂)_{q}SO₂—, —NR⁵—S(O)₂—, a group

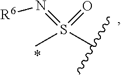

and a group

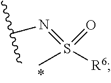

wherein
R⁴ and R⁵ are both hydrogen;
R⁶ is methyl or hydroxypyrrolidinyl-C(O)—NH—(CH₂)₃—;
the asterisk indicates the point of attachment of R¹ to X; and
the wavy line indicates the point of attachment of X to the remainder of formula (I);
p is 0 or 1; and
q is 0.

In a particularly preferred embodiment, the invention provides a compound of formula (I) as defined herein, or a pharmaceutically acceptable salt thereof, wherein X is selected from a covalent bond, carbonyl, —(CH₂)ₚ—C(O)—NR⁴—, —(CH₂)_{q}SO₂—, —NR⁵—S(O)₂—, a group

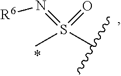

and a group

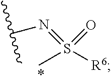

wherein
R⁴ and R⁵ are both hydrogen;
R⁶ is methyl or hydroxypyrrolidinyl-C(O)—NH—(CH₂)₃—;
the asterisk indicates the point of attachment of R¹ to X; and
the wavy line indicates the point of attachment of X to the remainder of formula (I);
p is 0 or 1; and
q is 0.

In one embodiment, the invention provides a compound of formula (I) as defined herein, or a pharmaceutically acceptable salt thereof, wherein
R¹ is selected from halogen, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, hydroxy-C₁-C₆-alkyl, amino-C₁-C₆-alkyl, (C₁-C₆-alkyl)₃N⁺—C₁-C₆-alkyl, (C₁-C₆-alkyl)₂N—, C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)—NH—C₁-C₆-alkoxy, and a group

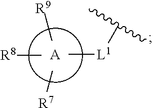

wherein
A is selected from 3- to 14-membered heterocyclyl, 5- to 14-membered heteroaryl, and C₃-C₁₀-cycloalkyl;
L¹ is a covalent bond or C₁-C₆-alkyl;
R⁷ is selected from hydrogen, C₁-C₆-alkyl, amino, C₁-C₆-alkyl, amino-C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl, and oxo; wherein said 3- to 14-membered heterocyclyl is optionally substituted with a substituent selected from hydroxy and hydroxy-(3- to 14-membered heterocyclyl)-C(O)—;
R⁸ is hydrogen or oxo;
R⁹ is hydrogen; and
r is 2 or 3.

In a preferred embodiment, the invention provides a compound of formula (I) as defined herein, or a pharmaceutically acceptable salt thereof, wherein
R¹ is selected from amino, C₁-C₆-alkyl, amino-C₁-C₆-alkoxy-C₁-C₆-alkyl, amino-(CH₂CH₂O)ᵣ—, and a group

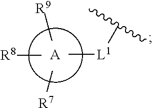

wherein
A is 3- to 14-membered heterocyclyl or C₃-C₁₀-cycloalkyl;
L¹ is a covalent bond;
R⁷ is selected from amino, amino-C₁-C₆-alkyl, (C₁-C₆-alkyl)₂N—C₁-C₆-alkyl, hydroxy-(3- to 14-membered heterocyclyl)-C(O)—, and oxo;
R⁸ is hydrogen or oxo;
R⁹ is hydrogen; and
r is 3.

In a particularly preferred embodiment, the invention provides a compound of formula (I) as defined herein, or a pharmaceutically acceptable salt thereof, wherein
R¹ is selected from amino, methyl, amino-(CH₂)₂—O—(CH₂)₂—, amino-(CH₂CH₂O)ᵣ—, and a group

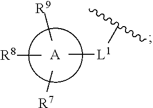

wherein
A is selected from pyrrolidinyl, imidazolidinyl, piperazinyl, piperidyl, isothiazolidinyl, and cyclohexyl;
L¹ is a covalent bond;
R⁷ is selected from amino, aminomethyl, aminobutyl, (CH₃)₂N—(CH₂)₂—, hydroxypyrrolidinyl-C(O)—, and oxo;
R⁸ is hydrogen or oxo;
R⁹ is hydrogen; and
r is 3.

In one embodiment, the invention provides a compound of formula (I) as defined herein, or a pharmaceutically acceptable salt thereof, wherein R² is selected from hydrogen, halogen, C₁-C₆-alkyl, hydroxy-C₁-C₆-alkyl, and C₁-C₆-alkyl-NH—C(O)—.

In a preferred embodiment, the invention provides a compound of formula (I) as defined herein, or a pharmaceutically acceptable salt thereof, wherein R² is selected from hydrogen, halogen, C₁-C₆-alkyl, and C₁-C₆-alkyl-NH—C(O)—.

In a particularly preferred embodiment, the invention provides a compound of formula (I) as defined herein, or a pharmaceutically acceptable salt thereof, wherein R² is selected from hydrogen, chloro, methyl, ethyl, and CH₃—NH—C(O)—.

In one embodiment, the invention provides a compound of formula (I) as defined herein, or a pharmaceutically acceptable salt thereof, wherein
R¹ and one occurrence of R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with amino-C₁-C₆-alkyl-NH—.

In a preferred embodiment, the invention provides a compound of formula (I) as defined herein, or a pharmaceutically acceptable salt thereof, wherein
R¹ and one occurrence of R², taken together with the atoms to which they are attached, form a C₃-C₁₀-cycloalkyl, wherein said C₃-C₁₀-cycloalkyl is substituted with amino-C₁-C₆-alkyl-NH—.

In a particularly preferred embodiment, the invention provides a compound of formula (I) as defined herein, or a pharmaceutically acceptable salt thereof, wherein
R¹ and one occurrence of R², taken together with the atoms to which they are attached, form a cyclopentene, wherein said cyclopentene is substituted with aminoethyl-NH—.

In one embodiment, the invention provides a compound of formula (I) as defined herein, or a pharmaceutically acceptable salt thereof, wherein R³ is selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy.

In a preferred embodiment, the invention provides a compound of formula (I) as defined herein, or a pharmaceutically acceptable salt thereof, wherein R³ is selected from hydrogen, halogen, C₁-C₆-alkoxy, halo-C₁-C₆-alkoxy, and (5- to 14-membered heteroaryl)oxy.

In a particularly preferred embodiment, the invention provides a compound of formula (I) as defined herein, or a pharmaceutically acceptable salt thereof, wherein R³ is selected from hydrogen, chloro, fluoro, methoxy, difluoromethoxy, and pyridyloxy.

In one embodiment, the invention provides a compound of formula (I) as defined herein, or a pharmaceutically acceptable salt thereof, wherein
R^{3A} and R^{3B} are independently selected from hydrogen and halogen; and
R^{3C} is selected from halogen, C₁-C₆-alkoxy, cyano-C₁-C₆-alkoxy, halo-C₁-C₆-alkoxy, C₂-C₆-alkynyloxy, and (5- to 14-membered heteroaryl)oxy.

In a preferred embodiment, the invention provides a compound of formula (I) as defined herein, or a pharmaceutically acceptable salt thereof, wherein
R^{3A} and R^{3B} are independently selected from hydrogen and halogen; and
R^{3C} is selected from C₁-C₆-alkoxy, halo-C₁-C₆-alkoxy, and (5- to 14-membered heteroaryl)oxy.

In a particularly preferred embodiment, the invention provides a compound of formula (I) as defined herein, or a pharmaceutically acceptable salt thereof, wherein
R^{3A} is selected from hydrogen and fluoro;
R^{3B} is selected from hydrogen, chloro and fluoro; and
R^{3C} is selected from methoxy, difluoromethoxy, and pyridyloxy.

In a preferred embodiment, the invention provides a compound of formula (I) as defined herein, or a pharmaceutically acceptable salt thereof, wherein
m is 1; and
n is an integer selected from 1, 2, and 3.

In one embodiment, the invention provides a compound of formula (I) as defined herein, or a pharmaceutically acceptable salt thereof, wherein
X is selected from a covalent bond, carbonyl, —(CH₂)ₚ—C(O)—NR⁴—, —NR⁴—C(O) CH₂)ₚ—, —(CH₂)_{q}SO₂—, —NR⁵—S(O)₂—, a group

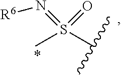

and a group

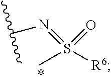

R¹ is selected from halogen, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, hydroxy-C₁-C₆-alkyl, amino-C₁-C₆-alkyl, (C₁-C₆-alkyl)₃N⁺—C₁-C₆-alkyl, (C₁-C₆-alkyl)₂N—, C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)—NH—C₁-C₆-alkoxy, and a group

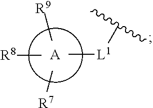

and
R² is selected from hydrogen, halogen, C₁-C₆-alkyl, hydroxy-C₁-C₆-alkyl, and C₁-C₆-alkyl-NH—C(O)—; or
R¹ and R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with amino-C₁-C₆-alkyl-NH—
R³ is selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy;
R⁴ and R⁵ are each independently selected from hydrogen and C₁-C₆-alkyl;
R⁶ is selected from C₁-C₆-alkyl, amino-C₁-C₆-alkyl, and hydroxy-(3- to 14-membered heterocyclyl)-C(O)—NH—C₁-C₆-alkyl;
R⁷ is selected from hydrogen, C₁-C₆-alkyl, amino, C₁-C₆-alkyl, amino-C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl, and oxo; wherein said 3- to 14-membered heterocyclyl is optionally substituted with a substituent selected from hydroxy and hydroxy-(3- to 14-membered heterocyclyl)-C(O)—;
R⁸ is hydrogen or oxo;
R⁹ is hydrogen;
A is selected from 3- to 14-membered heterocyclyl, 5- to 14-membered heteroaryl, and C₃-C₁₀-cycloalkyl;
L¹ is a covalent bond or C₁-C₆-alkyl;
the asterisk indicates the point of attachment of R¹ to X;
the wavy line indicates the point of attachment of X to the remainder of formula (I);
m is 1;
n is an integer selected from 1, 2, and 3;
p and q are each independently 0 or 1; and
r is 2 or 3.

In one embodiment, the invention provides a compound of formula (I) as defined herein, or a pharmaceutically acceptable salt thereof, wherein
X is selected from a covalent bond, carbonyl, —(CH₂)ₚ—C(O)—NR⁴—, —(CH₂)_{q}SO₂—, —NR⁵—S(O)₂—, a group

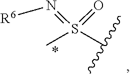

and a group

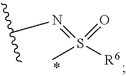

R¹ is selected from halogen, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, hydroxy-C₁-C₆-alkyl, amino-C₁-C₆-alkyl, (C₁-C₆-alkyl)₃N⁺—C₁-C₆-alkyl, (C₁-C₆-alkyl)₂N—, C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)NH—C₁-C₆-alkoxy, and a group

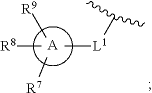

and
R² is selected from hydrogen, halogen, C₁-C₆-alkyl, hydroxy-C₁-C₆-alkyl, and C₁-C₆-alkyl-NH—C(O)—; or
R¹ and R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with amino-C₁-C₆-alkyl-NH—;
R³ is selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy;
R⁴ and R⁵ are each independently selected from hydrogen and C₁-C₆-alkyl;
R⁶ is selected from C₁-C₆-alkyl, amino-C₁-C₆-alkyl, and hydroxy-(3- to 14-membered heterocyclyl)-C(O)—NH—C₁-C₆-alkyl;
R⁷ is selected from hydrogen, C₁-C₆-alkyl, amino, C₁-C₆-alkyl, amino-C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl, and oxo; wherein said 3- to 14-membered heterocyclyl is optionally substituted with a substituent selected from hydroxy and hydroxy-(3- to 14-membered heterocyclyl)-C(O)—;
R⁸ is hydrogen or oxo;
R⁹ is hydrogen;
A is selected from 3- to 14-membered heterocyclyl, 5- to 14-membered heteroaryl, and C₃-C₁₀-cycloalkyl;
L¹ is a covalent bond or C₁-C₆-alkyl;
the asterisk indicates the point of attachment of R¹ to X;
the wavy line indicates the point of attachment of X to the remainder of formula (I);
m is 1;
n is an integer selected from 1, 2, and 3;
p and q are each independently 0 or 1; and
r is 2 or 3.

In a preferred embodiment, the invention provides a compound of formula (I) as defined herein, or a pharmaceutically acceptable salt thereof, wherein
X is selected from a covalent bond, carbonyl, —(CH₂)ₚ—C(O)—NR⁴—, —NR⁴—C(O)—(CH₂)ₚ—, —(CH₂)_{q}SO₂—, —NR⁵—S(O)₂—, a group

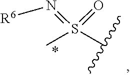

and a group

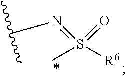

R¹ is selected from amino, C₁-C₆-alkyl, amino-C₁-C₆-alkoxy-C₁-C₆-alkyl, amino-(CH₂CH₂O)ᵣ—, and a group

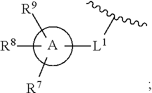

and
R² is selected from hydrogen, halogen, C₁-C₆-alkyl, and C₁-C₆-alkyl-NH—C(O)—; or
R¹ and R², taken together with the atoms to which they are attached, form a C₃-C₁₀-cycloalkyl, wherein said C₃-C₁₀-cycloalkyl is substituted with amino-C₁-C₆-alkyl-NH—;
R³ is selected from hydrogen, halogen, C₁-C₆-alkoxy, halo-C₁-C₆-alkoxy, and (5- to 14-membered heteroaryl)oxy;
R⁴ and R⁵ are both hydrogen;
R⁶ is C₁-C₆-alkyl or hydroxy-(3- to 14-membered heterocyclyl)-C(O)—NH—C₁-C₆-alkyl;
R⁷ is selected from amino, amino-C₁-C₆-alkyl, (C₁-C₆-alkyl)₂N—C₁-C₆-alkyl, hydroxy-(3- to 14-membered heterocyclyl)-C(O)—, and oxo;
R⁸ is hydrogen or oxo;
R⁹ is hydrogen;
A is 3- to 14-membered heterocyclyl or C₃-C₁₀-cycloalkyl;
L¹ is a covalent bond;
the asterisk indicates the point of attachment of R¹ to X;
the wavy line indicates the point of attachment of X to the remainder of formula (I);
m is 1;
n is an integer selected from 1, 2, and 3;
p is 0 or 1;
q is 0; and
r is 3.

In a preferred embodiment, the invention provides a compound of formula (I) as defined herein, or a pharmaceutically acceptable salt thereof, wherein
X is selected from a covalent bond, carbonyl, —(CH₂)ₚ—C(O)—NR⁴—, —(CH₂)_{q}SO₂—, —NR⁵—S(O)₂—, a group

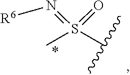

and a group

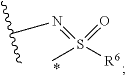

R¹ is selected from amino, C₁-C₆-alkyl, amino-C₁-C₆-alkoxy-C₁-C₆-alkyl, amino-(CH₂CH₂O)ᵣ—, and a group

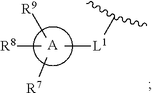

and
R² is selected from hydrogen, halogen, C₁-C₆-alkyl, and C₁-C₆-alkyl-NH—C(O)—; or
R¹ and R², taken together with the atoms to which they are attached, form a C₃-C₁₀-cycloalkyl, wherein said C₃-C₁₀-cycloalkyl is substituted with amino-C₁-C₆-alkyl-NH—;
R³ is selected from hydrogen, halogen, C₁-C₆-alkoxy, halo-C₁-C₆-alkoxy, and (5- to 14-membered heteroaryl)oxy;
R⁴ and R⁵ are both hydrogen;
R⁶ is C₁-C₆-alkyl or hydroxy-(3- to 14-membered heterocyclyl)-C(O)—NH—C₁-C₆-alkyl;
R⁷ is selected from amino, amino-C₁-C₆-alkyl, (C₁-C₆-alkyl)₂N—C₁-C₆-alkyl, hydroxy-(3- to 14-membered heterocyclyl)-C(O)—, and oxo;
R⁸ is hydrogen or oxo;
R⁹ is hydrogen;
A is 3- to 14-membered heterocyclyl or C₃-C₁₀-cycloalkyl;
L¹ is a covalent bond;
the asterisk indicates the point of attachment of R¹ to X;
the wavy line indicates the point of attachment of X to the remainder of formula (I);
m is 1;
n is an integer selected from 1, 2, and 3;
p is 0 or 1;
q is 0; and
r is 3.

In a particularly preferred embodiment, the invention provides a compound of formula (I) as defined herein, or a pharmaceutically acceptable salt thereof, wherein
X is selected from a covalent bond, carbonyl, —(CH₂)ₚ—C(O)—NR⁴—, —NR⁴—C(O)—(CH₂)ₚ—, —(CH₂)_{q}SO₂—, —NR⁵—S(O)₂—, a group

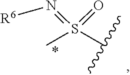

and a group

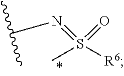

R¹ is selected from amino, methyl, amino-(CH₂)₂—O—(CH₂)₂—, amino-(CH₂CH₂O)ᵣ—, and a group

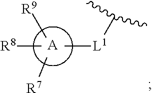

and
R² is selected from hydrogen, chloro, methyl, ethyl, and CH₃—NH—C(O)—; or
R¹ and R², taken together with the atoms to which they are attached, form a cyclopentene, wherein said cyclopentene is substituted with aminoethyl-NH—;
R³ is selected from hydrogen, chloro, fluoro, methoxy, difluoromethoxy, and pyridyloxy;
R⁴ and R⁵ are both hydrogen;
R⁶ is methyl or hydroxypyrrolidinyl-C(O)—NH—(CH₂)₃—;
R⁷ is selected from amino, aminomethyl, aminobutyl, (CH₃)₂N—(CH₂)₂—, hydroxypyrrolidinyl-C(O)—, and oxo;
R⁸ is hydrogen or oxo;
R⁹ is hydrogen;
A is selected from pyrrolidinyl, imidazolidinyl, piperazinyl, piperidyl, isothiazolidinyl, and cyclohexyl;
L¹ is a covalent bond;
the asterisk indicates the point of attachment of R¹ to X;
the wavy line indicates the point of attachment of X to the remainder of formula (I);
m is 1;
n is an integer selected from 1, 2, and 3;
p is 0 or 1;
q is 0; and
r is 3.

In a particularly preferred embodiment, the invention provides a compound of formula (I) as defined herein, or a pharmaceutically acceptable salt thereof, wherein
X is selected from a covalent bond, carbonyl, —(CH₂)ₚ—C(O)—NR⁴—, —(CH₂)_{q}SO₂—, —NR⁵—S(O)₂—, a group

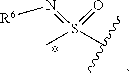

and a group

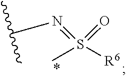

R¹ is selected from amino, methyl, amino-(CH₂)₂—O—(CH₂)₂—, amino-(CH₂CH₂O)ᵣ—, and a group

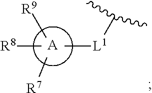

and
R² is selected from hydrogen, chloro, methyl, ethyl, and CH₃—NH—C(O)—; or
R¹ and R², taken together with the atoms to which they are attached, form a cyclopentene, wherein said cyclopentene is substituted with aminoethyl-NH—;
R³ is selected from hydrogen, chloro, fluoro, methoxy, difluoromethoxy, and pyridyloxy;
R⁴ and R⁵ are both hydrogen;
R⁶ is methyl or hydroxypyrrolidinyl-C(O)—NH—(CH₂)₃—;
R⁷ is selected from amino, aminomethyl, aminobutyl, (CH₃)₂N—(CH₂)₂—, hydroxypyrrolidinyl-C(O)—, and oxo;
R⁸ is hydrogen or oxo;
R⁹ is hydrogen;
A is selected from pyrrolidinyl, imidazolidinyl, piperazinyl, piperidyl, isothiazolidinyl, and cyclohexyl;
L¹ is a covalent bond;
the asterisk indicates the point of attachment of R¹ to X;
the wavy line indicates the point of attachment of X to the remainder of formula (I);
m is 1;
n is an integer selected from 1, 2, and 3;
p is 0 or 1;
q is 0; and
r is 3.

In one embodiment, the invention provides a compound of formula (I) as defined herein, or a pharmaceutically acceptable salt thereof, wherein the compound of formula (I) is selected from:
N-[4-[[3-(3-chloro-4-methoxy-phenyl)imidazo[1,2-a]pyrazin-8-yl]amino]-2-methyl-phenyl]acetamide;
1-[4-[[3-(3-fluoro-4-methoxy-phenyl)imidazo[1,2-a]pyrazin-8-yl]amino]phenyl]pyrrolidin-2-one;
1-[2-chloro-4-[[3-[4-(difluoromethoxy)phenyl]imidazo[1,2-a]pyrazin-8-yl]amino]phenyl]pyrrolidin-2-one;
N-[4-[[3-(4-methoxyphenyl)imidazo[1,2-a]pyrazin-8-yl]amino]-2-methyl-phenyl]acetamide;
N-[4-[[3-(4-chlorophenyl)imidazo[1,2-a]pyrazin-8-yl]amino]-2-methyl-phenyl]acetamide;
N-[4-[[3-[4-(difluoromethoxy)phenyl]imidazo[1,2-a]pyrazin-8-yl]amino]-2-methyl-phenyl]acetamide;
N-[2-chloro-4-[[3-(4-methoxyphenyl)imidazo[1,2-a]pyrazin-8-yl]amino]phenyl]acetamide;
N-[4-[[3-(3-fluoro-4-methoxy-phenyl)imidazo[1,2-a]pyrazin-8-yl]amino]-2-methyl-phenyl]acetamide;
N-[4-[[3-(3-chloro-4-methoxy-phenyl)imidazo[1,2-a]pyrazin-8-yl]amino]-2-methyl-phenyl]acetamide;
4-(4-aminobutyl)-1-[4-[[3-[4-(difluoromethoxy)phenyl]imidazo[1,2-a]pyrazin-8-yl]amino]phenyl]pyrrolidin-2-one;
1-[4-[[3-[4-(difluoromethoxy)-3-fluoro-phenyl]imidazo[1,2-a]pyrazin-8-yl]amino]phenyl]pyrrolidin-2-one;
1-[4-[[3-[4-(difluoromethoxy)phenyl]imidazo[1,2-a]pyrazin-8-yl]amino]phenyl]pyrrolidin-2-one;
N-[4-[[3-(3-chloro-4-methoxy-phenyl)imidazo[1,2-a]pyrazin-8-yl]amino]-2-ethyl-phenyl]acetamide;
N-[4-[[3-(4-chloro-3-fluoro-phenyl)imidazo[1,2-a]pyrazin-8-yl]amino]-2-methyl-phenyl]acetamide;
3-amino-N-[4-[[3-(2,3-difluoro-4-methoxy-phenyl)imidazo[1,2-a]pyrazin-8-yl]amino]-2-ethyl-phenyl]propanamide;
N-[2-ethyl-4-[[3-(4-methoxyphenyl)imidazo[1,2-a]pyrazin-8-yl]amino]phenyl]acetamide;
4-(5-aminopentyl)-1-[4-[[3-[4-(difluoromethoxy)phenyl]imidazo[1,2-a]pyrazin-8-yl]amino]phenyl]pyrrolidin-2-one;
N-[2-[2-[4-[[3-(4-methoxyphenyl)imidazo[1,2-a]pyrazin-8-yl]amino]-2-methyl-anilino]-2-oxo-ethoxy]ethyl]pentanamide;
1-[4-[[3-[4-(difluoromethoxy)-3-fluoro-phenyl]imidazo[1,2-a]pyrazin-8-yl]amino]-2-methyl-phenyl]pyrrolidin-2-one;
3-[4-[[3-[4-(difluoromethoxy)phenyl]imidazo[1,2-a]pyrazin-8-yl]amino]phenyl]oxazolidin-2-one;
2-(3-aminopyrrolidin-1-yl)-N-[4-[[3-[4-(difluoromethoxy)phenyl]imidazo[1,2-a]pyrazin-8-yl]amino]-2-methyl-phenyl]acetamide;
1-[4-[[3-(4-methoxyphenyl)imidazo[1,2-a]pyrazin-8-yl]amino]-2-methyl-phenyl]pyrrolidin-2-one;
N-[4-[[3-(2-chloro-4-methoxy-phenyl)imidazo[1,2-a]pyrazin-8-yl]amino]-2-methyl-phenyl]acetamide;
1-[4-[[3-(3-chloro-4-methoxy-phenyl)imidazo[1,2-a]pyrazin-8-yl]amino]phenyl]piperidin-2-one;
N-[2-methyl-4-[[3-(2,3,4-trifluorophenyl)imidazo[1,2-a]pyrazin-8-yl]amino]phenyl]acetamide;
N-[4-[[3-(2-fluoro-4-methoxy-phenyl)imidazo[1,2-a]pyrazin-8-yl]amino]-2-methyl-phenyl]acetamide;
N-[4-[[3-[4-(difluoromethoxy)phenyl]imidazo[1,2-a]pyrazin-8-yl]amino]-2-methyl-phenyl]-2-(4-ethylpiperazin-1-yl)acetamide;
2-(2-aminoethoxy)-N-[4-[[3-(4-methoxyphenyl)imidazo[1,2-a]pyrazin-8-yl]amino]-2-methyl-phenyl]acetamide;
4-(2-aminoethoxymethyl)-1-[4-[[3-[4-(difluoromethoxy)phenyl]imidazo[1,2-a]pyrazin-8-yl]amino]phenyl]pyrrolidin-2-one;
N-[4-[[3-(3-chloro-4-methoxy-phenyl)imidazo[1,2-a]pyrazin-8-yl]amino]-2-methyl-phenyl]-2-piperazin-1-yl-acetamide;
N-[4-[[3-(4-methoxyphenyl)imidazo[1,2-a]pyrazin-8-yl]amino]-2-methyl-phenyl]-2-piperazin-1-yl-acetamide;
1-[4-[[3-(4-methoxyphenyl)imidazo[1,2-a]pyrazin-8-yl]amino]phenyl]pyrrolidin-2-one;
N-[4-[[3-[4-(cyanomethoxy)phenyl]imidazo[1,2-a]pyrazin-8-yl]amino]-2-methyl-phenyl]acetamide;
N-[4-[[3-(4-methoxyphenyl)imidazo[1,2-a]pyrazin-8-yl]amino]-2-methyl-phenyl]-2-piperazin-1-yl-acetamide;
4-[4-[[3-[4-(difluoromethoxy)phenyl]imidazo[1,2-a]pyrazin-8-yl]amino]phenyl]morpholin-3-one;
1-[4-[[3-(3-chloro-4-methoxy-phenyl)imidazo[1,2-a]pyrazin-8-yl]amino]phenyl]pyrrolidin-2-one;
N-[4-[[3-(3,4-difluorophenyl)imidazo[1,2-a]pyrazin-8-yl]amino]-2-methyl-phenyl]acetamide;
N-[4-[[3-(4-fluorophenyl)imidazo[1,2-a]pyrazin-8-yl]amino]-2-methyl-phenyl]acetamide;
1-[4-[[3-[4-(difluoromethoxy)phenyl]imidazo[1,2-a]pyrazin-8-yl]amino]phenyl]-5-methyl-pyrrolidin-2-one;
1-[4-[[3-(3-fluoro-4-methoxy-phenyl)imidazo[1,2-a]pyrazin-8-yl]amino]phenyl]pyrrolidin-2-one;
N-[4-[[3-(3-fluoro-4-methoxy-phenyl)imidazo[1,2-a]pyrazin-8-yl]amino]-2-methyl-phenyl]acetamide;
N-[4-[[3-(3-fluoro-4-methoxy-phenyl)imidazo[1,2-a]pyrazin-8-yl]amino]phenyl]acetamide;
N-[4-[[3-(3-chloro-4-methoxy-phenyl)imidazo[1,2-a]pyrazin-8-yl]amino]phenyl]acetamide;
N-[4-[[3-[3-chloro-4-(difluoromethoxy)phenyl]imidazo[1,2-a]pyrazin-8-yl]amino]phenyl]acetamide;
N-[4-[[3-(2-fluoro-4-methoxy-phenyl)imidazo[1,2-a]pyrazin-8-yl]amino]phenyl]acetamide;
N-[2-(hydroxymethyl)-4-[[3-(4-methoxyphenyl)imidazo[1,2-a]pyrazin-8-yl]amino]phenyl]acetamide;
2-(2-aminoethoxy)-N-[4-[[3-(3-chloro-4-methoxy-phenyl)imidazo[1,2-a]pyrazin-8-yl]amino]phenyl]acetamide;
N-[4-[[3-(4-methoxyphenyl)imidazo[1,2-a]pyrazin-8-yl]amino]phenyl]acetamide;
N-[4-[[3-(2,4-difluorophenyl)imidazo[1,2-a]pyrazin-8-yl]amino]-2-methyl-phenyl]acetamide;
1-[4-[[3-[4-(difluoromethoxy)phenyl]imidazo[1,2-a]pyrazin-8-yl]amino]-2-methyl-phenyl]pyrrolidin-2-one;
2-chloro-N-[4-[[3-(4-methoxyphenyl)imidazo[1,2-a]pyrazin-8-yl]amino]phenyl]acetamide;
N-[4-[[3-(4-prop-2-ynoxyphenyl)imidazo[1,2-a]pyrazin-8-yl]amino]phenyl]acetamide;
1-[4-[[3-(4-chlorophenyl)imidazo[1,2-a]pyrazin-8-yl]amino]-2-methyl-phenyl]pyrrolidin-2-one;
2-methoxy-N-[4-[[3-(4-methoxyphenyl)imidazo[1,2-a]pyrazin-8-yl]amino]phenyl]acetamide;
2-[2-(2-aminoethoxy)ethoxy]-N-[4-[[3-(3-chloro-4-methoxy-phenyl)imidazo[1,2-a]pyrazin-8-yl]amino]phenyl]acetamide;
N-[4-[[3-(3-chloro-4-methoxy-phenyl)imidazo[1,2-a]pyrazin-8-yl]amino]phenyl]-2-hydroxy-acetamide;
N-[4-[[3-[4-(difluoromethoxy)phenyl]imidazo[1,2-a]pyrazin-8-yl]amino]phenyl]acetamide;
N-[4-[[3-(4-chlorophenyl)imidazo[1,2-a]pyrazin-8-yl]amino]phenyl]acetamide;
N-[4-[[3-(3-chloro-4-methoxy-phenyl)imidazo[1,2-a]pyrazin-8-yl]amino]phenyl]-2-morpholino-acetamide;
N-[4-[[3-(3-chloro-4-methoxy-phenyl)imidazo[1,2-a]pyrazin-8-yl]amino]phenyl]-N-methyl-acetamide;
7-[[3-(3-fluoro-4-methoxy-phenyl)imidazo[1,2-a]pyrazin-8-yl]amino]-1,3,4,5-tetrahydro-1-benzazepin-2-one;
N-[4-[[3-(2,3,4-trifluorophenyl)imidazo[1,2-a]pyrazin-8-yl]amino]phenyl]acetamide;
N-[2-chloro-4-[[3-(3-chloro-4-methoxy-phenyl)imidazo[1,2-a]pyrazin-8-yl]amino]phenyl]acetamide;
N-[4-[[3-(3-fluoro-4-methoxy-phenyl)imidazo[1,2-a]pyrazin-8-yl]amino]phenyl]-2-hydroxy-acetamide;
N-[4-[[3-(3-chloro-4-methoxy-phenyl)imidazo[1,2-a]pyrazin-8-yl]amino]phenyl]-N-methyl-acetamide;
1-[4-[[3-[4-(difluoromethoxy)phenyl]imidazo[1,2-a]pyrazin-8-yl]amino]phenyl]imidazolidin-2-one;
N4-[3-(2,3-difluoro-4-methoxy-phenyl)imidazo[1,2-a]pyrazin-8-yl]-2-ethyl-benzene-1,4-diamine;
N-[4-[(4-aminocyclohexyl)methylsulfonyl]-3-chloro-phenyl]-3-(2,3-difluoro-4-methoxy-phenyl)imidazo[1,2-a]pyrazin-8-amine;
N-(3-chloro-4-methylsulfonyl-phenyl)-3-(4-methoxyphenyl)imidazo[1,2-a]pyrazin-8-amine;
3-(4-methoxyphenyl)-N-(4-methylsulfonylphenyl)imidazo[1,2-a]pyrazin-8-amine;
N-[2-(2-aminoethoxy)ethyl]-2-chloro-4-[[3-(2,3-difluoro-4-methoxy-phenyl)imidazo[1,2-a]pyrazin-8-yl]amino]benzenesulfonamide;
N-[2-(2-aminoethoxy)ethyl]-4-[[3-(2,3-difluoro-4-methoxy-phenyl)imidazo[1,2-a]pyrazin-8-yl]amino]-2-methyl-benzenesulfonamide;
2-chloro-4-[[3-(2,3-difluoro-4-methoxy-phenyl)imidazo[1,2-a]pyrazin-8-yl]amino]-N-[2-[2-(dimethylamino)ethoxy]ethyl]benzenesulfonamide;
N-[3-chloro-4-[4-[3-(dimethylamino)propyl]piperazin-1-yl]sulfonyl-phenyl]-3-(2,3-difluoro-4-methoxy-phenyl)imidazo[1,2-a]pyrazin-8-amine;
[4-[4-[[3-(2,3-difluoro-4-methoxy-phenyl)imidazo[1,2-a]pyrazin-8-yl]amino]-2-ethyl-phenyl]sulfonylpiperazin-1-yl]-[(3R)-pyrrolidin-3-yl]methanone;
[4-[4-[[3-(2,3-difluoro-4-methoxy-phenyl)imidazo[1,2-a]pyrazin-8-yl]amino]-2-ethyl-phenyl]sulfonylpiperazin-1-yl]-(4-piperidyl)methanone;
30 [4-[4-[[3-(2,3-difluoro-4-methoxy-phenyl)imidazo[1,2-a]pyrazin-8-yl]amino]-2-ethyl-phenyl]sulfonylpiperazin-1-yl]-[(2S,4R)-4-hydroxypyrrolidin-2-yl]methanone;
4-[[3-(2,3-difluoro-4-methoxy-phenyl)imidazo[1,2-a]pyrazin-8-yl]amino]-N-[2-[2-(dimethylamino)ethoxy]ethyl]-2-methyl-benzenesulfonamide;
3-(2,3-difluoro-4-methoxy-phenyl)-N-[4-[4-[3-(dimethylamino)propyl]piperazin-1-yl]sulfonyl-3-methyl-phenyl]imidazo[1,2-a]pyrazin-8-amine;
[4-[4-[[3-(2,3-difluoro-4-methoxy-phenyl)imidazo[1,2-a]pyrazin-8-yl]amino]-2-ethyl-phenyl]sulfonylpiperazin-1-yl]-[(2S)-pyrrolidin-2-yl]methanone;
[2-[4-[4-[[3-(2,3-difluoro-4-methoxy-phenyl)imidazo[1,2-a]pyrazin-8-yl]amino]-2-ethyl-phenyl]sulfonylpiperazin-1-yl]-2-oxo-ethyl]-trimethyl-ammonium;
1-[4-[4-[[3-(2,3-difluoro-4-methoxy-phenyl)imidazo[1,2-a]pyrazin-8-yl]amino]-2-ethyl-phenyl]sulfonylpiperazin-1-yl]-2-(dimethylamino)ethanone;
N-[2-(2-aminoethoxy)ethyl]-4-[[3-(2,3-difluoro-4-methoxy-phenyl)imidazo[1,2-a]pyrazin-8-yl]amino]benzenesulfonamide;
3-(2,3-difluoro-4-methoxy-phenyl)-N-(3-ethyl-4-piperazin-1-ylsulfonyl-phenyl)imidazo[1,2-a]pyrazin-8-amine;
3-(4-methoxyphenyl)-N-(4-morpholinosulfonylphenyl)imidazo[1,2-a]pyrazin-8-amine;
4-[[3-(4-methoxyphenyl)imidazo[1,2-a]pyrazin-8-yl]amino]-N,N-dimethyl-benzenesulfonamide;
[4-[4-[[3-(2,3-difluoro-4-methoxy-phenyl)imidazo[1,2-a]pyrazin-8-yl]amino]-2-ethyl-phenyl]sulfonylpiperazin-1-yl]-(4-hydroxy-4-piperidyl)methanone;
N-[2-(2-aminoethoxy)ethyl]-4-[[3-(4-methoxyphenyl)imidazo[1,2-a]pyrazin-8-yl]amino]-2-methyl-benzenesulfonamide;
N,N-diethyl-2-[[4-[[3-(4-methoxyphenyl)imidazo[1,2-a]pyrazin-8-yl]amino]phenyl]sulfonylamino]acetamide;
4-[[3-(3-chloro-4-methoxy-phenyl)imidazo[1,2-a]pyrazin-8-yl]amino]-N-methyl-benzenesulfonamide;
3-(4-methoxyphenyl)-N-[4-(4-methylpiperazin-1-yl)sulfonylphenyl]imidazo[1,2-a]pyrazin-8-amine;
[4-[4-[[3-(2,3-difluoro-4-methoxy-phenyl)imidazo[1,2-a]pyrazin-8-yl]amino]-2-ethyl-phenyl]sulfonylpiperazin-1-yl]-[(3R)-1-[(2S,4R)-4-hydroxypyrrolidine-2-carbonyl]pyrrolidin-3-yl]methanone;
N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine;
2-[4-[8-[4-(N,S-dimethylsulfonimidoyl)-3-methyl-anilino]imidazo[1,2-a]pyrazin-3-yl]-2,3-difluoro-phenoxy]acetonitrile;
3-(2,3-difluoro-4-methoxy-phenyl)-N-[4-(N,S-dimethylsulfonimidoyl)-3-methyl-phenyl]imidazo[1,2-a]pyrazin-8-amine;
N-[4-[S-(3-aminopropyl)-N-methyl-sulfonimidoyl]phenyl]-3-(2,3-difluoro-4-methoxy-phenyl)imidazo[1,2-a]pyrazin-8-amine;
N-[3-chloro-4-(N,S-dimethylsulfonimidoyl)phenyl]-3-(2,3-difluoro-4-methoxy-phenyl)imidazo[1,2-a]pyrazin-8-amine;
N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine;
N-[4-[S-(3-aminopropyl)-N-methyl-sulfonimidoyl]-3-methyl-phenyl]-3-(2,3-difluoro-4-methoxy-phenyl)imidazo[1,2-a]pyrazin-8-amine;
rac-(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
3-(2,3-difluoro-4-methoxy-phenyl)-N-[4-(N,S-dimethylsulfonimidoyl)-3-fluoro-phenyl]imidazo[1,2-a]pyrazin-8-amine;
[4-[S-[4-[[3-(2,3-difluoro-4-methoxy-phenyl)imidazo[1,2-a]pyrazin-8-yl]amino]phenyl]-N-methyl-sulfonimidoyl]-1-piperidyl]-[rac-(2S,4R)-4-hydroxypyrrolidin-2-yl]methanone;
N-[3-chloro-4-(S-ethyl-N-methyl-sulfonimidoyl)phenyl]-3-(2,3-difluoro-4-methoxy-phenyl)imidazo[1,2-a]pyrazin-8-amine;
rac-(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
N-[4-[N-(3-aminopropyl)-S-methyl-sulfonimidoyl]-3-ethyl-phenyl]-3-(2,3-difluoro-4-methoxy-phenyl)imidazo[1,2-a]pyrazin-8-amine;
3-(2,3-difluoro-4-methoxy-phenyl)-N-[4-(N,S-dimethylsulfonimidoyl)phenyl]imidazo[1,2-a]pyrazin-8-amine;
[4-[S-[4-[[3-(2,3-difluoro-4-methoxy-phenyl)imidazo[1,2-a]pyrazin-8-yl]amino]-2-methyl-phenyl]-N-methyl-sulfonimidoyl]-1-piperidyl]-[rac-(2S,4R)-4-hydroxypyrrolidin-2-yl]methanone;
N-[4-[N-(3-aminopropyl)-S-methyl-sulfonimidoyl]phenyl]-3-(2,3-difluoro-4-methoxy-phenyl)imidazo[1,2-a]pyrazin-8-amine;
rac-(2S,4R)—N-[3-[[[4-[[3-(2,3-difluoro-4-methoxy-phenyl)imidazo[1,2-a]pyrazin-8-yl]amino]phenyl]-methyl-oxo-lambda6-sulfanylidene]amino]propyl]-4-hydroxy-pyrrolidine-2-carboxamide;
3-(2,3-difluoro-4-methoxy-phenyl)-N-[4-[S-(dimethylamino)-N-methyl-sulfonimidoyl]-3-methyl-phenyl]imidazo[1,2-a]pyrazin-8-amine;
N-[4-[4-(aminomethyl)-1-piperidyl]-3-methyl-phenyl]-3-(2,3-difluoro-4-methoxy-phenyl)imidazo[1,2-a]pyrazin-8-amine;
N-[4-[4-(aminomethyl)-1-piperidyl]-3-chloro-phenyl]-3-(2,3-difluoro-4-methoxy-phenyl)imidazo[1,2-a]pyrazin-8-amine;
N-[4-[4-(aminomethyl)-1-piperidyl]phenyl]-3-(2,3-difluoro-4-methoxy-phenyl)imidazo[1,2-a]pyrazin-8-amine;
N-[4-[4-(aminomethyl)-1-piperidyl]-3-ethyl-phenyl]-3-(2,3-difluoro-4-methoxy-phenyl)imidazo[1,2-a]pyrazin-8-amine;
N1-(2-aminoethyl)-N5-[3-(2,3-difluoro-4-methoxy-phenyl)imidazo[1,2-a]pyrazin-8-yl]indane-1,5-diamine;
5-[[3-(2,3-difluoro-4-methoxy-phenyl)imidazo[1,2-a]pyrazin-8-yl]amino]indan-1-one;
2-iodo-5-[[3-(4-methoxyphenyl)imidazo[1,2-a]pyrazin-8-yl]amino]-N-methyl-benzamide;
3-(2,3-difluoro-4-methoxy-phenyl)-N-[4-[[dimethyl(oxo)-lambda6-sulfanylidene]amino]-3-methyl-phenyl]imidazo[1,2-a]pyrazin-8-amine;
3-[4-(difluoromethoxy)phenyl]-N-[4-(1,1-dioxo-1,2-thiazolidin-2-yl)phenyl]imidazo[1,2-a]pyrazin-8-amine;
N-[4-[2-[2-(2-aminoethoxy)ethoxy]ethoxy]-3-methyl-phenyl]-3-[2,3-difluoro-4-(2-pyridyloxy)phenyl]imidazo[1,2-a]pyrazin-8-amine;
2-chloro-5-[[3-(4-methoxyphenyl)imidazo[1,2-a]pyrazin-8-yl]amino]-N-methyl-benzamide;
3-(2,3-difluoro-4-methoxy-phenyl)-N-[4-(imidazol-1-ylmethyl)-3-methyl-phenyl]imidazo[1,2-a]pyrazin-8-amine;
[4-[[3-[4-(difluoromethoxy)phenyl]imidazo[1,2-a]pyrazin-8-yl]amino]-2-methyl-phenyl]methanol;
N-[4-(imidazol-1-ylmethyl)-3-methyl-phenyl]-3-(4-methoxyphenyl)imidazo[1,2-a]pyrazin-8-amine;
N-[4-(imidazol-1-ylmethyl)phenyl]-3-(4-methoxyphenyl)imidazo[1,2-a]pyrazin-8-amine;
[4-[[3-(2,3-difluoro-4-methoxy-phenyl)imidazo[1,2-a]pyrazin-8-yl]amino]-2-ethyl-phenyl]methyl-trimethyl-ammonium; and
[4-[[3-(4-methoxyphenyl)imidazo[1,2-a]pyrazin-8-yl]amino]-2-methyl-phenyl]methanol.
In a preferred embodiment, the invention provides a compound of formula (I) as defined herein,
or a pharmaceutically acceptable salt thereof, wherein the compound of formula (I) is selected from:
N-[4-[[3-(3-chloro-4-methoxy-phenyl)imidazo[1,2-a]pyrazin-8-yl]amino]-2-methyl-phenyl]acetamide;
1-[4-[[3-(3-fluoro-4-methoxy-phenyl)imidazo[1,2-a]pyrazin-8-yl]amino]phenyl]pyrrolidin-2-one;
4-(4-aminobutyl)-1-[4-[[3-[4-(difluoromethoxy)phenyl]imidazo[1,2-a]pyrazin-8-yl]amino]phenyl]pyrrolidin-2-one;
1-[4-[[3-[4-(difluoromethoxy)phenyl]imidazo[1,2-a]pyrazin-8-yl]amino]phenyl]imidazolidin-2-one;
N4-[3-(2,3-difluoro-4-methoxy-phenyl)imidazo[1,2-a]pyrazin-8-yl]-2-ethyl-benzene-1,4-diamine;
N-[4-[(4-aminocyclohexyl)methylsulfonyl]-3-chloro-phenyl]-3-(2,3-difluoro-4-methoxy-phenyl)imidazo[1,2-a]pyrazin-8-amine;
N-[2-(2-aminoethoxy)ethyl]-2-chloro-4-[[3-(2,3-difluoro-4-methoxy-phenyl)imidazo[1,2-a]pyrazin-8-yl]amino]benzenesulfonamide;
N-[3-chloro-4-[4-[3-(dimethylamino)propyl]piperazin-1-yl]sulfonyl-phenyl]-3-(2,3-difluoro-4-methoxy-phenyl)imidazo[1,2-a]pyrazin-8-amine;
30 [4-[4-[[3-(2,3-difluoro-4-methoxy-phenyl)imidazo[1,2-a]pyrazin-8-yl]amino]-2-ethyl-phenyl]sulfonylpiperazin-1-yl]-[(2S,4R)-4-hydroxypyrrolidin-2-yl]methanone;
3-(2,3-difluoro-4-methoxy-phenyl)-N-[4-(N,S-dimethylsulfonimidoyl)-3-methyl-phenyl]imidazo[1,2-a]pyrazin-8-amine;
[4-[S-[4-[[3-(2,3-difluoro-4-methoxy-phenyl)imidazo[1,2-a]pyrazin-8-yl]amino]-2-methyl-phenyl]-N-methyl-sulfonimidoyl]-1-piperidyl]-[rac-(2S,4R)-4-hydroxypyrrolidin-2-yl]methanone;
rac-(2S,4R)—N-[3-[[[4-[[3-(2,3-difluoro-4-methoxy-phenyl)imidazo[1,2-a]pyrazin-8-yl]amino]phenyl]-methyl-oxo-lambda6-sulfanylidene]amino]propyl]-4-hydroxy-pyrrolidine-2-carboxamide;
N-[4-[4-(aminomethyl)-1-piperidyl]-3-methyl-phenyl]-3-(2,3-difluoro-4-methoxy-phenyl)imidazo[1,2-a]pyrazin-8-amine;
N1-(2-aminoethyl)-N5-[3-(2,3-difluoro-4-methoxy-phenyl)imidazo[1,2-a]pyrazin-8-yl]indane-1,5-diamine;
2-iodo-5-[[3-(4-methoxyphenyl)imidazo[1,2-a]pyrazin-8-yl]amino]-N-methyl-benzamide;
3-(2,3-difluoro-4-methoxy-phenyl)-N-[4-[[dimethyl(oxo)-lambda6-sulfanylidene]amino]-3-methyl-phenyl]imidazo[1,2-a]pyrazin-8-amine;
3-[4-(difluoromethoxy)phenyl]-N-[4-(1,1-dioxo-1,2-thiazolidin-2-yl)phenyl]imidazo[1,2-a]pyrazin-8-amine; and
N-[4-[2-[2-(2-aminoethoxy)ethoxy]ethoxy]-3-methyl-phenyl]-3-[2,3-difluoro-4-(2-pyridyloxy)phenyl]imidazo[1,2-a]pyrazin-8-amine.

In one embodiment, the present invention provides pharmaceutically acceptable salts of the compounds of formula (I) as described herein, especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I) as described herein (i.e., as “free bases” or “free acids”, respectively).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ², ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

Processes of Manufacturing

The preparation of compounds of formula (I) of the present invention may be carried out in sequential or convergent synthetic routes. Syntheses of the compounds of the invention are shown in the following schemes. The skills required for carrying out the reactions and purifications of the resulting products are known to those skilled in the art. The substituents and indices used in the following description of the processes have the significance given herein before unless indicated to the contrary. In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 3rd Edition, Richard C. Larock. John Wiley & Sons, New York, N.Y. 2018). We find it convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux temperature. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

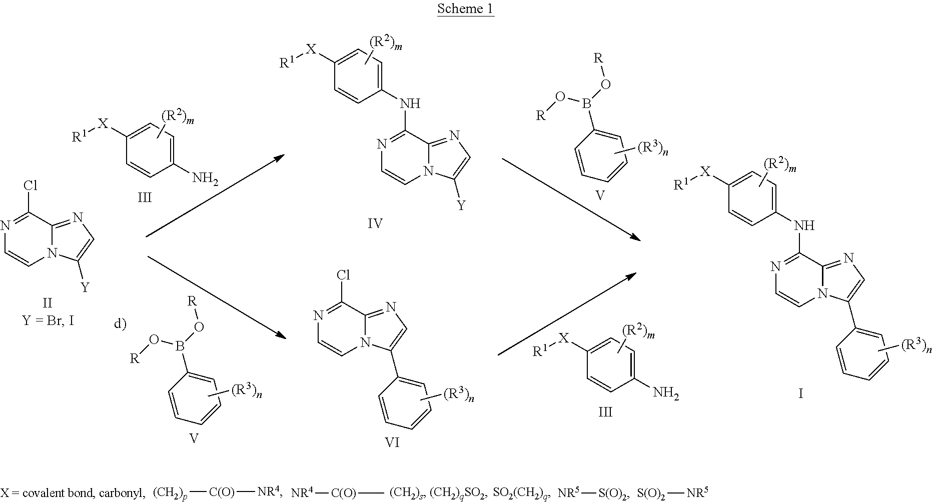

a) 8-chloro-3-iodoimidazo[1,2-a]pyrazine (resp. 8-chloro-3-bromoimidazo[1,2-a]pyrazine) II is commercially available and can conveniently be reacted with aniline derivatives III under acidic (e.g., AcOH) or basic conditions (DIPEA, carbonates and the like), in the presence or absence of a transition metal catalyst (such as Pd, depending on the nature and hence the reactivity of the chosen aniline derivative III) in a suitable solvent (acetonitrile, dioxane, NMP and the like), depending on the reagent chosen to access imidazo-pyrazine derivative IV. (Scheme 1) These compounds can be intermediates utilized in the subsequent Suzuki reaction with boronic acids (R═H) or esters (R=alkyl) V under transition metal catalysis (typical metal source: Pd and the like) in a solvent (dioxane, DMF, THF and the like) in the presence of a base (NEt₃, DIPEA, carbonates, and the like) to yield imidazopyrazine derivatives I.
b) Alternatively 8-chloro-3-iodoimidazo[1,2-a]pyrazine (resp. 8-chloro-3-bromoimidazo[1,2-a]pyrazine) II can conveniently be reacted in Suzuki reactions with boronic acids or esters V under transition metal catalysis (typical metal source: Pd and the like) in a solvent (dioxane, DMF, THF and the like) in the presence of a base (NEt₃, DIPEA, carbonates, and the like) to yield VI. These compounds can be intermediates and reacted with aniline derivatives III under acidic (AcOH and the like) or basic conditions (DIPEA, carbonates and the like) in the presence or absence of a transition metal catalyst (such as Pd, depending on the nature and hence the reactivity of the chosen aniline derivative III) in a suitable solvent (acetonitrile, dioxane, NMP and the like), depending on the reagent chosen to access imidazo-pyrazine derivative I.

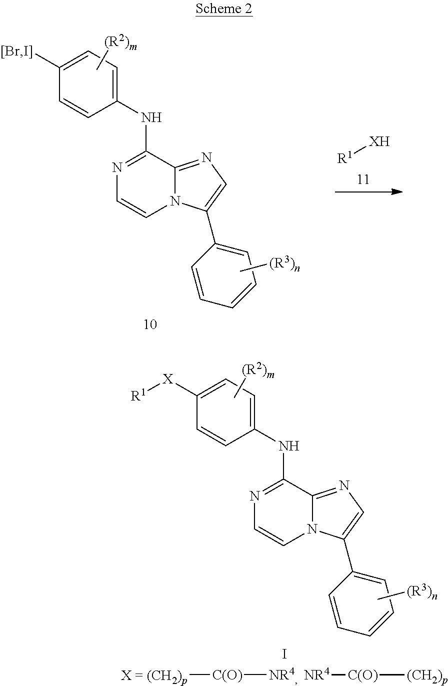

An intermediate halide 10 can be reacted with amines 11 using metal catalyzed reaction conditions, for example palladium catalyzed Buchwald reactions or copper catalyzed Ullmann-reaction conditions known well in the art, typically in the presence of a base and suitable solvent such as DMSO, 1-butanol, DMF and the like, to obtain molecules of structure I (Scheme 2)

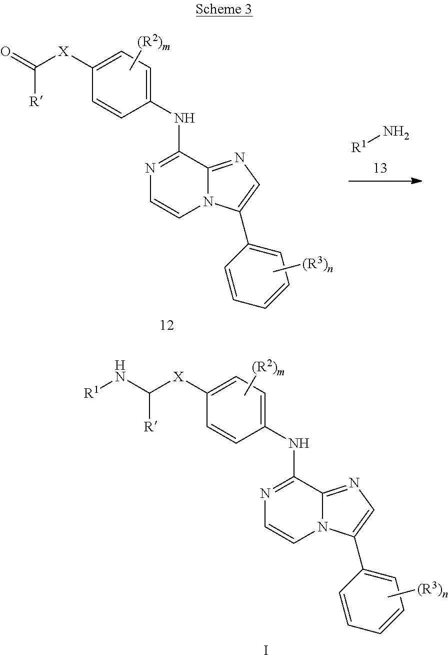

Intermediate ketones and aldehydes 12 can be reacted with amines 13 in the presence of a reducing agent and suitable solvent (reductive amination) to obtain structures of general formula I (Scheme 3).

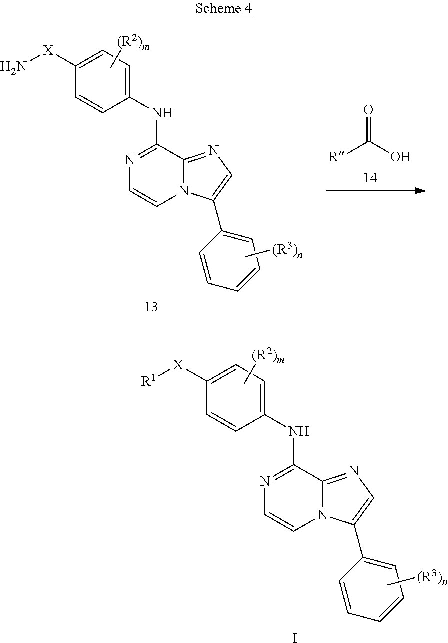

Intermediate amines of general structure 13 can be prepared in analogy to the methods described above and then reacted with an acid 14 in presence of well known amide coupling reagents such as HATU, T3P and the like typically in the presence of base and the like or with an activated derivative of acid 14 in a suitable solvent like acetonitrile, DMF, dichloromethane, THF or the like to give molecules of structure I (Scheme 4).

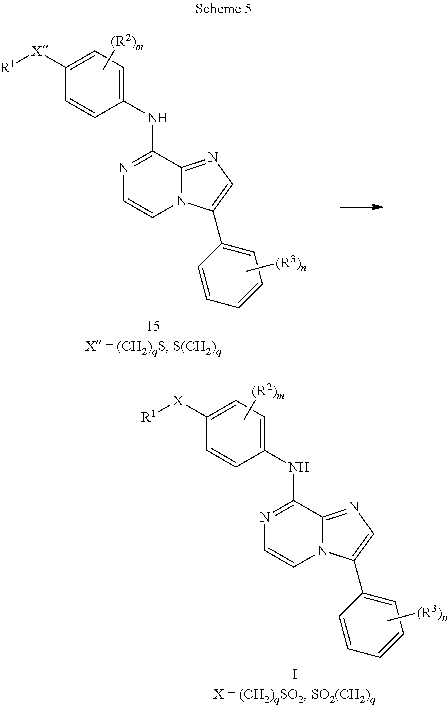

Thioethers of general structure 15 can be oxidized to molecules of general structure I using a suitable oxidizing reagent such as mCPBA and the like in a solvent like DCM, THF and the like.

In one aspect, the present invention provides a process of manufacturing the compounds of formula (I) described herein, comprising:
(i) reacting a heteroaryl halide (IV), wherein R¹, R², X and m are as defined herein and Y is bromo or iodo,

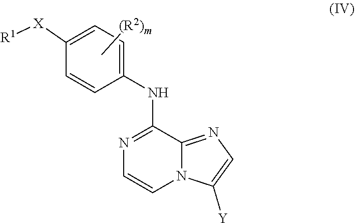

with a compound (V), wherein R³ and n are as defined herein and R is hydrogen or C₁-C₆-alkyl or the two R groups, taken together with the atoms to which they are attached, form a cyclic boronic acid ester,

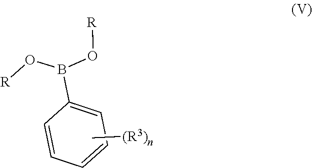

in the presence of a transition metal catalyst, such as 1,1′-bis(diphenylphosphino)ferrocene-palladium(II)dichloride dichloromethane complex, to afford said compound of formula (I); or
(ii) reacting a heteroaryl chloride (VI), wherein R³ and n are as defined herein,

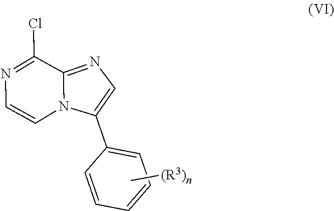

with an aniline derivative (III), wherein R¹, R², X, and m are as defined herein,

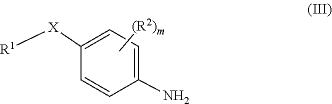

under acidic or basic conditions in the presence or absence of a transition metal catalyst (depending on the nature and hence the reactivity of the chosen aniline derivative III) in a suitable solvent, to afford said compound of formula (I); and
(iii) optionally converting said compound of formula (I) to a pharmaceutically acceptable salt thereof.

In a further aspect, the present invention provides a compound of formula (I) as described herein, or a pharmaceutically acceptable salt thereof, when manufactured according to the processes disclosed herein.

Using the Compounds of the Invention

As illustrated in the experimental section, the compounds of formula (I) and their pharmaceutically acceptable salts possess valuable pharmacological properties for the treatment or prevention of infections and resulting diseases, particularly bacteremia, pneumonia, meningitis, urinary tract infection, and wound infection, caused by pathogens, particularly by bacteria, more particularly by Acinetobacter species, most particularly by Acinetobacter baumannii.

The compounds of formula (I) and their pharmaceutically acceptable salts exhibit activity as antibiotics, particularly as antibiotics against Acinetobacter species, more particularly as antibiotics against Acinetobacter baumannii, most particularly as pathogen-specific antibiotics against Acinetobacter baumannii.

The compounds of formula (I) and their pharmaceutically acceptable salts can be used as antibiotics, i.e. as antibacterial pharmaceutical ingredients suitable in the treatment and prevention of bacterial infections, particularly in the treatment and prevention of bacterial infections caused by Acinetobacter species, more particularly in the treatment and prevention of bacterial infections caused by Acinetobacter baumannii.

The compounds of the present invention can be used, either alone or in combination with other drugs, for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In one aspect, the present invention provides compounds of formula (I) or their pharmaceutically acceptable salts as described herein for use as therapeutically active substances.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a particular embodiment, said nosocomial infections and resulting diseases are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Gram-negative bacteria.

In a particular embodiment, said infections and resulting diseases caused by Gram-negative bacteria are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a particular embodiment, said infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, are selected from bacteremia, pneumonia, meningitis, urinary tract infection and wound infection, or a combination thereof.

In a further aspect, the present invention provides compounds of formula (I) or their pharmaceutically acceptable salts as defined above for use in the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides a method for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii, which method comprises administering a compound of formula (I) or a pharmaceutically acceptable salt thereof as defined above to a mammal.

In a further aspect, the present invention provides the use of compounds of formula (I) or their pharmaceutically acceptable salts as defined above for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides the use of compounds of formula (I) or their pharmaceutically acceptable salts as defined above for the preparation of medicaments for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii. Such medicaments comprise compounds of formula (I) or their pharmaceutically acceptable salts as defined above.

Pharmaceutical Compositions and Administration

In one aspect, the present invention provides pharmaceutical compositions comprising compounds of formula (I) or their pharmaceutically acceptable salts as defined above and one or more pharmaceutically acceptable excipients. Exemplary pharmaceutical compositions are described in Examples 126-129.

In a further aspect, the present invention relates to pharmaceutical compositions comprising compounds of formula (I) or their pharmaceutically acceptable salts as defined above and one or more pharmaceutically acceptable excipients for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions or infusion solutions).

The compounds of formula (I) and their pharmaceutically acceptable salts can be processed with pharmaceutically inert, inorganic or organic excipients for the production of tablets, coated tablets, dragées and hard gelatin capsules. Lactose, corn starch or derivatives thereof, talc, stearic acid or its salts etc. can be used, for example, as such excipients for tablets, dragées and hard gelatin capsules.

Suitable excipients for soft gelatin capsules are, for example, vegetable oils, waxes, fats, semi-solid substances and liquid polyols, etc.

Suitable excipients for the production of solutions and syrups are, for example, water, polyols, saccharose, invert sugar, glucose, etc.

Suitable excipients for injection solutions are, for example, water, alcohols, polyols, glycerol, vegetable oils, etc.

Suitable excipients for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

Co-Administration of Compounds of Formula (I) and Other Agents

The compounds of formula (I) or salts thereof or a compound disclosed herein or a pharmaceutically acceptable salt thereof may be employed alone or in combination with other agents for treatment. For example, the second agent of the pharmaceutical combination formulation or dosing regimen may have complementary activities to the compound of formula (I) such that they do not adversely affect each other. The compounds may be administered together in a unitary pharmaceutical composition or separately. In one embodiment a compound or a pharmaceutically acceptable salt can be co-administered with an antibiotic, in particular with an antibiotic for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

The term “co-administering” refers to either simultaneous administration, or any manner of separate sequential administration, of a compound of formula (I) or a salt thereof or a compound disclosed herein or a pharmaceutically acceptable salt thereof and a further active pharmaceutical ingredient or ingredients, including antibiotic agents. If the administration is not simultaneous, the compounds are administered in a close time proximity to each other. Furthermore, it does not matter if the compounds are administered in the same dosage form, e.g. one compound may be administered intravenously and another compound may be administered orally.

Typically, any agent that has antimicrobial activity may be co-administered. Particular examples of such agents are Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified e.g. in WO 2017072062 A1, WO 2019185572 A1 and WO 2019206853 A1, and Macrolides (erythromycin).

In one aspect, the present invention provides a pharmaceutical composition described herein, further comprising an additional therapeutic agent.

In one aspect, the present invention provides a pharmaceutical combination comprising a compound of formula (I) described herein and an additional therapeutic agent.

In one embodiment, said additional therapeutic agent is an antibiotic agent.

In one embodiment, said additional therapeutic agent is an antibiotic agent that is useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In one embodiment, said additional therapeutic agent is an antibiotic agent selected from Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified in WO 2017072062 A1, WO 2019185572 A1 and WO 2019206853 A1, and Macrolides (erythromycin).

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

The following abbreviations are used in the present text:

(R)-BINAP=(R)-2,2′-bis(diphenylphosphino)-1,1′-binaphthyl, ACN=acetonitrile, aq.=aqueous, Boc=tert-butyloxycarbonyl, Boc-Glu-OtBu=Boc-L-glutamic acid 1-tert-butyl ester, Boc-Glu(OtBu)-OH═N-α-t.-Boc-L-glutamic acid 7-t.-butyl ester, Boc-Orn(Z)—OH=Nα-Boc-Nδ-Cbz-L-ornithine, Nα-Boc-Nδ-Z-L-ornithine, Nδ-Z-Nα-Boc-L-ornithine, BrettPhos-Pd-G3=[(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate, CAS=chemical abstracts registration number, Cs₂CO₃=cesium carbonate, DCM=dichloromethane, DIAD=diisopropyl azodicarboxylate, DIPEA=ethyl diisopropylamine, DMA=N,N-dimethylacetamide, DMAP=4-(dimethylamino)-pyridine, DMF=N,N-dimethylformamide, DMSO=dimethylsulfoxide, DMSO-d6=deuterated dimethylsulfoxide, EA=ethyl acetate, EDC=1-ethyl-3-(3-dimethylaminopropyl)carbodiimide, EDCI=1-ethyl-3-(3-dimethylaminopropyl)carbodiimide, EI=electron impact, ESI=electrospray ionization, ESI⁺=electrospray ionization positive (mode), ESP=electrospray ionization positive (mode), Et₂O=diethylether, Et₃N=triethylamine, EtOAc=ethyl acetate, EtOH=ethanol, FA=formic acid, Fmoc-Agp(Boc)₂-OH=N-α-Fmoc-N,Nω-γ-di-t.-butoxycarbonyl-L-diaminobutanoic acid, Fmoc-Arg(Boc)2-OH═N-α-Fmoc-N-ω,N-ωAE-bis-t-butoxycarbonyl-L-arginine, H2=hydrogen, h=hour(s), HATU=1-[bis(dimethylamino)methylene]-1H-1,2,3-triazolo[4,5-b]pyridinium-3-oxide hexafluorophosphate, HCl=hydrochloric acid, HFIP=1,1,1,3,3,3-hexafluoroisopropanol, H2O=water, HOBt=1-hydroxy-1H-benzotriazole, HPLC=high performance liquid chromatography, HV=high vacuum, ISN=ion spray negative (mode), K₂CO₃=potassium carbonate, KI=potassium iodide, KOH=potassium hydroxide, K₃PO₄=potassium phosphate tribasic, LC-MS=liquid chromatography coupled with mass spectroscopy, LiOH=lithium hydroxide, MeOH=methanol, MgSO₄=magnesium sulphate, min=minute(s), mL=milliliter, MS=mass spectrometry, MTBE=tert.-butyl methyl ether, N2=nitrogen, Na₂CO₃=sodium carbonate, Na₂SO₃=sodium sulfite, Na₂SO₄=sodium sulfate, Na₂SO₂O₃=sodium thiosulfate, NEt₃=triethylamine, NaHCO₃=sodium hydrogen carbonate, NaOH=sodium hydroxide, NH₄Cl=ammonium chloride, NiCl₂.6H₂O=nickel(II)chloride hexahydrate, NMO=N-methylmorpholine N-oxide, NMP=N-methyl-2-pyrrolidone, Pd/C=palladium on activated carbon, Pd₂(dba)₃=tris(dibenzylideneacetone)dipalladium(0), PdCl₂(PPh₃)₂=bis(triphenylphosphine)palladium(II) dichloride, Pd(dppf)Cl₂=[1,1′-bis(diphenylphosphino)ferrocene]dichloropalladium(II), PdCl₂(dppf)-CH₂Cl₂=[1,1′-bis(diphenylphosphino)ferrocene]dichloropalladium(II) dichloromethane complex, PE=petroleum ether, PhI(OAc)₂=(diacetoxyiodo)benzene, PPA=polyphosphoric acid, pTsOH=para toluenesulfonic acid, Rf=retention factor, RM=reaction mixture, RT=room temperature, SOCl₂=thionyl chloride, SFC=supercritical fluid chromatography, TBTU=2-(1H-Benzotriazole-1-yl)-1,1,3,3-tetramethylaminium tetrafluoroborate, T₃P=propylphosphonic anhydride, t-Bu-X-phos=2-di-tert-butylphosphino-2′,4′,6′-triisopropylbiphenyl, TEA=triethylamine, TEMPO=(2,2,6,6-tetramethylpiperidin-1-yl)oxyl, TFA=trifluoroacetic acid, THF=tetrahydrofurane, prep-TLC=preparative thin layer chromatography, UV=ultraviolet.

### Example 1

### 3-[4-(difluoromethoxy)phenyl]-N-[4-(1,1-dioxo-1,2-thiazolidin-2-yl)phenyl]imidazo[1,2-a]pyrazin-8-amine

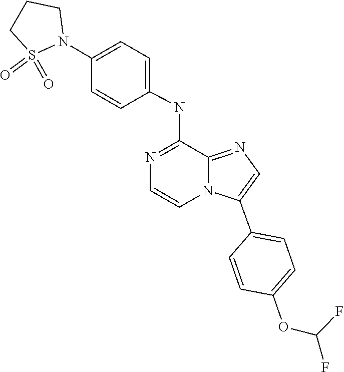

### Step 1) 2-(4-nitrophenyl)-1,2-thiazolidine 1,1-dioxide

A mixture of 1-iodo-4-nitrobenzene (500.0 mg, 2.01 mmol, 1 eq), isothiazolidine 1,1-dioxide (270.0 mg, 2.23 mmol, 1.11 eq), Copper(I) iodide (80.0 mg, 0.420 mmol, 0.210 eq), N,N-dimethyl-1,2-cyclohexanediamine (60.0 mg, 0.420 mmol, 0.210 eq) and K₃PO₄ (1280.0 mg, 6.03 mmol, 3 eq) in DMSO (10 mL) was stirred at 100° C. for 16 h. The mixture was diluted with EtOAc (100 mL), washed with water (30 mL), brine (30 mL×2), dried over Na₂SO₄ and concentrated under reduced pressure to give crude product. The product was triturated with EA/PE=3:1 (5 mL) for 5 min. The mixture was filtered. The filter cake was dried over high vacuum to give the title compound (420 mg, yield: 86.3%) as a yellow solid. ESI MS [M+H]⁺: 243.1

### Step 2) 4-(1,1-dioxo-1,2-thiazolidin-2-yl)aniline

A solution of 2-(4-nitrophenyl)-1,2-thiazolidine 1,1-dioxide (400.0 mg, 1.65 mmol, 1 eq) in MeOH (3 mL)/DCM (2 mL) was hydrogenated in the presence of Pd/C (100.0 mg) at 35° C. and 50 psi for 16 h. The mixture was filtered. The filtrate was concentrated under reduced pressure to give the title compound (320 mg, yield: 91.3%) as an off-white solid. MS obsd. ESI MS [M+H]⁺: 213

### Step 3) 3-[4-(difluoromethoxy)phenyl]-N-[4-(1,1-dioxo-1,2-thiazolidin-2-yl)phenyl]imidazo[1,2-a]pyrazin-8-amine

A mixture of 8-chloro-3-[4-(difluoromethoxy)phenyl]imidazo[1,2-a]pyrazine (100 mg, 0.340 mmol, 1 eq) and 4-(1,1-dioxo-1,2-thiazolidin-2-yl)aniline (79 mg, 0.370 mmol, 1.1 eq) in MeCN (1.8 mL)/AcOH (0.2 mL) was stirred at 100° C. for 3 h. After cooling to room temperature, the mixture was filtered. The filter cake was trituration twice with McCN (3 mL) and MeOH (3 mL) to give the title compound (21.4 mg, yield: 13.05%) as a gray solid.

¹H NMR (400 MHz, DMSO-d₆) δ ppm 8.01 (s, 1H) 7.96 (d, J=5.02 Hz, 1H) 7.89 (br d, J=8.78 Hz, 2H) 7.78 (d, J=8.66 Hz, 2H) 7.19-7.58 (m, 6H) 3.76 (t, J=6.53 Hz, 2H) 3.55-3.56 (m, 2H) 2.38-2.46 (m, 2H). MS obsd. ESI MS [M+H]⁺: 472.0

### Example 2

### 3-[4-[[3-[4-(difluoromethoxy)phenyl]imidazo[1,2-a]pyrazin-8-yl]amino]phenyl]oxazolidin-2-one

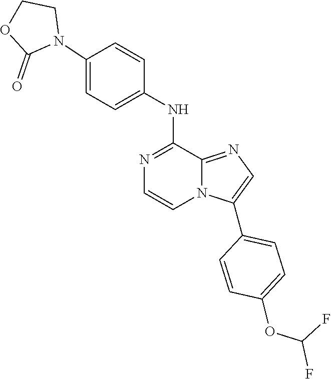 [SMILES: O=C1OCCN1c1ccc(Nc2nccn3c(-c4ccc(OC(F)F)cc4)cnc23)cc1]

### Step 1) 3-[4-[[3-[4-(difluoromethoxy)phenyl]imidazo[1,2-a]pyrazin-8-yl]amino]phenyl]oxazolidin-2-one

To a mixture of 3-[4-(difluoromethoxy)phenyl]-N-(4-iodophenyl)imidazo[1,2-a]pyrazin-8-amine (compound 7, 100 mg, 0.21 mmol) and 2-oxazolidone (22 mg, 0.25 mmol) in DMSO (2 mL) was added K₃PO₄ (134 mg, 0.63 mmol), CuI (8 mg, 0.04 mmol) and N,N′-dimethyl-1,2-cyclohexanediamine (6 mg, 0.04 mmol). The mixture was stirred at 100° C. for 16 h. The mixture was diluted with EtOAc (200 mL) and the organic layer was washed with water (50 mL), brine (100 mL×3), dried over anhydrous Na₂SO₄ and concentrated under reduced pressure to give the crude product. The crude product was further purified by prep-TLC and prep-HPLC (base) to give the title compound (9 mg, 9.5% yield) as a white solid.

¹H NMR (400 MHz, DMSO-d₆) δ 9.66 (s, 1H), 8.06 (br d, J=9.03 Hz, 2H), 7.93 (br d, J=4.52 Hz, 1H), 7.85 (s, 1H), 7.77 (br d, J=8.53 Hz, 2H), 7.16-7.56 (m, 6H), 4.44 (br t, J=7.78 Hz, 2H), 4.06 (br t, J=7.78 Hz, 2H). ESI MS [M+H]⁺: 438.1

### Example 3

### 1-[4-[[3-[4-(difluoromethoxy)phenyl]imidazo[1,2-a]pyrazin-8-yl]amino]phenyl]imidazolidin-2-one

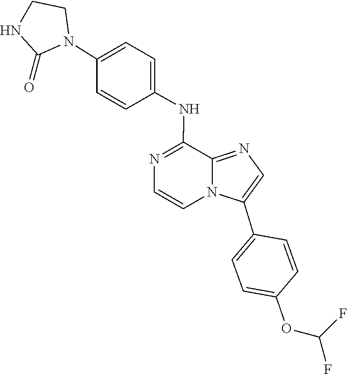 [SMILES: O=C1NCCN1c1ccc(Nc2nccn3c(-c4ccc(OC(F)F)cc4)cnc23)cc1]

### Step 1) 1-(4-aminophenyl)imidazolidin-2-one

A mixture of 4-iodoaniline (500 mg, 2.28 mmol), 2-imidazolidone (983 mg, 11.41 mmol), potassium carbonate (947 mg, 6.85 mmol), trans-(1r,2r)-N,N′-bismethyl-1,2-cyclohexane diamine (97 mg, 0.68 mmol) and copper(I) iodide (43 mg, 0.23 mmol) in 1-butanol (15 mL) was stirred at 100° C. for 5 h. The reaction was cooled and the solvent was removed. The residue was purified by the flash column chromatography (DCM:MeOH=50:1) to afford the title compound (370 mg, yield: 76.46%) as a brown solid. ESI MS [M+H]⁺: 178.1

### Step 2) 1-[4-[[3-[4-(difluoromethoxy)phenyl]imidazo[1,2-a]pyrazin-8-yl]amino]phenyl]imidazolidin-2-one

A mixture of compound 1-(4-aminophenyl)imidazolidin-2-one (108 mg, 0.51 mmol) and 8-chloro-3-[4-(difluoromethoxy)phenyl]imidazo[1,2-a]pyrazine (100 mg, 0.34 mmol) in ACN (2.7 mL)/acetic acid (0.3 mL) was stirred at 100° C. for 4 h. The reaction mixture was cooled to 25° C. slowly and the formed suspension was filtered. The collected solid was dried and purified by recrystallization (DMSO/H2O) to afford the title compound (17 mg, yield: 11.17%) as a brown solid.

¹H NMR (400 MHz, DMSO) □ □ 19.52 (s, 1H), 7.96 (d, J=9.03 Hz, 2H), 7.89 (d, J=4.77 Hz, 1H), 7.84 (s, 1H), 7.73-7.80 (m, 2H), 7.16-7.56 (m, 6H), 6.84 (s, 1H), 3.80-3.90 (m, 2H), 3.38-3.43 (m, 2H). ESI MS [M+H]⁺: 437.2

### Example 4

### 4-[4-[[3-[4-(difluoromethoxy)phenyl]imidazo[1,2-a]pyrazin-8-yl]amino]phenyl]morpholin-3-one

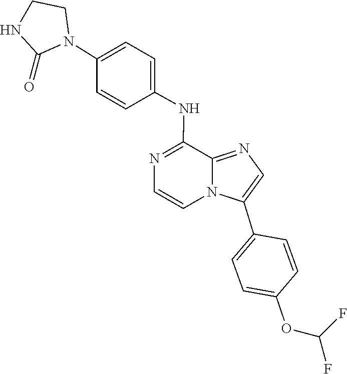 [SMILES: O=C1NCCN1c1ccc(Nc2nccn3c(-c4ccc(OC(F)F)cc4)cnc23)cc1]

### 4-[4-[[3-[4-(difluoromethoxy)phenyl]imidazo[1,2-a]pyrazin-8-yl]amino]phenyl]morpholin-3-one

A mixture of 3-[4-(difluoromethoxy)phenyl]-N-(4-iodophenyl)imidazo[1,2-a]pyrazin-8-amine (compound 7, 200 mg, 0.42 mmol), morpholin-3-one (63 mg, 0.63 mmol), trans-(1r, 2r)-N, N′-bismethyl-1, 2-cyclo hexanediamine (12 mg, 0.08 mmol), copper(I) iodide (16 mg, 0.08 mmol) and K₃PO₄ (266 mg, 1.25 mmol) in DMSO (2 mL) was stirred at 100° C. for 16 h. The reaction was cooled, diluted with H₂O (10 mL) and extracted with DCM (10 mL×3). The organic phase was washed with brine (10 mL×2), dried over anhydrous Na₂SO₄, concentrated under reduced pressure and purified by prep-HPLC to afford the title compound (16 mg, yield: 8.39%) as a white solid.

1H NMR (400 MHz, DMSO) □ 9.75 (s, 1H), 8.07 (d, J=8.80 Hz, 2H), 7.95 (d, J=4.77 Hz, 1H), 7.86 (s, 1H), 7.77 (d, J=8.56 Hz, 2H), 7.16-7.57 (m, 6H), 4.20 (s, 2H), 3.98 (t, J=5.01 Hz, 2H), 3.72 (t, J=5.01 Hz, 2H). ESI MS [M+H]⁺: 452.1

### Example 5

### 7-[[3-(3-fluoro-4-methoxy-phenyl)imidazo[1,2-a]pyrazin-8-yl]amino]-1,3,4,5-tetrahydro-1-benzazepin-2-one hydrochloride

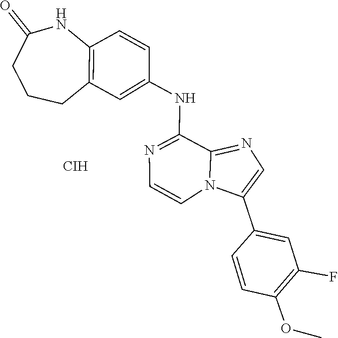 [SMILES: COc1ccc(-c2cnc3c(Nc4ccc5c(c4)CCCC(=O)N5)nccn23)cc1F ; "CIH"]

### 6-aminotetralin-1-one oxime

### Step 1) 6-aminotetralin-1-one oxime

A mixture of 6-amino-1,2,3,4-tetrahydronaphthalen-1-one (1.0 g, 6.2 mmol), hydroxylamine hydrochloride (474 mg, 6.82 mmol) and sodium acetate (1.12 g, 13.65 mmol) in ethanol (10 mL) and water (3.3 mL) was stirred at 90° C. for 4 h. The mixture was cooled to room temperature. water (20 mL) was added to the mixture, which was filtered, and the filter cake was washed with water (5 mL×2), then dried under high vacuum to give the title compound (2, 880 mg, yield: 78.08%) as a white solid. ESI MS [M+H]⁺: 177.1

### Step 2) 7-amino-2,3,4,5-tetrahydro-2-benzazepin-1-one (3) and 7-amino-1,3,4,5-tetrahydro-1-benzazepin-2-one

A mixture of 6-aminotetralin-1-one oxime (880 mg, 4.99 mmol) in PPA (10 mL) was stirred at 120° C. for 2 h. The mixture was cooled to 90° C., then poured into ice. Then the mixture was neutralized with 4N NaOH aq. (˜20 mL) to pH=8, and extracted with EtOAc (100 mL×2). The EtOAc layer was washed with brine (30 mL), dried over Na₂SO₄ and concentrated under reduced pressure to give a mixture of 7-amino-2,3,4,5-tetrahydro-2-benzazepin-1-one (3) and 7-amino-1,3,4,5-tetrahydro-1-benzazepin-2-one (850 mg) as brown solids ESI MS [M+H]⁺: 177.1

### Step 3) 7-[[3-(3-fluoro-4-methoxy-phenyl)imidazo[1,2-a]pyrazin-8-yl]amino]-1,3,4,5-tetrahydro-1-benzazepin-2-one hydrochloride

A mixture of 8-chloro-3-(3-fluoro-4-methoxy-phenyl)imidazo[1,2-a]pyrazine (150 mg, 0.540 mmol), a mixture of 7-amino-2,3,4,5-tetrahydro-2-benzazepin-1-one and 7-amino-1,3,4,5-tetrahydro-1-benzazepin-2-one (105 mg, 0.6 mmol) in ACN (1.8 mL) and acetic acid (0.200 mL) was stirred at 90° C. for 16 h. The mixture was concentrated under reduced pressure. The residue was purified by prep-TLC (DCM:MeOH=10:1) to give a crude product, which was further purified by prep-HPLC (HCl as additive) to give the title compound (8.1 mg) as a yellow solid.

ESI MS [M+H]⁺: 418.1

¹H NMR (400 MHz, DMSO-d₆) δ ppm 10.57 (br s, 1H) 8.14 (s, 1H) 8.07 (d, J=4.77 Hz, 1H) 7.99 (br d, J=6.53 Hz, 2H) 7.91 (s, 1H) 7.59-7.67 (m, 2H) 7.48-7.57 (m, 2H) 7.36-7.45 (m, 1H) 3.94 (s, 3H) 2.97 (br d, J=5.77 Hz, 2H) 2.76 (br t, J=6.90 Hz, 2H) 1.93 (br t, J=6.65 Hz, 2H).

### Example 125

### 5-[[3-(2,3-difluoro-4-methoxy-phenyl)imidazo[1,2-a]pyrazin-8-yl]amino]indan-1-one

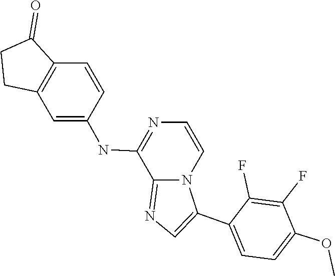

A mixture of 5-aminoindan-1-one (597.3 mg, 4.06 mmol) and 8-chloro-3-(2,3-difluoro-4-methoxy-phenyl)imidazo[1,2-a]pyrazine (1 g, 3.38 mmol) in DMF (42 mL) was stirred at 100° C. for 14 h. The mixture was concentrated and the residue was treated with EA (5 mL). The mixture was stirred at 25° C. for 5 min and the filter cake was purified by prep-HPLC to afford the title compound (227 mg) as a brown solid.

¹H NMR (400 MHz, DMSO) δ 10.90-10.13 (brs, 1H), 8.47 (s, 1H), 8.07 (dd, J=8.5, 1.6 Hz, 1H), 7.90-7.85 (m, 2H), 7.63-7.57 (m, 2H), 7.50-7.43 (m, 1H), 7.25 (t, J=7.8 Hz, 1H), 3.98 (s, 2H), 3.14-3.05 (m, 1H), 2.65-2.56 (m, 1H). ESI MS [M+H]⁺: 407.1

### Example 6

### N1-(2-aminoethyl)-N5-[3-(2,3-difluoro-4-methoxy-phenyl)imidazo[1,2-a]pyrazin-8-yl]indane-1,5-diamine formic acid

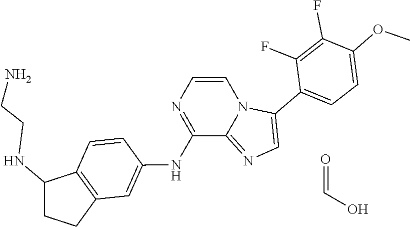 [SMILES: COc1ccc(-c2cnc3c(Nc4ccc5c(c4)CCC5NCCN)nccn23)c(F)c1F ; O=CO]

### Step 1) tert-butyl N-[2-[[5-[[3-(2,3-difluoro-4-methoxy-phenyl)imidazo[1,2-a]pyrazin-8-yl]amino]indan-1-yl]amino]ethyl]carbamate

A mixture of 5-[[3-(2,3-difluoro-4-methoxy-phenyl)imidazo[1,2-a]pyrazin-8-yl]amino]indan-1-one (156 mg, 0.384 mmol), tetraethyl titanate (131 mg, 0.576 mmol) and tert-butyl N-(2-aminoethyl)carbamate (123 mg, 0.768 mmol) in THF was stirred at 70° C. for 14 h. The mixture was cooled to rt and then sodium borohydride (29.05 mg, 0.768 mmol) and MeOH were added. The mixture was stirred at rt for another 1 h, then NaHCO₃ aq. (10 mL) was added. The resulting mixture was extracted with EtOAc (10 ml×3). The combined organic layers were dried over Na₂SO₄ and concentrated to afford the title compound (376 mg) as a black gum. ESI MS [M+H]⁺: 551.3

### Step 2) N1-(2-aminoethyl)-N5-[3-(2,3-difluoro-4-methoxy-phenyl)imidazo[1,2-a]pyrazin-8-yl]indane-1,5-diamine formic acid

A mixture of tert-butyl N-[2-[[5-[[3-(2,3-difluoro-4-methoxy-phenyl)imidazo[1,2-a]pyrazin-8-yl]amino]indan-1-yl]amino]ethyl]carbamate (376 mg, 0.382 mmol) and hydrochloric acid in EtOAc (2 M, 1 mL) in EtOAc (5 mL) was stirred at rt for 3 h. The mixture was concentrated. The residue was partitioned between EtOAc and NaHCO₃ aq. (10 mL). The combined organic layers were concentrated and the residue was purified by pre-HPLC to afford the title compound (17.5 mg) as a white solid. ¹H NMR (400 MHz, DMSO) δ 9.57-9.50 (brs, 1H), 8.39 (s, 1H), 7.98 (s, 1H), 7.85-7.74 (m, 2H), 7.72 (dd, J=4.7, 2.6 Hz, 1H), 7.51-7.39 (m, 2H), 7.32 (d, J=8.3 Hz, 1H), 7.24 (t, J=7.7 Hz, 1H), 4.14 (t, J=6.5 Hz, 1H), 3.97 (s, 4H), 2.98-2.92 (m, 1H), 2.87-2.80 (m, 52), 2.79-2.73 (m, 2H), 2.35-2.28 (m, 1H), 1.84-1.74 (m, 1H). ESI MS [M+H]⁺: 449.2

### Example 7

### N-[4-[[3-[4-(difluoromethoxy)phenyl]imidazo[1,2-a]pyrazin-8-yl]amino]phenyl]acetamide

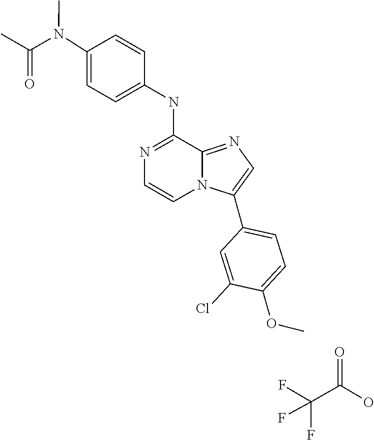

### Step 1) 8-chloro-3-(3-chloro-4-methoxy-phenyl)imidazo[1,2-a]pyrazine

A solution of 3-bromo-8-chloro-imidazo[1,2-a]pyrazine (785.0 mg, 2.8 mmol), (3-chloro-4-methoxy-phenyl)boronic acid (626.3 mg, 3.36 mmol), Pd(dppf)Cl₂ (205.4 mg, 0.28 mmol) and Na₂CO₃ (593.0 mg, 5.6 mmol) in Dioxane/H₂O (20.0/4.0 mL) was degassed and filled with N₂ for three times. The mixture was then stirred at 80° C. for 16 h. It was concentrated under reduced pressure to give the residue which was purified by column chromatograph (EtOAc:PE=1:3˜1:1) to give the title compound as a brown solid (300.0 mg, 36.4%). ESI MS [M+H]⁺: 294.1

### Step 2) N-[4-[[3-[4-(difluoromethoxy)phenyl]imidazo[1,2-a]pyrazin-8-yl]amino]phenyl]acetamide

A mixture of 8-chloro-3-(3-chloro-4-methoxy-phenyl)imidazo[1,2-a]pyrazine (29.4 mg, 0.1 mmol), IA (24.6 mg, 0.15 mmol), Binap (9.3 mg, 0.015 mmol), t-BuONa (19.2 mg, 0.2 mmol) and Pd₂(dba)₃ (9.1 mg, 0.01 mmol) in Toluene (5.0 mL) was degassed and filled with N₂ for three times. Then the mixture was stirred at 100° C. for 16h. It was purified by Prep-HPLC (Basic conditions) followed by another Prep-HPLC (TFA) to yield the title compound (2.0 mg, 4.7%) as a yellow solid. ESI MS [M+H]⁺: 422.2

The following compounds were prepared b analogy

| | | | ESI MS | starting |
|---|---|---|---|---|
| Example | Name | Structure | [M + H]⁺ | material |
| 8 | N-[4-[[3-[4- (difluoromethoxy) phenyl]imidazo [1,2-a]pyrazin-8- yl]amino]phenyl] acetamide | 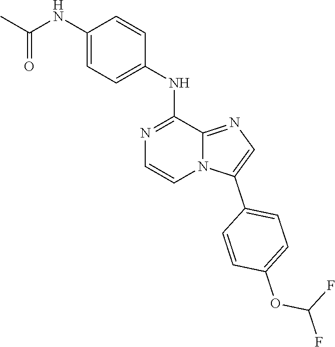 [SMILES: CC(=O)Nc1ccc(Nc2nccn3c(-c4ccc(OC(F)F)cc4)cnc23)cc1] | 410.0 | N-(4- aminophenyl) acetamide and 8-chloro-3- [4- (difluoro- methoxy)phenyl] imidazo [1,2-a]pyrazine |
| 9 | 1-[4-[[3-(3-chloro- 4-methoxy- phenyl)imidazo [1,2-a]pyrazin-8- yl]amino]phenyl] pyrrolidin-2-one; 2,2,2- trifluoroacetic acid | 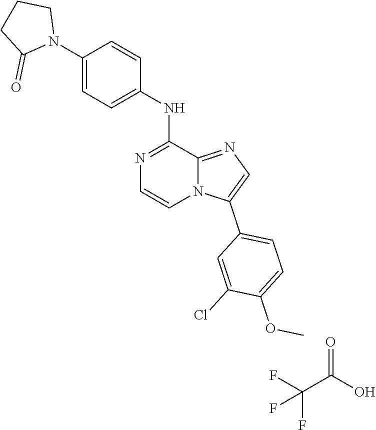 | 434.1 | 1-(4- aminophenyl) pyrrolidin- 2-one and 8-chloro-3- (3-chloro-4- methoxy- phenyl) imidazo [1,2-a]pyrazine |
| 10 | N-[4-[[3-(3- chloro-4-methoxy- phenyl)imidazo [1,2-a]pyrazin-8- yl]amino]phenyl]- 2-hydroxy- acetamide; 2,2,2- trifluoroacetic acid | 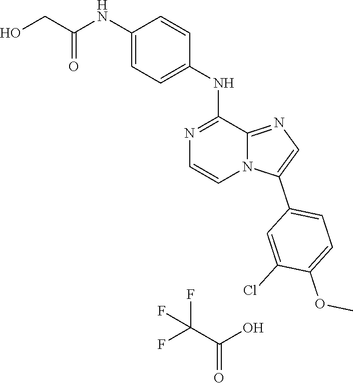 | 424.2 | N-(4- aminophenyl)- 2-methoxy- acetamide and 8-chloro-3- (3-chloro-4- methoxy- phenyl) imidazo[1,2- a]pyrazine followed by methyl ester hydrolysis |
| 11 | N-[4-[[3-(3- chloro-4-methoxy- phenyl)imidazo [1,2-a]pyrazin-8- yl]amino]-2- methyl- phenyl]acetamide; 2,2,2- trifluoroacetic acid | 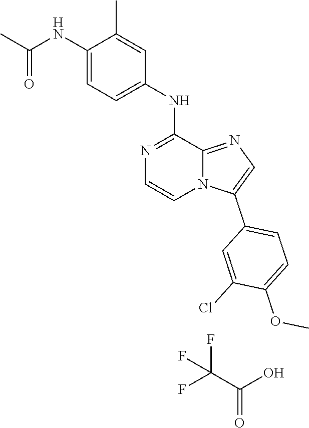 | 422.3 | N-(4-amino- 2-methyl- phenyl) acetamide and 8-chloro-3- (3-chloro-4- methoxy- phenyl) imidazo[1,2- a]pyrazine |
| 12 | N-[4-[[3-(3- fluoro-4-methoxy- phenyl)imidazo [1,2-a]pyrazin-8- yl]amino]phenyl]- 2-hydroxy- acetamide; 2,2,2- trifluoroacetic acid | 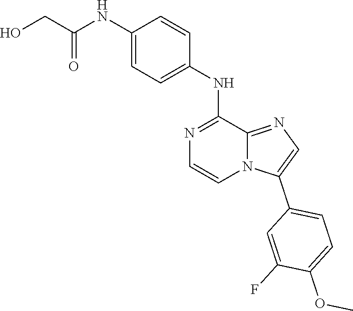 [SMILES: COc1ccc(-c2cnc3c(Nc4ccc(NC(=O)CO)cc4)nccn23)cc1F] | 408.0 | N-(4- aminophenyl)- 2-methoxy- acetamide and 8-chloro-3- (3-fluoro-4- methoxy- phenyl) imidazo[1,2- a]pyrazine followed by methyl ester hydrolysis |

### Example 13

### 2-[2-(2-aminoethoxy)ethoxy]-N-[4-[[3-(3-chloro-4-methoxy-phenyl)imidazo[1,2-a]pyrazin-8-yl]amino]phenyl]acetamide; 2,2,2-trifluoroacetic acid

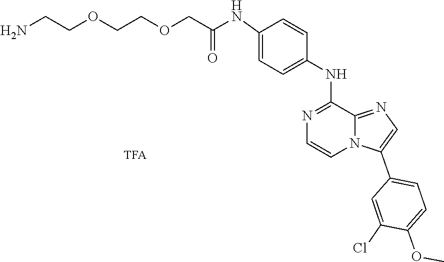 [SMILES: COc1ccc(-c2cnc3c(Nc4ccc(NC(=O)COCCOCCN)cc4)nccn23)cc1Cl]

### Step 1) tert-butyl N-[2-[2-[2-(4-aminoanilino)-2-oxo-ethoxy]ethoxy]ethyl]carbamate

A mixture of 2-[2-[2-(tert-butoxycarbonylamino)ethoxy]ethoxy]acetic acid (1.58 g, 6.0 mmol), TEA (1.21 g, 12.0 mmol) and HATU (4.56 g, 12.0 mmol) in THF (30.0 mL) was stirred at 20° C. for 0.5 h and 3B (1.94 g, 3.0 mmol) was added. The mixture was stirred at 20° C. for 15.5h. TLC (EtOAc:PE=1:2) showed new band. The mixture was concentrated to give the residue which was added water (100.0 mL) and extracted with EtOAc (200.0 mL). The organic phase was washed with brine (200.0 mL) and dried over Na₂SO₄ followed by concentration in vacuum to give the residue which was purified by column chromatograph (EtOAc:MeOH=50:1) to give 3 (2.1 g, crude) as dark green oil. ESI MS [M−99]⁺: 254.3.

### Step 2) tert-butyl N-[2-[2-[2-[4-[[3-(3-chloro-4-methoxy-phenyl)imidazo[1,2-a]pyrazin-8-yl]amino]anilino]-2-oxo-ethoxy]ethoxy]ethyl]carbamate

A mixture of tert-butyl N-[2-[2-[2-(4-aminoanilino)-2-oxo-ethoxy]ethoxy]ethyl]carbamate (294.1 mg, 1.0 mmol), 8-chloro-3-(3-chloro-4-methoxy-phenyl)imidazo[1,2-a]pyrazine (459.4 mg, 1.3 mmol), t-BuXPhos (55.1 mg, 0.13 mmol), K₂CO₃ (276.4 mg, 2.0 mmol) and Pd₂(dba)₃ (91.5 mg, 0.1 mmol) in t-BuOH (20.0 mL) was degassed and filled with N2 three times. The mixture was then stirred at 100° C. for 16h. TLC (EtOAc:MeOH=20:1) showed new spot. tert-butyl N-[2-[2-[2-(4-aminoanilino)-2-oxo-ethoxy]ethoxy]ethyl]carbamate was remaining. It was concentrated to give the residue which was purified by column chromatography (EtOAc:MeOH=50:1) to give tert-butyl N-[2-[2-[2-[4-[[3-(3-chloro-4-methoxy-phenyl)imidazo[1,2-a]pyrazin-8-yl]amino]anilino]-2-oxo-ethoxy]ethoxy]ethyl]carbamate (520.0 mg, 85.1%) as yellow gum. ESI MS [M+H]⁺: 611.3

### Step 3) 2-[2-(2-aminoethoxy)ethoxy]-N-[4-[[3-(3-chloro-4-methoxy-phenyl)imidazo[1,2-a]pyrazin-8-yl]amino]phenyl]acetamide; 2,2,2-trifluoroacetic acid

A mixture of tert-butyl N-[2-[2-[2-[4-[[3-(3-chloro-4-methoxy-phenyl)imidazo[1,2-a]pyrazin-8-yl]amino]anilino]-2-oxo-ethoxy]ethoxy]ethyl]carbamate (520.0 mg, 0.85 mmol) and TFA (2.9 g, 25.5 mmol) in CH₂Cl₂ (15.0 mL) was stirred at 20° C. for 16h. It was concentrated to give the crude product (500.0 mg, crude). 250.0 mg was purified by Prep-HPLC (TFA). The collected solution was lyophilized. After lyophilization, 2-[2-(2-aminoethoxy)ethoxy]-N-[4-[[3-(3-chloro-4-methoxy-phenyl)imidazo[1,2-a]pyrazin-8-yl]amino]phenyl]acetamide; 2,2,2-trifluoroacetic acid (60.3 mg, 13.8%) was obtained as yellow solid. ESI MS [M+H]⁺: 511.2

The following example was obtained in analogy:

| | | | ESI MS | starting |
|---|---|---|---|---|
| Example | Name | Structure | [M + H]⁺ | material |
| 14 | 2-(2- aminoethoxy)-N- [4-[[3-(3-chloro- 4-methoxy- phenyl)imidazo [1,2-a]pyrazin-8- yl]amino]phenyl] acetamide; 2,2,2- trifluoroacetic acid | 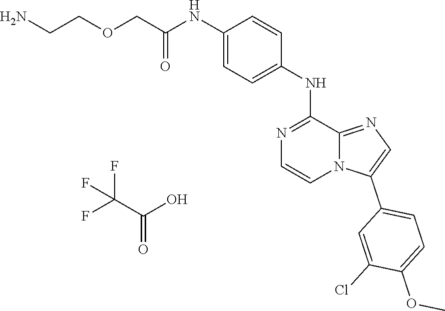 [SMILES: COc1ccc(-c2cnc3c(Nc4ccc(NC(=O)COCCN)cc4)nccn23)cc1Cl ; O=C(O)C(F)(F)F] | 467.2 | 2-[2- (tert- butoxy- carbonyl- amino) ethoxy] acetic acid |

### Example 15

### N-[4-[[3-(4-methoxyphenyl)imidazo[1,2-a]pyrazin-8-yl]amino]phenyl]acetamide

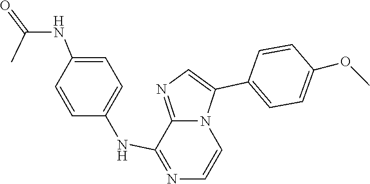 [SMILES: COc1ccc(-c2cnc3c(Nc4ccc(NC(C)=O)cc4)nccn23)cc1]

### Step 1) N-[4-(3-Bromo-imidazo[1,2-a]pyrazin-8-ylamino)-phenyl]-acetamide

To a solution of 3-bromo-8-chloro-imidazo[1,2-a]pyrazine (2 g, 8.62 mmol) and N-(4-amino-phenyl)-acetamide (2.58 g, 17.24 mmol) in NMP (10 ml) was added DIPEA (2.25 ml, 12.93 mmol) at 25° C. and the reaction mixture was stirred at 130° C. for 16h. The reaction mixture was cooled to 25° C., diluted with EtOAc and allowed to settle for 30 min. The resulting precipitate was filtered, washed with EtOAc and dried to afford N-[4-(3-bromo-imidazo[1,2-a]pyrazin-8-ylamino)-phenyl]-acetamide (1.21 g, 40%) as an off white solid ESI MS [M+H]⁺: 348.3

### Step 2) N-{4-[3-(4-Ethoxy-phenyl)-imidazo[1,2-a]pyrazin-8-ylamino]-phenyl}-acetamide

To a solution of N-[4-(3-bromo-imidazo[1,2-a]pyrazin-8-ylamino)-phenyl]-acetamide (100 mg, 0.289 mmol) in DMF and H₂O (8 ml) (3:1) at 25° C. were added (4-ethoxy-phenyl)-boronic acid (52.77 mg, 0.318 mmol), Na₂CO₃ (75.86 mg, 0.723 mmol), PPh₃ (7.57 mg, 0.02 mmol), Pd(OAc)₂ (4.53 mg, 0.02 mmol) and purged with argon for 10 min. The reaction mixture was stirred at 120° C. for 16h. The reaction mixture was cooled to 25° C., diluted with EtOAc and water and separated the organic layer. Aqueous layer was extracted with twice EtOAc. Combined organic layer was washed with brine solution and dried over Na₂SO₄. Organic layer was evaporated under reduced pressure and obtained residue was purified by prep-HPLC to afford N-{4-[3-(4-ethoxy-phenyl)-imidazo[1,2-a]pyrazin-8-ylamino]-phenyl}-acetamide (38 mg, 34%) as off white solid ESI MS [M+H]⁺: 388.0

### Example 16

### N-(4-((3-(3-fluoro-4-methoxyphenyl)imidazo[1,2-a]pyrazin-8-yl)amino)phenyl)acetamide

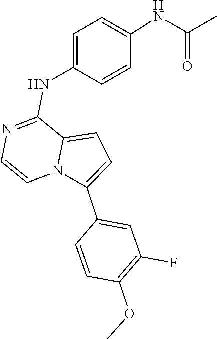 [SMILES: COc1ccc(-c2ccc3c(Nc4ccc(NC(C)=O)cc4)nccn23)cc1F]

### Step 1) N-[4-[(3-bromoimidazo[1,2-a]pyrazin-8-yl)amino]phenyl]acetamide

To 3-bromo-8-chloroimidazo[1,2-a]pyrazine (100 mg, 430 μmol, Eq: 1) in dioxane (422 μl) and Acetic Acid (422 μl) was added N-(4-amino-phenyl)-acetamide (84 mg, 559 μmol, Eq: 1.3). The reaction mixture was heated in a microwave vial to 100° C. overnight. It was then cooled to room temperature and filtered, followed by washing with diethyl ether to give a light brown powder.

ESI MS [M+H]⁺: 346.1

### Step 2) N-(4-((3-(3-fluoro-4-methoxyphenyl)imidazo[1,2-a]pyrazin-8-yl)amino)phenyl)acetamide

N-(4-((3-bromoimidazo[1,2-a]pyrazin-8-yl)amino)phenyl)acetamide (50 mg, 144 μmol, Eq: 1), 1,1′-bis(diphenylphosphino)ferrocenedichloro palladium(II) dichloromethane complex (10.6 mg, 14.4 μmol, Eq: 0.1), 3-fluoro-4-methoxyphenylboronic acid (36.8 mg, 217 μmol, Eq: 1.5) and potassium carbonate (49.9 mg, 361 μmol, Eq: 2.5) were added to Dioxane (900 μl) and water (100 μl) to form a suspension, which was degassed and shaken at 100° C. overnight. The reaction mixture was filtered over celite and purified by preparative HPLC to yield the title compound (13.9 mg, 25%). ESI MS [M+H]⁺: 392.2

### Example 17

### N-(4-((3-(3-chloro-4-methoxyphenyl)imidazo[1,2-a]pyrazin-8-yl)amino)phenyl)acetamide

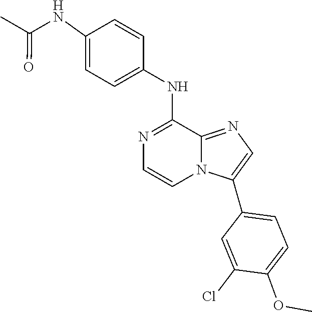 [SMILES: COc1ccc(-c2cnc3c(Nc4ccc(NC(C)=O)cc4)nccn23)cc1Cl]

### Step 1) 8-chloro-3-(3-chloro-4-methoxyphenyl)imidazo[1,2-a]pyrazine

In a 50 mL sealed tube, 8-chloro-3-iodoimidazo[1,2-a]pyrazine (2 g, 7.16 mmol, Eq: 1) was combined with Dioxane (20.3 ml) and water (10.1 ml) to give a light brown suspension. (3-chloro-4-methoxyphenyl)boronic acid (1.6 g, 8.59 mmol, Eq: 1.20), Na₂CO₃ (1.52 g, 14.3 mmol, Eq: 2.00) and 1,1′-bis(diphenylphosphino)ferrocenedichloro palladium(II) dichloromethane complex (262 mg, 358 μmol, Eq: 0.05) were added, and the reaction mixture was heated at 80° C. until completion. The reaction mixture was cooled to room temperature, diluted with AcOEt and water. Extraction with ethyl acetate. The combined organic layer was washed with brine solution, dried over Na₂SO₄ and evaporated. The obtained crude brown solid was purified by flash chromatography (silica gel, 80 g, 0% to 80% EtOAc in heptane) to yield the title compound (1.25 g, 60%). ESI MS [M+H]⁺: 294.1

### Step 2) N-(4-((3-(3-chloro-4-methoxyphenyl)imidazo[1,2-a]pyrazin-8-yl)amino)phenyl)acetamide

In a 5 mL sealed tube, 8-chloro-3-(3-chloro-4-methoxyphenyl)imidazo[1,2-a]pyrazine (60 mg, 204 μmol, Eq: 1) and N-(4-aminophenyl)acetamide (36.8 mg, 245 μmol, Eq: 1.2) were combined with acetonitrile (2 ml) to give a brown suspension. The reaction mixture was heated to 90° C. and stirred until completion. The reaction mixture was cooled to RT and the solid was collected by filtration to yield the title compound (22.4 mg, 27%). ESI MS [M+H]⁺: 408.1

### Example 18

### 1-[4-[[3-(4-methoxyphenyl)imidazo[1,2-a]pyrazin-8-yl]amino]phenyl]pyrrolidin-2-one

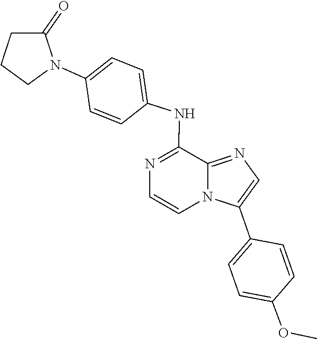 [SMILES: COc1ccc(-c2cnc3c(Nc4ccc(N5CCCC5=O)cc4)nccn23)cc1]

### Step 1) 1-[4-(3-Bromo-imidazo[1,2-a]pyrazin-8-ylamino)-phenyl]-pyrrolidin-2-one

To a solution of 3-bromo-8-chloro-imidazo[1,2-a]pyrazine (500 mg, 2.155 mmol) and 5 1-(4-amino-phenyl)-pyrrolidin-2-one (758.62 mg, 4.31 mmol) in NMP (10 ml) in sealed tube was added DIPEA (0.56 ml, 3.23 mmol) at 25° C. and the reaction mixture was stirred at 140° C. for 16h. The reaction mixture was cooled to 25° C. and total solvent was evaporated under reduced pressure. Resulting residue was washed with MeOH:DCM (5:1) to afford the title compound (160 mg, 20%) as off white solid. ESI MS [M+H]⁺: 374.0

### Step 2) 1-{4-[3-(4-Methoxy-phenyl)-imidazo[1,2-a]pyrazin-8-ylamino]-phenyl}-pyrrolidin-2-one

To a solution of 1-[4-(3-bromo-imidazo[1,2-a]pyrazin-8-ylamino)-phenyl]-pyrrolidin-2-one (150 mg, 0.403 mmol) in DMF and H₂O (8 ml) (3:1) at 25° C. were added (4-methoxy-phenyl)-boronic acid (66.97 mg, 0.444 mmol), Na₂CO₃ (105.84 mg, 1 mmol), PPh₃ (10.56 mg, 0.04 mmol) and Pd(OAc)₂ (6.32 mg, 0.028 mmol) and purged with argon for 10 min. The reaction mixture was stirred at 120° C. for 16h. The reaction mixture was cooled to 25° C., diluted with EtOAc and water and separated the organic layer. Aqueous layer was extracted twice with EtOAc. The combined organic layer was washed with brine solution and dried over Na₂SO₄. The organic layer was evaporated under reduced pressure and the obtained residue was purified by prep-HPLC to afford the title compound (70 mg, 43%) as off white solid. ESI MS [M+H]⁺: 400.0

### Example 19

### 2-Methoxy-N-{4-[3-(4-methoxy-phenyl)-imidazo[1,2-a]pyrazin-8-ylamino]-phenyl}-acetamide

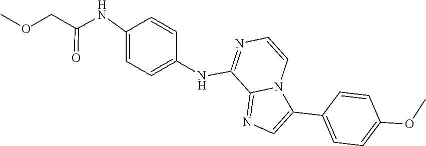

### Step 1) N-[4-(3-Bromo-imidazo[1,2-a]pyrazin-8-ylamino)-phenyl]-2-methoxy-acetamide

To a solution of 3-bromo-8-chloro-imidazo[1,2-a]pyrazine (500 mg, 2.155 mmol) and N-(4-amino-phenyl)-2-methoxy-acetamide (695.04 g, 3.233 mmol) in NMP (5 ml) in a sealed tube was added DIPEA (0.93 ml, 5.388 mmol) at 25° C. and the reaction mixture was stirred at 130° C. for 16h. The reaction mixture was cooled to 25° C., diluted with EtOAc and washed with water, brine solution dried over Na₂SO₄ and filtered. The filtrate was evaporated under reduced presser. Resulting residue was washed twice with DCM to afford the title compound (145 mg, 18%) as off white solid. ESI MS [M+H]⁺: 378.0

### Step 2) 2-Methoxy-N-{4-[3-(4-methoxy-phenyl)-imidazo[1,2-a]pyrazin-8-ylamino]-phenyl}-acetamide

To a solution of N-[4-(3-bromo-imidazo[1,2-a]pyrazin-8-ylamino)-phenyl]-2-methoxy-acetamide (140 mg, 0.372 mmol) in DMF and H₂O (8 ml) (3:1) at 25° C. were added (4-methoxy-phenyl)-boronic acid (61.84 mg, 0.41 mmol), Na₂CO₃ (97.73 mg, 0.931 mmol), PPh₃ (9.755 mg, 0.037 mmol) and Pd(OAc)₂ (5.83 mg, 0.026 mmol) and purged with argon for 10 min. The reaction mixture was stirred at 120° C. for 16h. The reaction mixture was cooled to 25° C., diluted with EtOAc and water and separated the organic layer. Aqueous layer was extracted with EtOAc twice. Combined organic layer was washed with brine solution and dried over Na₂SO₄. Organic layer was evaporated under reduced pressure and obtained residue was purified by prep-HPLC to afford 2-methoxy-N-{4-[3-(4-methoxy-phenyl)-imidazo[1,2-a]pyrazin-8-ylamino]-phenyl}-acetamide (36 mg, 24%) as off white solid. ESI MS [M+H]⁺: 404.1

### Example 20

### N-{4-[3-(4-Methoxy-phenyl)-imidazo[1,2-a]pyrazin-8-ylamino]-2-methyl-phenyl}-acetamide

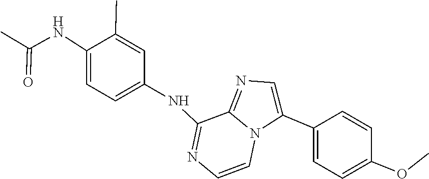 [SMILES: COc1ccc(-c2cnc3c(Nc4ccc(NC(C)=O)c(C)c4)nccn23)cc1]

### Step 1) N-[4-(3-Bromo-imidazo[1,2-a]pyrazin-8-ylamino)-2-methyl-phenyl]-acetamide

To a solution of 3-bromo-8-chloro-imidazo[1,2-a]pyrazine (500 mg, 2.155 mmol) and N-(4-Amino-2-methyl-phenyl)-acetamide (706.89 mg, 4.31 mmol) in NMP (5 ml) in sealed a tube was added DIPEA (0.56 ml, 3.23 mmol) at 25° C. and the reaction mixture was stirred at 140° C. for 16h. The reaction mixture was cooled to 25° C., diluted with EtOAc and washed with water, brine solution dried over Na₂SO₄ filtered. The filtrate was evaporated under reduced pressure. The resulting residue was purified by column chromatography (100-200) over silica gel to afford the title compound (95 mg, 12%) as brown solid. ESI MS [M+H]⁺: 360.2

### Step 2) N-{4-[3-(4-Methoxy-phenyl)-imidazo[1,2-a]pyrazin-8-ylamino]-2-methyl-phenyl}-acetamide

To a solution of N-[4-(3-bromo-imidazo[1,2-a]pyrazin-8-ylamino)-2-methyl-phenyl]-acetamide (90 mg, 0.25 mmol) in DMF and H₂O (8 ml) (3:1) at 25° C. were added (4-methoxy-phenyl)-boronic acid (41.52 mg, 0.275 mmol), Na₂CO₃ (65.62 mg, 0.625 mmol), PPh₃ (6.56 mg, 0.025 mmol) and Pd(OAc)₂ (3.92 mg, 0.07 mmol) and purged with argon for 10 min. The reaction mixture was stirred at 120° C. for 16h. The reaction mixture was cooled to 25° C., diluted with EtOAc and water and separated the organic layer. Aqueous layer was extracted with EtOAc twice. Combined organic layer was washed with brine solution and dried over Na₂SO₄. Organic layer was evaporated under reduced pressure and obtained residue was purified by prep-HPLC to afford the title compound (23 mg, 24%) as off white solid. ESI MS [M+H]⁺: 388.1

### Intermediate of Type I

### (4-bromo-2-methylphenyl)(imino)(methyl)-l6-sulfanone

To a brown solution of (4-bromo-2-methylphenyl)(methyl)sulfane (4.4 g, 20.3 mmol, Eq: 1) in methanol (40.5 ml) were added successively (diacetoxyiodo)benzene (16.3 g, 50.7 mmol, Eq: 2.5) in portions (endothermic, temp from 21° C.->17° C.) and ammonium carbamate (3.16 g, 40.5 mmol, Eq: 2) (brown yellow suspension). The reaction mixture was stirred at room temperature. After 5 min strong gas evolution, almost all in solution, strong exothermia (temp up to 32° C.). The temperature was regulated to 20° C. with an ice bath and stirring at this temperature continued. After 4 hours (yellow solution) the reaction mixture was concentrated in vacuo. The residue was treated with heptane and dichloromethane. The resulting suspension was filtered and washed with dichloromethane. The resulting solution was purified by silica gel chromatography to yield the title as yellow viscous oil with an assumed purity of 85% (total UV). It was used without further purification. ESI MS [M+H]⁺: 250.0.

Another intermediate of type 1 were prepared in analogy

| | | | ESI MS | starting |
|---|---|---|---|---|
| Intermediate | Name | Structure | [M + H]⁺ | material |
| 1 | (4-bromo-2- chlorophenyl) (methyl)(methylimino)- 16-sulfanone | 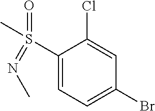 | 467.2 | (4-bromo-2- chlorophenyl) (methyl) sulfane |

### Example 21

### (+)-(4-((3-(2,3-difluoro-4-methoxyphenyl)imidazo[1,2-a]pyrazin-8-yl)amino)-2-methylphenyl)(methyl)(methylimino)-l6-sulfanone (8.9 mg, 18.9 μmol, 29.1% yield)

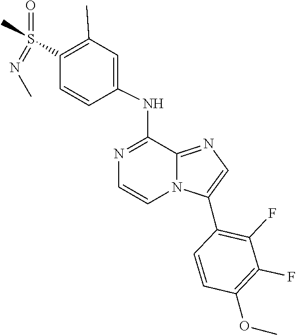

### Step 1) (+)-(4-bromo-2-methylphenyl)(imino)(methyl)-l6-sulfanone

A racemic mixture of (4-bromo-2-methylphenyl)(imino)(methyl)-l6-sulfanone was separated by chiral SFC chromatography to yield the title compound (458 mg, 1.74 mmol, 86.1% yield) as a brown oil. ESI MS [M+H]⁺: 250.0

### Step 2) (+)-(4-bromo-2-methylphenyl)(methyl)(methylimino)-l6-sulfanone

To a solution of (+)-(4-bromo-2-methylphenyl)(imino)(methyl)-l6-sulfanone (100 mg, 403 μmol, Eq: 1) in THF (4 ml) was added sodium hydride dispersion in mineral oil (25 mg, 625 μmol, Eq: 1.55) and the reaction mixture stirred at room temperature for 30 min. Then iodomethane (200 mg, 88.2 μl, 1.41 mmol, Eq: 3.5) was added and it was stirred for 15 h over night. The reaction mixture was quenched with 5 ml water and 2 ml saturated NH₄Cl, extracted 2× with ethyl acetate and brine, dried over Na₂SO₄ and concentrated in vacuo to yield the title compound (114 mg, 370 μmol, 91.7% yield) as an orange oil. ESI MS [M+H]⁺: 264.1

### Step 3) (+)-(4-((3-(2,3-difluoro-4-methoxyphenyl)imidazo[1,2-a]pyrazin-8-yl)amino)-2-methylphenyl)(methyl)(methylimino)-l6-sulfanone (8.9 mg, 18.9 μmol, 29.1% Yield)

In a microwave tube, (+)-(4-bromo-2-methylphenyl)(methyl)(methylimino)-l6-sulfanone (20 mg, 64.8 μmol, Eq: 1), 3-(2,3-difluoro-4-methoxyphenyl)imidazo[1,2-a]pyrazin-8-amine (26.9 mg, 97.3 μmol, Eq: 1.5) and potassium phosphate tribasic (41.3 mg, 195 μmol, Eq: 3) were combined with dry dioxane (1.6 ml) to give a dark brown suspension. The reaction mixture was sparged with argon for 10 min (ultrasonic bath). Then Josiphos SL-J009-1 Pd G3 (36 mg, 38.9 μmol, Eq: 0.6) was added and the tube was sparged again for 2 min. The reaction mixture was heated to 110° C. and stirred for 32 h. The reaction mixture was poured into H2O and extracted with EtOAc (2×). The organic layers were combined, washed with brine, dried over Na₂SO₄ and concentrated in vacuo. The crude material was purified by silica gel chromatography to yield the title compound (8.9 mg, 18.9 μmol, 29.1% yield) as a light brown ESI MS [M+H]⁺: 458.3.

The following examples were obtained in analogy.

| | | | ESI MS | Starting |
|---|---|---|---|---|
| Example | Name | Structure | [M + H]⁺ | material |
| 22 | 3-(2,3-difluoro-4- methoxy-phenyl)-N- [4-(N,S- dimethylsulfonimidoyl)- 3-methyl- phenyl]imidazo[l,2- a]pyrazin-8-amine | 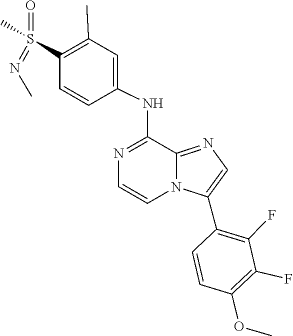 | 458.2 | (−)-(4-bromo- 2- methylphenyl) (methyl) (methylimino)- 16-sulfanone |

### Example 23

### N-[3-chloro-4-(N,S-dimethylsulfonimidoyl)phenyl]-3-(2,3-difluoro-4-methoxy-phenyl)imidazo[1,2-a]pyrazin-8-amine

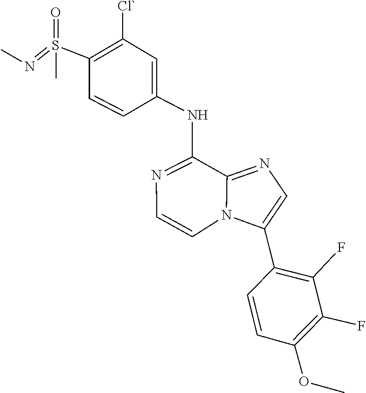

### Step 1) (4-bromo-2-chlorophenyl)(methyl)(methylimino)-l6-sulfanone

To a solution of 1 in THF (13.4 ml) was added NaH (129 mg, 3.22 mmol, Eq: 2.4) and the reaction mixture stirred at r.t. for 30 min. Then iodomethane (666 mg, 293 μl, 4.69 mmol, Eq: 3.5) was added and the reaction mixture was stirred overnight. The reaction mixture was quenched with water, extracted 3× with DCM, dried over Na₂SO₄ and concentrated in vacuo. The crude material was purified by silica gel chromatography to yield the title compound (4-bromo-2-chlorophenyl)(methyl)(methylimino)-l6-sulfanone (344 mg) along with a byproduct. The mixture was purified again by prep HPLC to yield the title compound (4-bromo-2-chlorophenyl)(methyl)(methylimino)-l6-sulfanone (149 mg, 517 μmol, 42.4% yield) as a light yellow oil. ESI MS [M+H]⁺: 284.0, along with (4-bromo-2-chlorophenyl)(ethyl)(methylimino)-l6-sulfanone (77 mg, 249 μmol, 20.5% yield) ESI MS [M+H]⁺: 296.0

### Step 2) (2-chloro-4-((3-(2,3-difluoro-4-methoxyphenyl)imidazo[1,2-a]pyrazin-s-yl)amino)phenyl)(methyl)(methylimino)-l6-sulfanone

A dark brown suspension of (4-bromo-2-chlorophenyl)(methyl)(methylimino)-l6-sulfanone (20 mg, 70.8 μmol, Eq: 1), 3-(2,3-difluoro-4-methoxyphenyl)imidazo[1,2-a]pyrazin-8-amine (29.3 mg, 106 μmol, Eq: 1.5) and potassium phosphate tribasic (45.1 mg, 212 μmol, Eq: 3) in dry dioxane (1.74 ml) in a pressure tube was sparged with argon for 5 minutes while sonicating the vessel in an ultrasonic bath. Then Josiphos SL-J009-1 Pd G3 (9.81 mg, 10.6 μmol, Eq: 0.15) was added (dark brown suspension) and degassing continued for 3 minutes. The tube was sealed and stirred at 110° C. for 4 hours. The reaction mixture was diluted with ethyl acetate and water. The mixture was extracted 2× with ethyl acetate and the organic layers were washed 1× with brine. The combined organic layers were dried with sodium sulfate, filtered and concentrated in vacuo. The crude material (drypack on silica gel) was purified by silica gel chromatography, then by preparative reversed phase HPLC to yield the title compound (19.8 mg, 38.5 μmol, 54.4% yield) as a white solid. ESI MS [M+H]⁺: 478.3

### Example 24

### (2-chloro-4-((3-(2,3-difluoro-4-methoxyphenyl)imidazo[1,2-a]pyrazin-s-yl)amino)phenyl)(ethyl)(methylimino)-l6-sulfanone

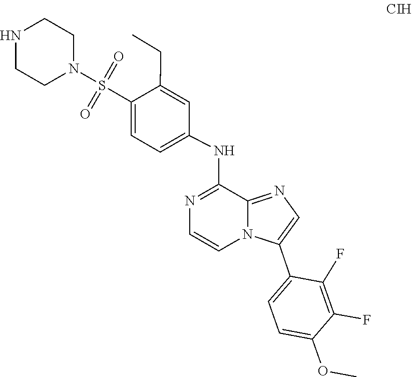 [SMILES: CCc1cc(Nc2nccn3c(-c4ccc(OC)c(F)c4F)cnc23)ccc1S(=O)(=O)N1CCNCC1]

A dark brown suspension of (4-bromo-2-chlorophenyl)(ethyl)(methylimino)-l6-sulfanone (20 mg, 67.4 μmol, Eq: 1), 3-(2,3-difluoro-4-methoxyphenyl)imidazo[1,2-a]pyrazin-8-amine (27.9 mg, 101 μmol, Eq: 1.5) and potassium phosphate tribasic (42.9 mg, 202 μmol, Eq: 3) in dry dioxane (1.66 ml) in a pressure tube was sparged with argon for 5 minutes while sonicating the vessel in an ultra sonic bath. Then Josiphos SL-J009-1 Pd G3 (9.35 mg, 10.1 μmol, Eq: 0.15) was added (light brown suspension) and degassing continued for 3 minutes. The tube was sealed and stirred at 110° C. for 4 hours. The reaction mixture was diluted with ethyl acetate and water. The mixture was extracted 2× with ethyl acetate and the organic layers were washed 1× with brine. The combined organic layers were dried with sodium sulfate, filtered and concentrated in vacuo. The crude material was purified by silica gel to yield the title compound (24.9 mg, 49.1 μmol, 72.8% yield) as an off-white solid ESI MS [M+H]⁺: 492.3.

Another example was obtained in analogy:

| | | | ESI MS | Starting |
|---|---|---|---|---|
| Example | Name | Structure | [M + H]⁺ | material |
| 25 | 2-(4-(8-((4-(N,S- dimethylsulfonimidoyl)- 3-methylphenyl)amino) imidazo[1,2- a]pyrazin-3-yl)-2,3- difluorophenoxy) acetonitrile | 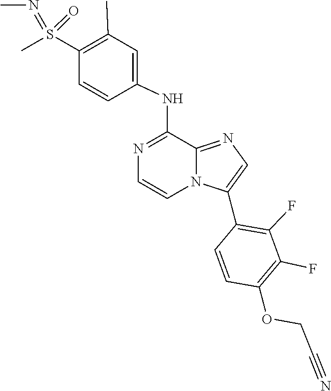 [SMILES: CN=S(C)(=O)c1ccc(Nc2nccn3c(-c4ccc(OCC#N)c(F)c4F)cnc23)cc1C] | 483.1 | (−)-(4-bromo-2- methylphenyl) (methyl) (methylimino)- 16-sulfanone and 2-(4-(8- aminoimidazo [1,2- a]pyrazin-3- yl)-2,3- difluorophenoxy) acetonitrile |

### Example 26

### (4-((3-(2,3-difluoro-4-methoxyphenyl)imidazol[1,2-a]pyrazin-4-yl)amino)-2-fluorophenyl)(methyl)(methylimino)-l6-sulfanone

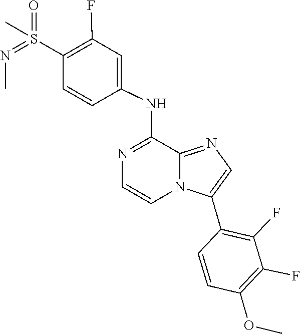 [SMILES: CN=S(C)(=O)c1ccc(Nc2nccn3c(-c4ccc(OC)c(F)c4F)cnc23)cc1F]

### Step 1) (2-fluoro-4-nitrophenyl)(imino)(methyl)-l6-sulfanone

In a 25 mL round-bottomed flask, (2-fluoro-4-nitrophenyl)(methyl)sulfane (500 mg, 2.67 mmol, Eq: 1), (diacetoxyiodo)benzene (2.15 g, 6.68 mmol, Eq: 2.5) and ammonium carbamate (417 mg, 5.34 mmol, Eq: 2) were combined with MeOH (5.34 ml) to give a red suspension. Reaction mixture stirred for 3 h at r.t. resulting a red solution. The reaction mixture was evaporated. The crude material was purified by silica gel chromatography to yield the title compound (541 mg, 2.48 mmol, 92.8% yield) as a yellow solid. ESI MS [M+H]⁺: 219.1

### Step 2) (2-fluoro-4-nitrophenyl)(methyl)(methylimino)-l6-sulfanone

To a solution of (2-fluoro-4-nitrophenyl)(imino)(methyl)-l6-sulfanone (490 mg, 2.25 mmol, Eq: 1) in THF (22.5 ml), NaH (216 mg, 5.39 mmol, Eq: 2.4) was added and the reaction mixture stirred at r.t. for 30 min. Then iodomethane (1.12 g, 491 μl, 7.86 mmol, Eq: 3.5) was added and it was stirred overnight. The reaction mixture was quenched with water, extracted 3× with DCM, dried over Na₂SO₄ and concentrated in vacuo. The crude material was purified to yield the compound (265 mg, 1.14 mmol, 50.8% yield) as a brown viscous oil ESI MS [M+H]⁺: 233.1

### Step 4) (4-amino-2-fluorophenyl)(methyl)(methylimino)-l6-sulfanone

To a heated solution of (2-fluoro-4-nitrophenyl)(methyl)(methylimino)-l6-sulfanone (260 mg, 1.12 mmol, Eq: 1) in EtOH (11.2 ml) and water (3.73 ml), ammonium chloride (192 mg, 3.58 mmol, Eq: 3.2) and iron (775 mg, 13.9 mmol, Eq: 12.4) were added and the mixture was stirred for 4 h at reflux. The reaction mixture was (still hot) filtrated over a celite pad, washed several times with EtOH and concentrated in vacuo. The crude material (drypack on Isolute) was purified by silica gel chromatography to yield the title compound (180 mg, 890 μmol, 79.5% yield) as a light red solid ESI MS [M+H]⁺: 203.1

### Step 5) (4-((3-(2,3-difluoro-4-methoxyphenyl)imidazo[1,2-a]pyrazin-8-yl)amino)-2-fluorophenyl)(methyl)(methylimino)-l6-sulfanone

A light brown suspension of (4-amino-2-fluorophenyl)(methyl)(methylimino)-l6-sulfanone (50 mg, 247 μmol, Eq: 1), 8-chloro-3-(2,3-difluoro-4-methoxyphenyl)imidazo[1,2-a]pyrazine (110 mg, 371 μmol, Eq: 1.5) and potassium phosphate tribasic (157 mg, 742 μmol, Eq: 3) in dry dioxane (6.09 ml) in a pressure tube was sparged with argon for 5 minutes while sonicating the vessel in an ultra sonic bath. Then Josiphos SL-J009-1 Pd G3 (34.3 mg, 37.1 μmol, Eq: 0.15) was added (light brown suspension) and degassing continued for 3 minutes. The tube was sealed and stirred at 110° C. over night. Additional Josiphos SL-J009-1 Pd G3 (34.3 mg, 37.1 μmol, Eq: 0.15) was added and reaction heated further at 110° C. overnight. The reaction mixture was diluted with ethyl acetate and water. The mixture was extracted 2× with ethyl acetate and the organic layers were washed 1× with brine. The combined organic layers were dried with sodium sulfate, filtered and concentrated in vacuo. The crude material (drypack on silica gel) was purified by silica gel chromatography. Then the obtained impure product was purified again by preparative reversed phase HPLC to yield the title compound (31.5 mg, 68.3 μmol, 27.6% yield) as a white solid ESI MS [M+H]⁺: 462.3

### Example 27

### (−)-((3-aminopropyl)imino)(4-((3-(2,3-difluoro-4-methoxyphenyl)imidazo[1,2-a]pyrazin-8-yl)amino)-2-methylphenyl)(methyl)-l6-sulfanone

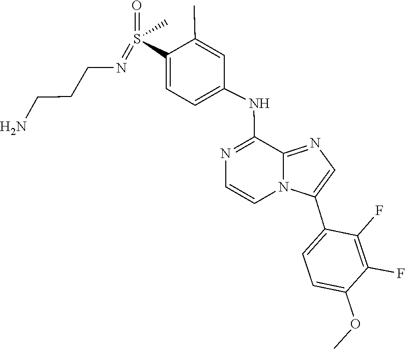 [SMILES: COc1ccc(-c2cnc3c(Nc4ccc([S@](C)(=O)=NCCCN)c(C)c4)nccn23)c(F)c1F]

### Step 1) tert-butyl (−)-(3-(((4-bromo-2-methylphenyl)(methyl)oxo)-l6-sulfaneylidene)amino)propyl)carbamate

In a microwave tube, (−)-(4-bromo-2-methylphenyl)(imino)(methyl)-l6-sulfanone (100 mg, 403 μmol, Eq: 1) and cesium carbonate (1.31 g, 4.03 mmol, Eq: 10) were combined with DMF (2 ml) to give an orange suspension. Then tert-butyl (3-bromopropyl)carbamate (960 mg, 4.03 mmol, Eq: 10) was added. The reaction mixture was heated to 70° C. and stirred for 3 d. LC-MS: product peak detected, reaction stopped at 50% conversion. The reaction mixture was poured into H₂O and extracted with EtOAc (2×). The organic layers were washed with brine, dried over Na₂SO₄ and concentrated in vacuo. The crude material was purified by silica gel chromatography. Then the impure product was purified again by preparative reverse phase HPLC to yield the title compound (58.7 mg, 143 μmol, 35.6% yield) as a colorless oil ESI MS [M+H]⁺: 407.2

### Step 2) tert-butyl (−)-(3-(((4-((3-(2,3-difluoro-4-methoxyphenyl)imidazo[1,2-a]pyrazin-8-yl)amino)-2-methylphenyl)(methyl)oxo)-l6-sulfaneylidene)amino)propyl)carbamate

In a microwave tube, tert-butyl (−)-(3-(((4-bromo-2-methylphenyl)(methyl)(oxo)-l6-sulfaneylidene)amino)propyl)carbamate (58.7 mg, 145 μmol, Eq: 1), 3-(2,3-difluoro-4-methoxyphenyl)imidazo[1,2-a]pyrazin-8-amine (60 mg, 217 μmol, Eq: 1.5) and potassium phosphate (92.2 mg, 434 μmol, Eq: 3) were combined with dry 1,4-dioxane (3.6 ml) to give a light brown suspension. The reaction mixture was sparged with argon for 10 min (ultrasonic bath). Then Josiphos SL-J009-1 Pd G3 (56.2 mg, 101 μmol, Eq: 0.7) was added and the tube was sparged again for 2 min. The reaction mixture was heated to 110° C. and stirred for 67 h. The reaction mixture was poured into H2O and extracted with EtOAc (2×). The organic layers were combined, washed with saturated_NaCl, dried over Na₂SO₄ and concentrated in vacuo. The crude material was purified by silica gel chromatography to yield the title compound (45.1 mg, 67.6 μmol, 46.7% yield) as an orange solid ESI MS [M+H]⁺: 601.4

### Step 3) (−)-((3-aminopropyl)imino)(4-((3-(2,3-difluoro-4-methoxyphenyl)imidazo[1,2-a]pyrazin-8-yl)amino)-2-methylphenyl)(methyl)-l6-sulfanone

In a 25 mL round-bottomed flask, tert-butyl (−)-(3-(((4-((3-(2,3-difluoro-4-methoxyphenyl)imidazo[1,2-a]pyrazin-8-yl)amino)-2-methylphenyl)(methyl)(oxo)-l6-sulfaneylidene)amino)propyl)carbamate (45.1 mg, 67.6 μmol, Eq: 1) was combined with 1,4-dioxane (338 μl) to give a yellow suspension. Then HCl in 1,4-dioxane 4 M (760 μl, 3.04 mmol, Eq: 45) was added. The reaction mixture was stirred for 2 h at rt. The crude reaction mixture was concentrated in vacuo. The crude material (drypack on amine silica gel) was purified by amine silica gel chromatography to yield the title compound (23.1 mg, 44.8 μmol, 66.2% yield) as a yellow solid ESI MS [M+H]⁺: 501.3

One example was obtained in analogy

| | | | ESI MS | Starting |
|---|---|---|---|---|
| Example | Name | Structure | [M + H]⁺ | material |
| 28 | (+)-((3-aminopropyl) imino) (4-((3-(2,3- difluoro-4- methoxyphenyl) imidazo[1,2- a]pyrazin-8- yl)amino)-2- methylphenyl) (methyl)-16- sulfanone | 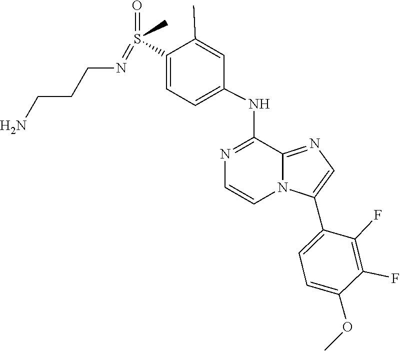 [SMILES: COc1ccc(-c2cnc3c(Nc4ccc([S@@](C)(=O)=NCCCN)c(C)c4)nccn23)c(F)c1F] | 501.3 | (+)-(4- bromo-2- methylphenyl) (imino) (methyl)-16- sulfanone |

### Example 29

### (2S,4R)—N-(3-(((−)-(4-((3-(2,3-difluoro-4-methoxyphenyl)imidazo[1,2-a]pyrazin-s-yl)amino)-2-methylphenyl)(methyl)(oxo)-l6-sulfaneylidene)amino)propyl)-4-hydroxypyrrolidine-2-carboxamide

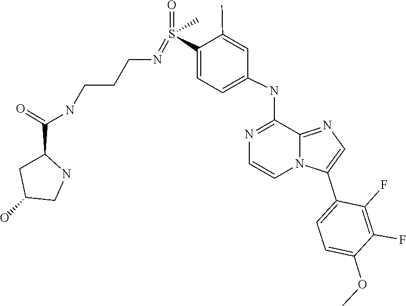

### Step 1) tert-butyl (2S,4R)-2-((3-(((−)-(4-((3-(2,3-difluoro-4-methoxyphenyl)imidazo[1,2-a]pyrazin-8-yl)amino)-2-methylphenyl)(methyl)oxo)-l6-sulfaneylidene)amino)propyl)carbamoyl)-4-hydroxypyrrolidine-1-carboxylate

In a microwave tube, (2S,4R)-1-(tert-butoxycarbonyl)-4-hydroxypyrrolidine-2-carboxylic acid (6.81 mg, 29.4 μmol, Eq: 1.1), HATU (12.7 mg, 33.5 μmol, Eq: 1.25) and (−)-((3-aminopropyl)imino)(4-((3-(2,3-difluoro-4-methoxyphenyl)imidazo[1,2-a]pyrazin-8-yl)amino)-2-methylphenyl)(methyl)-l6-sulfanone (13.4 mg, 26.8 μmol, Eq: 1) were combined with DMF dry (134 μl). Then DIPEA (19 mg, 25.7 μl, 147 μmol, Eq: 5.5) was added to give a yellow solution. The reaction mixture was stirred for 1 h at rt. The crude reaction mixture was concentrated in vacuo. The crude material was purified by silica gel chromatography to yield the title compound (12.1 mg, 14.1 μmol, 52.6% yield) as a white solid ESI MS [M+H]⁺: 714.4

### Step 2) (2S,4R)—N-(3-(((−)-(4-((3-(2,3-difluoro-4-methoxyphenyl)imidazo[1,2-a]pyrazin-8-yl)amino)-2-methylphenyl)(methyl)oxo)-l6-sulfaneylidene)amino)propyl)-4-hydroxypyrrolidine-2-carboxamide

In a 25 mL round-bottomed flask, tert-butyl (2S,4R)-2-((3-(((−)-(4-((3-(2,3-difluoro-4-methoxyphenyl)imidazo[1,2-a]pyrazin-8-yl)amino)-2-methylphenyl)(methyl)(oxo)-l6-sulfaneylidene)amino)propyl)carbamoyl)-4-hydroxypyrrolidine-1-carboxylate (11.5 mg, 16.1 μmol, Eq: 1) was combined with 1,4-dioxane (80.6 μl) to give a light yellow solution. Then HCl in 1,4-dioxane 4 M (181 μl, 725 μmol, Eq: 45) was added. The reaction mixture was stirred for 90 min. The reaction mixture was diluted in MeCN/H2O 1:1 and directly lyophilized. The crude material was purified by silica gel chromatography using dichloromethane/(CH2Cl2/MeOH/NH4OH 90:10:1) as eluent. to yield the title compound (7.1 mg, 11.2 μmol, 69.7% yield) as an off-white solid ESI MS [M+H]⁺: 614.3

One example was obtained in analogy

| | | | ESI MS | Starting |
|---|---|---|---|---|
| Example | Name | Structure | [M + H]⁺ | material |
| 30 | (2S,4R)-N-(3- (((+)-(4-((3-(2,3- difluoro-4- methoxyphenyl) imidazo[1,2- a]pyrazin-8- yl)amino)-2- methylphenyl) (methyl)(oxo)-16- sulfaneylidene) amino)propyl)-4- hydroxypyrrolidine- 2-carboxamide dihydrochloride | 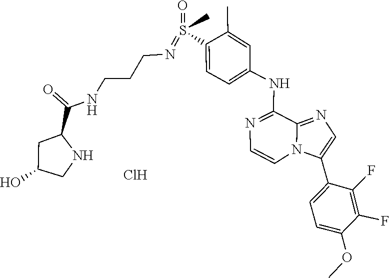 [SMILES: COc1ccc(-c2cnc3c(Nc4ccc([S@@](C)(=O)=NCCCNC(=O)[C@@H]5C[C@@H](O)CN5)c(C)c4)nccn23)c(F)c1F ; Cl] | 614.3 | (+)-((3- aminopropyl) imino) (4-((3-(2,3- difluoro-4- methoxyphenyl) imidazo [1,2- a]pyrazin-8- yl)amino)-2- methylphenyl) (methyl)- 16- sulfanone |

### Example 31

### 3-(2,3-difluoro-4-methoxy-phenyl)-N-[4-[S-(dimethylamino)-N-methyl-sulfonimidoyl]-3-methyl-phenyl]imidazo[1,2-a]pyrazin-8-amine

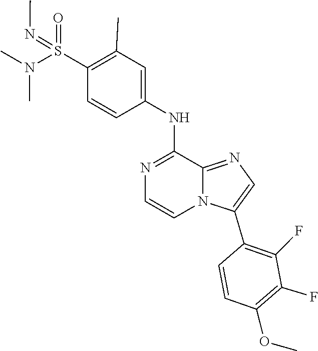 [SMILES: CN=S(=O)(c1ccc(Nc2nccn3c(-c4ccc(OC)c(F)c4F)cnc23)cc1C)N(C)C]

### Intermediate 3-(2,3-difluoro-4-methoxyphenyl)imidazo[1,2-a]pyrazin-8-amine (216.2 mg, 783 μmol, 77.1% Yield)

In a sealed pressure tube a suspension of 8-chloro-3-(2,3-difluoro-4-methoxyphenyl)imidazo[1,2-a]pyrazine (300 mg, 1.01 mmol, Eq: 1) in isopropanol (4.06 ml) and 25% aq ammonia (6.36 g, 7.06 ml, 93.3 mmol, Eq: 92) was heated to 115° C. over night. The reaction was let cooling down to RT. The reaction mixture was diluted with water, the suspension filtered and washed with water. The solid was collected and dried in vacuo to yield the title compound (216.2 mg, 783 μmol, 77.1% yield) as a dark brown solid. It was used without further purification ESI MS [M+H]⁺: 277.1

### Step 1) 4-bromo-N,N,2-trimethylbenzenesulfonimidamide

To a under argon flow dried flask a solution of 4-bromo-1-iodo-2-methylbenzene (1 g, 3.3 mmol, Eq: 1) and dry THF (15.7 ml) was cooled to −78° C. n-butyllithium in hexane (2.06 ml, 3.3 mmol, Eq: 1) was added dropwise (temperature was kept under −75° C. while adding) and the resulting yellow solution was stirred at −78° C. for 40 minutes. According to LC/MS no starting material detected, UV peak detected at the same retention time as 3-Bromotoluene. Then a solution of (tritylimino)-14-sulfanone (1.21 g, 3.96 mmol, Eq: 1.2) in dry THF (7.86 ml) was added dropwise (temperature raised to −75° C.) and stirred at −78° C. for 25 minutes. The reaction was placed in an ice bath at 0° C. After stirring for 5 minutes at 0° C., the flask was wrapped in aluminium foil and tert-butyl hypochlorite (376 mg, 392 μl, 3.47 mmol, Eq: 1.05) was added dropwise, temperature raised to 4.4° C. The mixture was stirred for 20 minutes while cooling with an ice bath. Then triethylamine (334 mg, 460 μl, 3.3 mmol, Eq: 1) and 1.4M dimethylamine in THF (3.54 ml, 4.95 mmol, Eq: 1.5) were added dropwise, temperature raised to 2.8° C. The reaction was stirred at RT overnight. Then methanesulfonic acid (1.59 g, 1.07 ml, 16.5 mmol, Eq: 5) was added dropwise while cooling with an water bath (temperature raised from 21° C. to 30° C.) and the reaction stirred vigorously for 15 minutes at RT. pH 2 was measured. The mixture was quenched with water (dropwise addition). The reaction mixture was diluted with water and TBME. The mixture was extracted 3× with TBME and the organic layers were washed 1× with brine. The combined organic layers were dried with sodium sulfate, filtered and concentrated in vacuo. According to LC-MS product peak was found in the organic phase and in the aqueous phase. The aquatic phase was basified with Na₂CO₃ (solid) to pH 11. The mixture was extracted 3× with dichloromethane. The combined organic layers were dried with sodium sulfate, filtered and concentrated in vacuo. The crude material (drypack on Isolute HM-N) was purified by silica gel chromatography to yield the title compound (322.7 mg, 1.16 mmol, 35.3% yield) as a light brown viscous oil that was used without further purification. ESI MS [M+H]⁺: 279.1

### Step 2) 4-bromo-N,N,N′,2-tetramethylbenzenesulfonimidamide

To a solution of 4-bromo-N,N,2-trimethylbenzenesulfonimidamide (25 mg, 90.2 μmol, Eq: 1) in dry THF (902 μl) was added NaH in mineral oil (8.66 mg, 216 μmol, Eq: 2.4). The reaction mixture was stirred at RT for 30 minutes. Then Mel (44.8 mg, 19.7 μl, 316 μmol, Eq: 3.5) was added and the reaction was stirred overnight. Remaining starting material left was observed by LC/MS. Additional NaH in mineral oil (3.61 mg, 90.2 μmol, Eq: 1) was added and stirring at RT continued. After 1 hour LC-MS showed no change. The reaction mixture was quenched with 25% ammonia solution, diluted with DCM and water, extracted 3× with DCM, the organic layers were combined, dried over Na₂SO₄ and concentrated in vacuo. The crude material was purified by silica gel chromatography to yield the title compound (18.7 mg, 64.2 μmol, 71.2% yield) as a colorless oil ESI MS [M+H]⁺: 293.1

### Step 3) 3-(2,3-difluoro-4-methoxy-phenyl)-N-[4-[S-(dimethylamino)-N-methyl-sulfonimidoyl]-3-methyl-phenyl]imidazo[1,2-a]pyrazin-8-amine

A dark brown suspension of 4-bromo-N,N,N′,2-tetramethylbenzenesulfonimidamide (10 mg, 34.3 μmol, Eq: 1), 3-(2,3-difluoro-4-methoxyphenyl)imidazo[1,2-a]pyrazin-8-amine (14.2 mg, 51.5 μmol, Eq: 1.5) and potassium phosphate tribasic (21.9 mg, 103 μmol, Eq: 3) in dry dioxane (846 μl) in a pressure tube was sparged with argon for 5 minutes while sonicating the vessel in an ultra sonic bath. Then Josiphos SL-J009-1 Pd G3 (4.76 mg, 5.15 μmol, Eq: 0.15) was added (dark brown suspension) and degassing continued for 3 minutes. The tube was sealed and stirred at 110° C. for 3 hours. Remaining starting material was observed by LC/MS. Additional Josiphos SL-J009-1 Pd G3 (4.76 mg, 5.15 μmol, Eq: 0.15) was added and the reaction heated to 110° C. overnight. The reaction mixture was diluted with water and ethyl acetate. The mixture was extracted 2× with ethyl acetate and the organic layers were washed 1× with brine. The combined organic layers were dried with sodium sulfate, filtered and concentrated in vacuo. The crude material was purified by silica gel chromatography to yield the title compound (13.1 mg, 26.1 μmol, 76.1% yield) as a light brown solid ESI MS [M+H]⁺: 487.4

### Example 32

### rac-(2R,4S)—N-(3-(((4-((3-(2,3-difluoro-4-methoxyphenyl)imidazo[1,2-a]pyrazin-8-yl)amino)-2-methylphenyl)(methyl)oxo)-l6-sulfaneylidene)amino)propyl)-4-hydroxypyrrolidine-2-carboxamide dihydrochloride

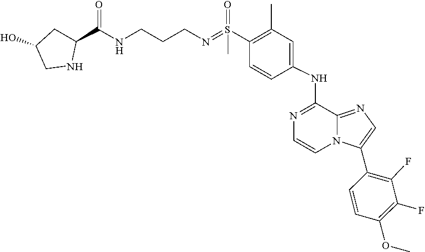 [SMILES: COc1ccc(-c2cnc3c(Nc4ccc(S(C)(=O)=NCCCNC(=O)[C@@H]5C[C@@H](O)CN5)c(C)c4)nccn23)c(F)c1F]

### Step 1) imino-methyl-(2-methyl-4-nitro-phenyl)-oxo-λ\hat{ }{6}-sulfane

To a stirred solution of 2-methyl-1-methylsulfinyl-4-nitro-benzene (2.4 g, 12.04 mmol, 1 eq) in Eaton's reagent (20.0 mL, 12.04 mmol, 1 eq) was added sodium azide (3.36 g, 51.68 mmol, 4.29 eq) at 60° C. and stirred for 0.5h. The mixture was poured into a NH₄OH solution (50 mL) and extracted with ethyl acetate (50 mL), washed with brine (50 mL) and concentrated to afford crude product 3.2 g as yellow solid. The crude was dissolved in Eaton's reagent (20.0 mL, 12.04 mmol, 1 eq) at 60° C., then sodium azide (5.11 g, 78.6 mmol, 6.53 eq) was added to the mixture in position and stirred for another 0.5h. LCMS showed the reaction was completed. The mixture was poured into a NH₄OH solution (50 mL) and extracted with ethyl acetate (50 mL), washed with brine (50 mL), concentrated and purified by silica column to afford the title compound (1.2 g, 5.6 mmol, 46.52% yield) as a yellow solid. ESI MS [M+H]⁺: 214.9

### Step 2) tert-butyl N-[3-[[methyl-(2-methyl-4-nitro-phenyl)-oxo-λ\hat{ }{6}-sulfanylidene]amino]propyl]carbamate

To a stirred solution of imino-methyl-(2-methyl-4-nitro-phenyl)-oxo-λ\hat{ }{6}-sulfane (0.4 g, 1.87 mmol, 1 eq) in cesium carbonate (1216.65 mg, 3.73 mmol, 2 eq) was added 3-(BOC-amino)propyl bromide (0.67 g, 2.8 mmol, 1.5 eq) at 60° C. and stirred for 0.5h. The mixture was poured into water (30 mL) and extracted with ethyl acetate (20 mL*3), washed with brine (50 mL*2), concentrated. The obtained residue was purified by silica column, then Prep-HPLC (FA) to afford the title compound (390 mg, 1.05 mmol, 56.23% yield) as a colorless oil. ESI MS [M+H]⁺: 372.0

### Step 3) tert-butyl N-[3-[[[4-[[3-(2,3-difluoro-4-methoxy-phenyl)imidazo[1,2-a]pyrazin-8-yl]amino]-2-methyl-phenyl]-methyl-oxo-λ\hat{ }{6}-sulfanylidene]amino]propyl]carbamate

A mixture of tert-butyl N-[3-[[(4-amino-2-methyl-phenyl)-methyl-oxo-λ\hat{ }{6}-sulfanylidene]amino]propyl]carbamate (200.0 mg, 0.590 mmol, 1 eq), 8-chloro-3-(2,3-difluoro-4-methoxy-phenyl)imidazo[1,2-a]pyrazine (173.17 mg, 0.590 mmol, 1 eq), cesium carbonate (572.5 mg, 1.76 mmol, 3 eq), tris(dibenzylideneacetone)dipalladium (0) (53.63 mg, 0.060 mmol, 0.100 eq) and 9,9-dimethyl-4,5-bis(diphenylphosphino)xanthene (33.89 mg, 0.060 mmol, 0.100 eq) in 1,4-dioxane (10 mL) was stirred under nitrogen atmosphere at 115° C. under microwave irradiation for 2h. The mixture was filtered and concentrated. The residue was purified by silica column to afford the title compound (195 mg, 0.320 mmol, 55.43% yield) as a light yellow solid.

ESI MS [M+H]⁺: 601.3

### Step 4) N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine; formic acid

To a mixture of tert-butyl N-[3-[[[4-[[3-(2,3-difluoro-4-methoxy-phenyl)imidazo[1,2-a]pyrazin-8-yl]amino]-2-methyl-phenyl]-methyl-oxo-λ\hat{ }{6}-sulfanylidene]amino]propyl]carbamate (195.0 mg, 0.320 mmol, 1 eq) in methanol (5 mL) was added hydrochloric acid (9.75 mL, 39 mmol, 120.14 eq) and stirred at 20° C. for 16h. The solution was concentrated and purified by Prep-HPLC (FA) to afford the title compound (64.7 mg, 0.120 mmol, 35.11% yield) as a white solid.

ESI MS [M+H]⁺: 501.1

### Step 5) rac-tert-butyl (2R,4S)-2-((3-(((4-((3-(2,3-difluoro-4-methoxyphenyl)imidazo[1,2-a]pyrazin-8-yl)amino)-2-methylphenyl)(methyl)oxo)-l6-sulfaneylidene)amino)propyl)carbamoyl)-4-hydroxypyrrolidine-1-carboxylate

To a solution of (2S,4R)-1-(tert-butoxycarbonyl)-4-hydroxypyrrolidine-2-carboxylic acid (22.9 mg, 99 μmol, Eq: 1.2) and DIPEA (42.7 mg, 57.6 μl, 330 μmol, Eq: 4) in dry DMF (367 μl) was added HATU (37.6 mg, 99 μmol, Eq: 1.2) and the mixture was stirred at RT for 10 minutes. Then a solution of ((3-aminopropyl)imino)(4-((3-(2,3-difluoro-4-methoxyphenyl)imidazo[1,2-a]pyrazin-8-yl)amino)-2-methylphenyl)(methyl)-l6-sulfanone formate (45.1 mg, 82.5 μmol, Eq: 1) in dry DMF (183 μl) was added (light yellow solution) and stirring at RT continued for 2 hours. The mixture was concentrated in vacuo. The crude material was purified by silica gel chromatography to yield the title compound (35.3 mg, 48.5 μmol, 58.7% yield) as a light yellow solid ESI MS [M+H]⁺: 714.5

### Step 6) rac-(2R,4S)—N-(3-(((4-((3-(2,3-difluoro-4-methoxyphenyl)imidazo[1,2-a]pyrazin-8-yl)amino)-2-methylphenyl)(methyl)oxo)-l6-sulfaneylidene)amino)propyl)-4-hydroxypyrrolidine-2-carboxamide dihydrochloride

To a light yellow solution of rac-tert-butyl (2R,4S)-2-((3-(((4-((3-(2,3-difluoro-4-methoxyphenyl)imidazo[1,2-a]pyrazin-8-yl)amino)-2-methylphenyl)(methyl)(oxo)-6-sulfaneylidene)amino)propyl)carbamoyl)-4-hydroxypyrrolidine-1-carboxylate (32.6 mg, 45.7 μmol, Eq: 1) in dioxane (304 μl) was added 4M HCl in dioxane (457 μl, 1.83 mmol, Eq: 40) and the reaction was stirred at RT for 1 hour. The reaction was diluted with dioxane and water, and directly lyophilized. To afford the title compound (26.3 mg, 36.8 μmol, 80.5% yield) as a light yellow solid ESI MS [M+H]: 614.2

One example was obtained in analogy

| | | | ESI MS | Starting |
|---|---|---|---|---|
| Example | Name | Structure | [M + H]⁺ | material |
| 33 | rac-(2R,4S)-N- (3-(((4-((3-(2,3- difluoro-4- methoxyphenyl) imidazo[1,2- a]pyrazin-8- yl)amino)phenyl) (methyl)(oxo)- 16- sulfaneylidene) amino)propyl)-4- hydroxypyrrolidine- 2- carboxamide dihydrochloride | 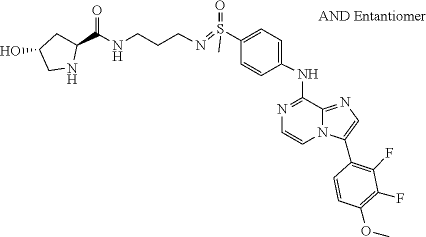 [SMILES: COc1ccc(-c2cnc3c(Nc4ccc(S(C)(=O)=NCCCNC(=O)[C@@H]5C[C@@H](O)CN5)cc4)nccn23)c(F)c1F] | 600.4 | imino- methyl- (4-nitro- phenyl)- oxo- λ^{∧}{6}- sulfane |

### Example 3

### 1-[4-[[3-[4-(difluoromethoxy)phenyl]imidazo[1,2-a]pyrazin-8-yl]amino]phenyl]imidazolidin-2-one

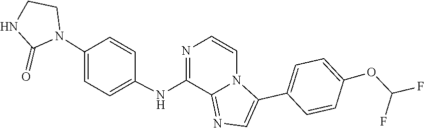 [SMILES: O=C1NCCN1c1ccc(Nc2nccn3c(-c4ccc(OC(F)F)cc4)cnc23)cc1]

A mixture of 1-(4-aminophenyl)imidazolidin-2-one (108.31 mg, 0.510 mmol, 1.5 eq) and 8-chloro-3-[4-(difluoromethoxy)phenyl]imidazo[1,2-a]pyrazine (100.0 mg, 0.340 mmol, 1 eq) in ACN (2.7 mL)/acetic acid (0.300 mL) was stirred at 100° C. for 4 h. The reaction mixture was cooled to 25° C. slowly and the formed suspension was filtered. The collected solid was dried and purified by recrystallization (DMSO/H₂O) to afford the title compound (17 mg, 0.040 mmol, 11.17% yield) as a brown solid ESI MS [M+H]⁺: 437.2

### Example 34

### N-(4-((3-(2-fluoro-4-methoxyphenyl)imidazo[1,2-a]pyrazin-8-yl)amino)phenyl)acetamide

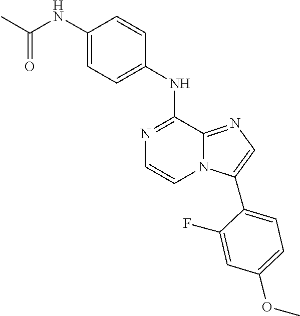 [SMILES: COc1ccc(-c2cnc3c(Nc4ccc(NC(C)=O)cc4)nccn23)c(F)c1]

### Step 1) 8-chloroimidazo[1,2-a]pyrazine

2-Bromo-1,1-diethoxyethane (17.4 mL, 115.8 mmol) was added dropwise to a 48% aqueous solution of HBr (4.5 mL, 38.6 mmol) at 5-15° C. The mixture was stirred at 80° C. for 2 h and then poured into a suspension of sodium hydrogen carbonate (74.5 g, 0.88 mol) in i-PrOH (220 mL). The mixture was stirred for a further 30 min and then filtered. To the filtrate, 3-chloropyrazin-2-amine (5.0 g, 38.7 mmol) was added, the mixture was heated to 80° C. for 4 h. The solvent was evaporated in vacuo and the obtained crude product was suspended in saturated NaHCO₃ aq. (500 mL) and extracted with DCM (100 mL*2). The combined organic phase was dried over sodium sulfate and concentrated. The crude product was triturated with MTBE (50 mL) to yield 8-chloroimidazo[1,2-a]pyrazine (4.0 g, 68%) as an off-white solid

MS [M+H]⁺: 154.1.

### Step 2) 3-bromo-8-chloroimidazo[1,2-a]pyrazine

To a stirred solution of 8-chloroimidazo[1,2-a]pyrazine (20 g, 130 mmol) in DMF (200 mL) was added NBS (23.1 g, 130 mmol) at 0° C. and the mixture was stirred at 0° C. for 1 h. water (1000 mL) was added slowly to the mixture and stirred for another 10 min. The mixture was filtered and the obtained solid was washed with MeOH (500 mL) to afford the title product (22.8 g, 75%) as a white solid. MS [M+H]+: 231.9.

### Step 3) N-(4-((3-bromoimidazo[1,2-a]pyrazin-8-yl)amino)phenyl)acetamide

To a stirred solution of 3-bromo-8-chloroimidazo[1,2-a]pyrazine (23.1 g, 0.1 mol) and DIPEA (26 mL, 0.15 mol) in NMP (115 mL) was added N-(4-aminophenyl)acetamide (30 g, 0.2 mol) at 0° C., and then the mixture was heated to 140° C. and stirred for 16 h. The mixture was diluted with EA (1.0 L), then the mixture was filtered and the obtained cake was washed with EA (500 mL) to afford the title product (30 g, 87%) as a light yellow solid.

MS [M+H]⁺: 345.9. ¹H NMR (400 MHz, DMSO-d₆) δ=9.86 (s, 1H), 9.61 (s, 1H), 7.92 (d, J=8.8 Hz, 2H), 7.80 (s, 1H), 7.77 (d, J=4.4 Hz, 1H), 7.57-7.49 (m, 3H), 2.03 (s, 3H) ppm.

### Step 4) N-(4-((3-(2-fluoro-4-methoxyphenyl)imidazo[1,2-a]pyrazin-8-yl)amino)phenyl)acetamide

To a solution of N-(4-((3-bromoimidazo[1,2-a]pyrazin-8-yl)amino) phenyl)acetamide (70 mg, 0.2 mmol), (2-fluoro-4-methoxyphenyl)boronic acid (51 mg, 0.3 mmol) and Na₂CO₃ (42 mg, 0.4 mmol) in dioxane/H₂O (4 mL, 9:1) was added Pd(dppf)Cl₂ (15 mg, 0.02 mmol) at 0° C., and then the mixture was heated to 80° C. and stirred for 16 h under nitrogen. After the completion of the reaction, the mixture was concentrated and diluted with DCM (50 mL). The solution was filtered through a thin layer of celite, the filtrate was concentrated to afford the crude product, which was recrystallized using MeOH (10 mL) to afford the title product (12 mg, 16%) as a white solid. ¹H NMR (400 MHz, CHLOROFORM-d) δ=8.01 (br s, 1H), 7.83 (d, J=8.6 Hz, 2H), 7.61 (s, 1H), 7.52 (d, J=8.8 Hz, 2H), 7.47 (d, J=4.6 Hz, 1H), 7.44-7.37 (m, 2H), 7.15 (br s, 1H), 6.89-6.79 (m, 2H), 3.90 (s, 3H), 2.19 (s, 3H) ppm.

MS [M+H]⁺: 392.1.

### Example 35

### N-(4-((6-(4-methoxyphenyl)pyrrolo[1,2-a]pyrazin-1-yl)amino)phenyl)acetamide

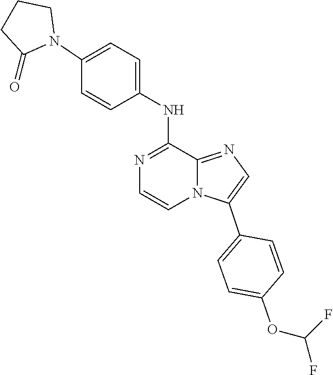 [SMILES: O=C1CCCN1c1ccc(Nc2nccn3c(-c4ccc(OC(F)F)cc4)cnc23)cc1]

### Step 1. 1-(4-((3-bromoimidazo[1,2-a]pyrazin-8-yl)amino)phenyl)pyrrolidin-2-one

A mixture of 1-(4-aminophenyl)pyrrolidin-2-one (352 mg, 2.0 mmol), 3-bromo-8-chloroimidazo[1,2-a]pyrazine (231 mg, 1.0 mmol), DIPEA (0.26 mL, 1.5 mmol) and NMP (5 mL) was stirred for 24 h at 120-130° C. under N2. The reaction mixture was cooled to room temperature and diluted with H₂O (100 mL), filtered and the obtained solid was dried in vacuum to give 1-(4-((3-bromoimidazo[1,2-a]pyrazin-8-yl)amino)phenyl)pyrrolidin-2-one (200 mg, crude) as a light brown solid. The crude product was used in the next step directly. MS [M+H]⁺: 372.0.

### Step 2. N-(4-((6-(4-methoxyphenyl)pyrrolo[1,2-a]pyrazin-1-yl)amino)phenyl)acetamide

A mixture of (4-(difluoromethoxy)phenyl)boronic acid (203 mg, 1.08 mmol), 1-(4-((3-bromoimidazo[1,2-a]pyrazin-8-yl)amino)phenyl)pyrrolidin-2-one (200 mg, 0.54 mmol), Pd(dppf)Cl₂ (19.7 mg, 0.027 mmol), Na₂CO₃ (172 mg, 1.62 mmol) and dioxane/H₂O (11 mL) was stirred for 16 h at 70-80° C. The reaction mixture was cooled to room temperature and the solvent was removed in vacuum. The residue was taken up in H₂O (20 mL) and extracted with DCM (50 mL*2). The combined organic layer was washed with H₂O (30 mL) and brine (30 mL), dried (Na₂SO₄), filtered and concentrated in vacuum to give the crude product. The crude product was purified by trituration with MeOH to give the desired product N-(4-((6-(4-methoxyphenyl)pyrrolo[1,2-a]pyrazin-1-yl)amino)phenyl)acetamide (63 mg, 26.9%) as a pale yellow solid. ¹H NMR (400 MHz, METHANOL-d₄) δ=7.81 (d, J=9.0 Hz, 2H), 7.70 (d, J=4.8 Hz, 1H), 7.61-7.52 (m, 4H), 7.50 (d, J=9.0 Hz, 1H), 7.40 (d, J=4.8 Hz, 1H), 7.25 (d, J=8.7 Hz, 2H), 6.93-6.49 (m, 1H), 3.86 (t, J=7.0 Hz, 2H), 2.54 (t, J=8.0 Hz, 2H), 2.13 (m, 2H) ppm. MS [M+H]⁺: 436.0.

### Example 36

### N-(4-((3-(3-chloro-4-methoxyphenyl)imidazo[1,2-a]pyrazin-8-yl)amino)phenyl)-2-morpholinoacetamide

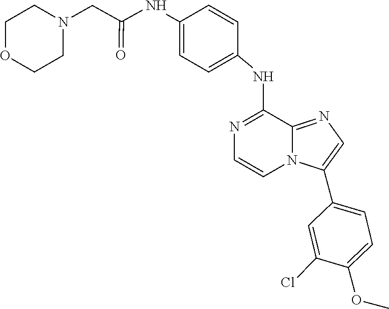 [SMILES: COc1ccc(-c2cnc3c(Nc4ccc(NC(=O)CN5CCOCC5)cc4)nccn23)cc1Cl]

### Step 1) 2-chloro-N-(4-nitrophenyl)acetamide

To an ice-cooled solution of 4-nitroaniline (2.0 g, 14.4 mmol) and TEA (2.2 g, 21.6 mmol) in DCM (30 mL) was added 2-chloroacetyl chloride (2.0 g, 17.4 mmol). The mixture was stirred at 15° C. for 2 h. The mixture was washed with 1 N HCl (50 mL), brine (50 mL), dried over sodium sulfate and concentrated to give a crude product, which was triturated with EA (20 mL) to give the title product (1.8 g, 60%) as a yellow solid.

¹H NMR (400 MHz, DMSO-d₆) δ=10.91 (s, 1H), 8.25 (d, J=8.3 Hz, 2H), 7.84 (d, J=8.2 Hz, 2H), 4.35 (s, 2H) ppm.

### Step 2) 2-morpholino-N-(4-nitrophenyl)acetamide

To a solution of 2-chloro-N-(4-nitrophenyl)acetamide (600 mg, 2.8 mmol) and morpholine (487 mg, 5.6 mmol) in DMF (10 mL) was added K₂CO₃ (772 mg, 5.6 mmol) at 15° C. The mixture was heated to 40° C. and stirred at that temperature for 2 h. The mixture was poured into water (100 mL) and extracted with DCM (50 mL*2). The combined organic layers were washed with brine (100 mL*2), dried over sodium sulfate and concentrated to give the crude product (600 mg, crude) as a yellow solid.

¹H NMR (400 MHz, CHLOROFORM-d) δ=9.46 (br s, 1H), 8.24 (d, J=8.2 Hz, 2H), 7.77 (d, J=8.2 Hz, 2H), 3.85-3.78 (m, 4H), 3.21 (s, 2H), 2.70-2.64 (m, 4H) ppm.

### Step 3) N-(4-aminophenyl)-2-morpholinoacetamide

To a solution of 2-morpholino-N-(4-nitrophenyl)acetamide (600 mg, 2.2 mmol) in MeOH (10 mL) was added Pd/C (10%, 50 mg). The mixture was hydrogenated at 15° C. under 1 atm H₂ for 15 h. The mixture was filtered and the filtrate was concentrated to give the crude product (550 mg) as a yellow solid, which was used in the next step without further purification.

¹H NMR (400 MHz, CHLOROFORM-d) δ=8.77 (br s, 1H), 7.32-7.19 (m, 2H), 6.62-6.57 (m, 2H), 3.75-3.68 (m, 4H), 3.05 (s, 2H), 2.60-2.53 (m, 4H) ppm.

### Step 4) 8-chloro-3-iodoimidazo[1,2-a]pyrazine

To a solution of 8-chloroimidazo[1,2-a]pyrazine (11 g, 71.8 mmol) in DMF (100 mL) was added NIS (16.1 g, 22.8 mmol) at 15° C. The mixture was heated to 30° C. and stirred at that temperature for 24 h. The formed precipitate was collected by filtration, the filter cake was washed with water (50 mL) and MeOH (30 mL), then dried in vacuo to give the title product (14 g) as a white solid.

MS [M+H]⁺: 279.7.

### Step 5) 8-chloro-3-(3-chloro-4-methoxyphenyl)imidazo[1,2-a]pyrazine

A mixture of 8-chloro-3-iodoimidazo[1,2-a]pyrazine (700 mg, 2.52 mmol), (3-chloro-4-methoxyphenyl)boronic acid (700 mg, 3.77 mmol), Na₂CO₃ (530 mg, 5.04 mmol) and Pd(dppf)Cl₂ (183 mg, 0.25 mmol) in dioxane/H₂ (20 mL) was heated to 80° C. and stirred for 15 h under nitrogen. The mixture was diluted with water (50 mL) and extracted with EA (50 mL*2). The combined organic layer was washed with brine (100 mL), dried over sodium sulfate and concentrated to give a residue, which was purified by silica gel chromatography eluting with PE:EA=3:1 to 1:1 to afford the title product (600 mg, 81.3%) as a brown solid. MS [M+H]⁺: 293.9.

### Step 6) N-(4-((3-(3-chloro-4-methoxyphenyl)imidazo[1,2-a]pyrazin-8-yl)amino)phenyl)-2-morpholinoacetamide

A mixture of 8-chloro-3-(3-chloro-4-methoxyphenyl)imidazo[1,2-a]pyrazine (80 mg, 0.27 mmol), N-(4-aminophenyl)-2-morpholinoacetamide (89 mg, 0.41 mmol), K₂CO₃ (74 mg, 0.54 mmol), t-Bu-Xphos (5.5 mg, 0.013 mmol) and Pd₂(dba)₃ (2.7 mg, 0.003 mmol) in t-BuOH (3 mL) was heated to 100° C. and stirred for 48 h. The mixture was diluted with water (50 mL) and extracted with EA (50 mL*2). The obtained organic layer was washed with brine (50 mL), dried over sodium sulfate and concentrated. The residue was purified by prep-HPLC (FA) to give the title product (26 mg, 19%) as a white solid. ¹H NMR (400 MHz, CHLOROFORM-d) δ=9.05 (s, 1H), 8.25 (s, 1H), 7.87 (d, J=8.9 Hz, 2H), 7.64 (d, J=4.8 Hz, 1H), 7.63-7.56 (m, 4H), 7.51 (d, J=4.8 Hz, 1H), 7.43 (dd, J=2.1, 8.5 Hz, 1H), 7.10 (d, J=8.5 Hz, 1H), 4.00 (s, 3H), 3.85-3.76 (m, 4H), 3.17 (s, 2H), 2.68-2.62 (m, 4H) ppm. MS [M+H]⁺: 493.1.

### Example 37

### N-(4-((3-(4-methoxyphenyl)imidazo[1,2-a]pyrazin-8-yl)amino)-2-methylphenyl)-2-(piperazin-1-yl)acetamide

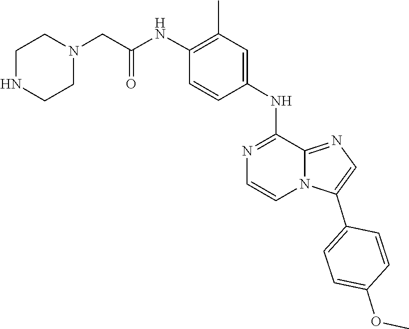 [SMILES: COc1ccc(-c2cnc3c(Nc4ccc(NC(=O)CN5CCNCC5)c(C)c4)nccn23)cc1]

### Step 1. tert-butyl 4-(2-((2-methyl-4-nitrophenyl)amino)-2-oxoethyl)piperazine-1-carboxylate

To a solution of 2-chloro-N-(2-methyl-4-nitrophenyl)acetamide (600 mg, 2.6 mmol) and tert-butyl piperazine-1-carboxylate (538 mg, 2.9 mmol) in DMF (10 mL) was added K₂CO₃ (717 mg, 5.2 mmol) at 5-15° C. The mixture was stirred at 40° C. for 2 h. The mixture was poured into water (100 mL), extracted with EA (50 mL×2), washed with brine (100 mL×2), dried over sodium sulfate and concentrated to afford tert-butyl 4-(2-((2-methyl-4-nitrophenyl)amino)-2-oxoethyl)piperazine-1-carboxylate (650 mg, 76.5%) as a yellow solid.

¹H NMR (400 MHz, CHLOROFORM-d) δ=9.67 (br. s., 1H), 8.56 (d, J=8.9 Hz, 1H), 8.16-8.11 (m, 2H), 3.55 (br. s., 4H), 3.27 (s, 2H), 2.66 (t, J=4.7 Hz, 4H), 2.40 (s, 3H), 1.50 (s, 9H) ppm.

### Step 2. tert-butyl 4-(2-((4-amino-2-methylphenyl)amino)-2-oxoethyl)piperazine-1-carboxylate

To a solution of tert-butyl 4-(2-((2-methyl-4-nitrophenyl)amino)-2-oxoethyl)piperazine-1-carboxylate (550 mg, 1.4 mmol) was added palladium 10% on Carbon (50 mg, 10% wt) at 5-15° C. The mixture was hydrogenated at 15° C. under 1 atm for 15 h. The mixture was filtered by Celite and the filtrate was concentrated to give 450 mg crude product as a yellow solid, which was used in the next step without further purification.

¹H NMR (400 MHz, CHLOROFORM-d) δ=8.88 (s, 1H), 7.65 (d, J=8.5 Hz, 1H), 6.60-6.50 (m, 2H), 3.55-3.43 (m, 6H), 3.16 (s, 2H), 2.59 (t, J=4.7 Hz, 4H), 2.18 (s, 3H), 1.47 (s, 9H) ppm.

### Step 3. tert-butyl 4-(2-((4-((3-(4-methoxyphenyl)imidazo[1,2-a]pyrazin-8-yl)amino)-2-methylphenyl)amino)-2-oxoethyl)piperazine-1-carboxylate

A mixture of 8-chloro-3-(4-methoxyphenyl)imidazo[1,2-a]pyrazine (80 mg, 0.3 mmol), tert-butyl 4-(2-((4-amino-2-methylphenyl)amino)-2-oxoethyl)piperazine-1-carboxylate (156 mg, 0.45 mmol), K₂CO₃ (83 mg, 0.6 mmol), t-Bu-Xphos (5.5 mg, 0.015) and Pd₂(dba)₃ (2.7 mg, 0.003 mmol) in t-BuOH (5 mL) was heated 100° C. for 3 days under nitrogen. The mixture was diluted with water (50 mL), extracted with EA (50 mL*2), washed with brine, dried over sodium sulfate and concentrated. the residue was purified by prep-HPLC (FA) to afford tert-butyl 4-(2-((4-((3-(4-methoxyphenyl)imidazo[1,2-a]pyrazin-8-yl)amino)-2-methylphenyl)amino)-2-oxoethyl)piperazine-1-carboxylate (55 mg, 32%) as a yellow solid.

MS [M+H]⁺: 572.3.

### N-(4-((3-(4-methoxyphenyl)imidazo[1,2-a]pyrazin-8-yl)amino)-2-methylphenyl)-2-(piperazin-1-yl)acetamide

To a solution of tert-butyl 4-(2-((4-((3-(4-methoxyphenyl)imidazo[1,2-a]pyrazin-8-yl)amino)-2-methylphenyl)amino)-2-oxoethyl)piperazine-1-carboxylate (55 mg, 0.096 mmol) in MeOH (2 mL) was added HCl/MeOH (2 mL). The resulting mixture was stirred at 5-15° C. for 15 h. The precipitate was collected by filtration and washed with MeOH (5 mL). The solid was re-dissolved in water (30 mL) and lyophilized to afford N-(4-((3-(4-methoxyphenyl)imidazo[1,2-a]pyrazin-8-yl)amino)-2-methylphenyl)-2-(piperazin-1-yl)acetamide (11.0 mg, 24%, HCl salt) product as a yellow solid

¹H NMR (400 MHz, DMSO-d₆) δ=10.11 (br. s., 1H), 9.72 (br. s., 2H), 8.07 (s, 1H), 7.96 (d, J=4.8 Hz, 1H), 7.86-7.72 (m, 2H), 7.64 (d, J=8.8 Hz, 2H), 7.55-7.42 (m, 2H), 7.18 (d, J=8.8 Hz, 2H), 3.86 (s, 3H), 3.75 (br. s., 4H), 3.53 (br. s., 4H), 3.43 (br. s., 2H), 2.28 (s, 3H) ppm.

MS [M+H]⁺: 472.2.

### Example 38

### N-(2-ethyl-4-((3-(4-methoxyphenyl)imidazo[1,2-a]pyrazin-8-yl)amino)phenyl)acetamide

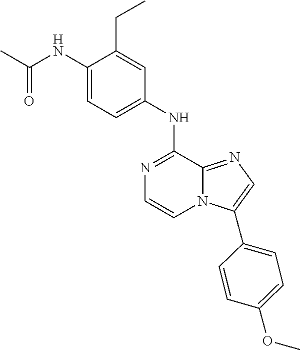 [SMILES: CCc1cc(Nc2nccn3c(-c4ccc(OC)cc4)cnc23)ccc1NC(C)=O]

### Step 1) N-(2-ethylphenyl)acetamide

A solution of 2-ethylaniline (2.42 g, 20.0 mmol) in Ac₂O (20 mL) was stirred at 16° C. for 12 h. The reaction was quenched with H₂O (100 mL) and extracted with EA (50 mL*3). The combined organic layer was dried with anhydrous Na₂SO₄ and concentrated to give the crude product (3.3 g, crude) which was used in next step directly.

### Step 2) N-(2-ethyl-4-nitrophenyl)acetamide

To a solution of N-(2-ethylphenyl)acetamide (1.63 g, 10 mmol) in THF (5 mL) was added NaNO₂ (0.69 mg, 10 mol) and sulfuric acid (25 mL) at −5° C. Then the reaction mixture was stirred at −5° C. for 3 h. The reaction was quenched with H₂O (50 mL) and extracted with EA (50 mL*3). The combined organic layer was washed with 1 N NaOH (30 mL*3), H₂O (50 mL*3) and brine (50 mL), dried with anhydrous Na₂SO₄ and concentrated to give the crude product, which was purified by column chromatography (DCM:MeOH=20:1) to give the product N-(2-ethyl-4-nitrophenyl)acetamide (600 mg, 30%) as a yellow solid. MS [M+H]⁺: 209.1.

### Step 3) N-(4-amino-2-ethylphenyl)acetamide

To a solution of N-(2-ethyl-4-nitrophenyl)acetamide (624 mg, 3.0 mmol) in MeOH (20 mL) was added wet Pd/C (10%, 63 mg), and the mixture was stirred under H₂ at 16° C. for 12 h. The reaction mixture was filtered and concentrated to give the crude product (400 mg, crude) which was used in the next step directly.

MS [M+H]⁺: 179.1.

### Step 4) 8-chloro-3-(4-methoxyphenyl)imidazo[1,2-a]pyrazine

To a solution of 8-chloro-3-iodoimidazo[1,2-a]pyrazine (882 mg, 3.0 mmol) in dioxane/H₂O (11 mL, 10:1) was added (4-methoxyphenyl)boronic acid (502 mg, 3.3 mmol), Pd(dppf)Cl₂ (274 mg, 0.3 mmol) and Na₂CO₃ (636 mg, 6.0 mmol). The suspension was degassed under vacuum and purged with N2 three times. Then the reaction mixture was heated to 80° C. and stirred for 12 h, then the mixture was concentrated and purified by column chromatography (PE/EA=1/1) to give the title product (450 mg, 57%) as a light red solid MS [M+H]⁺: 260.0.

### Step 5) N-(2-ethyl-4-((3-(4-methoxyphenyl)imidazo[1,2-a]pyrazin-8-yl)amino)phenyl)acetamide

To a solution of 8-chloro-3-(4-methoxyphenyl)imidazo[1,2-a]pyrazine (98 mg, 0.38 mmol) in t-BuOH (10 mL) was added N-(4-amino-2-ethylphenyl)acetamide (76 mg, 0.43 mmol), Pd₂(dba)₃ (4.0 mg, 0.0038 mmol), t-Bu-XPhos (8.0 mg, 0.02 mmol) and K₂CO₃ (63 mg, 0.46 mmol). The suspension was degassed under vacuum and purged with N2 three times. Then the reaction mixture was heated to 120° C. and stirred at that temperature for 72 h, and the mixture was concentrated to give the crude product which was purified by prep-HPLC (TFA) to give the title product (18.1 mg, 11.8%) as a yellow solid. MS [M+H]⁺: 402.1 ¹H NMR (400 MHz, METHANOL-d₄) δ=8.02-7.79 (m, 2H), 7.72-7.43 (m, 5H), 7.30-7.08 (m, 3H), 3.91 (s, 3H), 2.73 (d, J=7.8 Hz, 2H), 2.22 (s, 3H), 1.28 (t, J=7.5 Hz, 3H) ppm.

### Example 39

### N-(2-chloro-4-((3-(4-methoxyphenyl)imidazo[1,2-a]pyrazin-8-yl)amino)phenyl)acetamide

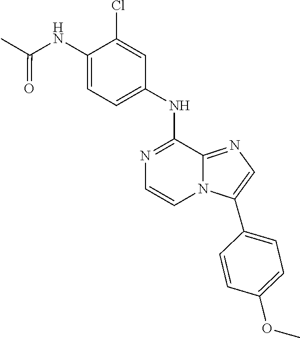 [SMILES: COc1ccc(-c2cnc3c(Nc4ccc(NC(C)=O)c(Cl)c4)nccn23)cc1]

To a solution of 8-chloro-3-(4-methoxyphenyl)imidazo[1,2-a]pyrazine (100 mg, 0.38 mmol) in t-BuOH (10 mL) was added N-(4-amino-2-chlorophenyl)acetamide (78 mg, 0.43 mmol), Pd₂(dba)₃ (4.0 mg, 0.0038 mmol), t-Bu-XPhos (8.0 mg, 0.02 mmol) and K₂CO₃ (63 mg, 0.46 mmol). The suspension was degassed under vacuum and purged with N₂ three times. Then the reaction mixture was heated to 80° C. and stirred at that temperature for 12 h, and then the mixture was concentrated and purified by prep-HPLC (FA) to give the title product (11.1 mg, 7.2%) as a white solid. MS [M+H]⁺: 408.0. ¹H NMR (400 MHz, CHLOROFORM-d) δ=8.33 (br s, 2H), 8.04 (s, 1H), 7.68 (d, J=4.2 Hz, 1H), 7.60-7.42 (m, 5H), 7.07 (d, J=8.3 Hz, 2H), 3.90 (s, 3H), 2.25 (s, 3H) ppm.

### Example 40

### N-(4-((6-(4-methoxyphenyl)pyrrolo[1,2-a]pyrazin-1-yl)amino)phenyl)acetamide

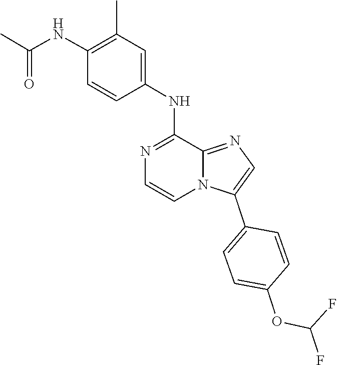 [SMILES: CC(=O)Nc1ccc(Nc2nccn3c(-c4ccc(OC(F)F)cc4)cnc23)cc1C]

### Step 1) N-(2-methyl-4-nitrophenyl)acetamide

To a solution of 2-methyl-4-nitroaniline (35.0 g, 230 mmol) in THF (500 mL) was added AcOH (50 g) and Ac₂O (35.2 g, 345 mmol) at 0° C. Then the reaction mixture was heated to 80° C. and stirred for 12 h. The reaction mixture was concentrated to give the crude product (42 g, crude) which was used in the next step directly.

### Step 2) N-(4-amino-2-methylphenyl)acetamide

To a solution of N-(2-methyl-4-nitrophenyl)acetamide (42.0 g, 201 mmol) in MeOH (500 mL) was added wet Pd/C (10%, 5.0 g) and stirred under H₂ (50 psi) at 16° C. for 12 h. The reaction mixture was filtered and the filtrate was concentrated to give the crude product (30 g, crude), which was used in next step directly.

### Step 3) N-(4-((3-bromoimidazo[1,2-a]pyrazin-8-yl)amino)-2-methylphenyl)acetamide

To a solution of 3-bromo-8-chloroimidazo[1,2-a]pyrazine (2.32 g, 10.0 mmol) in NMP (10 mL) was added N-(4-amino-2-methylphenyl)acetamide (3.0 g, 20.0 mmol) and DIPEA (1.94 g, 15.0 mmol) at 20° C. Then the reaction mixture was heated to 140° C. and stirred for 12 h, the reaction mixture was quenched with H₂O (20 mL). The mixture was filtered to give the crude product, which was recrystallized with MeOH (20 mL) to give the desired product (1.6 g, 44%) as a brown solid. ¹H NMR (400 MHz, DMSO-d₆) δ=9.57 (s, 1H), 9.25 (s, 1H), 7.95-7.63 (m, 4H), 7.56 (d, J=4.5 Hz, 1H), 7.28 (d, J=8.5 Hz, 1H), 2.19 (s, 3H), 2.05 (s, 3H) ppm MS [M+H]⁺: 360.9.

### Step 4) N-(4-((6-(4-methoxyphenyl)pyrrolo[1,2-a]pyrazin-1-yl)amino)phenyl)acetamide

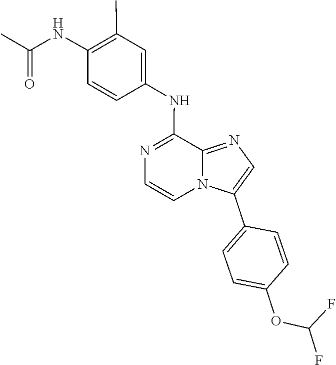 [SMILES: CC(=O)Nc1ccc(Nc2nccn3c(-c4ccc(OC(F)F)cc4)cnc23)cc1C]

A mixture of (4-(difluoromethoxy)phenyl)boronic acid (29 mg, 0.153 mmol), N-(4-((3-bromoimidazo[1,2-a]pyrazin-8-yl)amino)-2-methylphenyl)acetamide (46 mg, 0.128 mmol), Pd(dppf)Cl₂ (4.68 mg, 0.0064 mmol), Na₂CO₃ (27 mg, 0.256 mmol) and dioxane/H₂O (3.3 mL) was stirred for 16 h at 70-80° C. The reaction mixture was cooled to room temperature and the solvent was removed in vacuum. The residue was taken up in H₂O (20 mL) and extracted with DCM (30 mL*2). The combined organic layer was washed with H₂O (20 mL) and brine (20 mL), dried (Na₂SO₄), filtered and concentrated in vacuum to give crude product. The crude product was purified by prep-HPLC (TFA) to give the product N-(4-((6-(4-methoxyphenyl)pyrrolo[1,2-a]pyrazin-1-yl)amino)phenyl)acetamide (12.6 mg, 23.3%) as a pale white solid. ¹H NMR (400 MHz, DMSO-d₆) δ=9.49 (br s, 1H), 9.26 (br s, 1H), 7.92 (d, J=4.2 Hz, 1H), 7.86 (d, J=9.8 Hz, 2H), 7.83-7.74 (m, 3H), 7.49-7.23 (m, 5H), 2.20 (s, 3H), 2.05 (s, 3H) ppm. MS [M+H]⁺: 424.2.

### Example 41

### N-(4-((3-(3-chloro-4-(difluoromethoxy)phenyl)imidazo[1,2-a]pyrazin-8-yl)amino)phenyl)acetamide

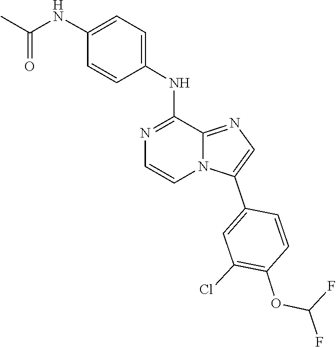 [SMILES: CC(=O)Nc1ccc(Nc2nccn3c(-c4ccc(OC(F)F)c(Cl)c4)cnc23)cc1]

### Step 1) 4-bromo-2-chloro-1-(difluoromethoxy)benzene

A mixture of sodium 2-chloro-2,2-difluoroacetate (8.0 g, 48 mmol), 4-bromo-2-chlorophenol (4.0 g, 19.2 mmol) and Cs₂CO₃ (12.4 g, 38.4 mmol) in DMF/H₂ (120 mL) was stirred for 2 h at 100° C. The reaction mixture was cooled to room temperature, diluted with H₂O (400 mL) and extracted with MTBE (100 mL*2). The combined organic layer was washed with H₂O (75 mL) and brine (75 mL*2), dried (MgSO₄), filtered and concentrated in vacuum to give crude product, which was purified by silica gel column chromatography with PE/EA=50/1 to 20/1 to give the title product 4-bromo-2-chloro-1-(difluoromethoxy)benzene (1.1 g, 22.4%) as a colorless oil. ¹H NMR (400 MHz, CHLOROFORM-d) δ=7.63 (d, J=2.3 Hz, 1H), 7.42 (dd, J=2.3, 8.7 Hz, 1H), 7.15 (d, J=8.7 Hz, 1H), 6.75-6.32 (m, 1H) ppm.

### Step 2) 2-(3-chloro-4-(difluoromethoxy)phenyl)-4,4,5,5-tetramethyl-1,3,2-dioxaborolane

A mixture of 4,4,4′,4′,5,5,5′,5′-octamethyl-2,2′-bi(1,3,2-dioxaborolane (1.1 g, 4.34 mmol), 4-bromo-2-chloro-1-(difluoromethoxy)benzene (1.0 g, 3.94 mmol), Pd(dppf)Cl₂ (144 mg, 0.197 mmol), AcOK (1.16 g, 11.82 mmol) and dioxane (20 mL) was stirred for 4.0 h at 70-80° C. under N2. The reaction mixture was cooled to room temperature and the solvent was removed in vacuum. The residue was taken up in H₂O (30 mL) and extracted with DCM (75 mL*2). The combined organic layer was washed with H₂O (50 mL) and brine (50 mL), dried (MgSO₄), filtered and concentrated in vacuum to give crude product, which was purified by silica gel column chromatography eluted with PE/EA=30/1 to 10/1 to give the product 2-(3-chloro-4-(difluoromethoxy)phenyl)-4,4,5,5-tetramethyl-1,3,2-dioxaborolane (1.05 g, 87.5%) as a colorless oil. ¹H NMR (400 MHz, CHLOROFORM-d) δ=7.91 (d, J=1.3 Hz, 1H), 7.71 (dd, J=1.4, 8.1 Hz, 1H), 7.23 (d, J=8.2 Hz, 1H), 6.80-6.34 (m, 1H), 1.36 (s, 12H) ppm.

### Step 3) N-(4-((3-(3-chloro-4-(difluoromethoxy)phenyl)imidazo[1,2-a]pyrazin-8-yl)amino)phenyl)acetamide

A mixture of 2-(3-chloro-4-(difluoromethoxy)phenyl)-4,4,5,5-tetramethyl-1,3,2-dioxaborolane (160 mg, 0.525 mmol), N-(4-((3-bromoimidazo[1,2-a]pyrazin-8-yl)amino)phenyl)acetamide (121 mg, 0.35 mmol), Pd(dppf)Cl₂ (12.8 mg, 0.0175 mmol), Na₂CO₃ (74.2 mg, 0.70 mmol) and dioxane/H₂ (8.25 mL) was stirred for 16 h at 70-80° C. The reaction mixture was cooled to room temperature and the solvent was removed in vacuum. The residue was taken up in H₂O (20 mL) and extracted with DCM (50 mL*2). The combined organic layer was washed with H₂O (30 mL) and brine (30 mL), dried (Na₂SO₄), filtered and concentrated in vacuum. The crude product was purified by prep-HPLC (TFA) to give the title product N-(4-((3-(3-chloro-4-(difluoromethoxy)phenyl)imidazo[1,2-a]pyrazin-8-yl)amino)phenyl)acetamide (96 mg, 61.9%) as a yellow solid. ¹H NMR (400 MHz, METHANOL-d₄) δ=7.79-7.74 (m, 2H), 7.71 (d, J=2.0 Hz, 1H), 7.68-7.60 (m, 4H), 7.54-7.51 (m, 1H), 7.50-7.45 (m, 1H), 7.37 (d, J=5.0 Hz, 1H), 6.98-6.58 (m, 1H), 2.15 (s, 3H) ppm. MS [M+H]⁺: 444.1.

### Example 42

### N-(4-((3-(4-chlorophenyl)imidazo[1,2-a]pyrazin-8-yl)amino)phenyl)acetamide

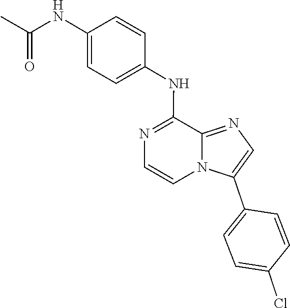 [SMILES: CC(=O)Nc1ccc(Nc2nccn3c(-c4ccc(Cl)cc4)cnc23)cc1]

To a stirred solution of N-(4-((3-bromoimidazo[1,2-a]pyrazin-8-yl)amino) phenyl)acetamide (70 mg, 0.2 mmol), (4-chlorophenyl)boronic acid (47 mg, 0.3 mmol) and Na₂CO₃ (42 mg, 0.4 mmol) in dioxane/H₂ (4 mL, 9:1) was added Pd(dppf)Cl₂ (15 mg, 0.02 mmol) at 0° C. and stirred under N2 at 80° C. for 16 h. The solution was filtered through a thin layer of celite, the filtrate was concentrated to afford crude product, which was recrystallized from MeOH (30 mL) to afford the title product (19.0 mg, 25.2%) as a white solid. ¹H NMR (400 MHz, CHLOROFORM-d) δ=7.99 (s, 1H), 7.82 (d, J=8.8 Hz, 2H), 7.67-7.61 (m, 2H), 7.56-7.47 (m, 7H), 7.13 (br s, 1H), 2.19 (s, 3H) ppm. MS [M+H]⁺: 378.0.

### Example 43

### N-(4-((3-(4-chlorophenyl)imidazo[1,2-a]pyrazin-8-yl)amino)-2-methylphenyl)acetamide

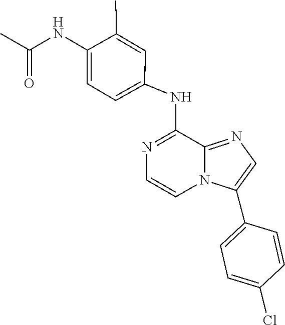 [SMILES: CC(=O)Nc1ccc(Nc2nccn3c(-c4ccc(Cl)cc4)cnc23)cc1C]

To a stirred solution of N-(4-((3-bromoimidazo[1,2-a]pyrazin-8-yl)amino)-2-methylphenyl)acetamide (108 mg, 0.3 mmol), (4-chlorophenyl)boronic acid (70 mg, 0.45 mmol) and Na₂CO₃ (64 mg, 0.6 mmol) in dioxane/H₂O (4.0 mL, 9:1) was added Pd(dppf)Cl₂ (22 mg, 0.03 mmol) at 0° C., the mixture was heated to 80° C. and stirred for 16 h. The mixture was concentrated and diluted with DCM (50 mL). The solution was filtered through a thin layer of Celite, the filtrate was concentrated to afford the crude product, which was recrystallized with MeOH (20 mL) to afford the title product (43 mg, 36.6%) as a white solid. ¹H NMR (400 MHz, CHLOROFORM-d) δ=7.98 (s, 1H), 7.78 (s, 1H), 7.74-7.61 (m, 4H), 7.5-7.54 (m, 5H), 6.94-6.82 (m, 1H), 2.32 (s, 3H), 2.22 (s, 3H) ppm. MS [M+H]⁺: 392.1.

### Example 44

### 1-(4-((3-(3-Chloro-4-methoxyphenyl)imidazo[1,2-a]pyrazin-8-yl)amino)phenyl)piperidin-2-one

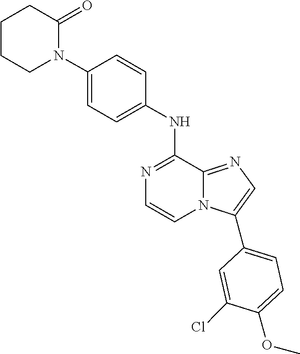 [SMILES: COc1ccc(-c2cnc3c(Nc4ccc(N5CCCCC5=O)cc4)nccn23)cc1Cl]

### Step 1) N-(3-(3-chloro-4-methoxyphenyl)imidazo[1,2-a]pyrazin-8-yl)benzene-1,4-diamine

A mixture of 8-chloro-3-(3-chloro-4-methoxyphenyl)imidazo[1,2-a]pyrazine (400 mg, 1.36 mmol), benzene-1,4-diamine (220 mg, 2.04 mmol) in MeCN (20 mL) was heated to 100° C. and stirred for 15 h. The reaction mixture was diluted with DCM (100 mL), washed with brine (100 mL), dried over sodium sulfate and concentrated to give the crude product (400 mg, crude) as a brown solid, which was used without further purification. MS [M+H]⁺: 366.0.

### Step 2) 5-bromo-N-(4-((3-(3-chloro-4-methoxyphenyl)imidazo[1,2-a]pyrazin-8-yl)amino)phenyl)pentanamide

To an ice-cooled mixture of N-(3-(3-chloro-4-methoxyphenyl)imidazo[1,2-a]pyrazin-8-yl)benzene-1,4-diamine (100 mg, 0.27 mmol), NaHCO₃ (45 mg, 0.54 mmol) in EA/H₂O (20 mL) was added 5-bromopentanoyl chloride (82 mg, 0.0.41 mmol) at 0° C. The mixture was stirred at 0° C. for 30 min. The mixture was diluted with EA (50 mL) and the organic phase was separated. The organic layer was washed with brine (50 mL), dried over sodium sulfate and concentrated to give the crude product (55 mg, crude) as brown solid, which was used directly in the next step.

MS [M+H]⁺: 530.0.

### Step 3) 1-(4-((3-(3-chloro-4-methoxyphenyl)imidazo[1,2-a]pyrazin-8-yl)amino)phenyl)piperidin-2-one

To an ice-cooled solution of 5-bromo-N-(4-((3-(3-chloro-4-methoxyphenyl)imidazo[1,2-a]pyrazin-8-yl)amino)phenyl)pentanamide (55 mg, 0.10 mmol) in THF (10 mL) was added t-BuOK (22 mg, 0.20 mmol) at 0° C. The mixture was stirred at 5-15° C. for 15 h. The mixture was diluted with water (50 mL) and extracted with EA (50 mL*2). The obtained organic layer was washed with brine (50 mL), dried over sodium sulfate and concentrated to give the crude product. The crude product was purified by prep-HPLC (FA) to give the title product (10.1 mg, 22.6%) as a white solid. ¹H NMR (400 MHz, CHLOROFORM-d) δ=8.09 (s, 1H), 7.92 (d, J=8.8 Hz, 2H), 7.67 (d, J=4.8 Hz, 1H), 7.63-7.58 (m, 2H), 7.55-7.51 (m, 1H), 7.45 (dd, J=2.1, 8.4 Hz, 1H), 7.30 (s, 2H), 7.11 (d, J=8.5 Hz, 1H), 4.01 (s, 3H), 3.67 (t, J=5.1 Hz, 2H), 2.60 (t, J=5.8 Hz, 2H), 2.02-1.95 (m, 4H) ppm. MS [M+H]⁺: 448.1.

### Example 45

### N-(4-((3-(3-chloro-4-methoxyphenyl)imidazo[1,2-a]pyrazin-8-yl)amino)-2-ethylphenyl)acetamide

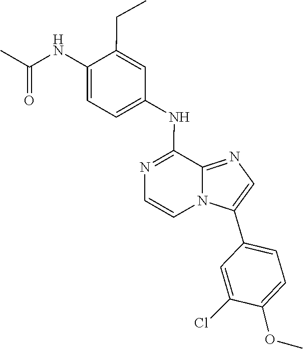 [SMILES: CCc1cc(Nc2nccn3c(-c4ccc(OC)c(Cl)c4)cnc23)ccc1NC(C)=O]

To a solution of 8-chloro-3-(3-chloro-4-methoxyphenyl)imidazo[1,2-a]pyrazine (147 mg, 0.5 mmol) in t-BuOH (10 mL) was added N-(4-amino-2-ethylphenyl)acetamide (98 mg, 0.55 mmol), Pd₂(dba)₃ (5.0 mg, 0.005 mmol), t-Bu-XPhos (106 mg, 0.025 mmol) and K₂CO₃ (83 mg, 0.6 mmol). The suspension was degassed under vacuum and purged with N2 three times at 20° C. Then the reaction mixture was heated to 120° C. and stirred for 72 h. The mixture was concentrated to give the crude product which was purified by prep-HPLC (TFA) to give the title product (8.8 mg, 4.0%) as a yellow solid. MS [M+H]⁺: 436.0. ¹H NMR (400 MHz, CHLOROFORM-d) δ=8.07 (s, 1H), 7.92-7.56 (m, 6H), 7.51 (d, J=4.4 Hz, 1H), 7.43 (d, J=8.3 Hz, 1H), 7.10 (d, J=8.3 Hz, 1H), 6.97-6.81 (m, 1H), 4.00 (s, 3H), 2.66 (d, J=7.3 Hz, 2H), 2.22 (s, 3H), 1.32-1.26 (m, 3H) ppm.

### Example 46

### N-(2-chloro-4-((3-(3-chloro-4-methoxyphenyl)imidazo[1,2-a]pyrazin-8-yl)amino)phenyl)acetamide

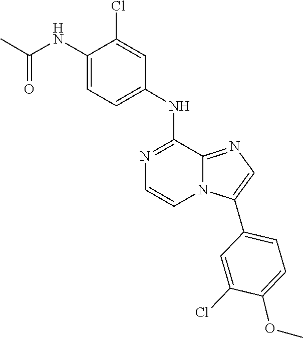 [SMILES: COc1ccc(-c2cnc3c(Nc4ccc(NC(C)=O)c(Cl)c4)nccn23)cc1Cl]

To a solution of 8-chloro-3-(3-chloro-4-methoxyphenyl)imidazo[1,2-a]pyrazine (111 mg, 0.38 mmol) in t-BuOH (10 mL) was added N-(4-amino-2-chlorophenyl)acetamide (79 mg, 0.43 mmol), Pd₂(dba)₃ (4.0 mg, 0.0038 mmol), t-Bu-XPhos (8.0 mg, 0.02 mmol) and K₂CO₃ (150 mg, 0.46 mmol). The suspension was degassed under vacuum and purged with N2 three times at 20° C. Then the reaction mixture was heated to 120° C. and stirred for 12 h. The mixture was concentrated and purified by Prep-HPLC (FA) to give the title product (7.1 mg, 4.2%) as a white solid. MS [M+H]⁺: 442.0. ¹H NMR (400 MHz, CHLOROFORM-d) δ=8.36-8.29 (m, 2H), 7.67 (d, J=4.6 Hz, 1H), 7.62 (s, 1H), 7.60-7.52 (m, 3H), 7.43 (dd, J=2.0, 8.6 Hz, 1H), 7.10 (d, J=8.6 Hz, 1H), 4.00 (s, 3H), 2.26 (s, 3H) ppm.

### Example 47

### N-(4-((3-(3-fluoro-4-methoxyphenyl)imidazo[1,2-a]pyrazin-8-yl)amino)-2-methylphenyl)acetamide

### Step 1) 8-chloro-3-(3-fluoro-4-methoxyphenyl)imidazo[1,2-a]pyrazine

A mixture of 8-chloro-3-iodo-imidazo[1,2-a]pyrazine (10.5 g, 37.57 mmol, 1 eq), 3-fluoro-4-methoxyphenylboronic acid (6.39 g, 37.57 mmol, 1 eq), [1,1′-BIS(DIPHENYLPHOSPHINO)FERROCENE]DICHLOROPALLADIUM(II) (1.37 g, 1.88 mmol, 0.050 eq) and sodium carbonate (7.96 g, 75.14 mmol, 2 eq) in 1,4-dioxane (108 mL)/water (12 mL) was stirred under N2 at 50° C. for 16h. The mixture was filtered, concentrated, and then purified by silica column chromatography (PE/EA=2:1) to afford 8-chloro-3-(3-fluoro-4-methoxy-phenyl)imidazo[1,2-a]pyrazine (3.1 g, 11.16 mmol, 29.71% yield) as a white solid.

MS [M+H]⁺: 278.0

### Step 2) N-(4-((3-(3-fluoro-4-methoxyphenyl)imidazo[1,2-a]pyrazin-8-yl)amino)-2-methylphenyl)acetamide

A mixture of 8-chloro-3-(3-fluoro-4-methoxyphenyl)imidazo[1,2-a]pyrazine (80 mg, 0.27 mmol), N-(4-amino-2-methylphenyl)acetamide (67 mg, 0.41 mmol), K₂CO₃ (74 mg, 0.54 mmol), t-Bu-Xphos (5.5 mg, 0.013 mmol) and Pd₂(dba)₃ (2.7 mg, 0.003 mmol) in t-BuOH (5 mL) was heated 100° C. and stirred for 4 d. The mixture was diluted with water (50 mL) and extracted with EA (50 mL*2). The obtained organic layer was washed with brine (50 mL), dried over sodium sulfate and concentrated to give a residue. The residue was purified by prep-HPLC (FA) to give the desired product (58.6 mg, 31.7%, HCCOH salt) as a white solid. ¹H NMR (400 MHz, DMSO-d₆) δ=9.48 (s, 1H), 9.26 (s, 1H), 8.36 (s, 1H), 7.92 (d, J=4.8 Hz, 1H), 7.88 (d, J=2.3 Hz, 1H), 7.84-7.78 (m, 2H), 7.60 (dd, J=2.1, 12.2 Hz, 1H), 7.51-7.45 (m, 2H), 7.41-7.32 (m, 1H), 7.27 (d, J=8.7 Hz, 1H), 3.93 (s, 3H), 2.20 (s, 3H), 2.05 (s, 3H) ppm. MS [M+H]⁺: 406.1.

### Example 48

### 1-(4-((3-(3-fluoro-4-methoxyphenyl)imidazo[1,2-a]pyrazin-8-yl)amino)phenyl)pyrrolidin-2-one

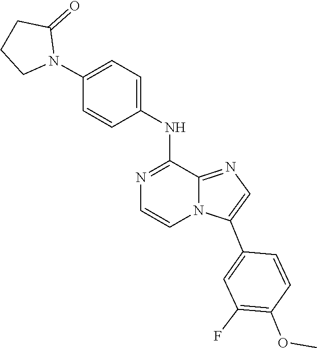 [SMILES: COc1ccc(-c2cnc3c(Nc4ccc(N5CCCC5=O)cc4)nccn23)cc1F]

A mixture of 8-chloro-3-(3-fluoro-4-methoxyphenyl)imidazo[1,2-a]pyrazine (80 mg, 0.27 mmol), 1-(4-aminophenyl)pyrrolidin-2-one (72 mg, 0.41 mmol), K₂CO₃ (74 mg, 0.54 mmol), t-Bu-Xphos (5.5 mg, 0.013 mmol) and Pd₂(dba)₃ (2.7 mg, 0.003 mmol) in t-BuOH (5 mL) was heated to 100° C. and stirred for 18 h. The mixture was diluted with water (50 mL) and extracted with EA (50 mL*2). The combined organic layer was washed with brine (50 mL), dried over sodium sulfate and concentrated. The residue was purified by prep-HPLC (FA) to give the title product (33.8 mg, 30%) as a white solid. ¹H NMR (400 MHz, DMSO-d₆) δ=9.65 (s, 1H), 8.05 (d, J=9.0 Hz, 2H), 7.94 (d, J=4.8 Hz, 1H), 7.83 (s, 1H), 7.60 (d, J=9.0 Hz, 3H), 7.53-7.44 (m, 2H), 7.41-7.32 (m, 1H), 3.93 (s, 3H), 3.84 (t, J=7.0 Hz, 2H), 2.47 (s, 2H), 2.07 (q, J=7.5 Hz, 2H) ppm. MS [M+H]⁺: 418.1.

### Example 49

### N-(4-((3-(3-chloro-4-methoxyphenyl)imidazo[1,2-a]pyrazin-8-yl)amino)-2-methylphenyl)-2-(piperazin-1-yl)acetamide

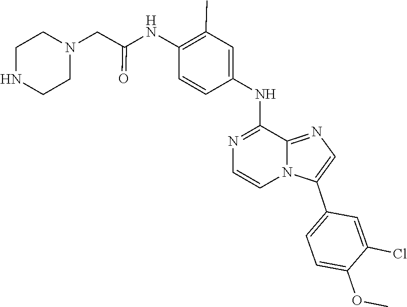 [SMILES: COc1ccc(-c2cnc3c(Nc4ccc(NC(=O)CN5CCNCC5)c(C)c4)nccn23)cc1Cl]

### Step 1) 2-chloro-N-(2-methyl-4-nitrophenyl)acetamide

To an ice-cooled solution of 2-methyl-4-nitroaniline (1.5 g, 10 mmol) and TEA (1.5 g, 15.0 mmol) in DCM (50 mL) was added 2-chloroacetyl chloride (5.5 g, 43.4 mmol). The mixture was stirred at 5-15° C. for 2 h. The mixture was washed with aq. 1 N HCl (50 mL), brine, dried over sodium sulfate and concentrated, the residue was triturated with EA (20 mL) to give 1.1 g of the title product as a brown solid. ¹H NMR (400 MHz, CHLOROFORM-d) δ=8.57 (br. s., 1H), 8.39 (d, J=8.7 Hz, 1H), 8.19-8.11 (m, 2H), 4.33-4.28 (m, 2H), 2.49-2.42 (m, 3H) ppm.

### Step 2) tert-butyl 4-(2-((2-methyl-4-nitrophenyl)amino)-2-oxoethyl)piperazine-1-carboxylate

To a solution of 2-chloro-N-(2-methyl-4-nitrophenyl)acetamide (600 mg, 2.6 mmol) and tert-butyl piperazine-1-carboxylate (538 mg, 2.9 mmol) in DMF (10 mL) was added K₂CO₃ (717 mg, 5.2 mmol) at 5-15° C. The mixture was stirred at 40° C. for 2 h. The mixture was poured into water (100 mL), extracted with EA (50 mL*2), washed with brine (100 mL*2), dried over sodium sulfate and concentrated to give 650 mg of the title product as a yellow solid. ¹H NMR (400 MHz, CHLOROFORM-d) δ=9.67 (br. s., 1H), 8.56 (d, J=8.9 Hz, 1H), 8.16-8.11 (m, 2H), 3.55 (br. s., 4H), 3.27 (s, 2H), 2.66 (t, J=4.7 Hz, 4H), 2.40 (s, 3H), 1.50 (s, 9H) ppm.

### Step 3) tert-butyl 4-(2-((4-amino-2-methylphenyl)amino)-2-oxoethyl)piperazine-1-carboxylate

To a solution of tert-butyl 4-(2-((2-methyl-4-nitrophenyl)amino)-2-oxoethyl)piperazine-1-carboxylate (550 mg, 1.4 mmol) was added Pd/C (50 mg, 5.2 mmol) at 5-15° C. The mixture was hydrogenated at 15° C. under 1 atm for 15 h. The mixture was filtered through celite and the filtrate was concentrated to give 450 mg crude product as a yellow solid, which was used in the next step without further purification. ¹H NMR (400 MHz, CHLOROFORM-d) δ=8.88 (s, 1H), 7.65 (d, J=8.5 Hz, 1H), 6.60-6.50 (m, 2H), 3.55-3.43 (m, 6H), 3.16 (s, 2H), 2.59 (t, J=4.7 Hz, 4H), 2.18 (s, 3H), 1.47 (s, 9H) ppm.

### Step 4) tert-butyl 4-(2-((4-((3-(3-chloro-4-methoxyphenyl)imidazo[1,2-a]pyrazin-8-yl)amino)-2-methylphenyl)amino)-2-oxoethyl)piperidine-1-carboxylate

A mixture of 8-chloro-3-(3-chloro-4-methoxyphenyl)imidazo[1,2-a]pyrazine (80 mg, 0.27 mmol), tert-butyl 4-(2-((4-amino-2-methylphenyl)amino)-2-oxoethyl)piperazine-1-carboxylate (142 mg, 0.41 mmol), K₂CO₃ (74 mg, 0.54 mmol), t-Bu-Xphos (5.5 mg, 0.013) and Pd₂(dba)₃ (2.7 mg, 0.003 mmol) in t-BuOH (5 mL) was heated at 100° C. for 2 d. The mixture was diluted with water (50 mL), extracted with EA (50 mL*2), washed with brine, dried over sodium sulfate and concentrated. The residue was purified by prep-HPLC (FA) to give 55 mg product as a yellow solid. MS [M+H]+: 606.2

### Step 5) N-(4-((3-(3-chloro-4-methoxyphenyl)imidazo[1,2-a]pyrazin-8-yl)amino)-2-methylphenyl)-2-(piperazin-1-yl)acetamide

To a solution of tert-butyl 4-(2-((4-((3-(3-chloro-4-methoxyphenyl)imidazo[1,2-a]pyrazin-8-yl)amino)-2-methylphenyl)amino)-2-oxoethyl)piperazine-1-carboxylate (55 mg, 0.1 mmol) in MeOH (2 mL) was added HCl/MeOH (2 mL). The resulting mixture was stirred at 5-20° C. for 15 h. The precipitate formed was collected by filtration and washed with MeOH (5 mL). The solid was re-dissolved in water (30 mL) and lyophilized to give 9.7 mg of the title product as a yellow solid ¹H NMR (400 MHz, DMSO-d6) δ=10.11 (br s, 1H), 9.71 (br s, 2H), 8.08 (s, 1H), 7.98 (d, J=4.9 Hz, 1H), 7.86-7.72 (m, 3H), 7.66 (dd, J=2.1, 8.5 Hz, 1H), 7.54-7.43 (m, 2H), 7.38 (d, J=8.8 Hz, 1H), 4.29-4.16 (m, 1H), 4.21 (br s, 1H), 3.96 (s, 3H), 3.55-3.49 (m, 8H), 3.46-3.40 (m, 1H), 3.45-3.38 (m, 1H), 3.43 (br s, 1H), 2.70-2.65 (m, 1H), 2.53 (br s, 4H), 2.28 (s, 3H) ppm.

MS [M+H]+: 506.2.

### Example 50

### N-(2-(hydroxymethyl)-4-((3-(4-methoxyphenyl)imidazo[1,2-a]pyrazin-8-yl)amino)phenyl)acetamide

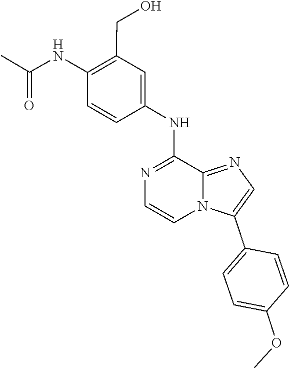 [SMILES: COc1ccc(-c2cnc3c(Nc4ccc(NC(C)=O)c(CO)c4)nccn23)cc1]

### Step 1) N-(2-(hydroxymethyl)phenyl)acetamide

To a solution of (2-aminophenyl)methanol (2.0 g, 16.3 mmol) and NaHCO₃ (2.7 g, 32.6 mmol) in EA/H2O (100 mL) was added AcCl (1.4 g, 19.5 mmol). The resulting mixture was stirred at 0° C. for 2 h. The organic phase was separated, dried over sodium sulfate and concentrated to give 1.2 g product as off-white solid, which was used in the next step without further purification. ¹H NMR (400 MHz, CHLOROFORM-d) δ=8.62 (br s, 1H), 8.00 (d, J=8.2 Hz, 1H), 7.37-7.31 (m, 1H), 7.20 (d, J=6.8 Hz, 1H), 7.13-7.08 (m, 1H), 4.69 (s, 2H), 2.80 (br s, 1H), 2.18 (s, 3H) ppm.

### Step 2) N-(2-formylphenyl)acetamide

To a solution of N-(2-(hydroxymethyl)phenyl)acetamide (6.0 g, 36.4 mmol) in DCM (200 mL) was added MnO₂ (30 g, 364 mmol). The resulting mixture was stirred at 20° C. for 15 h. The reaction mixture was filtered and concentrated to give 5.6 g crude product as a white solid ¹H NMR (400 MHz, CHLOROFORM-d) δ=11.04 (br. s., 1H), 9.83 (s, 1H), 8.65 (d, J=8.6 Hz, 1H), 7.63-7.46 (m, 2H), 7.14 (dt, J=0.9, 7.5 Hz, 1H), 2.17 (s, 3H) ppm.

### Step 3) N-(2-formyl-4-nitrophenyl)acetamide

N-(2-formylphenyl)acetamide (2.0 g, 12.2 mmol) in Ac₂O (10 mL) was added to 10 mL nitrating mixture, prepared from 5 mL conc. H₂SO₄ and 5 mL fuming HNO3 under ice cooling. The resulting mixture was stirred at 0° C. for 2 h. The reaction mixture was poured into ice water (100 mL), extracted with DCM (50 mL*2), dried over sodium sulfate and concentrated to give 1.8 g of a pale-yellow solid, which was used without further purification in the next step. ¹H NMR (400 MHz, CHLOROFORM-d) δ=11.38 (br. s., 1H), 10.01 (s, 1H), 8.93 (d, J=9.4 Hz, 1H), 8.61 (d, J=2.8 Hz, 1H), 8.43 (dd, J=2.7, 9.3 Hz, 1H), 2.32 (s, 3H) ppm.

### Step 4) N-(2-(hydroxymethyl)-4-nitrophenyl)acetamide

To an ice-cooled solution of N-(2-formyl-4-nitrophenyl)acetamide (200 mg, 1.0 mmol) in MeOH (10 mL) was added NaBH₄ (11 mg, 1.0 mmol). The resulting mixture was stirred at 0° C. for 2 h. The mixture was quenched with 1 N aq. HCl (5 mL), diluted with water (100 mL), extracted with EA (50 ml*2), washed with brine, dried over sodium sulfate and concentrated to give 155 mg title product as a yellow solid. ¹H NMR (400 MHz, CHLOROFORM-d) δ=9.11 (br. s., 1H), 8.51 (d, J=9.0 Hz, 1H), 8.23 (dd, J=2.6, 9.0 Hz, 1H), 8.08 (d, J=2.6 Hz, 1H), 4.87 (s, 2H), 2.28 (s, 3H) ppm.

### Step 5) N-(4-amino-2-(hydroxymethyl)phenyl)acetamide

To a solution of N-(2-(hydroxymethyl)-4-nitrophenyl)acetamide (155 mg, 0.74 mmol) in MeOH (10 mL) was added Pd/C (10%, 15 mg). The resulting mixture was stirred at 5-20° C. under 1 atm for 15 h. The mixture was filtered through celite and then concentrated to give 105 mg of the title product as off-white solid. ¹H NMR (400 MHz, DMSO-d6) δ=9.01 (s, 1H), 6.96 (d, J=8.3 Hz, 1H), 6.72 (d, J=2.4 Hz, 1H), 6.44 (dd, J=2.6, 8.3 Hz, 1H), 5.01 (br. s., 3H), 4.43-4.33 (m, 2H), 2.01 (s, 3H) ppm.

### Step 6) N-(2-(hydroxymethyl)-4-((3-(4-methoxyphenyl)imidazo[1,2-a]pyrazin-s-yl)amino)phenyl)acetamide

A mixture of 8-chloro-3-(4-methoxyphenyl)imidazo[1,2-a]pyrazine (80 mg, 0.3 mmol), N-(4-amino-2-(hydroxymethyl)phenyl)acetamide (81 mg, 0.45 mmol), K₂CO₃ (83 mg, 0.6 mmol), t-Bu-Xphos (5.5 mg, 0.013) and Pd₂(dba)₃ (2.7 mg, 0.003 mmol) in t-BuOH (5 mL) was heated at 100° C. for 15 h. The mixture was diluted with water (50 mL), extracted with EA (50 mL*2), washed with brine, dried over sodium sulfate and concentrated. The residue was purified flash column to give 20 mg N-(2-(hydroxymethyl)-4-((3-(4-methoxyphenyl)imidazo[1,2-a]pyrazin-8-yl)amino)phenyl)acetamide as a white solid ¹H NMR (400 MHz, DMSO-d₆) δ=9.28 (s, 1H), 8.00 (br s, 1H), 7.79 (br s, 1H), 7.89-7.76 (m, 2H), 7.77 (br s, 1H), 7.63 (d, J=8.8 Hz, 2H), 7.44-7.39 (m, 2H), 7.15 (d, J=8.8 Hz, 2H), 4.50 (s, 2H), 3.85 (s, 3H), 2.06 (s, 3H) ppm. MS [M+H]+: 404.1

### Example 51

### 2-chloro-N-(4-((3-(4-methoxyphenyl)imidazo[1,2-a]pyrazin-8-yl)amino)phenyl)acetamide

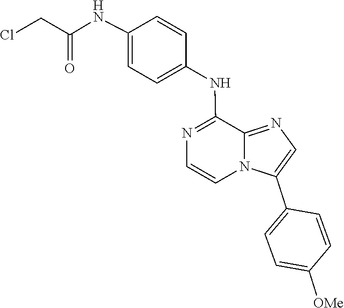 [SMILES: COc1ccc(-c2cnc3c(Nc4ccc(NC(=O)CCl)cc4)nccn23)cc1]

### Step 1) N¹-(3-(4-methoxyphenyl)imidazo[1,2-a]pyrazin-8-yl)benzene-1,4-diamine

A mixture of 3-(4-methoxyphenyl)imidazo[1,2-a]pyrazine (2.55 g, 7.7 mmol), benzene-1,4-diamine (1.0 g, 9.2 mmol) in MeCN (100 mL) was heated 100° C. for 15 h. The reaction mixture was concentrated and the residue was re-dissolved in DCM (100 mL), washed with brine, dried over sodium sulfate, concentrated to give 1.5 g product as a black solid, used directly in the next step.

### Step 2) 2-chloro-N-(4-((3-(4-methoxyphenyl)imidazo[1,2-a]pyrazin-8-yl)amino)phenyl)acetamide

To an ice-cooled mixture of N¹-(3-(4-methoxyphenyl)imidazo[1,2-a]pyrazin-8-yl)benzene-1,4-diamine (1.2 g, 3.6 mmol), NaHCO₃ (604 mg, 7.2 mmol) in EA/H₂O (200 mL) was added 2-chloroacetyl chloride (610 mg, 5.4 mmol). The mixture was stirred at 0° C. for 2 h. The organic phase was separated and washed with brine, dried over sodium sulfate and concentrated to give 800 mg crude product, 600 mg of which was purified by flash column to afford 283 mg of the title compound. MS [M+H]+: 408.1

### Example 52

### 3-(4-Methoxyphenyl)-N-(4-(methylsulfonyl)phenyl)imidazo[1,2-a]pyrazin-8-amine

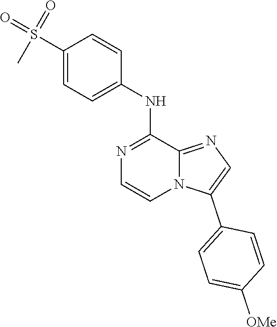 [SMILES: COc1ccc(-c2cnc3c(Nc4ccc(S(C)(=O)=O)cc4)nccn23)cc1]

A mixture of 8-chloro-3-(4-methoxyphenyl)imidazo[1,2-a]pyrazine (80 mg, 0.3 mmol), 4-(methylsulfonyl)aniline (150 mg, 0.45 mmol), K₂CO₃ (83 mg, 0.6 mmol), t-Bu-Xphos (5.5 mg, 0.013) and Pd₂(dba)₃ (2.7 mg, 0.003 mmol) in t-BuOH (5 mL) was heated 100° C. for 2 d under nitrogen. The formed precipitate was collected by filtration and then re-dissolved in DCM (100 mL), washed with water, dried over sodium sulfate and concentrated. The residue was triturated with MeOH to give 67.0 mg of the title product as an off-white solid. ¹H NMR (400 MHz, DMSO-d6) δ=10.18 (s, 1H), 8.36 (d, J=8.8 Hz, 2H), 8.00 (d, J=4.8 Hz, 1H), 7.90-7.82 (m, 3H), 7.63 (d, J=8.7 Hz, 2H), 7.54 (d, J=4.8 Hz, 1H), 7.15 (d, J=8.7 Hz, 2H), 3.85 (s, 3H), 3.18 (s, 3H) ppm. MS [M+H]+: 395.1

### Example 53

### N-(4-((3-(4-fluorophenyl)imidazo[1,2-a]pyrazin-8-yl)amino)-2-methylphenyl)acetamide

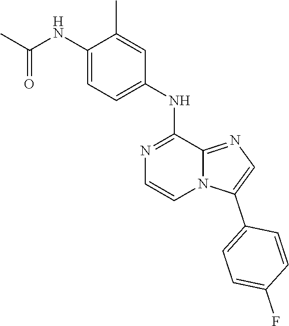 [SMILES: CC(=O)Nc1ccc(Nc2nccn3c(-c4ccc(F)cc4)cnc23)cc1C]

To a stirred solution of N-(4-((3-bromoimidazo[1,2-a]pyrazin-8-yl)amino)-2-methylphenyl)acetamide (108 mg, 0.3 mmol), (4-fluorophenyl)boronic acid (63 mg, 0.45 mmol) and Na₂CO₃ (64 mg, 0.6 mmol) in dioxane/H₂O (4.0 mL, 9:1) was added Pd(dppf)Clz (22 mg, 0.03 mmol) at 0° C., and then the mixture was heated to 80° C. and stirred for 16 h. The mixture was concentrated and diluted with DCM (50 mL). The solution was filtered through a thin layer of celite, the filtrate was concentrated to afford the crude product, which was recrystallized from MeOH (20 mL) to afford the title product (18.5 mg, 16.4%) as a white solid. ¹H NMR (400 MHz, CHLOROFORM-d) δ=8.01 (s, 1H), 7.78-7.82 (m, 2H), 7.73-7.60 (m, 4H), 7.55 (dd, J=5.4, 8.3 Hz, 2H), 7.50 (d, J=4.6 Hz, 1H), 7.26-7.22 (m, 1H), 6.90 (s, 1H), 2.32 (s, 3H), 2.23 (s, 3H) ppm. MS [M+H]+: 376.1.

### Example 54

### 1-(4-((3-(4-(difluoromethoxy)phenyl)imidazo[1,2-a] pyrazin-8-yl)amino)-2-methylphenyl)pyrrolidin-2-one

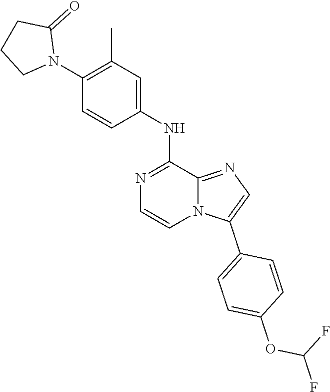 [SMILES: Cc1cc(Nc2nccn3c(-c4ccc(OC(F)F)cc4)cnc23)ccc1N1CCCC1=O]

### Step 1) 4-chloro-N-(2-methyl-4-nitrophenyl)butanamide

To an ice-cooled solution of 2-methyl-4-nitroaniline (0.5 g, 3.29 mmol) and triethylamine (0.92 mL, 6.57 mmol) in DCM (20 mL) was added 4-chlorobutanoyl chloride (0.7 g, 4.93 mmol). After addition, the reaction mixture was stirred at 25° C. for 15 h. The mixture was diluted with water (100 mL). The organic phase was separated, dried over sodium sulfate and concentrated to give 4-chloro-N-(2-methyl-4-nitro-phenyl)butanamide (0.650 g, crude) as a brown solid.

### Step 2) 1-(2-methyl-4-nitrophenyl)pyrrolidin-2-one

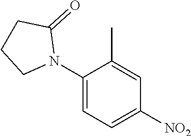 [SMILES: Cc1cc([N+](=O)[O-])ccc1N1CCCC1=O]

To an ice-cooled solution of 4-chloro-N-(2-methyl-4-nitrophenyl)butanamide (630.0 mg, 2.45 mmol) in THF (20 mL) was added potassium tert-butoxide (550.8 mg, 4.91 mmol). The resulting mixture was warmed up to 25° C. and stirred for 15 h. The mixture was quenched with 1 HCl (10 mL), extracted with EtOAc (100 mL*2). The combined organic phases were washed with brine, dried over sodium sulfate and concentrated to give 1-(2-methyl-4-nitro-phenyl)pyrrolidin-2-one (450 mg, 2.04 mmol, 83.26% yield) as a yellow solid, the crude product was used in the next step without further purification. ¹H NMR (400 MHz, CHLOROFORM-d) δ=8.48-8.37 (m, 1H), 8.17-8.03 (m, 2H), 2.51-2.49 (m, 3H), 2.09 (q, J=4.0 Hz, 1H), 1.94 (q, J=4.0 Hz, 1H), 1.65-1.55 (m, 1H), 1.17-1.13 (m, 2H), 0.99-0.97 (m, 1H) ppm.

### Step 3) 1-(4-amino-2-methylphenyl)pyrrolidin-2-one

To a solution of 1-(2-methyl-4-nitro-phenyl)pyrrolidin-2-one (400.0 mg, 1.82 mmol) in methanol (10 mL) was added palladium on carbon (40 mg, 10% wt). The resulting mixture was hydrogenated under 760 mmHg at 25° C. for 15 h. The catalyst was removed by filtration through celite and the filtrate was concentrated to afford 1-(4-amino-2-methyl-phenyl)pyrrolidin-2-one (320 mg, 1.68 mmol) ¹H NMR (400 MHz, MeOD) δ=7.28 (d, J=13.4 Hz, 0.42H), 6.96 (d, J=8.4 Hz, 0.49H), 6.62-6.51 (m, 2H), 2.20-2.15 (m, 3H), 1.82-1.76 (m, 2H), 1.06-0.83 (m, 4H) ppm.

### Step 4) 8-chloro-3-(4-(difluoromethoxy)phenyl)imidazo[1,2-a] pyrazine

A mixture of 8-chloro-3-iodoimidazo[1,2-a]pyrazine (300.0 mg, 1.07 mmol, (4-(difluoromethoxy)phenyl)boronic acid (262.27 mg, 1.4 mmol), sodium carbonate (227.55 mg, 2.15 mmol) and [1,1′-bis(diphenylphosphino)ferrocene]dichloropalladium(II) (78.55 mg, 0.110 mmol) in 1,4-dioxane (10 mL) and water (1 mL) was heated 80° C. for 15 h. The reaction mixture was concentrated and the residue was purified by silica gel chromatography eluting with PE:EA from 10:1 to 1:1 to afford 8-chloro-3-[4-(difluoromethoxy)phenyl]imidazo[1,2-a]pyrazine (305 mg, 1.03 mmol, 96.1%) as a grey solid. MS [M+H]+: 296.2.

### Step 5) 1-(4-((3-(4-(difluoromethoxy)phenyl)imidazo[1,2-a] pyrazin-8-yl)amino)-2-methylphenyl)pyrrolidin-2-one

A mixture of 1-(4-amino-2-methyl-phenyl)pyrrolidin-2-one (62.09 mg, 0.330 mmol), 8-chloro-3-[4-(difluoromethoxy)phenyl]imidazo[1,2-a]pyrazine (80.0 mg, 0.270 mmol), tris(dibenzylideneacetone)dipalladium (0) (7.47 mg, 0.010 mmol), 2-di-tert-butylphosphino-2′,4′,6′-triisopropylbiphenyl (11.55 mg, 0.030 mmol) and potassium carbonate (75.18 mg, 0.540 mmol) in tert-butanol (5 mL) was heated to 100° C. for 18 h. The reaction was concentrated to dryness and the residue was taken up in EtOAc (100 ml) and the organic layer was washed with 100 ml water then 1×100 mL brine. The organic layers were then separated and dried (Na₂SO₄) before concentration to dryness. The crude product was then purified by prep-HPLC (FA) to afford 1-[4-[[3-[4-(difluoromethoxy)phenyl]imidazo[1,2-a]pyrazin-8-yl]amino]-2-methyl-phenyl]pyrrolidin-2-one (28.5 mg, 0.060 mmol, 22.03%) as a white solid

¹H NMR (400 MHz, DMSO-d₆) δ=9.50 (d, J=3.6 Hz, 2H), 8.45 (s, 1H), 7.92 (d, J=4.8 Hz, 1H), 7.88 (d, J=2.4 Hz, 1H), 7.85 (s, 1H), 7.83-7.79 (m, 1H), 7.78-7.76 (m, 2H), 7.48-7.18 (m, 5H), 2.53-2.51 (m, 2H), 2.21 (s, 3H), 0.79-0.76 (m, 4H) ppm.

MS [M+H]+: 450.0

### Example 55

### N-(4-((3-(4-(difluoromethoxy)phenyl)imidazo[1,2-a] pyrazin-8-yl)amino)-2-methylphenyl)-2-(4-ethylpiperazin-1-yl)acetamide

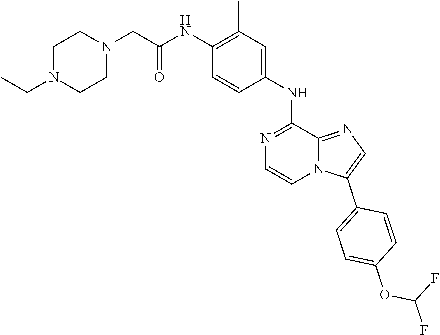 [SMILES: CCN1CCN(CC(=O)Nc2ccc(Nc3nccn4c(-c5ccc(OC(F)F)cc5)cnc34)cc2C)CC1]

### Step 1) 2-(4-ethylpiperazin-1-yl)-N-(2-methyl-4-nitrophenyl) acetamide

To a solution of 2-chloro-N-(2-methyl-4-nitro-phenyl)acetamide (373.78 mg, 1.63 mmol) and potassium carbonate (451.91 mg, 3.27 mmol) in DMF (20 mL) was added 1-ethylpiperazine (373.37 mg, 3.27 mmol). The resulting mixture was stirred at 25° C. for 4 h. The mixture was diluted with water (100 mL), extracted with EA (100 mL×2), washed with brine, dried over sodium sulfated and concentrated. The residue was purified by silica gel chromatography eluting with PE:EA=3:1 to DCM:MeOH=50:1 to afford 2-(4-ethylpiperazin-1-yl)-N-(2-methyl-4-nitro-phenyl)acetamide (450 mg, 1.47 mmol, 89.85%) as a yellow solid. ¹H NMR (400 MHz, CDCl₃) δ=9.69 (s, 1H), 8.46 (d, J=8.4 Hz, 1H), 8.05-8.02 (m, 2H), 3.15 (s, 2H), 2.66-2.42 (m, 8H), 2.39 (q, J=7.2 Hz, 2H), 2.32 (s, 3H), 1.04 (t, J=7.2 Hz, 3H) ppm.

### Step 2) N-(4-amino-2-methylphenyl)-2-(4-ethylpiperazin-1-yl) acetamide

To a solution of 2-(4-ethylpiperazin-1-yl)-N-(2-methyl-4-nitro-phenyl)acetamide (300.0 mg, 0.980 mmol) in methanol (10 mL) was added palladium 10% on carbon (30 mg, 0.280 mmol). The resulting mixture was hydrogenated under 760 mm Hg at 25° C. for 15 h. The catalyst was removed by celite filtration and the filtrate was concentrated to afford N-(4-amino-2-methyl-phenyl)-2-(4-ethylpiperazin-1-yl)acetamide (230 mg, 0.830 mmol, 84.98% yield), the crude product was used in the next step without further purification. MS [M+H]+: 277.2

### Step 3) N-(4-((3-(4-(difluoromethoxy)phenyl)imidazo[1,2-a] pyrazin-8-yl)amino)-2-methylphenyl)-2-(4-ethylpiperazin-1-yl)acetamide

A mixture of N-(4-amino-2-methyl-phenyl)-2-(4-ethylpiperazin-1-yl)acetamide (90.2 mg, 0.330 mmol), 8-chloro-3-[4-(difluoromethoxy)phenyl]imidazo[1,2-a]pyrazine (80.0 mg, 0.270 mmol), tris(dibenzylideneacetone)dipalladium (0) (7.47 mg, 0.010 mmol), 2-di-tert-butylphosphino-2′,4′,6′-triisopropylbiphenyl (11.55 mg, 0.030 mmol) and potassium carbonate (75.18 mg, 0.540 mmol) in tert-butanol (5 mL) was heated to 100° C. for 18 h. The reaction was concentrated to dryness and the residue was taken up in EtOAc (100 ml) and the organic layers were washed with 100 mL water then 1×100 mL brine. The organic layers were then separated and dried (Na₂SO₄) before concentration to dryness. The crude product was then purified by prep-HPLC (FA) to give N-[4-[[3-[4-(difluoromethoxy)phenyl]imidazo[1,2-a]pyrazin-8-yl]amino]-2-methyl-phenyl]-2-(4-ethylpiperazin-1-yl)acetamide (48.2 mg, 0.090 mmol, 33.02%, FA salt) as an off-white solid. ¹H NMR (400 MHz, DMSO-d₆) δ=9.50 (s, 1H), 9.29 (s, 1H), 8.31 (s, 1H), 7.92 (d, J=4.6 Hz, 2H), 7.88-7.82 (m, 2H), 7.77 (d, J=8.7 Hz, 2H), 7.67 (d, J=8.8 Hz, 1H), 7.55 (s, 0.27H), 7.48 (d, J=4.8 Hz, 1H), 7.41-7.35 (m, 2.7 H), 7.18 (s, 0.28H), 3.12 (s, 2H), 2.59-2.55 (m, 4H), 2.48-2.43 (m, 4H), 2.34 (q, J=7.2 Hz, 2H), 2.24 (s, 3H), 1.01 (t, J=7.2 Hz, 3H) ppm MS [M+H]+: 536.2.

### Example 56

### 1-(4-((3-(4-(difluoromethoxy)-3-fluorophenyl)imidazo [1,2-a]pyrazin-8-yl)amino)-2-methylphenyl)pyrrolidin-2-one

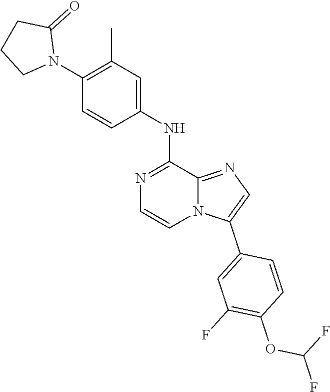 [SMILES: Cc1cc(Nc2nccn3c(-c4ccc(OC(F)F)c(F)c4)cnc23)ccc1N1CCCC1=O]

### Step 1) 2-(4-(difluoromethoxy)-3-fluorophenyl)-4,4,5,5-tetramethyl-1,3,2-dioxaborolane

A mixture of 4-bromo-1-(difluoromethoxy)-2-fluoro-benzene (0.9 g, 3.73 mmol), bis(pinacolato)diboron (1.14 g, 4.48 mmol), potassium acetate (1.1 g, 11.2 mmol) and [1,1′-bis(diphenylphosphino)ferrocene]dichloropalladium(II) (0.27 g, 0.4 mmol) in 1,4-dioxane (10 mL) was heated to 80° C. for 15 h under nitrogen protected. The mixture was diluted with water (20 mL). The precipitate was removed by filtration and the filtrate was extracted with EA (50 mL×2), washed with brine, dried over sodium sulfate and concentrated. The residue was purified by silica gel chromatography eluting with PE:EA=100:1 to afford 2-(4-(difluoromethoxy)-3-fluorophenyl)-4,4,5,5-tetramethyl-1,3,2-dioxaborolane (550 mg, 51.3%) as a colorless oil. ¹H NMR (400 MHz, CDCl3) δ=7.62-7.57 (m, 1H), 7.25-7.21 (m, 2H), 6.77-6.41 (m, 1H), 1.36 (s, 12H) ppm

### Step 2) 8-chloro-3-(4-(difluoromethoxy)-3-fluorophenyl)imidazo [1,2-a]pyrazine

A solution of 8-chloro-3-iodoimidazo[1,2-a]pyrazine (0.5 g, 1.79 mmol), 2-[4-(difluoromethoxy)-3-fluoro-phenyl]-4,4,5,5-tetramethyl-1,3,2-dioxaborolane (0.67 g, 2.33 mmol), sodium carbonate (0.38 g, 3.58 mmol, and [1,1′-bis(diphenylphosphino)ferrocene]dichloropalladium(II) (0.13 g, 0.18 mmol) in 1,4-dioxane (10 mL) and water (1 mL) was heated 80° C. for 15 h. The reaction mixture was concentrated and the residue was then purified by silica gel chromatography eluting with PE:EA=10:1 to 3:1 to 1:1 to give 8-chloro-3-[4-(difluoromethoxy)-3-fluoro-phenyl]imidazo[1,2-a]pyrazine (230 mg, 0.730 mmol, 41% yield) as a yellow oil. MS [M+H]+: 314.0.

### Step 3) 1-(4-((3-(4-(difluoromethoxy)-3-fluorophenyl)imidazo [1,2-a]pyrazin-8-yl)amino)-2-methylphenyl)pyrrolidin-2-one

A mixture of 8-chloro-3-[4-(difluoromethoxy)-3-fluoro-phenyl]imidazo[1,2-a]pyrazine (95.01 mg, 0.300 mmol), 1-(4-amino-2-methyl-phenyl)pyrrolidin-2-one (69.15 mg, 0.360 mmol), 2-di-tert-butylphosphino-2′,4′,6′-triisopropylbiphenyl (3.86 mg, 0.010 mmol), tris(dibenzylideneacetone)dipalladium (0) (27.74 mg, 0.030 mmol) and potassium carbonate (83.73 mg, 0.610 mmol) in tert-butanol (5 mL) was heated to 100° C. for 18 h under nitrogen. The reaction mixture was concentrated to dryness and the residue was taken up in EtOAc (50 mL) and the organic layer was washed with 2×50 mL water then 1×100 mL brine. The organic layers were then separated, dried (Na₂SO₄) and then concentration to dryness. The crude product was then purified by prep-HPLC (FA) to afford 1-[4-[[3-[4-(difluoromethoxy)-3-fluoro-phenyl]imidazo[1,2-a]pyrazin-8-yl]amino]-2-methyl-phenyl]pyrrolidin-2-one (12 mg, 0.030 mmol, 7.98% yield) as a white solid. ¹H NMR (400 MHz, MeOD) δ=8.00-7.95 (m, 2H), 7.75-7.62 (m, 3H), 7.56 (d, J=2.0 Hz, 2H), 7.38-7.36 (m, 2H), 7.18-6.81 (m, 1H), 3.85 (t, J=7.2 Hz, 2H), 2.63 (t, J=8.0 Hz, 2H), 7.55 (s, 0.27H), 2.37-2.27 (m, 5H) ppm. MS [M+H]+: 468.0.

### Example 57

### 1-(4-((3-(4-(difluoromethoxy)-3-fluorophenyl)imidazo [1,2-a]pyrazin-8-yl)amino)phenyl)pyrrolidin-2-one

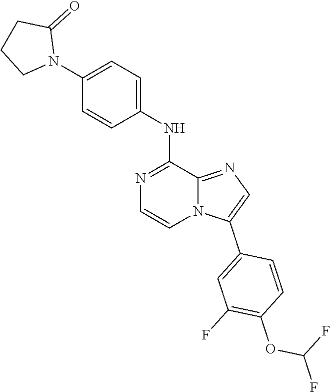 [SMILES: O=C1CCCN1c1ccc(Nc2nccn3c(-c4ccc(OC(F)F)c(F)c4)cnc23)cc1]

A mixture of 8-chloro-3-[4-(difluoromethoxy)-3-fluoro-phenyl]imidazo[1,2-a]pyrazine (80.0 mg, 0.260 mmol), 1-(4-aminophenyl)pyrrolidin-2-one (53.93 mg, 0.310 mmol), 2-di-tert-butylphosphino-2′,4′,6′-triisopropylbiphenyl (3.25 mg, 0.010 mmol), tris(dibenzylideneacetone)dipalladium (0) (23.36 mg, 0.030 mmol) and potassium carbonate (70.5 mg, 0.510 mmol) in tert-butanol (5 mL) was heated to 100° C. for 18 h under nitrogen. The reaction was concentrated to dryness and the residue was taken up in EtOAc (100 mL) and the organic layers were washed with 2×50 mL water then 1×50 mL brine. The organic layers were then separated and dried (Na₂SO₄) before concentration to dryness. The crude product was then purified by prep-HPLC (FA) to afford 1-[4-[[3-[4-(difluoromethoxy)-3-fluoro-phenyl]imidazo[1,2-a]pyrazin-8-yl]amino]phenyl]pyrrolidin-2-one (12.5 mg, 0.030 mmol, 10.48%, FA salt) as an off-white solid. ¹H NMR (400 MHz, DMSO-d₆) δ=9.67 (s, 1H), 8.44 (s, 1H), 8.06-8.00 (m, 3H), 7.93 (s, 1H), 7.60 (d, J=8.8 Hz, 1H), 7.55-7.18 (m, 6H), 3.84 (t, J=7.2 Hz, 2H), 2.50-2.47 (m, 2H), 2.11-2.203 (m, 2H) ppm. MS [M+H]+: 454.0.

### Example 58

### 1-(4-((3-(4-methoxyphenyl)imidazo[1,2-a]pyrazin-8-yl) amino)-2-methylphenyl)pyrrolidin-2-one

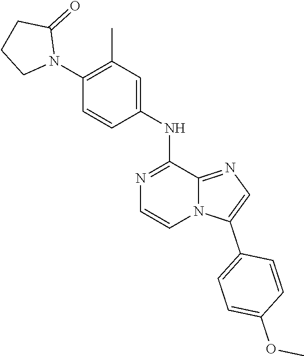 [SMILES: COc1ccc(-c2cnc3c(Nc4ccc(N5CCCC5=O)c(C)c4)nccn23)cc1]

A mixture of 8-chloro-3-(4-methoxyphenyl)imidazo[1,2-a]pyrazine (80.0 mg, 0.310 mmol), 1-(4-amino-2-methyl-phenyl)pyrrolidin-2-one (70.33 mg, 0.370 mmol), 2-di-tert-butylphosphino-2′,4′,6′-triisopropylbiphenyl (3.92 mg, 0.010 mmol), tris(dibenzylideneacetone)dipalladium (0) (28.21 mg, 0.030 mmol) and potassium carbonate (85.15 mg, 0.620 mmol) in tert-butanol (5 mL) was heated to 100° C. for 18 h under nitrogen. The mixture was diluted with water (50 mL) and then extracted with EA (50 mL×2). The combined organic phases were washed with brine, dried over sodium sulfate and then concentrated. The residue was purified by prep-HPLC (FA) to afford 1-[4-[[3-(4-methoxyphenyl)imidazo[1,2-a]pyrazin-8-yl]amino]-2-methyl-phenyl]pyrrolidin-2-one (23.2 mg, 0.060 mmol, 18.03% yield) as an off-white solid.

¹H NMR (400 MHz, DMSO-d₆) δ=7.91-7.81 (m, 4H), 7.62 (d, J=8.8 Hz, 2H), 7.43 (d, J=4.8 Hz, 1H), 7.18-7.14 (m, 3H), 3.85 (s, 3H), 3.68 (t, J=7.2 Hz, 2H), 2.42 (t, J=8.0 Hz, 2H), 2.17-2.10 (m, 5H) ppm. MS [M+H]+: 414.1.

### Example 59

### 2-(3-aminopyrrolidin-1-yl)-N-(4-((3-(4-(difluoromethoxy) phenyl)imidazo[1,2-a]pyrazin-8-yl)amino)-2-methylphenyl)acetamide

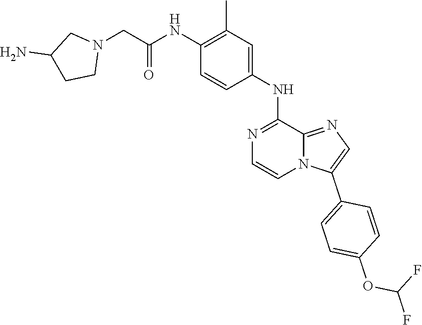 [SMILES: Cc1cc(Nc2nccn3c(-c4ccc(OC(F)F)cc4)cnc23)ccc1NC(=O)CN1CCC(N)C1]

### Step 1) tert-butyl (1-(2-((2-methyl-4-nitrophenyl)amino)-2-oxoethyl)pyrrolidin-3-yl)carbamate

A mixture of 2-chloro-N-(2-methyl-4-nitro-phenyl)acetamide (400.0 mg, 1.75 mmol), tert-butyl pyrrolidin-3-ylcarbamate (325.85 mg, 1.75 mmol) and potassium carbonate (483.61 mg, 3.5 mmol) in DMF (10 mL) was stirred at 25° C. for 4 h. The mixture was diluted with water (100 mL), extracted with EA (100 mL×2), washed with brine, dried over sodium sulfate and then concentrated to give tert-butyl N-[1-[2-(2-methyl-4-nitro-anilino)-2-oxo-ethyl]pyrrolidin-3-yl]carbamate (450 mg, 1.19 mmol, 67.97%) as a colorless oil. MS [M+H]+: 379.2

### Step 2) tert-butyl (1-(2-((4-amino-2-methylphenyl)amino)-2-oxoethyl)pyrrolidin-3-yl)carbamate

To a solution of tert-butyl N-[1-[2-(2-methyl-4-nitro-anilino)-2-oxo-ethyl]pyrrolidin-3-yl]carbamate (450.0 mg, 1.19 mmol) in methanol (10 mL) was added Palladium 10% on Carbon (45 mg, 10% wt). The resulting suspension was hydrogenated under 760 mm Hg at 25° C. for 15 h. The mixture was filtered through celite and the filtrate was concentrated to give tert-butyl N-[1-[2-(4-amino-2-methyl-anilino)-2-oxo-ethyl]pyrrolidin-3-yl]carbamate (385 mg, 1.1 mmol, 92.92% yield) as a white solid.

MS [M+H]+:

### Step 3) tert-butyl (1-(2-((4-((3-(4-(difluoromethoxy)phenyl) imidazo[1,2-a]pyrazin-8-yl)amino)-2-methylphenyl)amino)-2-oxoethyl)pyrrolidin-3-yl)carbamate

A mixture of 8-chloro-3-[4-(difluoromethoxy)phenyl]imidazo[1,2-a]pyrazine (80.0 mg, 0.310 mmol), tert-butyl N-[1-[2-(4-amino-2-methyl-anilino)-2-oxo-ethyl]pyrrolidin-3-yl]carbamate (128.81 mg, 0.370 mmol), tris(dibenzylideneacetone)dipalladium (0) (8.46 mg, 0.010 mmol), 2-di-tert-butylphosphino-2′,4′,6′-triisopropylbiphenyl (13.08 mg, 0.030 mmol) and potassium carbonate (85.15 mg, 0.620 mmol) in tert-butanol (5 mL) was heated to 100° C. for 18 h. The reaction was concentrated to dryness and the residue was taken up in EtOAc (100 mL) and the organic layers were washed with 100 mL water then 1×100 mL brine. The organic layers were then separated and dried (Na₂SO₄) before concentration to dryness. The crude was then purified by prep-HPLC (FA) to give tert-butyl N-[1-[2-[4-[[3-[4-(difluoromethoxy)phenyl]imidazo[1,2-a]pyrazin-8-yl]amino]-2-methyl-anilino]-2-oxo-ethyl]pyrrolidin-3-yl]carbamate (55 mg, 0.090 mmol, 29.38% yield) as an off-white solid. MS [M+H]+: 608.2.

### Step 4) 2-(3-aminopyrrolidin-1-yl)-N-(4-((3-(4-(difluoromethoxy) phenyl)imidazo[1,2-a]pyrazin-8-yl)amino)-2-methylphenyl)acetamide

To a solution of tert-butyl N-[1-[2-[4-[[3-[4-(difluoromethoxy)phenyl]imidazo[1,2-a]pyrazin-8-yl]amino]-2-methyl-anilino]-2-oxo-ethyl]pyrrolidin-3-yl]carbamate (55.0 mg, 0.090 mmol) in methanol (2 mL) was added hydrochloric acid in methanol (2.0 mL, 8 mmol, 4 M) slowly. The resulting mixture was stirred at 25° C. for 15 h. The residue was re-dissolved in water and then lyophilized to afford 2-(3-aminopyrrolidin-1-yl)-N-[4-[[3-[4-(difluoromethoxy)phenyl]imidazo[1,2-a]pyrazin-8-yl]amino]-2-methyl-phenyl]acetamide (11 mg, 0.020 mmol, 23.56% yield) as an off-white solid.

¹H NMR (400 MHz, DMSO-d₆) δ=9.52-9.48 (m, 4H), 8.41 (s, 1H), 7.92-7.88 (m, 4H), 7.76 (d, J=8.8 Hz, 1H), 7.50-7.28 (m, 5H), 3.34 (d, J=16 Hz, 1H), 3.22 (d, J=16 Hz, 1H), 3.05-3.04 (m, 1H), 2.87-2.85 (m, 1H), 2.71-2.68 (m, 1H), 2.44-2.42 (m, 1H), 2.21 (s, 3H), 2.19-2.17 (m, 1H), 1.77-1.76 (m, 1H) ppm. MS [M+H]+: 508.2.

### Example 60

### 1-(4-((3-(4-chlorophenyl)imidazo[1,2-a]pyrazin-8-yl) amino)-2-methylphenyl)pyrrolidin-2-one

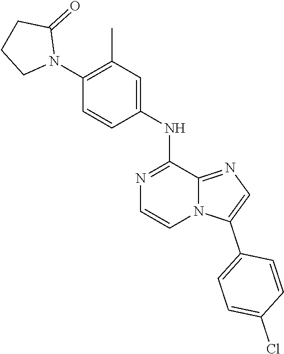 [SMILES: Cc1cc(Nc2nccn3c(-c4ccc(Cl)cc4)cnc23)ccc1N1CCCC1=O]

### Step 1) 8-chloro-3-(4-chlorophenyl)imidazo[1,2-a]pyrazine

A solution of 8-chloro-3-iodoimidazo[1,2-a]pyrazine (0.5 g, 1.79 mmol), (4-chlorophenyl)boronic acid (0.36 g, 2.33 mmol), sodium carbonate (0.38 g, 3.58 mmol) and [1,1′-bis(diphenylphosphino)ferrocene]dichloropalladium(II) (0.13 g, 0.180 mmol) in 1,4-dioxane (10 mL) and water (1 mL) was heated to 80° C. for 15 h under nitrogen. The reaction mixture was concentrated and the residue was purified by silica gel chromatography eluting with PE:EA=10:1 to 3:1 to 1:1 to give 8-chloro-3-(4-chlorophenyl)imidazo[1,2-a]pyrazine (350 mg, 1.33 mmol, 74.07%) as a white solid. MS obsd. (ESI+) [(M+H)⁺]:264.1.

### Step 2) 1-(4-((3-(4-chlorophenyl)imidazo[1,2-a]pyrazin-8-yl) amino)-2-methylphenyl)pyrrolidin-2-one

A mixture of 8-chloro-3-(4-chlorophenyl)imidazo[1,2-a]pyrazine (80.0 mg, 0.300 mmol), 1-(4-amino-2-methyl-phenyl)pyrrolidin-2-one (69.15 mg, 0.360 mmol), 2-di-tert-butylphosphino-2′,4′,6′-triisopropylbiphenyl (3.86 mg, 0.010 mmol), tris(dibenzylideneacetone)dipalladium (0) (27.74 mg, 0.030 mmol) and potassium carbonate (83.73 mg, 0.610 mmol) in tert-butanol (5 mL) was heated to 100° C. for 18 h. The reaction was concentrated to dryness and the residue was taken up in EtOAc (100 mL) and the organic layer was washed with 2×100 mL water then 1×100 mL brine. The organic layers were then separated and dried (MgSO₄) before concentration to dryness. The crude product was then purified by prep-HPLC (FA) to afford 1-[4-[[3-(4-chlorophenyl)imidazo[1,2-a]pyrazin-8-yl]amino]-2-methyl-phenyl]pyrrolidin-2-one (7.8 mg, 0.020 mmol, 5.61%) as an off-white solid. ¹H NMR (400 MHz, MeOD) δ=7.94 (d, J=4.2 Hz, 1H), 7.82-7.80 (m, 3H), 7.69 (d, J=8.4 Hz, 1H), 7.62 (d, J=8.8 Hz, 1H), 7.47 (d, J=4.8 Hz, 1H), 7.27 (d, J=8.4 Hz, 1H), 3.83 (d, J=7.2 Hz, 2H), 2.62 (d, J=8.0 Hz, 2H), 2.34-2.28 (m, 5H) ppm. MS obsd. (ESI+) [(M+H)⁺]:418.1.

### Example 61

### N-(2-(2-aminoethoxy)ethyl)-4-((3-(4-methoxyphenyl) imidazo[1,2-a]pyrazin-8-yl)amino)-2-methylbenzenesulfonamide

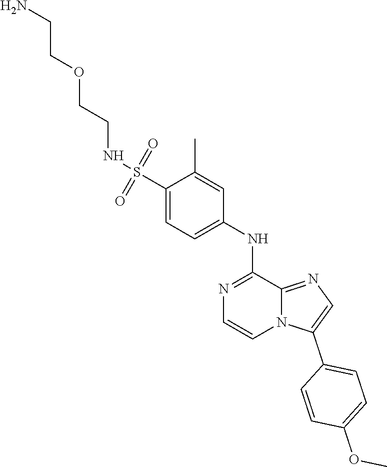 [SMILES: COc1ccc(-c2cnc3c(Nc4ccc(S(=O)(=O)NCCOCCN)c(C)c4)nccn23)cc1]

### Step 1) tert-butyl (2-(2-(2-methyl-4-nitrophenylsulfonamido) ethoxy)ethyl)carbamate

To an ice-cooled solution of N—BOC-2-(2-amino-ethoxy)-ethylamine (1.04 g, 5.09 mmol) and triethylamine (0.89 mL, 6.37 mmol) in DCM (10 mL) was added 2-methyl-4-nitro-benzenesulfonyl chloride (1.0 g, 4.24 mmol). The resulting mixture was stirred at 30° C. for 2 h, the mixture was washed with water, dried over sodium sulfate and concentrated. The residue was purified by silica gel chromatography eluting with PE:EA=5:1 to 1:1 to give tert-butyl N-[2-[2-[(2-methyl-4-nitro-phenyl)sulfonylamino]ethoxy]ethyl]carbamate (600 mg, 1.49 mmol, 35.04%) as a colorless oil MS obsd. (ESI+) [(M+Na)⁺]:426.2

### Step 2) tert-butyl (2-(2-(4-amino-2-methylphenylsulfonamido) ethoxy)ethyl)carbamate

A solution of tert-butyl N-[2-[2-[(2-methyl-4-nitro-phenyl)sulfonylamino]ethoxy]ethyl]carbamate (600.0 mg, 1.49 mmol) and palladium on carbon (60 mg, 10% wt) in methanol (20 mL) was hydrogenated under 760 mm Hg at 25° C. for 30 h. The catalyst was removed by filtration and the filtrate was concentrated, the residue was purified by prep-HPLC to afford tert-butyl N-[2-[2-[(4-amino-2-methyl-phenyl)sulfonylamino]ethoxy]ethyl]carbamate (300 mg, 0.800 mmol, 48.61%) as a colorless oil.

MS obsd. (ESI+) [(M+Na)⁺]:396.1.

### Step 3) tert-butyl (2-(2-(4-((3-(4-methoxyphenyl)imidazo[1,2-a] pyrazin-8-yl)amino)-2-methylphenylsulfonamido)ethoxy)ethyl)carbamate

A solution of tert-butyl N-[2-[2-[(4-amino-2-methyl-phenyl)sulfonylamino]ethoxy]ethyl]carbamate (215.72 mg, 0.580 mmol), 8-chloro-3-(4-methoxyphenyl)imidazo[1,2-a]pyrazine (150.0 mg, 0.580 mmol), Brettphos Pd G3 (49.53 mg, 0.060 mmol) and potassium carbonate (239.49 mg, 1.73 mmol) in tert-butanol (3 mL) was heated to 100° C. for 15 h under nitrogen. The mixture was diluted with water, extracted with EA (50 mL×2), washed with brine, dried over sodium sulfate and concentrated. The residue was purified by flash column to afford tert-butyl N-[2-[2-[[4-[[3-(4-methoxyphenyl)imidazo[1,2-a]pyrazin-8-yl]amino]-2-methyl-phenyl]sulfonylamino]ethoxy]ethyl]carbamate (90 mg, 0.150 mmol, 23.5%) as a light yellow solid. MS obsd. (ESI+) [(M+H)⁺]:597.3

### Step 4) N-(2-(2-aminoethoxy)ethyl)-4-((3-(4-methoxyphenyl) imidazo[1,2-a]pyrazin-8-yl)amino)-2-methylbenzenesulfonamide

To a solution of tert-butyl N-[2-[2-[[4-[[3-(4-methoxyphenyl)imidazo[1,2-a]pyrazin-8-yl]amino]-2-methyl-phenyl]sulfonylamino]ethoxy]ethyl]carbamate (70.0 mg, 0.120 mmol) in methanol (10 mL) was added hydrogen chloride in MeOH (5.0 mL, 0.120 mmol, 4M). The resulting mixture was stirred at 25° C. for 15 h. The mixture was concentrated and purified by prep-HPLC to afford N-[2-(2-aminoethoxy)ethyl]-4-[[3-(4-methoxyphenyl)imidazo[1,2-a]pyrazin-8-yl]amino]-2-methyl-benzenesulfonamide (50 mg, 0.100 mmol, 74.43%, FA salt) as a white solid.

¹H NMR (400 MHz, DMSO-d₆) δ=9.96 (br. s., 1H), 8.42 (s, 1H), 8.12-8.07 (m, 2H), 7.99 (d, J=4.8 Hz, 1H), 7.82-7.76 (m, 2H), 7.63 (d, J=8.8 Hz, 2H), 7.54 (d, J=4.8 Hz, 1H), 7.15 (d, J=8.8 Hz, 2H), 3.85 (s, 3H), 3.43 (br. s., 2H), 3.35 (t, J=5.6 Hz, 2H), 2.95 (t, J=5.6 Hz, 2H), 2.82 (br. s., 2H), 2.58 (s, 3H) ppm.

MS obsd. (ESI+) [(M+H)⁺]:497.2.

### Example 62

### 4-(5-aminopentyl)-1-(4-((3-(4-(difluoromethoxy)phenyl) imidazo[1,2-a]pyrazin-s-yl)amino)phenyl)pyrrolidin-2-one

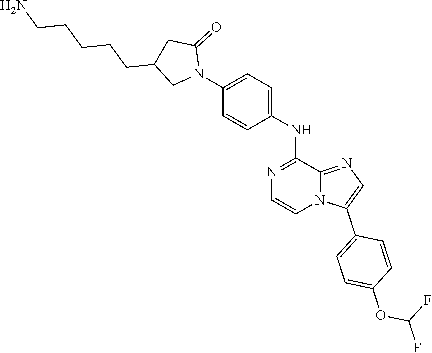 [SMILES: NCCCCCC1CC(=O)N(c2ccc(Nc3nccn4c(-c5ccc(OC(F)F)cc5)cnc34)cc2)C1]

### Step 1) (E)-methyl 8-((tert-butoxycarbonyl)amino)oct-2-enoate

to an ice-cooled solution of trimethyl phosphonoacetate (2.03 g, 11.15 mmol) in THF (20 mL) was added sodium hydride (0.56 g, 13.93 mmol, 60% in oil). The resulting mixture was stirred for 15 min and then a solution of tert-butyl N-(6-oxohexyl)carbamate (2.0 g, 9.29 mmol) in THF (20 mL) was added. The resulting mixture was stirred at 0° C. for 1 h. The mixture was quenched with sat NH₄Cl aq and extracted with EA (50 mL×2). The organic phase was separated, washed with brine, dried over sodium sulfate and concentrated. The residue was purified by silica gel chromatography eluting with PE:EA=20:1 to afford methyl (E)-8-(tert-butoxycarbonylamino)oct-2-enoate (1.2 g, 4.42 mmol, 47.6% yield) as a colorless oil

¹H NMR (400 MHz, CDCl₃) δ=6.97-6.95 (m, 1H), 5.83 (d, J=16.4 Hz, 1H), 4.45 (br. s, 1H), 3.74 (s, 3H), 3.12 (d, J=6.4 Hz, 2H), 2.25-2.19 (m, 2H) 1.57-1.52 (m, 4H), 1.49 (s, 9H), 1.37-1.34 (m, 2H) ppm.

### Step 2) methyl 8-((tert-butoxycarbonyl)amino)-3-(nitromethyl) octanoate

A solution of methyl (E)-8-(tert-butoxycarbonylamino)oct-2-enoate (500.0 mg, 1.84 mmol) and 1,1,3,3-tetramethylguanidine (21.22 mg, 0.180 mmol) in nitromethane (5 mL) was stirred at 25° C. for 24 h. The solvent was removed under reduced pressure and the mixture was diluted with DCM and then neutralized with 1 N HCl aq. The organic phase was separated, dried over sodium sulfate and concentrated. The residue was purified by silica gel chromatography eluting with PE:EA=15:1 to afford methyl 8-(tert-butoxycarbonylamino)-3-(nitromethyl)octanoate (300 mg, 0.900 mmol, 48.98% yield) as a colorless oil.

¹H NMR (400 MHz, CDCl₃) δ=4.55-4.45 (m, 3H), 3.72 (s, 3H), 3.15-3.11 (m, 2H), 2.67-2.60 (m, 1H), 2.47 (d, J=6.4 Hz, 2H), 1.46 (s, 9H), 1.45-1.25 (m, 8H) ppm.

### Step 3) tert-butyl (5-(5-oxopyrrolidin-3-yl)pentyl)carbamate

A mixture of methyl 8-(tert-butoxycarbonylamino)-3-(nitromethyl)octanoate (300.0 mg, 0.900 mmol) and Raney Ni (30.0 mg, 10% wt) in methanol (20 mL) was hydrogenated under 760 mmHg at 60° C. for 15 h. The catalyst was removed by filtration and the filtrate was concentrated. The filtrate was concentrated to give tert-butyl N-[5-(5-oxopyrrolidin-3-yl)pentyl]carbamate (150 mg, 0.550 mmol, 61.47% yield) as a colorless oil which was used directly in the next step.

### Step 4) tert-butyl (5-(1-(4-nitrophenyl)-5-oxopyrrolidin-3-yl) pentyl)carbamate

A mixture of 1-iodo-4-nitrobenzene (500.0 mg, 2.01 mmol), tert-butyl N-[5-(5-oxopyrrolidin-3-yl)pentyl]carbamate (597.18 mg, 2.21 mmol), copper(I) iodide (38.24 mg, 0.200 mmol), trans-(1R,2R)—N,N′-bismethyl-1,2-cyclohexanediamine (28.56 mg, 0.200 mmol), phosphoric acid, potassium salt (852.4 mg, 4.02 mmol) in DMSO (10 mL) was heated to 100° C. for 15 h. The mixture was diluted with water (100 mL), extracted with EA (50 mL×2), washed with brine, dried over sodium sulfate and then concentrated. The residue was purified by silica gel chromatography eluting with PE:EA=3:1 to 1:1 to afford tert-butyl N-[5-[1-(4-nitrophenyl)-5-oxo-pyrrolidin-3-yl]pentyl]carbamate (200 mg, 0.510 mmol, 25.44% yield) as a colorless oil.

MS obsd. (ESI+) [(M+Na)⁺]:414.1

### Step 5) tert-butyl (5-(1-(4-aminophenyl)-5-oxopyrrolidin-3-yl) pentyl)carbamate

A mixture of tert-butyl N-[5-[1-(4-nitrophenyl)-5-oxo-pyrrolidin-3-yl]pentyl]carbamate (200.0 mg, 0.510 mmol) and palladium 10% on carbon (20 mg, 10% wt) in MeOH (10 mL) was hydrogenated under 760 mmHg at 15° C. for 12 h. The catalyst was removed by filtration. The filtrate was concentrated to afford tert-butyl N-[5-[1-(4-aminophenyl)-5-oxo-pyrrolidin-3-yl]pentyl]carbamate (130 mg, 0.360 mmol, 70.39% yield) as a brown oil.

MS obsd. (ESI+) [(M+H)⁺]:362.4.

### Step 6) tert-butyl (5-(1-(4-((3-(4-(difluoromethoxy)phenyl)imidazo [1,2-a]pyrazin-8-yl)amino)phenyl)-5-oxopyrrolidin-3-yl)pentyl)carbamate

A mixture of tert-butyl N-[5-[1-(4-aminophenyl)-5-oxo-pyrrolidin-3-yl]pentyl]carbamate (120.0 mg, 0.330 mmol), 8-chloro-3-[4-(difluoromethoxy)phenyl]imidazo[1,2-a]pyrazine (98.15 mg, 0.330 mmol), Brettphos Pd G3 (28.46 mg, 0.030 mmol) and potassium carbonate (91.76 mg, 0.660 mmol) was heated to 110° C. for 15 h. The mixture was diluted with water, extracted with EA (50 mL×2), dried over sodium sulfate and concentrated. the residue was triturated with MeOH (10 mL) to afford tert-butyl N-[5-[1-[4-[[3-[4-(difluoromethoxy)phenyl]imidazo[1,2-a]pyrazin-8-yl]amino]phenyl]-5-oxo-pyrrolidin-3-yl]pentyl]carbamate (120 mg, 0.190 mmol, 58.24% yield) as a white solid. MS obsd. (ESI+) [(M+H)⁺]:621.2.

### Step 7) 4-(5-aminopentyl)-1-(4-((3-(4-(difluoromethoxy)phenyl) imidazo[1,2-a]pyrazin-s-yl)amino)phenyl)pyrrolidin-2-one

To a solution of tert-butyl N-[5-[1-[4-[[3-[4-(difluoromethoxy)phenyl]imidazo[1,2-a]pyrazin-8-yl]amino]phenyl]-5-oxo-pyrrolidin-3-yl]pentyl]carbamate (100.0 mg, 0.160 mmol) in methanol (5 mL) was added hydrogen chloride in MeOH (5.0 mL, 20 mmol). The resulting mixture was stirred at 10° C. for 15 h. The mixture was concentrated and the crude product was purified by flash column chromatography to afford 4-(5-aminopentyl)-1-[4-[[3-[4-(difluoromethoxy)phenyl]imidazo[1,2-a]pyrazin-8-yl]amino]phenyl]pyrrolidin-2-one (45.4 mg, 0.090 mmol, 54.13% yield) as a white solid. ¹H NMR (400 MHz, DMSO-d6) δ=9.64 (s, 1H), 8.43 (s, 1H), 8.04 (d, J=8.8 Hz, 2H), 7.93 (d, J=4.4 Hz, 1H), 7.86 (s, 1H), 7.77 (d, J=8.8 Hz, 2H), 7.60-7.18 (m, 6H), 3.92 (t, J=8.4 Hz, 1H), 3.52-3.49 (m, 1H), 2.74-2.58 (m, 2H), 2.43-2.36 (m, 2H), 2.28-2.20 (m, 1H), 1.58-1.42 (m, 4H), 1.39-1.30 (m, 4H) ppm.

MS obsd. (ESI+) [(M+H)⁺]:521.2.

### Example 63

### 4-(4-aminobutyl)-1-(4-((3-(4-(difluoromethoxy)phenyl) imidazo[1,2-a]pyrazin-8-yl)amino)phenyl)pyrrolidin-2-one

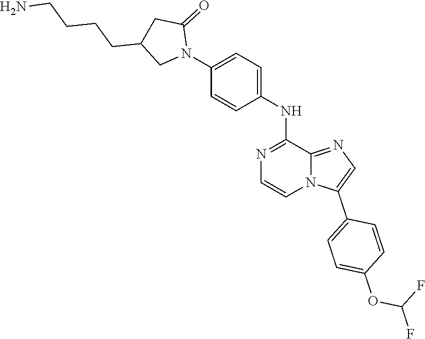 [SMILES: NCCCCC1CC(=O)N(c2ccc(Nc3nccn4c(-c5ccc(OC(F)F)cc5)cnc34)cc2)C1]

### Step 1) 5-((tert-butyldiphenylsilyl)oxy)pentan-1-ol

To a mixture of 1,5-pentanediol (5.0 g, 48.01 mmol) and imidazole (3.92 g, 57.61 mmol) in DCM (100 mL) was added tert-butylchlorodiphenylsilane (13.2 g, 48.01 mmol). The resulting mixture was stirred at 10° C. for 15 h. The mixture was washed with water, dried over sodium sulfate and concentrated. The residue was purified by silica gel chromatography eluting with PE:EA=20:1 to 10:1 to afford 5-[tert-butyl(diphenyl)silyl]oxypentan-1-ol (8 g, 23.35 mmol, 48.65% yield) as a colorless oil.

### Step 2) 5-((tert-butyldiphenylsilyl)oxy)pentanal

To a mixture of 5-[tert-butyl(diphenyl)silyl]oxypentan-1-ol (8.0 g, 23.35 mmol), sodium bromide (0.24 g, 2.34 mmol), sodium hydrogen carbonate (0.78 g, 9.34 mmol) and 2,2,6,6-tetramethylpiperidin-1-ol (72.98 mg, 0.470 mmol) in DCM (100 mL) and water (100 mL) was added sodium hypochlorite (26.08 g, 35.03 mmol) aq. slowly at 0° C. After addition, the mixture was stirred at 0° C. for 0.5 h. The organic phase was separated, dried over sodium sulfate and concentrate. The residue was purified by silica gel chromatography eluting with PE:EA=20:1 to afford 5-[tert-butyl(diphenyl)silyl]oxypentanal (5 g, 14.68 mmol, 62.87%) as a colorless oil.

¹H NMR (400 MHz, CDCl₃) δ=9.77 (s, 1H), 7.70-7.68 (m, 4H), 7.46-7.41 (m, 6H), 3.72-3.68 (m, 2H), 2.45-2.42 (m, 2H), 1.76-1.74 (m, 2H), 1.64-1.62 (m, 2H), 1.08 (s, 9H) ppm.

### Step 3) (E)-methyl 7-((tert-butyldiphenylsilyl)oxy)hept-2-enoate

To an ice-cooled solution of trimethyl phosphonoacetate (3.21 g, 17.62 mmol) in THF (100 mL) was slowly added sodium hydride (880.9 mg, 22.02 mmol, 60% in oil). After addition, a solution of 5-[tert-butyl(diphenyl)silyl]oxypentanal (5.0 g, 14.68 mmol) in THF (20 mL) was added. The resulting suspension was stirred at 0° C. for 0.5 h. The mixture was quenched with 1N HCl, extracted with EA (100 mL×2), washed with brine, dried over sodium sulfate and concentrated. The residue was purified by silica gel chromatography eluting with PE:EA=20:1 to afford methyl (E)-7-[tert-butyl(diphenyl)silyl]oxyhept-2-enoate (4.5 g, 11.35 mmol, 77.28%) as a colorless oil

¹H NMR (400 MHz, CDCl₃) δ=7.70-7.68 (m, 4H), 7.43-7.41 (m, 6H), 7.01-6.96 (m, 1H), 5.85 (d, J=16.0, 1H), 3.76 (s, 3H), 3.72-3.68 (m, 2H), 1.63-1.60 (m, 6H), 1.07 (s, 9H) ppm.

### Step 4) methyl 7-((tert-butyldiphenylsilyl)oxy)-3-(nitromethyl) heptanoate

A mixture of methyl (E)-7-[tert-butyl(diphenyl)silyl]oxyhept-2-enoate (4.48 g, 11.29 mmol) and 1,1,3,3-tetramethylguanidine (0.14 mL, 1.13 mmol) in nitromethane (24.46 mL, 451.61 mmol). The resulting mixture was stirred at 10° C. for 72 h. The mixture was concentrated to removed excessive nitromethane and then diluted with DCM (100 mL), neutralized with 1 N HCl, washed with brine, dried over sodium sulfate and then concentrated. The residue was purified by silica gel chromatography eluting with PE:EA=20:1 to afford methyl 7-[tert-butyl(diphenyl)silyl]oxy-3-(nitromethyl)heptanoate (3.58 g, 7.82 mmol, 69.29%) as a colorless oil.

¹H NMR (400 MHz, CDCl₃) δ=7.68-7.65 (m, 4H), 7.45-7.39 (m, 6H), 4.53-4.51 (m, 2H), 3.71-3.67 (m, 5H), 2.65-2.59 (m, 1H), 2.45 (d, J=6.8, 2H), 1.57-1.55 (m, 2H), 1.44-1.42 (m, 4H), 1.07 (s, 9H) ppm.

### Step 5) 4-(4-((tert-butyldiphenylsilyl)oxy)butyl)pyrrolidin-2-one

A mixture of methyl 7-[tert-butyl(diphenyl)silyl]oxy-3-(nitromethyl)heptanoate (1.0 g, 2.19 mmol) and Raney nickel (68.09 mg, 7% wt) in ethanol (20 mL) was hydrogenated under 3800 mm Hg at 55° C. for 15 h. Raney nickel was removed by filtration and the filtrate was concentrated to give 4-[4-[tert-butyl(diphenyl)silyl]oxybutyl]pyrrolidin-2-one (800 mg, 2.02 mmol, 92.54% yield), the crude product was used directly in the next step. MS obsd. (ESI+) [(M+Na)⁺]:418.2.

### Step 6) 4-(4-((tert-butyldiphenylsilyl)oxy)butyl)-1-(4-nitrophenyl) pyrrolidin-2-one

A mixture of 4-[4-[tert-butyl(diphenyl)silyl]oxybutyl]pyrrolidin-2-one (0.8 g, 2.02 mmol), 1-iodo-4-nitrobenzene (0.5 g, 2.02 mmol), copper(I) iodide (38.51 mg, 0.200 mmol), trans-(1r,2r)-N,N′-bismethyl-1,2-cyclohexanediamine (28.76 mg, 0.200 mmol) and phosphoric acid, potassium salt (858 mg, 4.04 mmol) in DMSO (10 mL) was heated to 100° C. for 15 h under nitrogen. The mixture was diluted water (20 mL) and then extracted with EtOAc (100 mL×2). The combined organic phases were washed with brine, dried over sodium sulfate and concentrated. The residue was purified by silica gel chromatography eluting with PE:EA=3:1 to afford 4-[4-[tert-butyl(diphenyl)silyl]oxybutyl]-1-(4-nitrophenyl)pyrrolidin-2-one (420 mg, 0.810 mmol, 40.2% yield) as a yellow solid. MS obsd. (ESI+) [(M+Na)⁺]:539.2

### Step 7) 4-(4-hydroxybutyl)-1-(4-nitrophenyl)pyrrolidin-2-one

To a solution of 4-[4-[tert-butyl(diphenyl)silyl]oxybutyl]-1-(4-nitrophenyl)pyrrolidin-2-one (1.6 g, 3.1 mmol) in THF (10 mL) was added tetrabutylammonium fluoride (1.62 g, 6.19 mmol). The resulting mixture was stirred at 10° C. for 15h. The mixture was diluted with water, extracted with EA (50 mL×2), washed with brine, dried over sodium sulfate and concentrated. The residue was purified by flash column chormatography to afford 4-(4-hydroxybutyl)-1-(4-nitrophenyl)pyrrolidin-2-one (600 mg, 2.16 mmol, 69.62%) as a yellow solid. MS obsd. (ESI+) [(M+H)⁺]:279.3

### Step 8) 4-(1-(4-nitrophenyl)-5-oxopyrrolidin-3-yl)butyl methanesulfonate

To a solution of 4-(4-hydroxybutyl)-1-(4-nitrophenyl)pyrrolidin-2-one (600.0 mg, 2.16 mmol) and triethylamine (1.24 mL, 3.23 mmol) in DCM (10 mL) was slowly added methanesulfonyl chloride (0.2 mL, 2.59 mmol). After being stirred for 2 h at 0° C., the mixture was diluted with water (100 mL), the organic phase separated and then dried over sodium sulfate and concentrated to afford 4-[1-(4-nitrophenyl)-5-oxo-pyrrolidin-3-yl]butyl methanesulfonate (600 mg, 1.68 mmol, 78.09% yield). The crude product was used in the next step without further purification.

MS obsd. (ESI+) [(M+H)⁺]:357.0.

### Step 9) 4-(4-azidobutyl)-1-(4-nitrophenyl)pyrrolidin-2-one

A mixture of sodium azide (0.3 mL, 8.42 mmol) and 4-[1-(4-nitrophenyl)-5-oxo-pyrrolidin-3-yl]butyl methanesulfonate (600.0 mg, 1.68 mmol, 1 eq) in DMF (10 mL) was heated to 50° C. for 15 h. The mixture was diluted with water, extracted with EA (100 mL×2), washed with brine, dried over sodium sulfate and concentrated to give 4-(4-azidobutyl)-1-(4-nitrophenyl)pyrrolidin-2-one (500 mg, 1.65 mmol, 97.91% yield). The crude product was used in the next step without further purification.

MS obsd. (ESI+) [(M+H)⁺]:304.2

### Step 10) tert-butyl (4-(1-(4-nitrophenyl)-5-oxopyrrolidin-3-yl) butyl)carbamate

To a solution of 4-(4-azidobutyl)-1-(4-nitrophenyl)pyrrolidin-2-one (700.0 mg, 2.31 mmol) in THF (10 mL) and water (2 mL) was added triphenylphosphine (726.36 mg, 2.77 mmol). The resulting mixture was stirred at 10° C. for 15 h and then di-t-butyldicarbonate (604.41 mg, 2.77 mmol) was added. The resulting mixture was stirred for 6 h. The mixture was concentrated and the residue was purified by silica gel chromatography eluting with PE:EA=5:1 to 3:1 afford tert-butyl N-[4-[1-(4-nitrophenyl)-5-oxo-pyrrolidin-3-yl] butyl]carbamate (650 mg, 1.72 mmol, 74.62% yield) as a colorless oil. MS obsd. (ESI+) [(M+Na)⁺]:400.1.

### Step 10) tert-butyl (4-(1-(4-aminophenyl)-5-oxopyrrolidin-3-yl) butyl)carbamate

A mixture of tert-butyl N-[4-[1-(4-nitrophenyl)-5-oxo-pyrrolidin-3-yl]butyl]carbamate (600.0 mg, 1.59 mmol) and palladium 10% on carbon (60 mg, 10⁰/owt) in methanol (10 mL) was hydrogenated under 760 mm Hg for 4 h. The catalyst was removed by filtration and the filtrate was concentrated to afford tert-butyl N-[4-[1-(4-aminophenyl)-5-oxo-pyrrolidin-3-yl]butyl]carbamate (450 mg, 1.3 mmol, 81.47%) as a colorless oil. MS obsd. (ESI+) [(M+H)⁺]:348.2

### Step 11) tert-butyl (4-(1-(4-((3-(4-(difluoromethoxy)phenyl) imidazo[1,2-a]pyrazin-8-yl)amino)phenyl)-5-oxopyrrolidin-3-yl)butyl)carbamate

A mixture of tert-butyl N-[4-[1-(4-aminophenyl)-5-oxo-pyrrolidin-3-yl]butyl]carbamate (100.0 mg, 0.290 mmol), 8-chloro-3-[4-(difluoromethoxy)phenyl]imidazo[1,2-a]pyrazine (85.1 mg, 0.290 mmol), Brettphos Pd G3 (24.68 mg, 0.030 mmol) and potassium carbonate (79.55 mg, 0.580 mmol) was heated to 110° C. for 15 h. The mixture was diluted with water, extracted with EA (50 mL×2), washed with brine, dried over sodium sulfate and concentrated. the residue was purified by prep-TLC (DCM:MeOH=10:1) to afford tert-butyl N-[4-[1-[4-[[3-[4-(difluoromethoxy)phenyl]imidazo[1,2-a]pyrazin-8-yl]amino]phenyl]-5-oxo-pyrrolidin-3-yl]butyl]carbamate (100 mg, 0.160 mmol, 57.27%) as a white solid.

MS obsd. (ESI+) [(M+H)⁺]:607.3.

### Step 12) 4-(4-aminobutyl)-1-(4-((3-(4-(difluoromethoxy)phenyl) imidazo[1,2-a]pyrazin-s-yl)amino)phenyl)pyrrolidin-2-one

To a solution of tert-butyl N-[4-[1-[4-[[3-[4-(difluoromethoxy)phenyl]imidazo[1,2-a]pyrazin-8-yl]amino]phenyl]-5-oxo-pyrrolidin-3-yl]butyl]carbamate (100.0 mg, 0.160 mmol) in methanol (3 mL) was added hydrogen chloride in MeOH 3 mL, 12 mmol, 4 M). The resulting mixture was stirred at 10° C. for 15 h. The mixture was concentrated and the crude was purified by reversed phase HPLC (FA) to afford 4-(4-aminobutyl)-1-[4-[[3-[4-(difluoromethoxy)phenyl]imidazo[1,2-a]pyrazin-8-yl]amino]phenyl]pyrrolidin-2-one; formic acid (24.5 mg, 0.040 mmol, 29.25%) as a white solid. ¹H NMR (400 MHz, DMSO-d₆) δ=9.65 (s, 1H), 8.45 (s, 1H), 8.04 (d, J=8.8, 2H), 7.93 (d, J=4.8, 1H), 7.86 (s, 1H), 7.77 (d, J=8.8, 2H), 7.61-7.17 (m, 6H), 3.92 (t, J=8.6 Hz, 1H), 3.51 (dd, J=7.2, 9.4 Hz, 1H), 2.75 (t, J=6.4 Hz, 1H), 2.65-2.57 (m, 1H), 2.44-2.37 (m, 1H), 2.27-2.21 (m, 1H), 1.55-1.36 (m, 6H) ppm. MS obsd. (ESI+) [(M+H)⁺]:507.2.

### Example 64

### 4-((2-aminoethoxy)methyl)-1-(4-((3-(4-(difluoromethoxy)phenyl)imidazo[1,2-a]pyrazin-8-yl)amino)phenyl)pyrrolidin-2-one

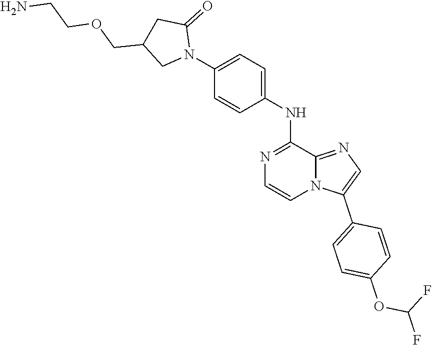 [SMILES: NCCOCC1CC(=O)N(c2ccc(Nc3nccn4c(-c5ccc(OC(F)F)cc5)cnc34)cc2)C1]

### Step 1) 2-(2-((tert-butyldiphenylsilyl)oxy)ethoxy)ethanol

tert-Butylchlorodiphenylsilane (12.95 g, 47.12 mmol) was added to a mixture of diethylene glycol (5.0 g, 47.12 mmol) and imidazole (3.85 g, 56.54 mmol) in DCM (100 mL). The resulting mixture was stirred at 10° C. for 15 h. The mixture was washed with water (100 mL), dried over sodium sulfate and then concentrated. The residue was purified by silica gel chromatography eluting with PE:EA=20:1 to 5:1 to afford 2-[2-[tert-butyl(diphenyl)silyl]oxyethoxy]ethanol (6.7 g, 19.45 mmol, 41.28% yield) as a colorless oil.

### Step 2) 2-(2-((tert-butyldiphenylsilyl)oxy)ethoxy)acetaldehyde

to a mixture of 2-[2-[tert-butyl(diphenyl)silyl]oxyethoxy]ethanol (6.7 g, 19.45 mmol), sodium bromide (0.2 g, 1.94 mmol), sodium hydrogen carbonate (0.65 g, 7.78 mmol, 0.400 eq) and 2,2,6,6-tetramethylpiperidin-1-ol (60.77 mg, 0.390 mmol) in DCM (100 mL) and water (100 mL) cooled to 0° C. was added sodium hypochlorite (21.71 g, 29.17 mmol, 10% wt aq.) slowly.

After addition, the mixture was stirred at 0° C. for 0.5 h. The organic phase was separated, dried over sodium sulfate and concentrate. The residue was purified by silica gel chromatography eluting with PE:EA=20:1 to afford 2-[2-[tert-butyl(diphenyl)silyl]oxyethoxy]acetaldehyde (5 g, 14.6 mmol, 75.07%) as a colorless oil. ¹H NMR (400 MHz, CDCl₃) δ=9.45 (s, 1H), 7.72-7.70 (m, 4H), 7.47-7.41 (m, 6H), 4.18 (s, 2H), 3.89 (t, J=4.8, 2H), 3.71 (t, J=4.8, 2H), 1.09 (s, 9H) ppm

### Step 3) (E)-methyl 4-(2-((tert-butyldiphenylsilyl)oxy)ethoxy)but-2-enoate

To an ice-cooled solution of trimethyl phosphonoacetate (3.19 g, 17.52 mmol) in THF (100 mL) was added sodium hydride (0.88 g, 21.9 mmol, 60% in oil) slowly. After addition, a solution of 2-[2-[tert-butyl(diphenyl)silyl]oxyethoxy]acetaldehyde (5.0 g, 14.6 mmol) in THF (20 mL) was added. The resulting suspension was stirred at 0° C. for 0.5 h. The mixture was quenched with 1N HCl, extracted with EA (100 mL×2), washed with brine, dried over sodium sulfate and concentrated. The residue was purified by silica gel chromatography eluting with PE:EA=20:1 to afford methyl (E)-4-[2-[tert-butyl(diphenyl)silyl]oxyethoxy]but-2-enoate (4.5 g, 11.29 mmol, 77.34% yield) as a colorless oil. ¹H NMR (400 MHz, CDCl₃) δ=7.73-7.71 (m, 4H), 7.46-7.41 (m, 6H), 7.00-6.97 (m, 1H), 6.16 (d, J=16.0, 1H), 4.23 (q, J=2.0, 2H), 3.85 (t, J=5.2, 2H), 3.63 (t, J=5.2, 2H), 1.08 (s, 9H) ppm.

### Step 4) methyl 4-(2-((tert-butyldiphenylsilyl)oxy)ethoxy)-3-(nitromethyl)butanoate

A mixture of (E)-methyl 4-(2-((tert-butyldiphenylsilyl)oxy)ethoxy)but-2-enoate (4.5 g, 11.29 mmol) and 1,1,3,3-tetramethylguanidine (0.14 mL, 1.13 mmol) in nitromethane (24.46 mL, 451.61 mmol). The resulting mixture was stirred at 10° C. for 72 h. The mixture was concentrated to removed excessive nitromethane and then diluted with DCM (100 mL), neutralized with 1 N HCl, washed with brine, dried over sodium sulfate and concentrated. The residue was purified by silica gel chromatography eluting with PE:EA=20:1 to afford methyl 3-[2-[tert-butyl(diphenyl)silyl]oxyethoxymethyl]-4-nitro-butanoate (4.5 g, 9.79 mmol, 86.72%) as a yellow oil. ¹H NMR (400 MHz, CDCl₃) δ=7.70-7.68 (m, 4H), 7.44-7.41 (m, 6H), 4.59-4.54 (m, 2H), 3.80 (t, J=4.8 Hz, 1H), 3.70 (s, 3H), 3.57-3.52 (m, 4H), 2.94-2.91 (m, 2H), 2.54-2.44 (m, 2H), 1.07 (s, 9H) ppm.

### Step 5) 4-((2-((tert-butyldiphenylsilyl)oxy)ethoxy)methyl) pyrrolidin-2-one

A mixture of methyl 3-[2-[tert-butyl(diphenyl)silyl]oxyethoxymethyl]-4-nitro-butanoate (1.0 g, 2.18 mmol) and Raney Ni (67.8 mg, 1.16 mmol) in ethanol (20 mL) was hydrogenated under 3600 mm Hg at 55° C. for 15. Raney Ni was removed by filtration and the filtrate was concentrated to give 4-[2-[tert-butyl(diphenyl)silyl]oxyethoxymethyl]pyrrolidin-2-one (0.750 g, 1.89 mmol, 86.7%), the crude product was used in the next step without further purification.

MS obsd. (ESI+) [(M+Na)⁺]:420.1

### Step 6) 4-((2-((tert-butyldiphenylsilyl)oxy)ethoxy)methyl)-1-(4-nitrophenyl)pyrrolidin-2-one

A mixture of 1-iodo-4-nitrobenzene (2.19 g, 8.8 mmol), 4-[2-[tert-butyl(diphenyl)silyl]oxyethoxymethyl]pyrrolidin-2-one (3.5 g, 8.8 mmol), copper(I) iodide (167.66 mg, 0.880 mmol), trans-(1r,2r)-N,N′-bismethyl-1,2-cyclohexanediamine (125.22 mg, 0.880 mmol) and phosphoric acid, potassium salt (3.74 g, 17.61 mmol) in DMSO (20 mL) was heated to 100° C. for 15 h. The mixture was diluted water (20 mL), extracted with EA (100 mL×2), washed with brine, dried over sodium sulfate and concentrated. the residue was purified by silica gel chromatography eluting with PE:EA=3:1 to afford 4-[2-[tert-butyl(diphenyl)silyl]oxyethoxymethyl]-1-(4-nitrophenyl)pyrrolidin-2-one (2.6 g, 5.01 mmol, 56.94%) as a yellow oil.

MS obsd. (ESI+) [(M+Na)⁺]:541.0.

### Step 7) 4-((2-hydroxyethoxy)methyl)-1-(4-nitrophenyl)pyrrolidin-2-one

To a solution of 4-[2-[tert-butyl(diphenyl)silyl]oxyethoxymethyl]-1-(4-nitrophenyl)pyrrolidin-2-one (2.3 g, 4.43 mmol) in THF (10 mL) was added tetrabutylammonium fluoride (2.32 g, 8.87 mmol). The resulting mixture was stirred at 10° C. for 15 h. The mixture was diluted with water, extracted with EA (100 mL×2), washed with brine, dried over sodium sulfate and concentrated.

The residue was purified by flash column to afford 4-(2-hydroxyethoxymethyl)-1-(4-nitrophenyl)pyrrolidin-2-one (1 g, 3.57 mmol, 80.46%) as a yellow solid.

MS obsd. (ESI+) [(M+H)⁺]:281.0

### Step 8) 2-((1-(4-nitrophenyl)-5-oxopyrrolidin-3-yl)methoxy)ethyl methanesulfonate

To a solution of 4-(2-hydroxyethoxymethyl)-1-(4-nitrophenyl)pyrrolidin-2-one (700.0 mg, 2.5 mmol) and methanesulfonyl chloride (0.19 mL, 2.5 mmol) in DCM (10 mL) was added triethylamine (0.35 mL, 2.5 mmol) slowly. After stirring for 2 h at 0° C. the mixture was diluted with water (100 mL), the organic phase separated was dried over sodium sulfate and concentrated to afford 2-[[1-(4-nitrophenyl)-5-oxo-pyrrolidin-3-yl] methoxy]ethyl methanesulfonate (800 mg, 2.23 mmol, 89.38%), the crude product was used in the next step without further purification.

MS obsd. (ESI+) [(M+H)⁺]:359.0.

### Step 9) 4-((2-azidoethoxy)methyl)-1-(4-nitrophenyl)pyrrolidin-2-one

A mixture of 2-[[1-(4-nitrophenyl)-5-oxo-pyrrolidin-3-yl]methoxy]ethyl methanesulfonate (800.0 mg, 2.23 mmol) and sodium azide (725.6 mg, 11.16 mmol) in DMF (10 mL) was heated to 50° C. for 15 h. The mixture was diluted with water, extracted with EA (50 mL×2), washed with brine, dried over sodium sulfate and concentrated to afford 4-(2-azidoethoxymethyl)-1-(4-nitrophenyl)pyrrolidin-2-one (600 mg, 1.97 mmol, 88.04% yield) yellow solid.

MS obsd. (ESI+) [(M+H)⁺]:306.2.

### Step 10) tert-butyl (2-((1-(4-nitrophenyl)-5-oxopyrrolidin-3-yl) methoxy)ethyl)carbamate

To a solution of 4-(2-azidoethoxymethyl)-1-(4-nitrophenyl)pyrrolidin-2-one (700.0 mg, 2.29 mmol) in THF (10 mL) and water (2 mL) was added triphenylphosphine (721.67 mg, 2.75 mmol). The resulting mixture was stirred at 10° C. for 15 h, then di-t-butyldicarbonate (600.51 mg, 2.75 mmol) was added. The resulting mixture was stirred for another 6 h. The mixture was concentrated and the residue was purified by flash column to afford tert-butyl N-[2-[[1-(4-nitrophenyl)-5-oxo-pyrrolidin-3-yl]methoxy]ethyl]carbamate (750 mg, 1.98 mmol, 86.21% yield) as a colorless oil.

MS obsd. (ESI+) [(M+Na)⁺]:402.1.

### Step 11) tert-butyl (2-((1-(4-aminophenyl)-5-oxopyrrolidin-3-yl) methoxy)ethyl)carbamate

A mixture of tert-butyl N-[2-[[1-(4-nitrophenyl)-5-oxo-pyrrolidin-3-yl]methoxy]ethyl]carbamate (750.0 mg, 1.98 mmol) and palladium on carbon (75 mg, 10% wt) in methanol (10 mL) was hydrogenated under 760 mmHg for 4 h. The catalyst was removed by filtration and the filtrate was concentrated to afford tert-butyl N-[2-[[1-(4-aminophenyl)-5-oxo-pyrrolidin-3-yl]methoxy]ethyl]carbamate (500 mg, 1.43 mmol, 72.39%) as a colorless oil.

MS obsd. (ESI+) [(M+H)⁺]:350.4.

### Step 12) tert-butyl (2-((1-(4-((3-(4-(difluoromethoxy)phenyl) imidazo[1,2-a]pyrazin-8-yl)amino)phenyl)-5-oxopyrrolidin-3-yl)methoxy)ethyl)carbamate

A mixture of tert-butyl N-[2-[[1-(4-aminophenyl)-5-oxo-pyrrolidin-3-yl]methoxy]ethyl]carbamate (100.0 mg, 0.290 mmol), 8-chloro-3-[4-(difluoromethoxy)phenyl]imidazo[1,2-a]pyrazine (84.62 mg, 0.290 mmol), Brettphos Pd G3 (24.54 mg, 0.030 mmol) and potassium carbonate (79.11 mg, 0.570 mmol) was heated to 110° C. for 15 h. The mixture was diluted with water, extracted with EA (100 mL×2). The combined organic phases were washed with brine, dried over sodium sulfate and concentrated. the residue was purified by prep-TLC (DCM:MeOH=10:1) to afford tert-butyl N-[2-[[1-[4-[[3-[4-(difluoromethoxy)phenyl]imidazo[1,2-a]pyrazin-8-yl]amino]phenyl]-5-oxo-pyrrolidin-3-yl]methoxy]ethyl]carbamate (80 mg, 0.130 mmol, 45.93%) as a colorless oil.

MS obsd. (ESI+) [(M+H)⁺]:609.2.

### Step 13) 4-((2-aminoethoxy)methyl)-1-(4-((3-(4-(difluoromethoxy)phenyl)imidazo[1,2-a]pyrazin-8-yl)amino)phenyl)pyrrolidin-2-one

To a solution of tert-butyl N-[2-[[1-[4-[[3-[4-(difluoromethoxy)phenyl]imidazo[1,2-a]pyrazin-8-yl]amino]phenyl]-5-oxo-pyrrolidin-3-yl]methoxy]ethyl]carbamate (75.0 mg, 0.120 mmol) in methanol (5 mL) was added hydrogen chloride in MeOH (3.0 mL, 12 mmol, 4 M). The resulting mixture was stirred at 10° C. for 15 h. The mixture was concentrated and the crude was purified by prep-HPLC to afford 4-(2-aminoethoxymethyl)-1-[4-[[3-[4-(difluoromethoxy)phenyl]imidazo[1,2-a]pyrazin-8-yl]amino]phenyl]pyrrolidin-2-one (44.3 mg, 0.090 mmol, 70.41%) as a white solid

¹H NMR (400 MHz, DMSO-d₆) δ=9.66 (s, 1H), 8.37 (s, 1H), 8.05 (d, J=8.8 Hz, 2H), 7.94 (d, J=4.8 Hz, 1H), 7.86 (s, 1H), 7.77 (d, J=8.8 Hz, 2H), 7.61 (d, J=8.8 Hz, 2H), 7.48-7.18 (m, 4H), 3.93 (t, J=8.8 Hz, 1H), 3.68-3.64 (m, 1H), 3.54 (t, J=5.6, 2H), 3.50 (d, J=6.4 Hz, 2H), 2.91 (t, J=4.8 Hz, 2H), 2.74-2.59 (m, 2H), 2.41-2.36 (m, 1H) ppm.

MS obsd. (ESI+) [(M+H)⁺]:509.2.

### Example 65

### N-(4-((3-(4-(prop-2-yn-1-yloxy)phenyl)imidazo[1,2-a]pyrazin-8-yl)amino)phenyl)acetamide RW-11-007S

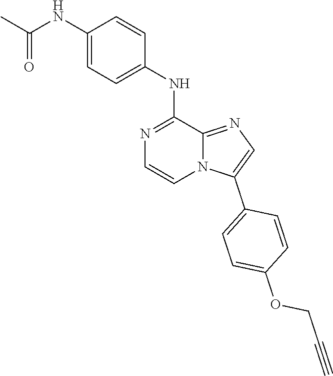 [SMILES: C#CCOc1ccc(-c2cnc3c(Nc4ccc(NC(C)=O)cc4)nccn23)cc1]

### Step 1) 4-(8-chloroimidazo[1,2-a]pyrazin-3-yl)phenol

A mixture of 8-chloro-3-iodo-imidazo[1,2-a]pyrazine (2.79 g, 10 mmol, 1 eq), (4-hydroxyphenyl)boronic acid (1.66 g, 12 mmol, 1.2 eq), [1,1′-BIS(DIPHENYLPHOSPHINO)FERROCENE]DICHLOROPALLADIUM(II) (731.71 mg, 1 mmol, 0.100 eq) and Sodium carbonate (2.12 g, 20 mmol, 2 eq) in 1,4-dioxane (36 mL)/water (4 mL) was stirred under N2 at 80° C. for 16 h.

The mixture was filtered and purified by silica column PE/EA=2:1 to afford 4-(8-chloroimidazo[1,2-a]pyrazin-3-yl)phenol 1.3 g as a red solid.

MS [M+H]+: 246.0

### Step 2) 8-chloro-3-(4-(prop-2-yn-1-yloxy)phenyl)imidazo[1,2-a]pyrazine

A mixture of 4-(8-chloroimidazo[1,2-a]pyrazin-3-yl)phenol (100.0 mg, 0.410 mmol, 1 eq), 3-BROMO-1-(TRIMETHYLSILYL)-1-PROPYNE (93.37 mg, 0.490 mmol, 1.2 eq), potassium carbonate (67.51 mg, 0.490 mmol, 1.2 eq) in DMF (5 mL) was stirred under nitrogen at 80° C. for 3 h. The mixture was poured into water, extracted with EtOAc, concentrated and purified by flash column (FA) to afford 8-chloro-3-(4-(prop-2-yn-1-yloxy)phenyl)imidazo[1,2-a]pyrazine (60 mg) as a yellow solid.

MS [M+H]+: 283.5

### Step 3) N-(4-((3-(4-(prop-2-yn-1-yloxy)phenyl)imidazo[1,2-a]pyrazin-8-yl)amino)phenyl)acetamide

A mixture of 8-chloro-3-(4-prop-2-ynoxyphenyl)imidazo[1,2-a]pyrazine (30.0 mg, 0.110 mmol, 1 eq), 4′-AMINOACETANILIDE (31.76 mg, 0.210 mmol, 2 eq), N,N-diisopropylethylamine (0.04 mL, 0.210 mmol, 2 eq) in DMF (3 mL) was stirred under N2 at 110° C. for 16 h. The solution was poured into water (30 mL), extracted with EtOAc (30 mL*2), concentrated and purified by prep-HPLC (FA) and prep-TLC (DCM/MeOH=10:1) to afford N-(4-((3-(4-(prop-2-yn-1-yloxy)phenyl)imidazo[1,2-a]pyrazin-8-yl)amino)phenyl)acetamide (15.0 mg) as a white solid.

MS [M+H]+: 398.1

### Example 66

### 3-(2,3-difluoro-4-methoxyphenyl)-N-(4-(N,S-dimethylsulfonimidoyl)-3-methylphenyl)imidazo[1,2-a]pyrazin-8-amine

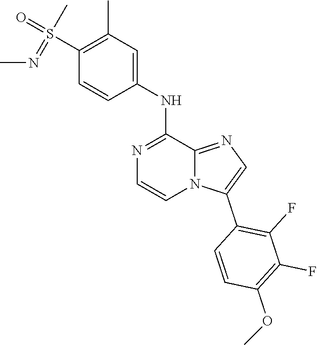 [SMILES: CN=S(C)(=O)c1ccc(Nc2nccn3c(-c4ccc(OC)c(F)c4F)cnc23)cc1C]

### Step 1) methyl(2-methyl-4-nitrophenyl)sulfane

A mixture of 2-fluoro-5-nitrotoluene (4.0 g, 25.78 mmol, 1 eq) and sodium thiomethoxide (1.81 g, 25.78 mmol, 1 eq) in DMF (20 mL) was stirred at 100° C. for 5 h. The mixture was poured into water (100 mL) and extracted with EtOAc (50 mL*3), washed with brine (100 mL*2), dried over Na₂SO₄ and purified by silica column chromatography (PE/EA=5:1) to afford 2-methyl-1-methylsulfanyl-4-nitro-benzene (2.68 g, 14.63 mmol, 56.72% yield) as a yellow solid.

MS [M+H]+: 184.1

### Step 2) 2-methyl-1-(methylsulfinyl)-4-nitrobenzene

A stirred solution of 2-methyl-1-methylsulfanyl-4-nitro-benzene (2.68 g, 14.63 mmol, 1 eq) in THF (50 ml) was added dropwise a solution of 3-chloroperoxybenzoic acid (3.12 g, 15.36 mmol, 1.05 eq) in THF (20 mL) at 0° C. The mixture was purified by silica column chromatography (PE/EA=3:1) to afford 2-methyl-1-methylsulfinyl-4-nitro-benzene (2.23 g, 11.19 mmol, 76.02% yield) as a white solid.

MS [M+H]+: 200.1

### Step 3) 2-methyl-1-(S-methylsulfonimidoyl)-4-nitrobenzene

A stirred solution of 2-methyl-1-methylsulfinyl-4-nitro-benzene (2.4 g, 12.04 mmol, 1 eq) in Eatons reagent (20.0 mL, 12.04 mmol, 1 eq) was added sodium azide (3.36 g, 51.68 mmol, 4.29 eq) at 60° C. and stirred for 0.5 h. The mixture was poured into NH4OH solution (50 mL) and extracted with EA (50 mL), washed with brine (50 mL), concentrated and purified by silica column chromatography (PE/EA=1:1) to afford 2-methyl-1-(S-methylsulfonimidoyl)-4-nitrobenzene (1.2 g, 5.6 mmol, 46.52% yield) as a yellow solid.

MS [M+H]+: 214.9

### Step 4) methyl-methylimino-(2-methyl-4-nitro-phenyl)-oxo-λ\hat{ }{6}-sulfane

A stirred solution of 2-methyl-1-(S-methylsulfonimidoyl)-4-nitrobenzene (0.4 g, 1.87 mmol, 1 eq) and cesium carbonate (1216.65 mg, 3.73 mmol, 2 eq) in DMF (5 mL) was added iodomethane (0.8 mL, 12.96 mmol, 6.94 eq) at 60° C. and stirred for 0.5h. The mixture was poured into water (30 mL) and extracted with EA (20 mL*3), washed with brine (50 mL*2), concentrated, purified by silica column chromatography (PE/EA=1:2) to afford methyl-methylimino-(2-methyl-4-nitro-phenyl)-oxo-λ\hat{ }{6}-sulfane (50 mg, 0.220 mmol, 11.73% yield) as a light yellow solid.

MS [M+H]+: 229.0

### Step 5) 4-(N,S-dimethylsulfonimidoyl)-3-methylaniline

A stirred solution of nickel(II) chloride (26.03 mg, 0.110 mmol, 0.500 eq) in methanol (3 mL) was added sodium borohydride (4.14 mg, 0.110 mmol, 0.500 eq) at 0° C. and stirred for 10 min, then methyl-methylimino-(2-methyl-4-nitro-phenyl)-oxo-λ\hat{ }(6)-sulfane (50.0 mg, 0.220 mmol, 1 eq) was added to the mixture at 0° C., after 10 min, sodium borohydride (24.86 mg, 0.660 mmol, 3 eq) was added to the mixture and stirred for 0.5h. The mixture was poured into water (30 mL) and extracted with EA (20 mL*3), washed with brine (50 mL*2), concentrated to afford 4-(N,S-dimethylsulfonimidoyl)-3-methyl-aniline (40 mg, 0.200 mmol, 92.1% yield) as a light yellow oil.

MS [M+H]+: 199.0

### Step 6) 3-(2,3-difluoro-4-methoxyphenyl)-N-(4-(N,S-dimethylsulfonimidoyl)-3-methylphenyl)imidazo[1,2-a]pyrazin-8-amine

A mixture of 4-(N,S-dimethylsulfonimidoyl)-3-methyl-aniline (40.0 mg, 0.200 mmol, 1 eq), 8-chloro-3-(2,3-difluoro-4-methoxy-phenyl)imidazo[1,2-a]pyrazine (89.47 mg, 0.300 mmol, 1.5 eq), cesium carbonate (197.18 mg, 0.610 mmol, 3 eq), tris(dibenzylideneacetone)dipalladium (0) (18.47 mg, 0.020 mmol, 0.100 eq) and 9,9-dimethyl-4,5-bis(diphenylphosphino)xanthene (11.67 mg, 0.020 mmol, 0.100 eq) in 1,4-dioxane (5 mL) was stirred under N2 at 115° C. in a microwave for 2 h. The mixture was filtered and concentrated, purified by silica column chromatography (DCM/MeOH=50:1) and prep-HPLC (FA) to afford 3-(2,3-difluoro-4-methoxy-phenyl)-N-[4-(N,S-dimethylsulfonimidoyl)-3-methyl-phenyl]imidazo[1,2-a]pyrazin-8-amine (9.3 mg, 0.020 mmol, 9.68% yield) as a white solid.

MS [M+H]+: 458.1

### Example 67

### N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine; formic acid

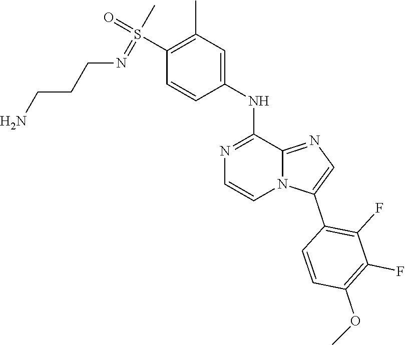 [SMILES: COc1ccc(-c2cnc3c(Nc4ccc(S(C)(=O)=NCCCN)c(C)c4)nccn23)c(F)c1F]

### Step 1) tert-butyl N-[3-[[methyl-(2-methyl-4-nitro-phenyl)-oxo-λ\hat{ }{6}-sulfanylidene]amino]propyl]carbamate

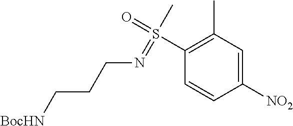 [SMILES: Cc1cc([N+](=O)[O-])ccc1S(C)(=O)=NCCCNC(=O)OC(C)(C)C]

A stirred solution of 2-methyl-1-(S-methylsulfonimidoyl)-4-nitrobenzene (0.4 g, 1.87 mmol, 1 eq) in cesium carbonate (1216.65 mg, 3.73 mmol, 2 eq) was added 3-(BOC-amino)propyl bromide (0.67 g, 2.8 mmol, 1.5 eq) at 60° C. and stirred for 16 h. The mixture was poured into water (30 mL) and extracted with EA (20 mL*3), washed with brine (50 mL*2), concentrated, purified by silica column chromatography (PE/EA=1:2) and reversed phase-HPLC (FA) to afford N-[3-[[methyl-(2-methyl-4-nitro-phenyl)-oxo-λ\hat{ }{6}-sulfanylidene]amino]propyl]carbamate (390 mg, 1.05 mmol, 56.23% yield) as a yellow oil.

MS [M+H]+: 372.0

### Step 2) tert-butyl N-[3-[[(4-amino-2-methyl-phenyl)-methyl-oxo-λ\hat{ }{6}-sulfanylidene]amino]propyl]carbamate

A stirred solution of nickel(II) chloride (118.38 mg, 0.500 mmol, 0.500 eq) in methanol (10 mL) was added sodium borohydride (18.84 mg, 0.500 mmol, 0.500 eq) at 0° C., then tert-butyl N-[3-[[methyl-(2-methyl-4-nitro-phenyl)-oxo-λ\hat{ }{6}-sulfanylidene]amino]propyl]carbamate (370.0 mg, 1 mmol, 1 eq) and sodium borohydride (113.05 mg, 2.99 mmol, 3 eq) was added to the mixture and stirred at 0° C. for 0.5 h. The mixture was poured into water (30 mL) and extracted with EA (20 mL*3), washed with brine (50 mL*2), concentrated to afford crude tert-butyl N-[3-[[(4-amino-2-methyl-phenyl)-methyl-oxo-λ\hat{ }{6}-sulfanylidene]amino]propyl]carbamate (330 mg, 0.970 mmol, 97.02% yield) as a light yellow oil.

MS [M+H]+: 342.3

### Step 3) tert-butyl N-[3-[[(4-amino-2-methyl-phenyl)-methyl-oxo-λ\hat{ }{6}-sulfanylidene]amino]propyl]carbamate

A mixture of tert-butyl N-[3-[[(4-amino-2-methyl-phenyl)-methyl-oxo-λ\hat{ }{6}-sulfanylidene]amino]propyl]carbamate (200.0 mg, 0.590 mmol, 1 eq), 8-chloro-3-(2,3-difluoro-4-methoxy-phenyl)imidazo[1,2-a]pyrazine (173.17 mg, 0.590 mmol, 1 eq), cesium carbonate (572.5 mg, 1.76 mmol, 3 eq), tris(dibenzylideneacetone)dipalladium(0) (53.63 mg, 0.060 mmol, 0.100 eq) and 9,9-dimethyl-4,5-bis(diphenylphosphino)xanthene (33.89 mg, 0.060 mmol, 0.100 eq) in 1,4-dioxane (10 mL) was stirred under N2 at 115° C. in a microwave for 2 h. The mixture was filtered and concentrated, purified by silica column chromatography (DCM/MeOH=50:1) to afford tert-butyl N-[3-[[[4-[[3-(2,3-difluoro-4-methoxy-phenyl)imidazo[1,2-a]pyrazin-8-yl]amino]-2-methyl-phenyl]-methyl-oxo-λ\hat{ }{6}-sulfanylidene]amino]propyl]carbamate (195 mg, 0.320 mmol, 55.43% yield) as a light yellow solid.

MS [M+H]+: 601.3

### Step 4) N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine; formic acid RW-11-014T-6

A mixture of tert-butyl N-[3-[[[4-[[3-(2,3-difluoro-4-methoxy-phenyl)imidazo[1,2-a]pyrazin-8-yl]amino]-2-methyl-phenyl]-methyl-oxo-λ\hat{ }{6}-sulfanylidene]amino]propyl]carbamate (195.0 mg, 0.320 mmol, 1 eq) in methanol (5 mL) was added hydrochloric acid in MeOH (5 mL, 4N) and stirred at 20° C. for 16 h. The solution was concentrated and purified by prep-HPLC (FA) to afford N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine; formic acid (64.7 mg, 0.120 mmol, 35.11% yield) as a white solid.

MS [M+H]+: 501.1

### Example 68

### N-[4-[N-(3-aminopropyl)-S-methyl-sulfonimidoyl]phenyl]-3-(2,3-difluoro-4-methoxy-phenyl)imidazo[1,2-a]pyrazin-8-amine; formic acid

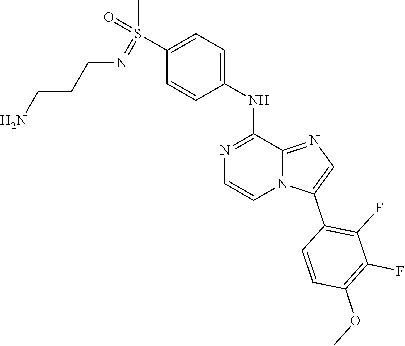 [SMILES: COc1ccc(-c2cnc3c(Nc4ccc(S(C)(=O)=NCCCN)cc4)nccn23)c(F)c1F]

### Step 1) 1-(methylsulfinyl)-4-nitrobenzene

A stirred solution of 1-methylsulfanyl-4-nitro-benzene (5.0 g, 29.55 mmol, 1 eq) in THF (50 ml) was added dropwise to a solution of 3-chloroperoxybenzoic acid (6.3 g, 31.03 mmol, 1.05 eq) in THF (20 mL) at 0° C., the solution was stirred for 0.5 h. The mixture was filtered and the residue was washed with THF (50 mL), and then dried to afford 1-methylsulfinyl-4-nitro-benzene (2.1 g, 11.34 mmol, 38.37% yield) as a white solid.

MS obsd. (ESI⁺) [(M+H)⁺]: 186.1

### Step 2) 1-(S-methylsulfonimidoyl)-4-nitrobenzene

A stirred solution of 1-methylsulfinyl-4-nitro-benzene (1.9 g, 10.26 mmol, 1 eq) in EATONS REAGENT (20.0 mL, 10.26 mmol, 1 eq) was added sodium azide (2.67 g, 41.04 mmol, 4 eq) at 60° C. and stirred for 0.5 h. The mixture was poured into sat. aq. NH4OH solution (50 mL) and extracted with EA (50 mL), washed with brine (50 mL), concentrated and purified by silica column chromatography (PE/EA=1:1) to afford 1-(S-methylsulfonimidoyl)-4-nitrobenzene (0.800 g, 4 mmol, 38.95% yield) as a yellow solid.

MS obsd. (ESI⁺) [(M+H)⁺]: 200.9

### Step 3) tert-butyl N-[3-[[methyl-(4-nitrophenyl)-oxo-λ\hat{ }{6}-sulfanylidene]amino]propyl]carbamate

To mixture of 1-(S-methylsulfonimidoyl)-4-nitrobenzene (0.3 g, 1.5 mmol, 1 eq) and cesium carbonate (976.39 mg, 3 mmol, 2 eq) in DMF (5 mL) was added 3-(BOC-amino)propyl bromide (0.54 g, 2.25 mmol, 1.5 eq) and stirred at 70° C. for 16 h. The mixture was poured into water (30 mL) and extracted with EA (20 mL*3), washed with brine (50 mL*2), concentrated, purified by silica column chromatography (PE/EA=1:2) to afford tert-butyl N-[3-[[methyl-(4-nitrophenyl)-oxo-λ\hat{ }{6}-sulfanylidene]amino]propyl]carbamate (210 mg, 0.590 mmol, 39.21% yield) as light yellow oil.

MS [M+H]+: 358.0

### Step 4) tert-butyl N-[3-[[(4-aminophenyl)-methyl-oxo-λ\hat{ }{6}-sulfanylidene]amino]propyl]carbamate

A stirred solution of nickel(II) chloride (69.82 mg, 0.290 mmol, 0.500 eq) in methanol (5 mL) was added sodium borohydride (11.11 mg, 0.290 mmol, 0.500 eq) at 0° C., then tert-butyl N-[3-[[methyl-(4-nitrophenyl)-oxo-λ\hat{ }{6}-sulfanylidene]amino]propyl]carbamate (210.0 mg, 0.590 mmol, 1 eq) and sodium borohydride (66.68 mg, 1.76 mmol, 3 eq) was added to the mixture and stirred at 0° C. for 0.5 h. The mixture was poured into water (30 mL) and extracted with EA (20 mL*3), concentrated to afford tert-butyl N-[3-[[(4-aminophenyl)-methyl-oxo-λ\hat{ }{6}-sulfanylidene]amino]propyl]carbamate (120 mg, 0.370 mmol, 62.38% yield) as a light yellow oil.

MS [M+H]+: 328.2

### Step 5) tert-butyl N-[3-[[[4-[[3-(2,3-difluoro-4-methoxy-phenyl)imidazo[1,2-a]pyrazin-8-yl]amino]phenyl]-methyl-oxo-λ\hat{ }{6}-sulfanylidene]amino]propyl]carbamate

A mixture of tert-butyl N-[3-[[(4-aminophenyl)-methyl-oxo-λ\hat{ }{6}-sulfanylidene]amino]propyl]carbamate (120.0 mg, 0.370 mmol, 1 eq), 8-chloro-3-(2,3-difluoro-4-methoxy-phenyl)imidazo[1,2-a]pyrazine (108.36 mg, 0.370 mmol, 1 eq), cesium carbonate (358.22 mg, 1.1 mmol, 3 eq), tris(dibenzylideneacetone)dipalladium(0) (33.56 mg, 0.040 mmol, 0.100 eq) and 9,9-dimethyl-4,5-bis(diphenylphosphino)xanthene (21.21 mg, 0.040 mmol, 0.100 eq) in 1,4-dioxane (10 mL) was stirred under N2 at 115° C. in a microwave for 2 h. The mixture was filtered and concentrated, purified by silica column chromatography (DCM/MeOH=50:1) and prep-TLC (DCM/MeOH=10:1) to afford tert-butyl N-[3-[[[4-[[3-(2,3-difluoro-4-methoxy-phenyl)imidazo[1,2-a]pyrazin-8-yl]amino]phenyl]-methyl-oxo-λ\hat{ }{6}-sulfanylidene]amino]propyl]carbamate (122 mg, 0.210 mmol, 56.75% yield) as a light yellow oil.

MS [M+H]+: 587.1

### Step 6) N-[4-[N-(3-aminopropyl)-S-methyl-sulfonimidoyl]phenyl]-3-(2,3-difluoro-4-methoxy-phenyl)imidazo[1,2-a]pyrazin-8-amine; formic acid

To a stirred solution of tert-butyl N-[3-[[[4-[[3-(2,3-difluoro-4-methoxy-phenyl)imidazo[1,2-a]pyrazin-8-yl]amino]phenyl]-methyl-oxo-λ\hat{ }{6}-sulfanylidene]amino]propyl]carbamate (122.0 mg, 0.210 mmol, 1 eq) in methanol (5 mL) was added hydrochloric acid in MeOH (5 mL, 4N) and then stirred at 20° C. for 16 h. The solution was concentrated and purified by prep-HPLC (FA) to afford N-[4-[N-(3-aminopropyl)-S-methyl-sulfonimidoyl]phenyl]-3-(2,3-difluoro-4-methoxy-phenyl)imidazo[1,2-a]pyrazin-8-amine; formic acid (35.4 mg, 0.070 mmol, 31.26% yield) as a white solid.

MS [M+H]+: 487.1

### Example 69

### 3-(2,3-difluoro-4-methoxyphenyl)-N-(4-(N,S-dimethylsulfonimidoyl)phenyl)imidazo[1,2-a]pyrazin-8-amine

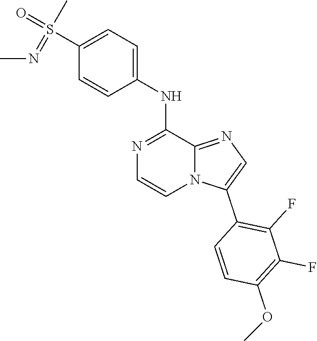 [SMILES: CN=S(C)(=O)c1ccc(Nc2nccn3c(-c4ccc(OC)c(F)c4F)cnc23)cc1]

### Step 1) methyl-methylimino-(4-nitrophenyl)-oxo-λ\hat{ }{6}-sulfane

To a stirred solution of 1-(S-methylsulfonimidoyl)-4-nitrobenzene (0.3 g, 1.5 mmol, 1 eq) and cesium carbonate (976.39 mg, 3 mmol, 2 eq) in DMF (5 mL) was added iodomethane (0.46 mL, 7.49 mmol, 5 eq) at 20° C. and stirred for 16 h. The mixture was poured into water (50 mL) and extracted with EA (30 mL*3), washed with brine (50 mL*2), concentrated and purified by Prep-TLC (PE/EA=1:2) to afford methyl-methylimino-(4-nitrophenyl)-oxo-λ\hat{ }{6}-sulfane (65 mg, 0.300 mmol, 20.25% yield) as a light yellow solid.

MS [M+H]+: 215.1

### Step 2) 4-(N,S-dimethylsulfonimidoyl)aniline

To a stirred solution of nickel(II) chloride (36.06 mg, 0.150 mmol, 0.500 eq) in methanol (3 mL) was added sodium borohydride (5.74 mg, 0.150 mmol, 0.500 eq) at 0° C. and stirred for 10 min, then methyl-methylimino-(4-nitrophenyl)-oxo-λ\hat{ }{6}-sulfane (65.0 mg, 0.300 mmol, 1 eq) and sodium borohydride (34.43 mg, 0.910 mmol, 3 eq) were added to the mixture and stirred at 0° C. for 0.5h. The mixture was poured into water (30 mL) and extracted with EA (20 mL*3), washed with brine (50 mL*2), concentrated to afford 4-(N,S-dimethylsulfonimidoyl)aniline (50 mg, 0.270 mmol, 89.44% yield) as a light yellow oil.

MS [M+H]+: 184.9

### Step 3) 3-(2,3-difluoro-4-methoxyphenyl)-N-(4-(N,S-dimethylsulfonimidoyl)phenyl)imidazo[1,2-a]pyrazin-8-amine

A mixture of 4-(N,S-dimethylsulfonimidoyl)aniline (50.0 mg, 0.270 mmol, 1 eq), 8-chloro-3-(2,3-difluoro-4-methoxy-phenyl)imidazo[1,2-a]pyrazine (80.23 mg, 0.270 mmol, 1 eq), cesium carbonate (265.24 mg, 0.810 mmol, 3 eq), tris(dibenzylideneacetone)dipalladium(0) (24.85 mg, 0.030 mmol, 0.100 eq) and 9,9-dimethyl-4,5-bis(diphenylphosphino)xanthene (15.7 mg, 0.030 mmol, 0.100 eq) in 1,4-dioxane (5 mL) was stirred under N2 at 115° C. in a microwave for 2 h. The mixture was filtered and concentrated, purified by silica column chromatography (DCM/MeOH=50:1) and prep-HPLC (FA) to afford 3-(2,3-difluoro-4-methoxy-phenyl)-N-[4-(N,S-dimethylsulfonimidoyl)phenyl]imidazo[1,2-a]pyrazin-8-amine (22.8 mg, 0.050 mmol, 18.32% yield) as a white solid.

MS [M+H]+: 444.2

### Example 70

### N-[4-[N-(3-aminopropyl)-S-methyl-sulfonimidoyl]-3-ethyl-phenyl]-3-(2,3-difluoro-4-methoxy-phenyl)imidazo[1,2-a]pyrazin-8-amine; formic acid

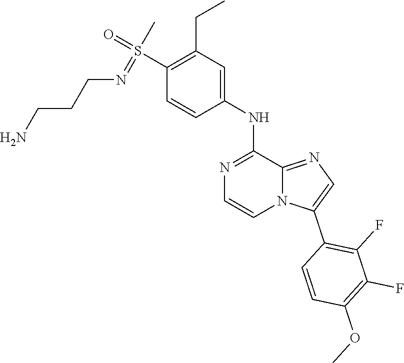 [SMILES: CCc1cc(Nc2nccn3c(-c4ccc(OC)c(F)c4F)cnc23)ccc1S(C)(=O)=NCCCN]

### Step 1) 2-bromo-1-methylsulfanyl-4-nitro-benzene

A mixture of 1-bromo-2-fluoro-5-nitrobenzene (5.0 g, 22.73 mmol, 1 eq) and sodium thiomethoxide (1.59 g, 22.73 mmol, 1 eq) in DMF (30 mL) was stirred at 0° C. for 5 h. The mixture was poured into water (100 mL) and extracted with EtOAc (50 mL*3), washed with brine (100 mL*2), dried over Na₂SO₄ and purified by silica column chromatography (PE/EA=10:1) to afford 2-bromo-1-methylsulfanyl-4-nitro-benzene (3 g, 12.09 mmol, 53.2% yield) as a yellow solid.

MS obsd. (ESI⁺) [(M)]: 248.9 (GCMS)

### Step 2) 2-bromo-1-(methylsulfinyl)-4-nitrobenzene

A stirred solution of 2-bromo-1-methylsulfanyl-4-nitro-benzene (3.0 g, 12.09 mmol, 1 eq) in THF (50 ml) was added dropwise to a solution of 3-chloroperoxybenzoic acid (2.45 g, 12.09 mmol, 1 eq) in THF (20 mL) at 0° C. and stirred at 0° C. for 0.5 h. The mixture was purified by silica column chromatography (PE/EA=5:1) to afford 2-bromo-1-methylsulfinyl-4-nitro-benzene (3 g, 11.36 mmol, 93.94% yield) as a yellow solid.

MS [M+H]+: 265.9

### Step 3) 2-bromo-1-(S-methylsulfonimidoyl)-4-nitrobenzene

A stirred solution of 2-bromo-1-methylsulfinyl-4-nitro-benzene (2.9 g, 10.98 mmol, 1 eq) in EATONS REAGENT (25.0 mL, 10.98 mmol, 1 eq) was added sodium azide (2.86 g, 43.92 mmol, 4 eq) at 60° C. and stirred at 60° C. for 0.5h. The mixture was poured into sat. aq. NH4OH solution (50 mL) and extracted with EA (50 mL), washed with brine (50 mL), concentrated and purified by silica column chromatography (PE/EA=3:1 to 1:1) and prep-HPLC (FA) to afford 2-bromo-1-(S-methylsulfonimidoyl)-4-nitrobenzene (1.8 g, 6.45 mmol, 58.73% yield) as a light yellow solid.

MS [M+H]+: 281.0

### Step 4) 1-(S-methylsulfonimidoyl)-4-nitro-2-vinylbenzene

A solution of 2-bromo-1-(S-methylsulfonimidoyl)-4-nitrobenzene (1.2 g, 4.3 mmol, 1 eq), tin vinyltributyl (1.3 mL, 4.45 mmol, 1.03 eq), tetrakis(triphenylphosphine)palladium(0) (248.41 mg, 0.210 mmol, 0.050 eq) in toluene (15 mL) was stirred under N2 at 100° C. for 16 h. The mixture was poured into KF solution (100 mL) and stirred for 0.5 h at 20° C., then extracted with EtOAc (40 mL*2), washed with brine (100 mL), concentrated and purified by silica column chromatography (PE/EA=5:1) to afford 1-(S-methylsulfonimidoyl)-4-nitro-2-vinylbenzene (0.710 g, 3.14 mmol, 72.99% yield) as a yellow solid.

MS [M+H]+: 227.2

### Step 5) tert-butyl N-[3-[[methyl-(4-nitro-2-vinyl-phenyl)-oxo-λ\hat{ }{6}-sulfanylidene]amino]propyl]carbamate

A stirred mixture of 1-(S-methylsulfonimidoyl)-4-nitro-2-vinylbenzene (0.3 g, 1.33 mmol, 1 eq), cesium carbonate (864.05 mg, 2.65 mmol, 2 eq) in DMF (5 mL) was added 3-(BOC-amino)propyl bromide (0.47 g, 1.99 mmol, 1.5 eq) at 70° C. and stirred for 16 h. The mixture was poured into water (30 mL) and extracted with EA (20 mL*3), washed with brine (50 mL*2), concentrated, purified by silica column chromatography (PE/EA=3:1 to 1:2) and reversed phase-HPLC (FA) to afford tert-butyl N-[3-[[methyl-(4-nitro-2-vinyl-phenyl)-oxo-λ\hat{ }{6}-sulfanylidene]amino]propyl]carbamate (220 mg, 0.570 mmol, 43.27% yield) as a light yellow oil.

MS [M+H]+: 384.2

### Step 6) tert-butyl N-[3-[[(4-amino-2-ethyl-phenyl)-methyl-oxo-λ\hat{ }{6}-sulfanylidene]amino]propyl]carbamate

A solution of tert-butyl N-[3-[[methyl-(4-nitro-2-vinyl-phenyl)-oxo-λ\hat{ }{6}-sulfanylidene]amino]propyl]carbamate (220.0 mg, 0.570 mmol, 1 eq) in methanol (10 mL) was added wet Pd on activated carbon (10%, 20.0 mg) and stirred under H₂ (2280 mmHg) at 25° C. for 16h. The mixture was filtered, concentrated and purified by reversed phase-HPLC (FA) to afford tert-butyl N-[3-[[(4-amino-2-ethyl-phenyl)-methyl-oxo-λ\hat{ }{6}-sulfanylidene]amino]propyl]carbamate (80 mg, 0.230 mmol, 39.22% yield) as a colorless oil.

MS [M+H]+: 356.1

### Step 7) tert-butyl N-[3-[[[4-[[3-(2,3-diluoro-4-methoxy-phenyl)imidazo[1,2-a]pyrazin-8-yl]amino]-2-ethyl-phenyl]-methyl-oxo-λ\hat{ }{6}-sulfanylidene]amino]propyl]carbamate

A mixture of tert-butyl N-[3-[[(4-amino-2-ethyl-phenyl)-methyl-oxo-λ\hat{ }{6}-sulfanylidene]amino]propyl]carbamate (80.0 mg, 0.230 mmol, 1 eq), 8-chloro-3-(2,3-difluoro-4-methoxy-phenyl)imidazo[1,2-a]pyrazine (73.19 mg, 0.250 mmol, 1.1 eq), cesium carbonate (219.96 mg, 0.680 mmol, 3 eq), tris(dibenzylideneacetone)dipalladium(0) (20.61 mg, 0.020 mmol, 0.100 eq) and 9,9-dimethyl-4,5-bis(diphenylphosphino)xanthene (13.02 mg, 0.020 mmol, 0.100 eq) in 1,4-dioxane (5 mL) was stirred under N2 at 115° C. in a microwave for 2 h. The mixture was filtered and concentrated, purified by silica column chromatography (DCM/MeOH=50:1) to afford tert-butyl N-[3-[[[4-[[3-(2,3-difluoro-4-methoxy-phenyl)imidazo[1,2-a]pyrazin-8-yl]amino]-2-ethyl-phenyl]-methyl-oxo-λ\hat{ }{6})-sulfanylidene]amino]propyl]carbamate (86 mg, 0.140 mmol, 62.17% yield) as a yellow solid.

MS [M+H]+: 615.4

### Step 8) N-[4-[N-(3-aminopropyl)-S-methyl-sulfonimidoyl]-3-ethyl-phenyl]-3-(2,3-difluoro-4-methoxy-phenyl)imidazo[1,2-a]pyrazin-8-amine; formic acid

A solution of tert-butyl N-[3-[[[4-[[3-(2,3-difluoro-4-methoxy-phenyl)imidazo[1,2-a]pyrazin-8-yl]amino]-2-ethyl-phenyl]-methyl-oxo-λ\hat{ }{6}-sulfanylidene]amino]propyl]carbamate (86.0 mg, 0.140 mmol, 1 eq) in methanol (3 mL) was added hydrochloric acid in MeOH (3.0 mL, 4N) and stirred at 25° C. for 16h. The solution was concentrated and purified by Prep-HPLC (FA) to afford N-[4-[N-(3-aminopropyl)-S-methyl-sulfonimidoyl]-3-ethyl-phenyl]-3-(2,3-difluoro-4-methoxy-phenyl)imidazo[1,2-a]pyrazin-8-amine; formic acid (37.2 mg, 0.070 mmol, 46.43% yield) as a white solid.

MS [M+H]+: 515.1

### Example 71

### N-(4-(3-amino-N-methylpropylsulfonimidoyl)phenyl)-3-(2,3-difluoro-4-methoxyphenyl)imidazo[1,2-a]pyrazin-8-amine RW-11-014T-4

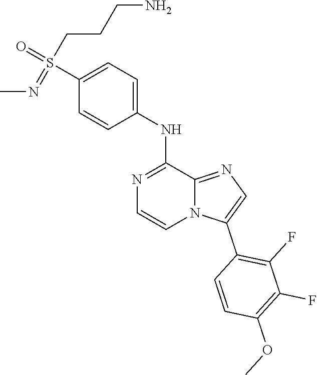 [SMILES: CN=S(=O)(CCCN)c1ccc(Nc2nccn3c(-c4ccc(OC)c(F)c4F)cnc23)cc1]

### Step 1) benzyl (3-((4-nitrophenyl)thio)propyl)carbamate

To a mixture of 4-nitrothiophenol (2.0 g, 12.89 mmol, 1 eq) and potassium carbonate (3.56 g, 25.78 mmol, 2 eq) in ACN (20 mL) was added benzyl N-(3-bromopropyl)carbamate (2.62 mL, 15.47 mmol, 1.2 eq) and stirred at 50° C. for 16 h. The mixture was filtered and concentrated to get a residue. The residue was washed with MTBE to give benzyl N-[3-(4-nitrophenyl)sulfanylpropyl]carbamate (3.7 g, 10.68 mmol, 77.29% yield) as a light yellow solid.

MS [M+H]+: 347.1

### Step 2) benzyl (3-(4-nitrophenylsulfonimidoyl)propyl)carbamate

A solution of benzyl N-[3-(4-nitrophenyl)sulfanylpropyl]carbamate (3.0 g, 8.66 mmol, 1 eq) and iodobenzene diacetate (2.62 mL, 17.32 mmol, 2 eq) in methanol (30 mL) was added ammonium carbamate (1.01 g, 12.99 mmol, 1.5 eq) at 25° C., then the mixture was stirred at 25° C. for 5 h.

The mixture was filtered and the residue was dried to afford benzyl N-[3-[(4-nitrophenyl)sulfonimidoyl]propyl]carbamate (1.98 g, 5.25 mmol, 60.58% yield) as a white solid.

MS [M+H]+: 378.1

### Step 3) benzyl (3-(N-methyl-4-nitrophenylsulfonimidoyl)propyl)carbamate

A solution of benzyl N-[3-[(4-nitrophenyl)sulfonimidoyl]propyl]carbamate (1.98 g, 5.25 mmol, 1 eq) and cesium carbonate (3.42 g, 10.49 mmol, 2 eq) in DMF (50 mL) was added iodomethane (2.94 mL, 47.22 mmol, 9 eq) and stirred at 50° C. for 16 h.

The mixture was poured into water, extracted with EtOAc (100 ml*3), washed with brine, dried over sodium sulfate and concentrated. The product was purified by silica gel chromatography eluting with petroleum ether/EtOAc=3/1-1/1 to give benzyl N-[3-[N-methyl-S-(4-nitrophenyl)sulfonimidoyl]propyl]carbamate (624 mg, 1.59 mmol, 30.39% yield) as a black oil.

MS [M+H]+: 392.0

### Step 4) benzyl (3-(4-amino-N-methylphenylsulfonimidoyl)propyl)carbamate

To a stirred solution of NICKEL(II) CHLORIDE HEXAHYDRATE (189.45 mg, 0.800 mmol, 0.500 eq) in methanol (49.87 mL) was added sodium borohydride (30.15 mg, 0.800 mmol, 0.500 eq) at 0° C. and stirred for 10 min, then benzyl N-[3-[N-methyl-S-(4-nitrophenyl)sulfonimidoyl]propyl]carbamate (624.0 mg, 1.59 mmol, 1 eq) and sodium borohydride (180.93 mg, 4.78 mmol, 3 eq) was added to the mixture and stirred at 0° C. for 0.5 h.

The mixture was concentrated to give a residue, which was purified by prep-HPLC (FA) to afford benzyl N-[3-[S-(4-aminophenyl)-N-methyl-sulfonimidoyl]propyl]carbamate (338 mg, 0.940 mmol, 58.66% yield) as black oil.

MS [M+H]+: 362.1

### Step 5) benzyl (3-(4-((3-(2,3-difluoro-4-methoxyphenyl)imidazo[1,2-a]pyrazin-8-yl)amino)-N-methylphenylsulfonimidoyl)propyl)carbamate

A stirred solution of 8-chloro-3-(2,3-difluoro-4-methoxy-phenyl)imidazo[1,2-a]pyrazine (276.48 mg, 0.940 mmol, 1 eq), benzyl N-[3-[S-(4-aminophenyl)-N-methyl-sulfonimidoyl]propyl]carbamate (338.0 mg, 0.940 mmol, 1 eq) tris(dibenzylideneacetone)dipalladium (0) (85.63 mg, 0.090 mmol, 0.100 eq), cesium carbonate (914.02 mg, 2.81 mmol, 3 eq) and 9,9-dimethyl-4,5-bis(diphenylphosphino)xanthene (54.11 mg, 0.090 mmol, 0.100 eq) in 1,4-dioxane (10 mL) was stirred at 115° C. in a microwave for 2 h. The mixture was filtered and concentrated to get a residue, which was purified by prep-TLC (dichloromethane/EtOAc=1:1) to get benzyl N-[3-[S-[4-[[3-(2,3-difluoro-4-methoxy-phenyl)imidazo[1,2-a]pyrazin-8-yl]amino]phenyl]-N-methyl-sulfonimidoyl]propyl]carbamate (260 mg, 0.420 mmol, 44.8% yield) as a light yellow solid.

MS [M+H]+: 621.1

### Step 6) N-(4-(3-amino-N-methylpropylsulfonimidoyl)phenyl)-3-(2,3-difluoro-4-methoxyphenyl)imidazo[1,2-a]pyrazin-8-amine RW-11-014T-4

A solution of benzyl N-[3-[S-[4-[[3-(2,3-difluoro-4-methoxy-phenyl)imidazo[1,2-a]pyrazin-8-yl]amino]phenyl]-N-methyl-sulfonimidoyl]propyl]carbamate (130.0 mg, 0.210 mmol, 1 eq) and palladium hydroxide (29.41 mg, 0.210 mmol, 1 eq) in methanol (10 mL) was stirred under H2 (2280 mmHg) at 25° C. for 16 h. The mixture was filtered to get a residue, which was purified by prep-HPLC (FA) to afford the title compound (6.4 mg, 0.010 mmol, 5.58% yield) as a white solid.

MS [M+H]+: 487.1

### Example 72

### N-(4-(3-amino-N-methylpropylsulfonimidoyl)-3-methylphenyl)-3-(2,3-difluoro-4-methoxyphenyl)imidazo[1,2-a]pyrazin-8-amine

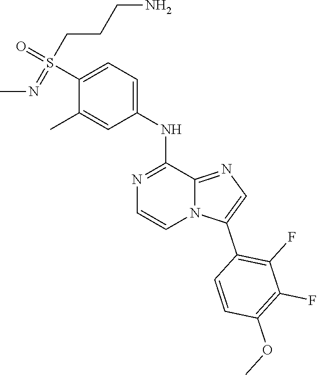 [SMILES: CN=S(=O)(CCCN)c1ccc(Nc2nccn3c(-c4ccc(OC)c(F)c4F)cnc23)cc1C]

### Step 1) tert-butyl (3-((2-methyl-4-nitrophenyl)thio)propyl)carbamate

To a mixture of 2-methyl-4-nitro-benzenethiol (2.5 g, 14.78 mmol, 1 eq) and potassium carbonate (4.08 g, 29.55 mmol, 2 eq) in ACN (50 mL) was added 3-(BOC-amino)propyl bromide (2.62 mL, 17.73 mmol, 1.2 eq) and stirred at 70° C. for 16 h. The mixture was filtered and concentrated to get a residue, which was purified by silica gel chromatography eluting with petroleum ether/EtOAC=50/1-10/1 to afford the title compound (1.16 g, 3.54 mmol, 25.05% yield) as a yellow solid.

MS obsd. (ESI⁺) [(M+H)⁺−100]: 227.0

### Step 2) tert-butyl (3-(2-methyl-4-nitrophenylsulfonimidoyl)propyl)carbamate

To a solution of tert-butyl N-[3-(2-methyl-4-nitro-phenyl)sulfanylpropyl]carbamate (1.16 g, 3.55 mmol, 1 eq) and iodobenzene diacetate (2.62 mL, 7.1 mmol, 2 eq) in methanol (12.3 mL) was added ammonium carbamate (0.42 g, 5.33 mmol, 1.5 eq) at 25° C., then the mixture was stirred at 25° C. for 5 h. The mixture was poured into water, extracted with EtOAc (50*3), washed with brine, dried over sodium sulfate and concentrated to get a residue. The mixture was purified by silica gel chromatography eluting with petroleum ether/EtOAc=20/1-1/1 to afford the title compound (1 g, 2.8 mmol, 78.79% yield) as a yellow oil.

MS [M+H]+: 358.0

### Step 3) tert-butyl (3-(N,2-dimethyl-4-nitrophenylsulfonimidoyl)propyl)carbamate

A solution of tert-butyl N-[3-[(2-methyl-4-nitro-phenyl)sulfonimidoyl]propyl]carbamate (1.0 g, 2.8 mmol, 1 eq) and cesium carbonate (1.82 g, 5.6 mmol, 2 eq) in DMF (31.68 mL) was added iodomethane (1.0 mL, 16.06 mmol, 5.74 eq) and stirred at 70° C. for 16 h. The mixture was poured into water, extracted with EtOAc (50 ml*3), washed with brine, dried over sodium sulfate and concentrated to get residue. The product was purified by silica gel chromatography eluting with petroleum ether/EtOAc=10/1-1/1 to get the title compound (639 mg, 1.72 mmol, 61.49% yield) as a yellow oil.

MS [M+H]+: 372.1

### Step 4) tert-butyl (3-(4-amino-N,2-dimethylphenylsulfonimidoyl)propyl)carbamate

A stirred solution of NICKEL(II) CHLORIDE HEXAHYDRATE (204.45 mg, 0.860 mmol, 0.500 eq) in methanol (20 mL) was added sodium borohydride (32.54 mg, 0.860 mmol, 0.500 eq) at 0° C. and stirred for 10 min, then tert-butyl N-[3-[N-methyl-S-(2-methyl-4-nitro-phenyl)sulfonimidoyl]propyl]carbamate (639.0 mg, 1.72 mmol, 1 eq) and sodium borohydride (195.25 mg, 5.16 mmol, 3 eq) was added to the mixture and stirred at 0° C. for 0.5h. The mixture was filtered to get a residue, which was purified by prep-HPLC (FA) to afford the title compound (184 mg, 0.540 mmol, 31.32% yield) as brown solid.

MS [M+H]+: 342.3

### Step 5) tert-butyl (3-(4-((3-(2,3-difluoro-4-methoxyphenyl)imidazo[1,2-a]pyrazin-s-yl)amino)-N,2-dimethylphenylsulfonimidoyl)propyl)carbamate

A stirred solution of 8-chloro-3-(2,3-difluoro-4-methoxy-phenyl)imidazo[1,2-a]pyrazine (86.59 mg, 0.290 mmol, 1 eq), tert-butyl N-[3-[S-(4-amino-2-methyl-phenyl)-N-methyl-sulfonimidoyl]propyl]carbamate (100.0 mg, 0.290 mmol, 1 eq), tris(dibenzylideneacetone)dipalladium (0) (26.82 mg, 0.030 mmol, 0.100 eq), cesium carbonate (286.25 mg, 0.880 mmol, 3 eq) and 9,9-dimethyl-4,5-bis(diphenylphosphino)xanthene (16.95 mg, 0.030 mmol, 0.100 eq) in 1,4-dioxane (3 mL) was stirred at 115° C. in a microwave for 2 h. The mixture was filtered and concentrated to get a residue, which was purified by prep-TLC (dichloromethane/EtOAc=1:1) to give the title compound (104 mg, 0.170 mmol, 59.12% yield) as a yellow oil.

MS [M+H]+: 601.1

### Step 6) N-(4-(3-amino-N-methylpropylsulfonimidoyl)-3-methylphenyl)-3-(2,3-difluoro-4-methoxyphenyl)imidazo[1,2-a]pyrazin-8-amine RW-11-014T-8

A stirred solution of tert-butyl N-[3-[S-[4-[[3-(2,3-difluoro-4-methoxy-phenyl)imidazo[1,2-a]pyrazin-8-yl]amino]-2-methyl-phenyl]-N-methyl-sulfonimidoyl]propyl]carbamate (104.0 mg, 0.170 mmol, 1 eq), trifluoroacetic acid (0.5 mL, 6.49 mmol, 37.48 eq) in DCM (5 mL) was stirred at 15° C. for 2h. The mixture was concentrated to get a residue, which was purified by prep-HPLC (FA) to give the title compound (7.43 mg, 0.010 mmol, 8.57% yield) as a white solid.

MS [M+H]+: 501.3

### Example 73

### (4-(4-((3-(2,3-difluoro-4-methoxyphenyl)imidazo[1,2-a]pyrazin-8-yl)amino)-N,2-dimethylphenylsulfonimidoyl)piperidin-1-yl)((2S,4R)-4-hydroxypyrrolidin-2-yl)methanone

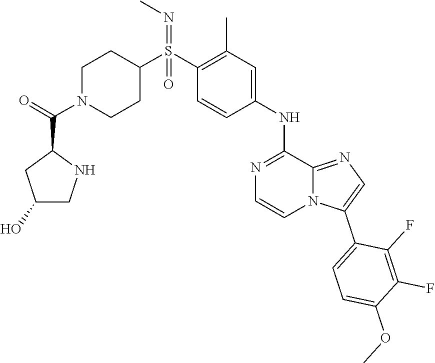 [SMILES: CN=S(=O)(c1ccc(Nc2nccn3c(-c4ccc(OC)c(F)c4F)cnc23)cc1C)C1CCN(C(=O)[C@@H]2C[C@@H](O)CN2)CC1]

### Step 1: tert-butyl 4-((2-methyl-4-nitrophenyl)thio)piperidine-1-carboxylate

A mixture of 2-methyl-4-nitro-benzenethiol (3.2 g, 18.93 mmol, 1 eq), potassium carbonate (3.92 g, 28.39 mmol, 1.5 eq) and N—BOC-4-bromopiperidine (5.0 g, 18.93 mmol, 1 eq) in ACN (12.81 mL) was stirred at 70° C. for 16 h. The mixture was poured into water, extracted with EtOAc (100 ml*3), washed with brine, dried over sodium sulfate and concentrated to get a residue. The product was purified by silica gel chromatography eluting with petroleum ether/EtOAc=20/1-10/1 to afford tert-butyl the title compound (6.5 g, 18.44 mmol, 97.43% yield) as a yellow oil.

MS obsd. (ESI⁺) [(M+H)⁺−56]: 297.0

### Step 2) 4-((2-methyl-4-nitrophenyl)thio)piperidine

A mixture of HCl in 1,4-dioxane (20.0 mL, 80 mmol, 4.34 eq) and tert-butyl 4-(2-methyl-4-nitro-phenyl)sulfanylpiperidine-1-carboxylate (6.5 g, 18.44 mmol, 1 eq) in 1,4-dioxane (5 mL) was stirred at 16° C. for 16 h. The mixture was filtered and the residue was dried to afford the title compound (2.35 g, 9.31 mmol, 50.5% yield) as a white solid.

MS [M+H]+: 253.6

### Step 3) (2S,4R)-tert-butyl 4-hydroxy-2-(4-((2-methyl-4-nitrophenyl)thio)piperidine-1-carbonyl)pyrrolidine-1-carboxylate

A mixture of 4-(2-methyl-4-nitro-phenyl)sulfanylpiperidine (2.35 g, 9.31 mmol, 1 eq), PROPYLPHOSPHONICANHYDRIDE 50% in EtOAc (11.08 mL, 18.63 mmol, 2 eq), triethylamine (12.98 mL, 93.13 mmol, 10 eq) and BOC-HYP-OH (2.15 g, 9.31 mmol, 1 eq) in THF (50 mL) was stirred at 16° C. for 16 h. The mixture was poured into water, extracted with EtOAc (100 ml*3), washed with brine, dried over sodium sulfate and concentrated to afford the title compound (4.02 g, 8.63 mmol, 92.72% yield) as a yellow oil.

MS [M+H]+: 466.1

### Step 4) (2S,4R)-tert-butyl 4-hydroxy-2-(4-(2-methyl-4-nitrophenylsulfonimidoyl)piperidine-1-carbonyl)pyrrolidine-1-carboxylate

A mixture of ammonium carbamate (1.01 g, 12.95 mmol, 1.5 eq), tert-butyl (2S,4R)-4-hydroxy-2-[4-(2-methyl-4-nitro-phenyl)sulfanylpiperidine-1-carbonyl]pyrrolidine-1-carboxylate (4.02 g, 8.63 mmol, 1 eq), iodobenzene diacetate (5.56 g, 17.27 mmol, 2 eq) in methanol (50 mL) was stirred at 16° C. for 16 h. The mixture was concentrated and purified by prep-HPLC (FA) to afford the title compound (3.06 g, 6.16 mmol, 64.61% yield) as a white solid.

MS [M+H]+: 497.2

### Step 5) (2S,4R)-tert-butyl 2-(4-(N,2-dimethyl-4-nitrophenylsulfonimidoyl)piperidine-1-carbonyl)-4-hydroxypyrrolidine-1-carboxylate

A mixture of tert-butyl (2S,4R)-4-hydroxy-2-[4-[(2-methyl-4-nitro-phenyl)sulfonimidoyl]piperidine-1-carbonyl]pyrrolidine-1-carboxylate (3.06 g, 6.16 mmol, 1 eq), iodomethane (3.84 mL, 61.62 mmol, 10 eq), cesium carbonate (4.02 g, 12.32 mmol, 2 eq) in DMF (30 mL) was stirred at 70° C. for 16 h. The mixture was poured into water, extracted with EtOAc (100 ml*3), washed with brine, dried over sodium sulfate and concentrated to get a residue. The product was purified by silica gel chromatography eluting with petroleum ether/EtOAc=2/1-dichloromethane/methanol=20/1 to afford tert-butyl the title compound (2.05 g, 4.01 mmol, 65.15% yield) as a yellow solid.

MS [M+H]+: 511.2

### Step 6) (2S,4R)-tert-butyl 2-(4-(4-amino-N,2-dimethylphenylsulfonimidoyl)piperidine-1-carbonyl)-4-hydroxypyrrolidine-1-carboxylate

To a stirred solution of NICKEL(II) CHLORIDE HEXAHYDRATE (477.15 mg, 2.01 mmol, 0.500 eq) in methanol (50 mL) was added sodium borohydride (75.95 mg, 2.01 mmol, 0.500 eq) at 0° C. and then stirred for 10 min, then tert-butyl (2S,4R)-4-hydroxy-2-[4-[N-methyl-S-(2-methyl-4-nitro-phenyl)sulfonimidoyl]piperidine-1-carbonyl]pyrrolidine-1-carboxylate (2.05 g, 4.01 mmol, 1 eq) and sodium borohydride (455.68 mg, 12.04 mmol, 3 eq) was added to the mixture and stirred at 0° C. for 0.5 h. The mixture was filtered to get a residue, which was purified by prep-HPLC (FA) to afford the title compound (518 mg, 1.08 mmol, 26.84% yield) as a yellow solid.

MS [M+H]+: 481.2

### Step 7) (2S,4R)-tert-butyl 2-(4-(4-((3-(2,3-difluoro-4-methoxyphenyl)imidazo[1,2-a]pyrazin-8-yl)amino)-N,2-dimethylphenylsulfonimidoyl)piperidine-1-carbonyl)-4-hydroxypyrrolidine-1-carboxylate

A stirred solution of tert-butyl (2S,4R)-2-[4-[S-(4-amino-2-methyl-phenyl)-N-methyl-sulfonimidoyl]piperidine-1-carbonyl]-4-hydroxy-pyrrolidine-1-carboxylate (100.0 mg, 0.210 mmol, 1 eq), 8-chloro-3-(2,3-difluoro-4-methoxy-phenyl)imidazo[1,2-a]pyrazine (61.52 mg, 0.210 mmol, 1 eq), tris(dibenzylideneacetone)dipalladium (0) (19.05 mg, 0.020 mmol, 0.100 eq), cesium carbonate (203.37 mg, 0.620 mmol, 3 eq) and 9,9-dimethyl-4,5-bis(diphenylphosphino)xanthene (12.04 mg, 0.020 mmol, 0.100 eq) in 1,4-dioxane (5 mL) was stirred at 115° C. in a microwave for 2 h. The mixture was filtered and concentrated to get a residue, which was purified by prep-TLC (dichloromethane/methanol=10:1) to get the title compound (63 mg, 0.090 mmol, 40.93% yield) as a yellow oil.

MS [M+H]+: 740.3

### Step 8) (4-(4-((3-(2,3-difluoro-4-methoxyphenyl)imidazo[1,2-a]pyrazin-8-yl)amino)-N,2-dimethylphenylsulfonimidoyl)piperidin-1-yl)((2S,4R)-4-hydroxypyrrolidin-2-yl)methanone

A stirred solution of tert-butyl (2S,4R)-2-[4-[S-[4-[[3-(2,3-difluoro-4-methoxy-phenyl)imidazo[1,2-a]pyrazin-8-yl]amino]-2-methyl-phenyl]-N-methyl-sulfonimidoyl]piperidine-1-carbonyl]-4-hydroxy-pyrrolidine-1-carboxylate (63.0 mg, 0.090 mmol, 1 eq) in trifluoroacetic acid (0.5 mL, 6.49 mmol, 76.21 eq) in DCM (10 mL) was stirred at 10° C. for 2 h. The mixture was concentrated to get a residue, which was purified by prep-HPLC (FA) to give the title compound (11.52 mg, 0.020 mmol, 21.15% yield) as a white solid.

MS [M+H]+: 640.1

### Example 74

### (4-(4-((3-(2,3-difluoro-4-methoxyphenyl)imidazo[1,2-a]pyrazin-8-yl)amino)-N-methylphenylsulfonimidoyl)piperidin-1-yl)((2S,4R)-4-hydroxypyrrolidin-2-yl)methanone

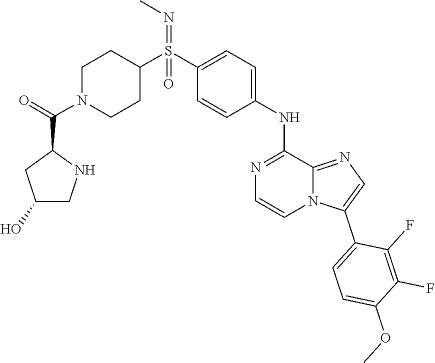 [SMILES: CN=S(=O)(c1ccc(Nc2nccn3c(-c4ccc(OC)c(F)c4F)cnc23)cc1)C1CCN(C(=O)[C@@H]2C[C@@H](O)CN2)CC1]

### Step 1) tert-butyl 4-((4-nitrophenyl)thio)piperidine-1-carboxylate

A mixture of 4-nitrothiophenol (2.94 g, 18.93 mmol, 1 eq), potassium carbonate (3.92 g, 28.39 mmol, 1.5 eq) and N—BOC-4-bromopiperidine (5.0 g, 18.93 mmol, 1 eq) in ACN (12.81 mL) was stirred at 70° C. for 16 h. The mixture was poured into water, extracted with EtOAc (100 ml*3), washed with brine, dried over sodium sulfate and concentrated to give a residue. The product was purified by silica gel chromatography eluting with petroleum ether/EtOAc=40/1-10/1 to afford the crude title compound (5.5 g, 16.25 mmol, 85.86% yield) as a yellow oil.

MS obsd. (ESI⁺) [(M+H)⁺−56]: 283.1

### Step 2) 4-((4-nitrophenyl)thio)piperidine

A mixture of 1,4-dioxane/HCl (20.0 mL, 80 mmol, 4.92 eq) and tert-butyl 4-(4-nitrophenyl)sulfanylpiperidine-1-carboxylate (5.5 g, 16.25 mmol, 1 eq) in 1,4-Dioxane (10 mL) was stirred at 16° C. for 16 h. The mixture was filtered and the residue was dried to afford the title compound (3.78 g, 15.86 mmol, 97.6% yield) as a yellow solid.

MS [M+H]+: 239.0

### Step 3) (2S,4R)-tert-butyl 4-hydroxy-2-(4-((4-nitrophenyl)thio)piperidine-1-carbonyl)pyrrolidine-1-carboxylate

A mixture of 4-(4-nitrophenyl)sulfanylpiperidine (3.58 g, 15.02 mmol, 1 eq), PROPYLPHOSPHONICANHYDRIDE 50% in EtOAc (17.87 mL, 30.04 mmol, 2 eq), triethylamine (20.94 mL, 150.22 mmol, 10 eq) and BOC-HYP-OH (3.47 g, 15.02 mmol, 1 eq) in THF (50 mL) was stirred at 16° C. for 16 h. The mixture was poured into water, extracted with EtOAc (100 ml*3), washed with brine, dried over sodium sulfate and concentrated to afford the title compound (6.15 g, 13.62 mmol, 90.66% yield) as a yellow oil.

MS [M+H]+: 452.0

### Step 4) (2S,4R)-tert-butyl 4-hydroxy-2-(4-(4-nitrophenylsulfonimidoyl)piperidine-1-carbonyl)pyrrolidine-1-carboxylate

A mixture of ammonium carbamate (1.59 g, 20.43 mmol, 1.5 eq), tert-butyl (2S,4R)-4-hydroxy-2-[4-(4-nitrophenyl)sulfanylpiperidine-1-carbonyl]pyrrolidine-1-carboxylate (6.15 g, 13.62 mmol, 1 eq), iodobenzene diacetate (8.77 g, 27.24 mmol, 2 eq) in methanol (50 mL) was stirred at 16° C. for 16 h. The mixture was concentrated and purified by prep-HPLC (FA) to afford the title compound (4 g, 8.29 mmol, 60.86% yield) as a white solid.

MS [M+H]+: 483.1

### Step 5) (2S,4R)-tert-butyl 4-hydroxy-2-(4-(N-methyl-4-nitrophenylsulfonimidoyl)piperidine-1-carbonyl)pyrrolidine-1-carboxylate

A mixture of tert-butyl (2S,4R)-4-hydroxy-2-[4-[(4-nitrophenyl)sulfonimidoyl]piperidine-1-carbonyl]pyrrolidine-1-carboxylate (2.6 g, 5.39 mmol, 1 eq), iodomethane (3.35 mL, 53.88 mmol, 10 eq), cesium carbonate (3.51 g, 10.78 mmol, 2 eq) in DMF (30 mL) was stirred at 70° C. for 16 h. The mixture was poured into water, extracted with EtOAc (100 ml*3), washed with brine, dried over sodium sulfate and concentrated to get a residue. The residue was purified by silica gel chromatography eluting with petroleum ether/EtOAc=2/1-dichloromethane/methanol=20/1 to afford the title compound (1.2 g, 2.42 mmol, 44.85% yield) as a white solid.

MS [M+H]+: 497.1

### Step 6) (2S,4R)-tert-butyl 2-(4-(4-amino-N-methylphenylsulfonimidoyl)piperidine-1-carbonyl)-4-hydroxypyrrolidine-1-carboxylate

A stirred solution of NICKEL(II) CHLORIDE HEXAHYDRATE (287.19 mg, 1.21 mmol, 0.500 eq) in methanol (20 mL) was added sodium borohydride (45.71 mg, 1.21 mmol, 0.500 eq) at 0° C. and stirred for 10 min, then tert-butyl (2S,4R)-4-hydroxy-2-[4-[N-methyl-S-(4-nitrophenyl)sulfonimidoyl]piperidine-1-carbonyl]pyrrolidine-1-carboxylate (1.2 g, 2.42 mmol, 1 eq) and sodium borohydride (274.27 mg, 7.25 mmol, 3 eq) was added to the mixture and stirred at 0° C. for 0.5 h. The mixture was filtered to get a residue, which was purified by prep-HPLC (FA) to afford the title compound (505 mg, 1.08 mmol, 44.79% yield) as a white solid.

MS [M+H]+: 467.2

### Step 7) (2S,4R)-tert-butyl 2-(4-(4-((3-(2,3-difluoro-4-methoxyphenyl)imidazo[1,2-a]pyrazin-8-yl)amino)-N-methylphenylsulfonimidoyl)piperidine-1-carbonyl)-4-hydroxypyrrolidine-1-carboxylate

A stirred solution of tert-butyl (2S,4R)-2-[4-[S-(4-aminophenyl)-N-methyl-sulfonimidoyl]piperidine-1-carbonyl]-4-hydroxy-pyrrolidine-1-carboxylate (100.0 mg, 0.210 mmol, 1 eq), 8-chloro-3-(2,3-difluoro-4-methoxy-phenyl)imidazo[1,2-a]pyrazine (63.37 mg, 0.210 mmol, 1 eq), tris(dibenzylideneacetone)dipalladium (0) (19.63 mg, 0.020 mmol, 0.100 eq), cesium carbonate (209.49 mg, 0.640 mmol, 3 eq) and 9,9-dimethyl-4,5-bis(diphenylphosphino)xanthene (12.4 mg, 0.020 mmol, 0.100 eq) in 1,4-dioxane (5 mL) was stirred at 115° C. in a microwave for 2h. The mixture was filtered and concentrated to get a residue, which was purified by prep-TLC (dichloromethane/EtOAc=1:1) to give the title compound (37 mg, 0.050 mmol, 23.79% yield) as a yellow oil.

MS [M+H]+: 726.3

### Step 8) (4-(4-((3-(2,3-difluoro-4-methoxyphenyl)imidazo[1,2-a]pyrazin-8-yl)amino)-N-methylphenylsulfonimidoyl)piperidin-1-yl)((2S,4R)-4-hydroxypyrrolidin-2-yl)methanone

A stirred solution of tert-butyl (2S,4R)-2-[4-[S-[4-[[3-(2,3-difluoro-4-methoxy-phenyl)imidazo[1,2-a]pyrazin-8-yl]amino]phenyl]-N-methyl-sulfonimidoyl]piperidine-1-carbonyl]-4-hydroxy-pyrrolidine-1-carboxylate (37.0 mg, 0.050 mmol, 1 eq) in trifluoroacetic acid (0.5 mL, 6.49 mmol, 127.31 eq) in DCM (10 mL) was stirred at 10° C. for 16 h. The mixture was concentrated to a residue, which was purified by prep-HPLC (FA condition) to give the title compound (9.31 mg, 0.010 mmol, 27.19% yield) as a light yellow solid.

MS obsd. (ESI⁺) [(M+H)⁺]:626.1

### Intermediate 2

### N-(4-((3-bromoimidazo[1,2-a]pyrazin-8-yl)amino)-2-methylphenyl)acetamide

### Step 1) N-(2-methyl-4-nitrophenyl)acetamide

To a solution of 2-methyl-4-nitroaniline (35.0 g, 230 mmol) in THF (500 mL) was added AcOH (50 g) and Ac₂O (35.2 g, 345 mmol) at 0° C. Then the reaction mixture was heated to 80° C. and stirred for 12 h. The reaction mixture was concentrated to give the crude title compound (42 g, crude) which was used next step directly.

### Step 2) N-(4-amino-2-methylphenyl)acetamide

To a solution of N-(2-methyl-4-nitrophenyl)acetamide (42.0 g, 201 mmol) in MeOH (500 mL) was added wet Pd/C (10%, 5.0 g) and stirred under H₂ (50 psi) at 16° C. for 12 h. The reaction mixture was filtered and the filtrate was concentrated to give the crude title compound (30 g), which was used next step indirectly.

### Step 3) N-(4-((3-bromoimidazo[1,2-a]pyrazin-8-yl)amino)-2-methylphenyl)acetamide

To a solution of 3-bromo-8-chloroimidazo[1,2-a]pyrazine (2.32 g, 10.0 mmol) in NMP (10 mL) was added N-(4-amino-2-methylphenyl)acetamide (3.0 g, 20.0 mmol) and DIPEA (1.94 g, 15.0 mmol) at 20° C. Then the reaction mixture was heated to 140° C. and stirred for 12 h, the reaction mixture was quenched with H₂O (20 mL). The mixture was filtered to give the crude product, which was recrystallized with MeOH (20 mL) to give the title compound (1.6 g, 44%) as a brown solid.

¹H NMR (400 MHz, DMSO-d₆) δ=9.57 (s, 1H), 9.25 (s, 1H), 7.95-7.63 (m, 4H), 7.56 (d, J=4.5 Hz, 1H), 7.28 (d, J=8.5 Hz, 1H), 2.19 (s, 3H), 2.05 (s, 3H) ppm.

MS [M+H]+: 360.9.

### Example 75

### 2-(2-aminoethoxy)-N-(4-((3-(4-methoxyphenyl)imidazo[1,2-a]pyrazin-8-yl)amino)-2-methylphenyl)acetamide hydrochloride

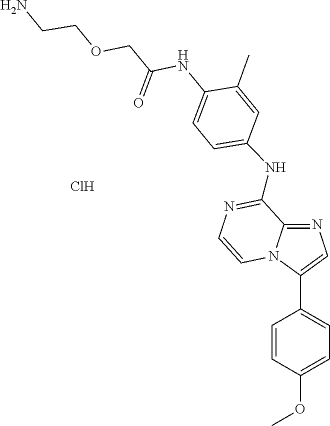 [SMILES: COc1ccc(-c2cnc3c(Nc4ccc(NC(=O)COCCN)c(C)c4)nccn23)cc1 ; Cl]

### Step 1) 8-chloro-3-(4-methoxyphenyl)imidazo[1,2-a]pyrazine

8-chloro-3-iodoimidazo[1,2-a]pyrazine (2.7 g, 9.66 mmol, Eq: 1) CAS 1049677-32-8 was combined with dioxane (30 ml) and water (15 ml). (4-methoxyphenyl)boronic acid CAS 5720-07-0 (1.76 g, 11.6 mmol, Eq: 1.20), Na₂CO₃ (2.05 g, 19.3 mmol, Eq: 2.00) and 1,1′-bis(diphenylphosphino)ferrocenedichloro palladium(II) dichloromethane complex (353 mg, 483 μmol, Eq: 0.05) were added, and the reaction mixture was heated at 80° C. for 2h, and over the weekend at RT. The reaction mixture was diluted with AcOEt and water. After phase separation, the aqueous layer was extracted with AcEtOH several times. Combined organic layers were washed with brine solution, then dried over Na₂SO₄. The crude obtained was purified by flash chromatography (silica gel, 80 g 0% to 80% EtOAc in heptane) to give 1.43 g (57%) of the title compound as a brown solid. MS (ESI, m/z): 260.1 [M+H]⁺

### Step 2) (N-(4-((3-(4-methoxyphenyl)imidazo[1,2-a]pyrazin-8-yl)amino)-2-methylphenyl)acetamide

N-(4-amino-2-methylphenyl)acetamide (31.6 mg, 193 μmol, Eq: 1) CAS 56891-59-9 and 8-chloro-3-(4-methoxyphenyl)imidazo[1,2-a]pyrazine (50 mg, 193 μmol, Eq: 1) were heated in acetonitrile (2 ml) at 90° C. O/N. Acetic acid (210 mg, 200 μl, 3.49 mmol, Eq: 18.1) was added followed by stirring at 90° C. O/N. The RM was concentrated and precipitated with diethyl ether to give 69.5 mg (93%) of the title compound as a light brown solid. MS (ESI, m/z): 388.2 [M+H]⁺

### Step 3) N1-(3-(4-methoxyphenyl)imidazo[1,2-a]pyrazin-8-yl)-3-methylbenzene-1,4-diamine hydrochloride

To N-(4-((3-(4-methoxyphenyl)imidazo[1,2-a]pyrazin-8-yl)amino)-2-methylphenyl)acetamide (68 mg, 176 μmol, Eq: 1) were added HCl (2.4 g, 2 ml, 24.4 mmol, Eq: 139) and ethanol (2 ml). The RM was heated to 80° C. and stirred O/N. The RM was concentrated and 66.5 mg (99%) of the title product were obtained as a light yellow solid which was used without further purification in the next step. MS (ESI, m/z): 346.2 [M+H]⁺

### Step 4) tert-butyl (2-(2-((4-((3-(4-methoxyphenyl)imidazo[1,2-a]pyrazin-8-yl)amino)-2-methylphenyl)amino)-2-oxoethoxy)ethyl)carbamate

To N1-(3-(4-methoxyphenyl)imidazo[1,2-a]pyrazin-8-yl)-3-methylbenzene-1,4-diamine hydrochloride (66.5 mg, 174 μmol, Eq: 1) in DMF (2 ml) was added DIPEA (67.5 mg, 91.2 μl, 522 μmol, Eq: 3), 2-(2-((tert-butoxycarbonyl)amino)ethoxy)acetic acid (42 mg, 192 μmol, Eq: 1.1) CAS 142929-49-5 and finally HATU (132 mg, 348 μmol, Eq: 2) with stirring at RT O/N. The RM was concentrated and purified by prep. HPLC to give 60.1 mg (63%) of the title product as a white solid. MS (ESI, m/z): 547.3 [M+H]⁺

### Step 5) 2-(2-aminoethoxy)-N-(4-((3-(4-methoxyphenyl)imidazo[1,2-a]pyrazin-8-yl)amino)-2-methylphenyl)acetamide hydrochloride

HCl in dioxane (7.8 g, 6.5 ml, 26 mmol, Eq: 236) was added to tert-butyl (2-(2-((4-((3-(4-methoxyphenyl)imidazo[1,2-a]pyrazin-8-yl)amino)-2-methylphenyl)amino)-2-oxoethoxy)ethyl)carbamate (60.1 mg, 110 μmol, Eq: 1) followed by stirring at RT for 8 h. The mixture was concentrated. The residue was triturated with diethyl ether and filtered to give 60.0 mg (102%) of the title product as a yellow solid. MS (ESI, m/z): 447.2 [M+H]⁺

### Example 76

### 2-chloro-5-((3-(4-methoxyphenyl)imidazo[1,2-a]pyrazin-8-yl)amino)-N-methylbenzamide

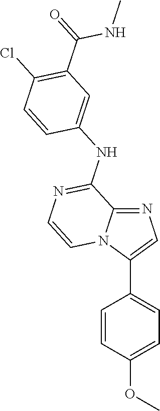 [SMILES: CNC(=O)c1cc(Nc2nccn3c(-c4ccc(OC)cc4)cnc23)ccc1Cl]

### Step 1) 8-chloro-3-(4-methoxyphenyl)imidazo[1,2-a]pyrazine

The title compound was obtained in analogy to Example 75, Step 1. MS (ESI, m/z): 260.1 [M+H]⁺

### Step 2) 2-chloro-5-((3-(4-methoxyphenyl)imidazo[1,2-a]pyrazin-8-yl)amino)-N-methylbenzamide

8-chloro-3-(4-methoxyphenyl)imidazo[1,2-a]pyrazine (26 mg, 0.10 mmol, Eq: 1), 5-amino-2-chloro-N-methylbenzamide (27.7 mg, 0.15 mmol, Eq: 1.5), cesium carbonate (65.2 mg, 200 μmol, Eq: 2.00), t-Bu-X-phos (4.25 mg, 10 μmol, Eq: 0.10) and Pd₂(dba)₃ (4.58 mg, 5 μmol, Eq: 0.05) were suspended in t-BuOH, and the mixture was stirred at 100° C. for 48 h. After removal of t-BuOH, 1 ml DMSO was added to the dry reaction mixture, and after stirring for 20 min it was filtered over decalite. The crude obtained was purified by prep HPLC to give 23.2 mg (57%) of the title product. MS (ESI, m/z): 408.2 [M+H]+

The following Examples were obtained in analogy:

| | | | ESI MS | Starting |
|---|---|---|---|---|
| Example | Name | Structure | [M + H]⁺ | material |
| 77 | 4-((3-(4- methoxyphenyl) imidazo[1,2-a]pyrazin- 8-yl)amino)-N,N- dimethylbenzenesulfonamide | 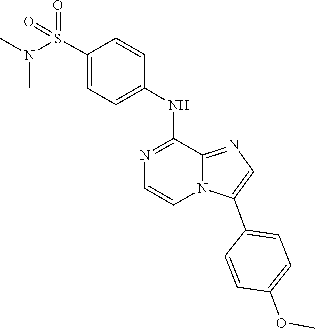 | 424.2 | 4-amino- N,N- dimethylbenzenesulfonamide |
| 78 | 3-(4- methoxyphenyl)-N- (4- (morpholinosulfonyl) phenyl)imidazo[1, 2-a]pyrazin-8-amine | 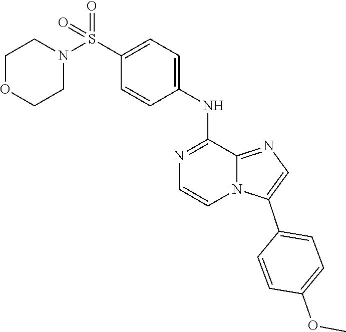 [SMILES: COc1ccc(-c2cnc3c(Nc4ccc(S(=O)(=O)N5CCOCC5)cc4)nccn23)cc1] | 466.2 | 4- (morpholinosulfonyl) aniline |
| 79 | 3-(4- methoxyphenyl)-N- (4-((4- methylpiperazin-1 - yl)sulfonyl)phenyl) imidazo[1,2- a]pyrazin-8-amine | 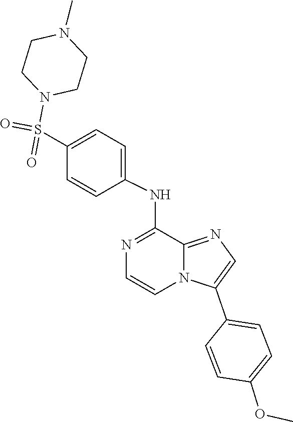 [SMILES: COc1ccc(-c2cnc3c(Nc4ccc(S(=O)(=O)N5CCN(C)CC5)cc4)nccn23)cc1] | 477.4 | 4-((4- methylpiperazin-1- yl)sulfonyl) aniline |
| 80 | N,N-diethyl-2-(4- ((3-(4- methoxyphenyl) imidazo[1,2-a]pyrazin- 8-yl)amino) phenylsulfonamido) acetamide | 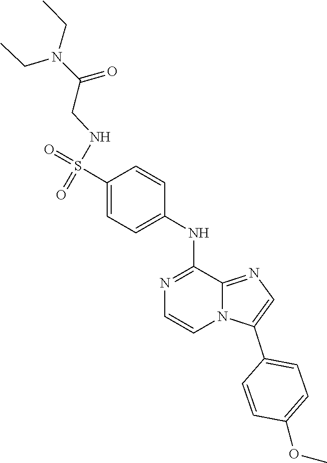 [SMILES: CCN(CC)C(=O)CNS(=O)(=O)c1ccc(Nc2nccn3c(-c4ccc(OC)cc4)cnc23)cc1] | 509.3 | 2-(4- aminophenylsulfonamido)- N,N- diethylacetamide |
| 81 | N-(3-chloro-4- (methylsulfonyl) phenyl)-3-(4- methoxyphenyl) imidazo [1,2-a]pyrazin-8- amine | 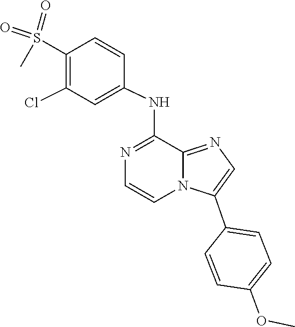 [SMILES: COc1ccc(-c2cnc3c(Nc4ccc(S(C)(=O)=O)c(Cl)c4)nccn23)cc1] | 429.1 | 2-(4- aminophenylsulfonamido)- N,N-diethylacetamide |

### Example 82

### N-(4-((3-(2,3,4-trifluorophenyl)imidazo[1,2-a]pyrazin-8-yl)amino)phenyl)acetamide

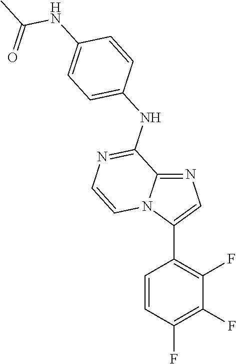 [SMILES: CC(=O)Nc1ccc(Nc2nccn3c(-c4ccc(F)c(F)c4F)cnc23)cc1]

### Step 1) N-(4-((3-bromoimidazo[1,2-a]pyrazin-8-yl)amino)phenyl)acetamide

To a solution of 3-bromo-8-chloroimidazo[1,2-a]pyrazine (100 mg, 430 μmol, Eq: 1) CAS 143591-61-1 in dioxane (422 μl) and acetic acid (422 μl), N-(4-amino-phenyl)-acetamide (84 mg, 559 μmol, Eq: 1.3) CAS 56891-59-9 was added. The RM was heated in at 100° C. O/N.

After cooling down to room temperature, the RM was filtrated, followed by washing with diethyl ether to give 95 mg (56%) of the title product as a light brown powder. MS (ESI, m/z): 348.0 [M+H]⁺

### Step 2) N-(4-((3-(2,3,4-trifluorophenyl)imidazo[1,2-a]pyrazin-8-yl)amino)phenyl)acetamide

A solution of N-(4-((3-bromoimidazo[1,2-a]pyrazin-8-yl)amino)phenyl)acetamide (34.6 mg, 0.1 mmol, Eq: 1), (2,3,4-trifluorophenyl)boronic acid (26.4 mg, 150 μmol, Eq: 1.5), 1,1′-bis(diphenylphosphino)ferrocenedichloro palladium(II) dichloromethane complex (7.32 mg, 10 μmol, Eq: 0.1) and potassium carbonate (34.6 mg, 250 μmol, Eq: 2.5) in dioxane (0.9 ml) and water (100 μl) was degassed and shaken at 105° C. O/N. After cooling down, the RM was concentrated, dissolved in 2 ml DMSO, shaken with SiliaMetS Thiourea at 70° C. for 1h, filtered over celite and purified by prep. HPLC to give 19 mg (48%) of the title product. MS (ESI, m/z): 398.2 [M+H]⁺

### Example 83

### N-(4-((3-(2,4-difluorophenyl)imidazo[1,2-a]pyrazin-8-yl)amino)-2-methylphenyl)acetamide

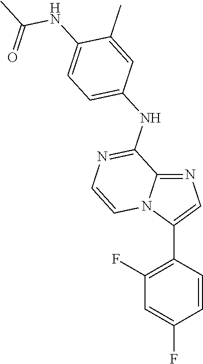 [SMILES: CC(=O)Nc1ccc(Nc2nccn3c(-c4ccc(F)cc4F)cnc23)cc1C]

A solution of Intermediate 2, (2,4-difluorophenyl)boronic acid (23.7 mg, 150 μmol, Eq: 1.5), 1,1′-bis(diphenylphosphino)ferrocenedichloro palladium(II) dichloromethane complex (7.32 mg, 10 μmol, Eq: 0.1) and potassium carbonate (34.6 mg, 250 μmol, Eq: 2.5) in dioxane (0.9 ml) and water (100 μl) was degassed and shaken at 105° C. O/N. After cooling down, the RM was concentrated, dissolved in 2 ml DMSO, shaken with SiliaMetS Thiourea at 70° C. for 1h, filtered over celite and purified by prep. HPLC to give 24.0 mg (61%) of the title product. MS (ESI, m/z): 394.2 [M+H]+

The following Examples were obtained in analogy:

| | | | ESI MS | Starting |
|---|---|---|---|---|
| Ex. | Name | Structure | [M + H]⁺ | Material |
| 84 | N-(4-((3-(4-chloro- 3- fluorophenyl) imidazo[1,2-a]pyrazin-8- yl)amino)-2- methylphenyl) | 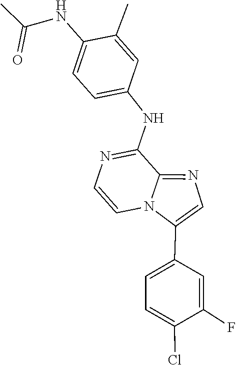 [SMILES: CC(=O)Nc1ccc(Nc2nccn3c(-c4ccc(Cl)c(F)c4)cnc23)cc1C] | 410.1 | (4-chloro-3- fluorophenyl) boronic acid |
| 85 | N-(4-((3-(3,4- difluorophenyl) imidazo[1,2-a]pyrazin-8- yl)amino)-2- methylphenyl) acetamide | 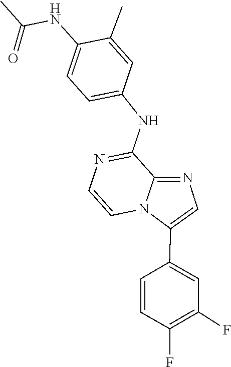 [SMILES: CC(=O)Nc1ccc(Nc2nccn3c(-c4ccc(F)c(F)c4)cnc23)cc1C] | 394.2 | (3,4- difluorophenyl) boronic acid |
| 86 | N-(4-((3-(2-chloro- 4- methoxyphenyl)imidazo [1,2-a]pyrazin-8- yl)amino)-2- methylphenyl)acetamide | 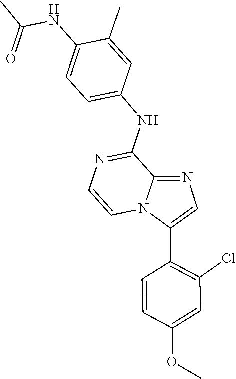 [SMILES: COc1ccc(-c2cnc3c(Nc4ccc(NC(C)=O)c(C)c4)nccn23)c(Cl)c1] | 422.2 | (2-chloro-4- methoxyphenyl) boronic acid |
| 87 | N-(2-methyl-4-((3- (2,3,4- trifluorophenyl) imidazo[1,2-a]pyrazin-8- yl)amino)phenyl) acetamide | 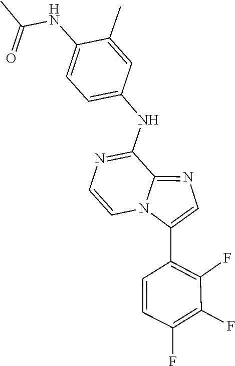 [SMILES: CC(=O)Nc1ccc(Nc2nccn3c(-c4ccc(F)c(F)c4F)cnc23)cc1C] | 412.0 | (2,3,4- trifluorophenyl) boronic acid |
| 88 | N-(4-((3-(4- (cyanomethoxy) phenyl)imidazo[1,2- a]pyrazin-8- yl)amino)-2- methylphenyl) acetamide | 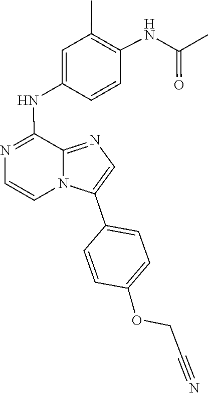 [SMILES: CC(=O)Nc1ccc(Nc2nccn3c(-c4ccc(OCC#N)cc4)cnc23)cc1C] | 413.3 | 4- Cyanomethoxyphenylboronic acid, CAS 947533-23-5 |
| 89 | N-(4-((3-(2-fluoro- 4- methoxyphenyl)imidazo [1,2-a]pyrazin-8- yl)amino)-2- methylphenyl) acetamide | 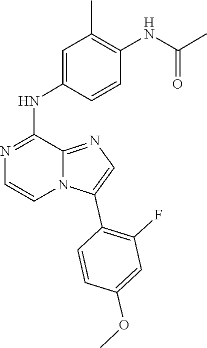 [SMILES: COc1ccc(-c2cnc3c(Nc4ccc(NC(C)=O)c(C)c4)nccn23)c(F)c1] | 406.2 | 2- FLUORO- 4- METHOXYPHENYLBORONIC ACID, CAS 162101-31- 7 |

### Example 90

### N-(2-(2-((4-((3-(4-methoxyphenyl)imidazo[1,2-a]pyrazin-8-yl)amino)-2-methylphenyl)amino)-2-oxoethoxy)ethyl)pentanamide

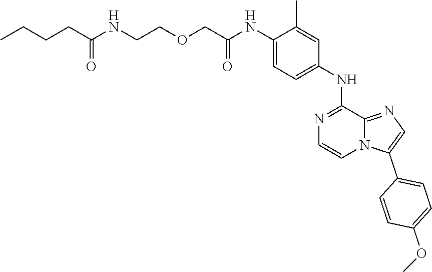 [SMILES: CCCCC(=O)NCCOCC(=O)Nc1ccc(Nc2nccn3c(-c4ccc(OC)cc4)cnc23)cc1C]

### Step 1) 8-chloro-3-(4-methoxyphenyl)imidazo[1,2-a]pyrazine

The title compound was obtained in analogy to Example 75, Step 1. MS (ESI, m/z): 260.1 [M+H]⁺

### Step 2) N1-(3-(4-methoxyphenyl)imidazo[1,2-a]pyrazin-8-yl)-3-methylbenzene-1,4-diamine

To 8-chloro-3-(4-methoxyphenyl)imidazo[1,2-a]pyrazine (100 mg, 385 μmol, Eq: 1) in acetonitrile (3 ml) was added 2-methylbenzene-1,4-diamine sulfate (127 mg, 578 μmol, Eq: 1.5) with stirring at 90° C. O/N. DIPEA (149 mg, 202 μl, 1.16 mmol, Eq: 3) was added followed by stirring at 90° C. O/N. Acetic acid (210 mg, 200 μl, 3.49 mmol, Eq: 9.07) was added. The RM was left to stir at RT for 2d. The RM was concentrated and purified by prep. HPLC to give 64.6 mg (49%) of the title product as a light grey solid. MS (ESI, m/z): 346.2 [M+H]+

### Step 3) tert-butyl (2-(2-((4-((3-(4-methoxyphenyl)imidazo[1,2-a]pyrazin-8-yl)amino)-2-methylphenyl)amino)-2-oxoethoxy)ethyl)carbamate

To N1-(3-(4-methoxyphenyl)imidazo[1,2-a]pyrazin-8-yl)-3-methylbenzene-1,4-diamine (25.3 mg, 73.2 μmol, Eq: 1) in DMF (1 ml) was added DIPEA (28.4 mg, 38.4 μl, 220 μmol, Eq: 3), followed by 2-(2-((tert-butoxycarbonyl)amino)ethoxy)acetic acid (17.7 mg, 80.6 μmol, Eq: 1.1) and finally HATU (55.7 mg, 146 μmol, Eq: 2) with stirring at RT for 6h. The RM was concentrated and purified by prep. HPLC to give 28.2 mg (70%) of the title product as a yellow solid. MS (ESI, m/z): 547.3 [M+H]+

### Step 4) 2-(2-aminoethoxy)-N-(4-((3-(4-methoxyphenyl)imidazo[1,2-a]pyrazin-8-yl)amino)-2-methylphenyl)acetamide 2,2,2-trifluoroacetate

To tert-butyl (2-(2-((4-((3-(4-methoxyphenyl)imidazo[1,2-a]pyrazin-8-yl)amino)-2-methylphenyl)amino)-2-oxoethoxy)ethyl)carbamate (28.2 mg, 51.6 μmol, Eq: 1) was added TFA (1.48 g, 1 ml, 13 mmol, Eq: 252) and DCM (1 ml) followed by stirring at RT for 4h. The RM was concentrated and dried under vacuum O/N to give 83.5 mg of the title product as an orange oil which was used without further purification in the next step. MS (ESI, m/z): 447.3 [M+H]+

### Step 5) N-(2-(2-((4-((3-(4-methoxyphenyl)imidazo[1,2-a]pyrazin-8-yl)amino)-2-methylphenyl)amino)-2-oxoethoxy)ethyl)pentanamide

To 2-(2-aminoethoxy)-N-(4-((3-(4-methoxyphenyl)imidazo[1,2-a]pyrazin-8-yl)amino)-2-methylphenyl)acetamide 2,2,2-trifluoroacetate (28.9 mg, 51.6 μmol, Eq: 1) in DMF (1 ml) was added pentanoic acid (5.79 mg, 6.23 μl, 56.7 μmol, Eq: 1.1), DIPEA (20 mg, 27 μl, 155 μmol, Eq: 3) and HATU (39.2 mg, 103 μmol, Eq: 2) followed by stirring at RT for 3h. The RM was purified by prep. HPLC to give 20.3 mg (74%) of the title product as white solid. MS (ESI, m/z): 531.4 [M+H]+

### Example 91

### 4-((3-(3-chloro-4-methoxyphenyl)imidazo[1,2-a]pyrazin-8-yl)amino)-N-methylbenzenesulfonamide

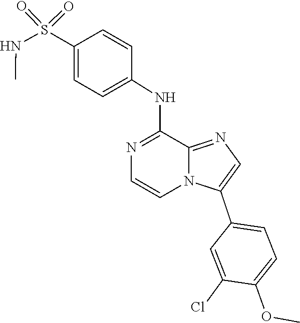

### Step 1) 8-chloro-3-(3-chloro-4-methoxyphenyl)imidazo[1,2-a]pyrazine

8-chloro-3-bromoimidazo[1,2-a]pyrazine (2 g, 7.16 mmol, Eq: 1) was combined with dioxane (20 ml) and water (10 ml). (3-chloro-4-methoxyphenyl)boronic acid (1.6 g, 8.59 mmol, Eq: 1.20), Na₂CO₃ (1.52 g, 14.3 mmol, Eq: 2.00) and 1,1′-bis(diphenylphosphino)ferrocenedichloro palladium(II) dichloromethane complex (262 mg, 358 μmol, Eq: 0.05) were added, and the RM was heated at 80° C. After cooling down to RT, the RM was diluted with AcOEt and water and separated the organic layer. Aqueous layer was extracted with twice AcOEt. Combined organic layer was washed with brine solution and dried over Na₂SO₄. Organic layer was evaporated under reduced pressure. The brown solid obtained was purified by flash chromatography (silica gel, 80 g, 0% to 80% EtOAc in heptane) to give 1.1 g (54%) of the title product as a light brown solid. MS (ESI, m/z): 294.1 [M+H]⁺

### Step 2) 4-((3-(3-chloro-4-methoxyphenyl)imidazo[1,2-a]pyrazin-8-yl)amino)-N-methylbenzenesulfonamide

A mixture of 4-amino-N-methylbenzenesulfonamide (30 mg, 161 μmol, Eq: 1.9) and 8-chloro-3-(3-chloro-4-methoxyphenyl)imidazo[1,2-a]pyrazine (25 mg, 85 μmol, Eq: 1) in 10/1 ACN/AcOH (1 ml) was heated in a sealed tube at 80° C. overnight.

The mixture was purified by prep. HPLC to give 2.7 mg (7%) of the title product as a white powder. MS (ESI, m/z): 444.1 [M+H]⁺

### Example 92

### N1-(3-(2,3-difluoro-4-methoxyphenyl)imidazo[1,2-a]pyrazin-8-yl)-3-ethylbenzene-1,4-diamine

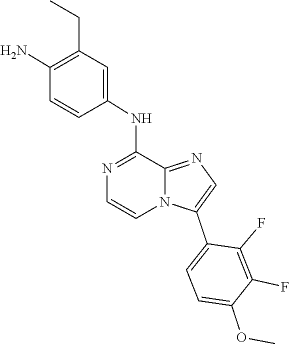 [SMILES: CCc1cc(Nc2nccn3c(-c4ccc(OC)c(F)c4F)cnc23)ccc1N]

### Step 1) methyl 4-amino-2-vinylbenzoate

Methyl 4-amino-2-bromobenzoate (1 g, 4.35 mmol, Eq: 1) CAS 98545-64-3, 4,4,5,5-tetramethyl-2-vinyl-1,3,2-dioxaborolane (1 g, 1.11 ml, 6.52 mmol, Eq: 1.5) CAS 75927-49-0, sodium methoxide (470 mg, 484 μl, 8.69 mmol, Eq: 2) and tetrakis(triphenylphosphine)palladium (0) (251 mg, 217 μmol, Eq: 0.05) were added in THF (8 ml) and water (800 μl). The reaction mixture was heated in the microwave at 110° C. for 30 min. The reaction mixture was poured into 20 mL H₂O and extracted with EtOAc. The organic layers were dried over MgSO₄ and concentrated in vacuo. The crude material was purified by flash chromatography (silica gel, 12 g, 0% to 50% EtOAc in heptane) to give 345.7 mg (45%) of the title product as an orange oil. MS (ESL, m/z): 178.2 [M+H]+

### Step 2) methyl 4-amino-2-ethylbenzoate

Methyl 4-amino-2-vinylbenzoate (665 mg, 3.75 mmol, Eq: 1) was dissolved in MeOH (2 ml) and palladium on carbon (39.9 mg, 375 μmol, Eq: 0.1) was added. The reaction was stirred under hydrogen. The reaction mixture was carefully filtered under argon through celite. The crude reaction mixture was concentrated in vacuo to give 601.3 mg (89%) of the title product as a dark brown oil which was used without further purification. MS (ESI, m/z): 180.2 [M+H]+

### Step 3) methyl 2-ethyl-4-((3-iodoimidazo[1,2-a]pyrazin-8-yl)amino)benzoate

8-chloro-3-iodoimidazo[1,2-a]pyrazine (468 mg, 1.67 mmol, Eq: 1.5) CAS 1049677-32-8 and methyl 4-amino-2-ethylbenzoate (200 mg, 1.12 mmol, Eq: 1) were combined in acetonitrile (5 ml) and acetic acid (500 μl). The reaction mixture was stirred over night at 90° C. The reaction mixture was filtered through sintered glass to give 553.9 mg (118%) of the title product as a white solid. MS (ESI, m/z): 423.2 [M+H]+

### Step 4) methyl 4-((3-(2,3-difluoro-4-methoxyphenyl)imidazo[1,2-a]pyrazin-8-yl)amino)-2-ethylbenzoate

To a 10 mL microwave vial were added methyl 2-ethyl-4-((3-iodoimidazo[1,2-a]pyrazin-8-yl)amino)benzoate (538 mg, 1.27 mmol, Eq: 1), (2,3-difluoro-4-methoxyphenyl)boronic acid (335 mg, 1.78 mmol, Eq: 1.40), Na₂CO₃ (270 mg, 2.55 mmol, Eq: 2.00) and 1,1′-bis(diphenylphosphino)ferrocene-palladium(II)dichloride dichloromethane complex (104 mg, 127 μmol, Eq: 0.10) in dioxane (7.5 ml) and water (750 μl). The vial was capped and heated in the microwave at 110° C. for 30 min. The reaction mixture was diluted with AcOEt, and filtered over celite. After removal of the volatiles, the crude material was purified by flash chromatography (silica gel, 50 g, 0% to 80% EtOAc in heptane) to give 486.0 mg (81%) of the title product as a brown solid. MS (ESI, m/z): 439.4 [M+H]+

### Step 5) 4-((3-(2,3-difluoro-4-methoxyphenyl)imidazo[1,2-a]pyrazin-8-yl)amino)-2-ethylbenzoic acid

Methyl 4-((3-(2,3-difluoro-4-methoxyphenyl)imidazo[1,2-a]pyrazin-8-yl)amino)-2-ethylbenzoate (485 mg, 1.11 mmol, Eq: 1) was combined with THF (5 ml) and MeOH (2.5 ml). LiOH 1M (2.21 ml, 2.21 mmol, Eq: 2.00) was added and the reaction mixture was stirred at 60° C. After cooling down to RT, the volatiles were removed, the residue taken in water and acidified with 1N HCl solution. The product has precipitated. It was filtered and dried under HV to give 459.3 mg (93%) of the title product as a brown solid. MS (ESI, m/z): 425.3 [M+H]+

### Step 6) N1-(3-(2,3-difluoro-4-methoxyphenyl)imidazo[1,2-a]pyrazin-8-yl)-3-ethylbenzene-1,4-diamine

To a solution of 4-((3-(2,3-difluoro-4-methoxyphenyl)imidazo[1,2-a]pyrazin-8-yl)amino)-2-ethylbenzoic acid (100 mg, 236 μmol, Eq: 1) in THF (2.36 ml) was added triethylamine (35.8 mg, 49 μl, 353 μmol, Eq: 1.5), AZIDOTRIMETHYLSILANE (32.6 mg, 37.4 μl, 283 μmol, Eq: 1.2) and 1-PROPANEPHOSPHONIC ANHYDRIDE (180 mg, 168 μl, 283 μmol, Eq: 1.2).

RM was stirred at reflux for 1h. water (1 g, 1 ml, 55.5 mmol, Eq: 236) was added and RM was reflux for additional 1h. The mixture was diluted with 1M aq. NaOH and DCM and extracted 5 times with DCM. Organic layers were combined, washed with brine and filtered through a phase separator (allows to obtain a clear solution). RM was concentrated to give 42 mg (41%) of the title product as an orange waxy solid. MS (ESI, m/z): 396.2 [M+H]⁺

### Example 93

### 3-amino-N-(4-((3-(2,3-difluoro-4-methoxyphenyl)imidazo[1,2-a]pyrazin-8-yl)amino)-2-ethylphenyl)propanamide hydrochloride

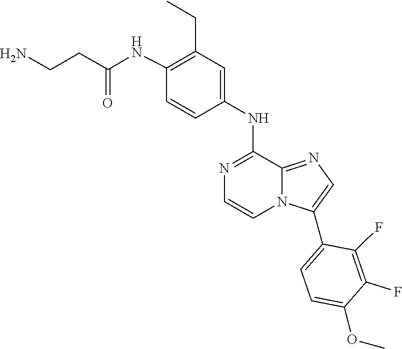 [SMILES: CCc1cc(Nc2nccn3c(-c4ccc(OC)c(F)c4F)cnc23)ccc1NC(=O)CCN]

### Step 1) tert-butyl (3-((4-((3-(2,3-difluoro-4-methoxyphenyl)imidazo[1,2-a]pyrazin-s-yl)amino)-2-ethylphenyl)amino)-3-oxopropyl)carbamate

N1-(3-(2,3-difluoro-4-methoxyphenyl)imidazo[1,2-a]pyrazin-8-yl)-3-ethylbenzene-1,4-diamine (42 mg, 106 μmol, Eq: 1) 92, 3-((tert-butoxycarbonyl)amino)propanoic acid (40.2 mg, 212 μmol, Eq: 2), 2-(3H-[1,2,3]triazolo[4,5-b]pyridin-3-yl)-1,1,3,3-tetramethylisouronium hexafluorophosphate(V) (80.8 mg, 212 μmol, Eq: 2), DIPEA (54.9 mg, 74.2 μl, 425 μmol, Eq: 4) were mixed together and stirred at RT. The mixture was purified by prep. HPLC to give 32 mg (51%) of the title product as an off-white solid. MS (ESI, m/z): 567.3 [M+H]⁺

### Step 2) 3-amino-N-(4-((3-(2,3-difluoro-4-methoxyphenyl)imidazo[1,2-a]pyrazin-s-yl)amino)-2-ethylphenyl)propanamide hydrochloride

HCl in dioxane (137 μl, 547 μmol, Eq: 10) was added to a solution of tert-butyl (3-((4-((3-(2,3-difluoro-4-methoxyphenyl)imidazo[1,2-a]pyrazin-8-yl)amino)-2-ethylphenyl)amino)-3-oxopropyl)carbamate (31 mg, 54.7 μmol, Eq: 1) in DCM (150 μL). RM was stirred at RT. RM was dried with HV and the product finally freeze-dried to give 28.0 mg (81%) of the title product as an off-white amorphous freeze-dried solid. MS (ESI, m/z): 467.3 [M+H]⁺

### Example 94

### 1-(2-chloro-4-((3-(4-(difluoromethoxy)phenyl)imidazo[1,2-a]pyrazin-8-yl)amino)phenyl)pyrrolidin-2-one

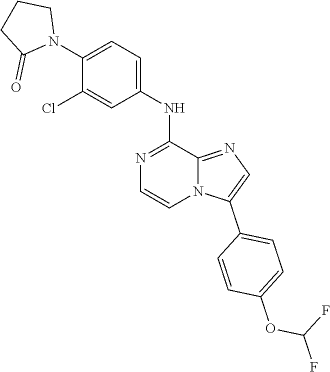 [SMILES: O=C1CCCN1c1ccc(Nc2nccn3c(-c4ccc(OC(F)F)cc4)cnc23)cc1Cl]

### Step 1) 8-chloro-3-(4-(difluoromethoxy)phenyl)imidazo[1,2-a]pyrazine

8-chloro-3-iodoimidazo[1,2-a]pyrazine (250 mg, 895 μmol, Eq: 1) CAS 1049677-32-8 was combined with dioxane (3 ml) and water (1.5 ml). 2-(4-(difluoromethoxy)phenyl)-4,4,5,5-tetramethyl-1,3,2-dioxaborolane (290 mg, 1.07 mmol, Eq: 1.2), Na₂CO₃ (190 mg, 1.79 mmol, Eq: 2.00) and 1,1′-bis(diphenylphosphino)ferrocenedichloro palladium(II) dichloromethane complex (32.7 mg, 44.7 μmol, Eq: 0.05) were added, and the reaction mixture was heated at 80° C. with stirring over night. The reaction mixture was diluted with AcOEt and water and separated the organic layer. The aqueous layer was extracted with EtOAc several times. The combined organic layers were washed with brine. The organic layers were dried over Na₂SO₄ and concentrated in vacuo. The crude material was purified by flash chromatography (silica gel, 20 g, 0% to 80% EtOAc in heptane) to give 174.9 mg (66%) of the title product as an off-white solid. MS (ESI, m/z): 296.1 [M+H]⁺

### Step 2) 1-(2-chloro-4-((3-(4-(difluoromethoxy)phenyl)imidazo[1,2-a]pyrazin-8-yl)amino)phenyl)pyrrolidin-2-one

8-chloro-3-(4-(difluoromethoxy)phenyl)imidazo[1,2-a]pyrazine (30 mg, 101 μmol, Eq: 1) was combined with acetonitrile (1 ml). 1-(4-amino-2-chlorophenyl)pyrrolidin-2-one (32 mg, 152 μmol, Eq: 1.5) and acetic acid (105 mg, 100 μl, 1.75 mmol, Eq: 17.2) were added, with stirring over the weekend at 80° C.

After concentration, the crude was purified by prep HPLC to give 31.4 mg (66%) of the title product. MS (ESI, m/z): 470.1 [M+H]⁺

The following Example was obtained in analogy:

| | | | ESI MS | Starting |
|---|---|---|---|---|
| Ex. | Name | Structure | [M + H]⁺ | Material |
| 95 | 1-(4-((3-(4- (difluoromethoxy) phenyl)imidazo[1,2- a]pyrazin-8- yl)amino)phenyl)-5- methylpyrrolidin-2- one | 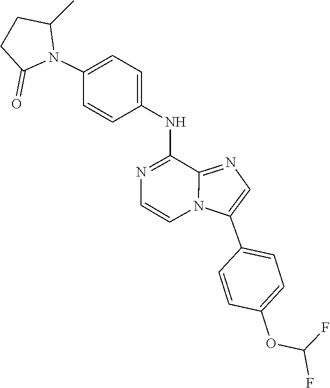 [SMILES: CC1CCC(=O)N1c1ccc(Nc2nccn3c(-c4ccc(OC(F)F)cc4)cnc23)cc1] | 448.4 (M + H)+ | 1-4- aminophenyl)- 5- methylpyrrolidin- 2-one |

### Example 96

### N-(3-chloro-4-((4-(3-(dimethylamino)propyl)piperazin-1-yl)sulfonyl)phenyl)-3-(2,3-difluoro-4-methoxyphenyl)imidazo[1,2-a]pyrazin-8-amine

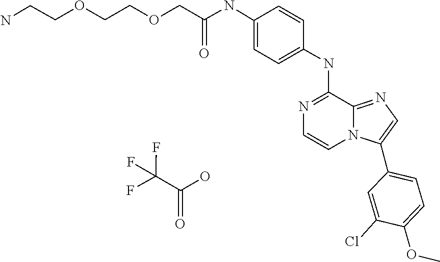

### Step 1) 3-(4-((4-bromo-2-chlorophenyl)sulfonyl)piperazin-1-yl)-N,N-dimethylpropan-1-amine

To a solution of 4-bromo-2-chlorobenzenesulfonyl chloride (0.25 mmol, 1 Eq.) in dichloromethane (1 ml) and DIPEA (175 ul, 1 mmol, 4 Eq.) a solution of N,N-dimethyl-3-(piperazin-1-yl)propan-1-amine (51.4 mg, 1.2 Eq) was added and the mixture was stirred for 3 h at room temperature. The solvents were removed under vacuum to afford the title compound (ESI MS [M+H]⁺: 424.1) as crude product that was directly used for the next step.

Seven intermediates were obtained in analogy

| | | ESI MS | |
|---|---|---|---|
| Int. | Name | [M + H]⁺ | Starting material |
| A | 4-amino-2-chloro-N-(2-(2- | 385.1 | a) 4-bromo-2- |
| | (dimethylamino)ethoxy)ethyl)benzenesulfonamide | | chlorobenzenesulfonyl chloride |
| | | | b) 2-(2-aminoethoxy)-N,N- |
| | | | dimethylethan-1-amine |
| B | tert-butyl (2-(2-((4-bromo-2- | 435.2 | a) 4-bromo-2- |
| | methylphenyl)sulfonamido)ethoxy)ethyl)carbamate | (M − H)− | methylbenzenesulfonyl chloride |
| | | | b) tert-butyl (2-(2- |
| | | | aminoethoxy)ethyl)carbamate |
| C | tert-butyl (2-(2-((4-bromo-2- | 455.1 | a) 4-bromo-2- |
| | chlorophenyl)sulfonamido)ethoxy)ethyl)carbamate | (M − H) | chlorobenzenesulfonyl chloride |
| | | | b) tert-butyl (2-(2- |
| | | | aminoethoxy)ethyl)carbamate |
| D | 3-(4-((4-bromo-2- | 405.2 | a)4-bromo-2- |
| | methylphenyl)sulfonyl)piperazin-1-yl)-N,N- | | methylbenzenesulfonyl chloride |
| | dimethylpropan-1-amine | | b) N,N-dimethyl-3-(piperazin- |
| | | | 1-yl)propan-1-amine |
| E | 4-bromo-N-(2-(2- | 365.1 | a) 4-bromo-2- |
| | (dimethylamino)ethoxy)ethyl)- | | methylbenzenesulfonyl chloride |
| | 2-methylbenzenesulfonamide | | b) 2-(2-aminoethoxy)-N,N- |
| | | | dimethylethan-1-amine |
| F | tert-butyl N-[2-[2-[(4- | 423.1 | a) 4-bromo-benzenesulfonyl |
| | bromophenyl)sulfonylamino]ethoxy]ethyl]carbamate | | chloride |
| | | | b) tert-butyl (2-(2- |
| | | | aminoethoxy)ethyl)carbamate |
| G | tert-butyl 4-((4-bromo-2- | 333.1 | a) 4-bromo-2- |
| | ethylphenyl)sulfonyl)piperazine-1-carboxylate | (M − BOC + H)+ | ethylbenzenesulfonyl chloride |
| | | | b) tert-butyl piperazine-1- |
| | | | carboxylate |

### Step 2) N-(3-chloro-4-((4-(3-(dimethylamino)propyl)piperazin-1-yl)sulfonyl)phenyl)-3-(2,3-difluoro-4-methoxyphenyl)imidazo[1,2-a]pyrazin-amine

To a brown suspension of 3-(2,3-difluoro-4-methoxyphenyl)imidazo[1,2-a]pyrazin-8-amine (69.1 mg, 250 μmol, Eq: 1), 3-(4-((4-bromo-2-chlorophenyl)sulfonyl)piperazin-1-yl)-N,N-dimethylpropan-1-amine (106 mg, 250 μmol, Eq: 1) and sodium tert-butoxide (36 mg, 375 μmol, Eq: 1.5) in THF (2.5 ml) under argon 1,1′-bis(diphenylphosphino)ferrocene (16.6 mg, 30 μmol, Eq: 0.12) and tris(dibenzylideneacetone)dipalladium (0) (9.16 mg, 10 μmol, Eq: 0.04) are added and degassing continued for 1 minute with argon. The tube is sealed and heated to 130° for 2.5h hours. The mixture is filtered through a 4 g SiO2 column with THF/MeOH and the residue is concentrated in vacuo. The crude is resolved in DMF and purified by Prep. HPLC to afford the title compound (5.1 mg, 8.22 μmol, 3.29% yield) as a waxy light brown solid ESI MS [M+H]⁺: 620.3

Seven examples were obtained in analogy

| | | | ESI | |
|---|---|---|---|---|
| Example/ | | | MS | Starting |
| Int. | Name | Structure | [M + H]⁺ | material |
| 97 | 2-chloro-4-((3- 2,3-difluoro-4- oxyphenyl)imid 1,2-a]pyrazin-8- amino)-N-(2-(2- methylamino)eth) ethyl) benzenesulfonamide | 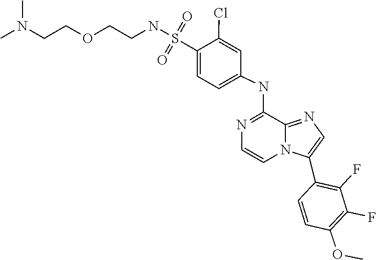 | 581.2 | 4- amino- 2- chloro- N-(2-(2- (dimethylamino) ethoxy) ethyl) benzenesulfonamide |
| H | tert-butyl (2-(2- ((4-((3-(2,3- difluoro-4- methoxyphenyl) imidazo[1,2- a]pyrazin-8- yl)amino)-2- methylphenyl) sulfonamido)ethoxy) ethyl)carbamate | 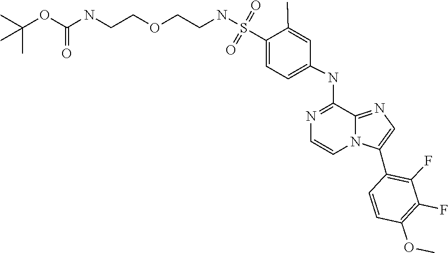 | 633.4 | tert-butyl (2-(2- ((4- bromo- 2- methylphenyl) sulfonamido) ethoxy) ethyl) carbamate |
| I | tert-butyl N-[2- [2-[[2-chloro-4- [3-(2,3- difluoro-4- methoxy- phenyl)imidazo [1,2-a]pyrazin- 8-yl]amino]phenyl] sulfonylamino] ethoxy]ethyl] carbamate | 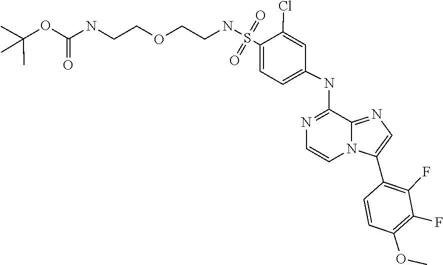 | 653.3 | tert- butyl (2-(2- ((4- bromo- 2-chlorophenyl) sulfonamido) ethoxy) ethyl) carbamate |
| 98 | 3-(2,3-difluoro- 4- methoxyphenyl)- N-(4-((4-((3- (dimethylamino) propyl)piperazin- 1- yl)sulfonyl)-3- methylphenyl) imidazo[1,2- a]pyrazin-8- amine | 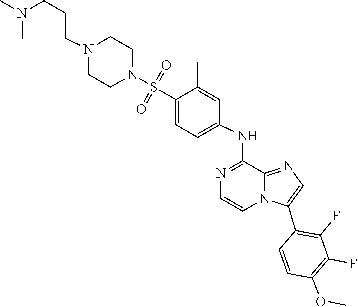 [SMILES: COc1ccc(-c2cnc3c(Nc4ccc(S(=O)(=O)N5CCN(CCCN(C)C)CC5)c(C)c4)nccn23)c(F)c1F] | 600.4 | 3-(4- ((4- bromo- 2- methylphenyl) sulfonyl) piperazin-1-yl)- N,N- dimethylpropan- 1-amine |
| 99 | 4-((3-(2,3- difluoro-4- methoxyphenyl) imidazo[1,2- a]pyrazin-8- yl)amino)-N- (2-(2- (dimethylamino) ethoxy)ethyl)- methylbenzene sulfonamide | 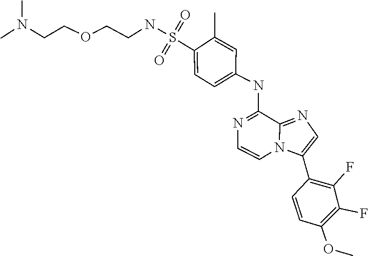 | 561.3 | 4- bromo- N-(2-(2- (dimethylamino) ethoxy) ethyl)-2- methylbenzenesulfonamide |
| J | tert-butyl (2-(2- (4-((3-(2,3- difluoro-4- methoxyphenyl) imidazo[1,2- a]pyrazin-8- yl)amino)phenyl) sulfonamido) ethoxy)ethyl) carbamate | 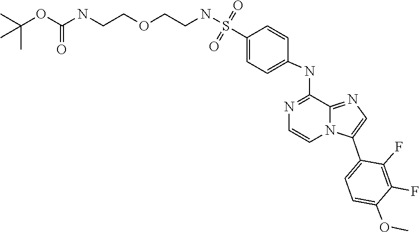 | 619.3 | tert- butyl (2-(2- ((4- bromophenyl) sulfonamido) ethoxy) ethyl)carbamate |
| K | tert-butyl 4-((4- ((3-(2,3- difluoro-4- methoxyphenyl) imidazo[1,2- a]pyrazin-8- yl)amino)-2- ethylphenyl) sulfonyl) piperazine-1- carboxylate | 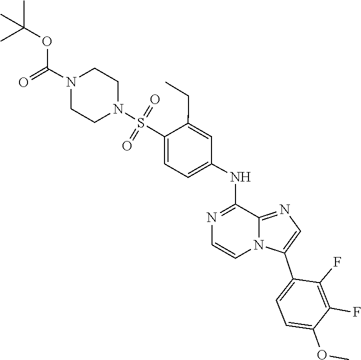 [SMILES: CCc1cc(Nc2nccn3c(-c4ccc(OC)c(F)c4F)cnc23)ccc1S(=O)(=O)N1CCN(C(=O)OC(C)(C)C)CC1] | | tert- butyl 4- ((4- bromo- 2- ethylphenyl) sulfonyl) piperazine- 1-carboxylate |

### Example 100

### Preparation of N-(2-(2-aminoethoxy)ethyl)-4-((3-(2,3-difluoro-4-methoxyphenyl)imidazo[1,2-a]pyrazin-8-yl)amino)-2-methylbenzenesulfonamide hydrochloride

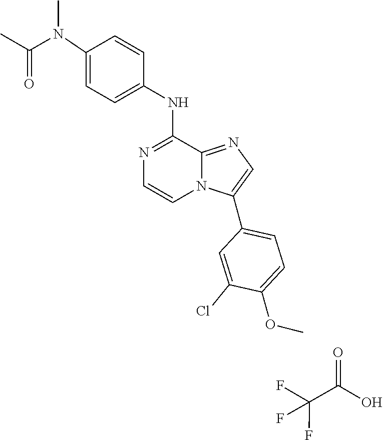

To a solution of tert-butyl (2-(2-((4-((3-(2,3-difluoro-4-methoxyphenyl)imidazo[1,2-a]pyrazin-8-yl)amino)-2-methylphenyl)sulfonamido)ethoxy)ethyl)carbamate (35.2 mg, 55.6 μmol, Eq: 1) in dichloromethane (3 ml), HCl (4 M in dioxane (100 μl, 400 μmol, Eq: 7.19)) is added and the mixture was stirred over night at 22° C. All solvents were removed and the residue was dried under vacuum (<5 mbar, 3h, 45° C.) to obtain the title compound (31.5 mg, 55.4 μmol, 99.5% yield) as a white solid ESI MS [M+H]⁺: 533.2.

Three examples were obtained in analogy

| | | | ESI MS | Starting |
|---|---|---|---|---|
| Example | Name | Structure | [M + H]⁺ | material |
| 101 | N-(2-(2- aminoethoxy) ethyl)-2- chloro-4-((3- (2,3- difluoro-4- methoxyphenyl) imidazo[1,2- a]pyrazin-8- yl)amino) benzenesulfonamide hydrochloride | 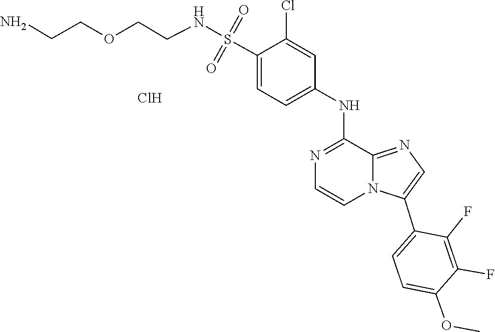 [SMILES: COc1ccc(-c2cnc3c(Nc4ccc(S(=O)(=O)NCCOCCN)c(Cl)c4)nccn23)c(F)c1F ; Cl] | 553.2 | tert-butyl N-[2-[2-[[2- chloro-4- [[3-(2,3- difluoro-4- methoxy- phenyl) imidazo[1,2- a]pyrazin-8- yl]amino] phenyl] sulfonylamino] ethoxy]ethyl] carbamate |
| 102 | N-(2-(2- aminoethoxy) ethyl)-4- (3-(2,3- difluoro-4- methoxyphenyl) imidazo [1,2- a]pyrazin-8- yl)amino) benzenesulfonamide hydrochloride | 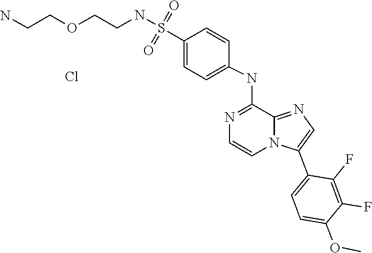 | 519.3 | tert-butyl (2-(2-((4- ((3-(2,3- difluoro-4- methoxyphenyl) imidazo[1,2- a]pyrazin-8- yl)amino) phenyl) sulfonamido) ethoxy)ethyl) carbamate |
| 103 | 3-(2,3- difluoro-4- methoxyphenyl)- N-(3-ethyl-4- (piperazin-1- ylsulfonyl) phenyl) imidazo[1,2- a]pyrazin-8- amine hydrochloride | 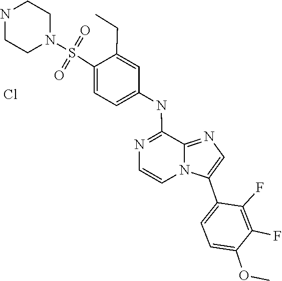 | 527.4 | tert-butyl 4- (4-((3-(2,3- difluoro-4- methoxyphenyl) imidazo [1,2- a]pyrazin-8- yl)amino)-2- ethylphenyl) sulfonyl) piperazine-1- carboxylate |

### Example 104

### 1-(4-((4-((3-(2,3-difluoro-4-methoxyphenyl)imidazo[1,2-a]pyrazin-8-yl)amino)-2-ethylphenyl)sulfonyl)piperazin-1-yl)-2-(dimethylamino)ethan-1-one

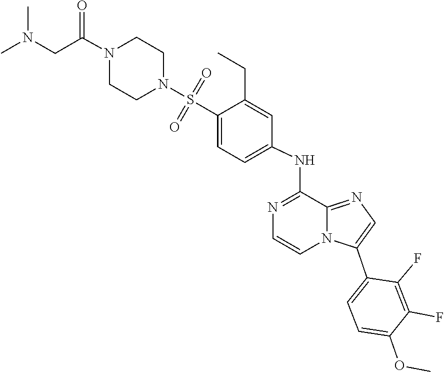 [SMILES: CCc1cc(Nc2nccn3c(-c4ccc(OC)c(F)c4F)cnc23)ccc1S(=O)(=O)N1CCN(C(=O)CN(C)C)CC1]

A solution of dimethylglycine (6.5 mg, 63 umol, Eq: 1.05) and TBTU (20.2 mg, 63 μmol, Eq: 1.05) in DMF (0.5 ml) was stirred for 20 min at room temperature. Then a solution of 3-(2,3-difluoro-4-methoxyphenyl)-N-(3-ethyl-4-(piperazin-1-ylsulfonyl)phenyl)imidazo[1,2-a]pyrazin-8-amine hydrochloride (33.9 mg, 0.06 mmol, Eq: 1) in DMF (1 ml) and DIPEA (33.3 mg, 45 μl, 258 μmol, Eq: 4.29) were added and the mixture was stirred for 2.5 h at room temperature. The mixture was directly purified by Prep. HPLC to afford the title compound (29.5 mg, 48.1 μmol, 80% yield) as a waxy solid. ESI MS [M+H]+: 614.4

Five intermediates were obtained in analogy

| | ESI MS | |
|---|---|---|
| Name | [M + H]⁺ | Starting material |
| tert-butyl (S)-2-(4-((4-((3- | 726.5 | (tert-butoxycarbonyl)- |
| (2,3-difluoro-4- | | L-proline |
| methoxyphenyl)imidazo[1,2- | | |
| a]pyrazin-8-yl)amino)-2- | | |
| ethylphenyl)sulfonyl)piperazine- | | |
| 1-carbonyl)pyrrolidine- | | |
| 1-carboxylate | | |
| tert-butyl (2S,4R)-2-(4-((4-((3- | 742.5 | (2S,4R)-1-(tert- |
| (2,3-difluoro-4- | | butoxycarbonyl)-4- |
| methoxyphenyl)imidazo[1,2- | | hydroxypyrrolidine-2- |
| a]pyrazin-8-yl)amino)-2- | | carboxylic acid |
| ethylphenyl)sulfonyl)piperazine- | | |
| 1-carbonyl)-4- | | |
| hydroxypyrrolidine- | | |
| 1-carboxylate | | |
| tert-butyl (R)-3-(4-((4-((3- | 726.5 | (R)-1-(tert- |
| (2,3-difluoro-4- | | butoxycarbonyl)pyrrolidine- |
| methoxyphenyl)imidazo[1,2- | | 3-carboxylic acid |
| a]pyrazin-8-yl)amino)-2- | | |
| ethylphenyl)sulfonyl)piperazine- | | |
| 1-carbonyl)pyrrolidine- | | |
| 1-carboxylate | | |
| tert-butyl 4-(4-((4-((3- | 740.6 | 1-(tert- |
| (2,3-difluoro-4- | | butoxycarbonyl)piperidine- |
| methoxyphenyl)imidazo[1,2- | | 4-carboxylic acid |
| a]pyrazin-8-yl)amino)-2- | | |
| ethylphenyl)sulfonyl)piperazine- | | |
| 1-carbonyl)piperidine- | | |
| 1-carboxylate | | |
| tert-butyl 4-(4-((4-((3- | 756.6 | 1-(tert-butoxycarbonyl)- |
| (2,3-difluoro-4- | | 4-hydroxypiperidine-4- |
| methoxyphenyl)imidazo[1,2- | | carboxylic acid |
| a]pyrazin-8-yl)amino)-2- | | |
| ethylphenyl)sulfonyl)piperazine- | | |
| 1-carbonyl)-4- | | |
| hydroxypiperidine- | | |
| 1-carboxylate | | |

### Example 105

### (S)(4-(4-((3-(2,3-difluoro-4-methoyphenyl)imidazo[1,2-a]pyrazin-8-yl)amino)-2-ethylphenyl)sulfonyl)piperazin-1-yl)(pyrrolidin-2-yl)methanone hydrochloride

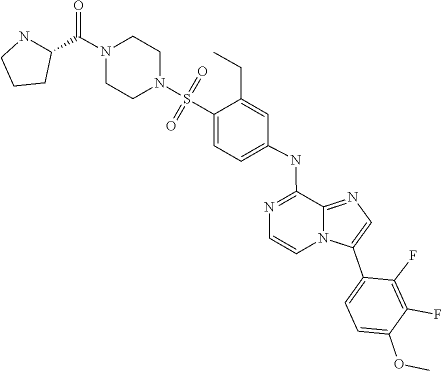

To a solution of tert-butyl (S)-2-(4-((4-((3-(2,3-difluoro-4-methoxyphenyl) imidazo[1,2-a]pyrazin-8-yl)amino)-2-ethylphenyl)sulfonyl)piperazine-1-carbonyl)pyrrolidine-1-carboxylate and (40.9 mg, Eq: 1) in CHCl2 (4 ml), HCl 4 M in Dioxan (141 ul, Eq: 10) was added and the mixture was stirred for 16 h at room temperature. The solvent was removed and the white crystals were dried under vacuum (<0.05 mbar, 3h, rt) to obtain the title compound (35.4 mg, 53.5 μmol, 94% yield) as a white solid ESI MS [M+H]⁺: 626.5

Four examples were obtained in analogy

| | | | ESI MS | Starting |
|---|---|---|---|---|
| Example | Name | Structure | [M + H]⁺ | material |
| 106 | (4-((4-((3- (2,3- difluoro-4- methoxyphenyl) imidazo [1,2- a]pyrazin-8- yl)amino)-2- ethylphenyl) sulfonyl) piperazin-1- yl)((2S,4R)- 4- hydroxypyrrolidin- 2-yl)methanone hydrochloride | 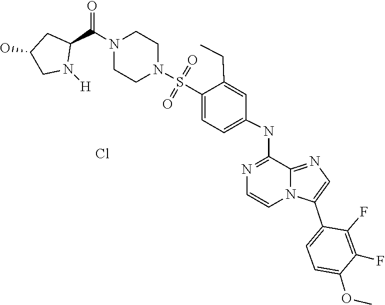 | 642.5 | tert-butyl (2S,4R)-2- (4-((4-((3- (2,3- difluoro-4- methoxyphenyl) imidazo [1,2- a]pyrazin-8- yl)amino)-2- ethylphenyl) sulfonyl) piperazine-1- carbonyl)-4- hydroxypyrrolidine- 1-carboxylate |
| 107 | (R)-(4-(4- ((3-(2,3- difluoro-4- methoxyphenyl) imidazo[1,2- a]pyrazin-8- yl)amino)-2- ethylphenyl) sulfonyl) piperazin-1- yl)(pyrrolidin-3- yl)methanone hydrochloride | 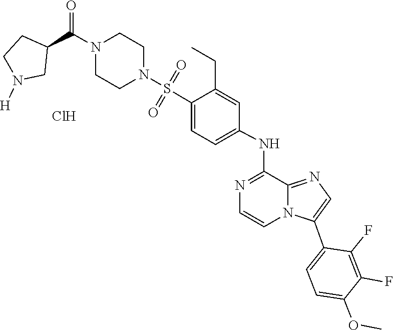 [SMILES: CCc1cc(Nc2nccn3c(-c4ccc(OC)c(F)c4F)cnc23)ccc1S(=O)(=O)N1CCN(C(=O)[C@@H]2CCNC2)CC1 ; Cl] | 626.4 | tert-butyl (R)-3-(4- ((4-((3-(2,3- difluoro-4- methoxyphenyl) imidazo[1,2- a]pyrazin-8- yl)amino)-2- ethylphenyl) sulfonyl) piperazine-1- carbonyl) pyrrolidine-1- carboxylate |
| 108 | (4-((4-((3- (2,3- difluoro-4- methoxyphenyl) imidazo[1,2- a]pyrazin-8- ylamino)-2- ethylphenyl) sulfonyl) piperazin-1- yl)(piperidin-4- yl)methanone hydrochloride | 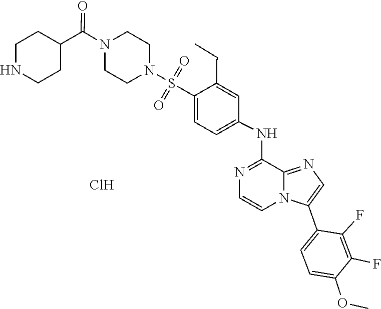 [SMILES: CCc1cc(Nc2nccn3c(-c4ccc(OC)c(F)c4F)cnc23)ccc1S(=O)(=O)N1CCN(C(=O)C2CCNCC2)CC1 ; Cl] | 640.5 | tert-butyl 4- (4-((4-((3- (2,3- difluoro-4- methoxyphenyl) imidazo [1,2- a]pyrazin-8- yl)amino)- 2- ethylphenyl) sulfonyl) piperazine-1- carbonyl) piperidine-1- carboxylate |
| 109 | (4-((4-((3- (2,3- difluoro-4- methoxyphenyl) imidazo [1,2- alpyrazin-8- yl)amino)-2- ethylphenyl) sulfonyl) piperazin-1-yl)(4- hydroxypiperidin- 4- yl)methanon bydrochloride | 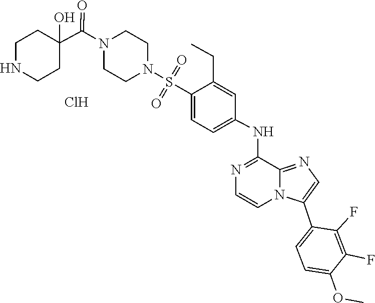 [SMILES: CCc1cc(Nc2nccn3c(-c4ccc(OC)c(F)c4F)cnc23)ccc1S(=O)(=O)N1CCN(C(=O)C2(O)CCNCC2)CC1 ; Cl] | 656.5 | tert-butyl 4- (4-((4-((3- (2,3- difluoro-4- methoxyphenyl) imidazo [1,2- a]pyrazin-8- yl)amino)- 2- ethylphenyl) sulfonyl) piperazine-1- carbonyl)-4- hydroxypiperidine- 1-carboxylate |

### Example 110

### 2-(4-((4-((3-(2,3-difluoro-4-methoxyphenyl)imidazo[1,2-a]pyrazin-8-yl)amino)-2-ethylphenyl)sulfonyl)piperazin-1-yl)-N,N,N-trimethyl-2-oxoethan-1-aminium iodide

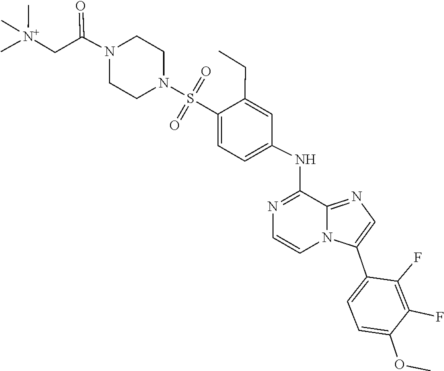 [SMILES: CCc1cc(Nc2nccn3c(-c4ccc(OC)c(F)c4F)cnc23)ccc1S(=O)(=O)N1CCN(C(=O)C[N+](C)(C)C)CC1]

To a solution of 1-(4-((4-((3-(2,3-difluoro-4-methoxyphenyl)imidazo[1,2-a]pyrazin-8-yl)amino)-2-ethylphenyl)sulfonyl)piperazin-1-yl)-2-(dimethylamino)ethan-1-one (24.5 mg, 39.9 μmol, Eq: 1) in dichloromethane (3 ml) iodomethane (0.1 M in CH₂Cl₂) (1.32 ml, 132 μmol, Eq: 3.3) was added and the clear colorless mixture was stirred over night at room temperature. The solvent was removed under reduced pressure, the residue was redissolved in 200 ul H₂O and some drops of CH₃CN and freeze-dried (<0.05 mbar, 5 h, 23° C.) to afford the title compound (19.2 mg, 25.4 μmol, 63.6% yield) as a white solid.

### tert-butyl (2S,4R)-2-((R)-3-(4-((4-((3-(2,3-difluoro-4-methoxyphenyl)imidazo[1,2-a]pyrazin-8-yl)amino)-2-ethylphenyl)sulfonyl)piperazine-1-carbonyl)pyrrolidine-1-carbonyl)-4-hydroxypyrrolidine-1-carboxylate

A solution of (2S,4R)-1-(tert-butoxycarbonyl)-4-hydroxypyrrolidine-2-carboxylic acid (7.63 mg, 33 μmol, Eq: 1.1) and TBTU (10.6 mg, 33 μmol, Eq: 1.1) in DMF (1.0 ml) was stirred for 15 min at room temperature. Then a solution of (R)-(4-((4-((3-(2,3-difluoro-4-methoxyphenyl)imidazo[1,2-a]pyrazin-8-yl)amino)-2-ethylphenyl)sulfonyl)piperazin-1-yl)(pyrrolidin-3-yl)methanone hydrochloride (17.4 mg, 26.3 μmol, Eq:1) in DMF (1 ml) and DIPEA (11.1 mg, 15 μl, 85.9 μmol, Eq: 2.9) were added and the mixture was stirred over night at room temperature. The mixture was directly purified by Prep. HPLC to afford the title compound (15.1 mg, 18 μmol, 68% yield) as a waxy solid ESI MS [M+H]+: 839.6

### Example 111

### (4-((4-((3-(2,3-difluoro-4-methoxyphenyl)imidazo[1,2-a]pyrazin-s-yl)amino)-2-ethylphenyl)sulfonyl)piperazin-1-yl)((R)-1-((2S,4R)-4-hydroxypyrrolidine-2-carbonyl)pyrrolidin-3-yl)methanone hydrochloride

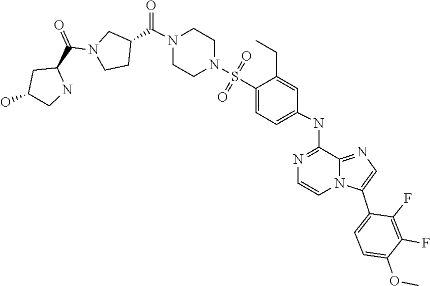

To a solution of tert-butyl (2S,4R)-2-((R)-3-(4-((4-((3-(2,3-difluoro-4-methoxyphenyl)imidazo[1,2-a]pyrazin-8-yl)amino)-2-ethylphenyl)sulfonyl) piperazine-1-carbonyl)pyrrolidine-1-carbonyl)-4-hydroxypyrrolidine-1-carboxylate (15.1 mg, 18 μmol, Eq: 1) HCl 4 M in Dioxan (68 ul, Eq: 15) was added and the mixture was stirred for 16 h at room temperature. The solvent was removed and the white crystals were dried under vacuum (<0.05 mbar, 3h, rt) to obtain the title compound (13.8 mg, 17.8 μmol, 99% yield) as a white solid ESI MS [M+H]⁺: 739.6

### Example 112

### Preparation of N-(4-(4-(aminomethyl)piperidin-1-yl)phenyl)-3-(2,3-difluoro-4-methoxyphenyl)imidazo[1,2-a]pyrazin-8-amine hydrochloride

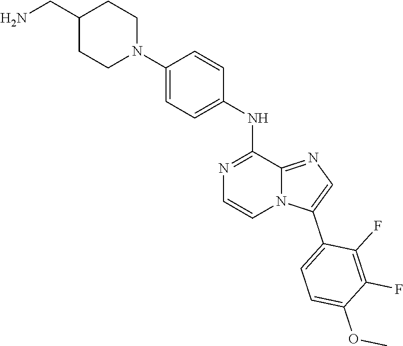 [SMILES: COc1ccc(-c2cnc3c(Nc4ccc(N5CCC(CN)CC5)cc4)nccn23)c(F)c1F]

### Step 1a

To a solution of tert-butyl (piperidin-4-ylmethyl)carbamate (1 g, 4.67 mmol, Eq: 1) in DMF (10 ml) at 80° C. K₂CO₃ (967 mg, 7 mmol, Eq: 1.5) was added. Then a solution of 2-chloro-1-fluoro-4-nitrobenzene (901 mg, 5.13 mmol, Eq: 1.1) in DMF (5 ml) was added. The mixture was stirred for 3 h at 80° C. The crude was transferred to a 250 ml round-bottomed flask and dropwise diluted with 100 ml water at 80° C. The yellow suspension was cooled to ambient temperature, filtered off and washed with water (3*10 ml. 0° C.). The yellow crystals were dried under vac. (<5 mbar, 50° C., 3h) to afford tert-butyl ((1-(2-chloro-4-nitrophenyl)piperidin-4-yl)methyl)carbamate (1.704 g, 4.61 mmol, 98.7% yield) ESI MS [M+H]⁺: 370.3

### Step 1b) 1-fluoro-4-nitro-2-vinylbenzene

A solution of 2-bromo-1-fluoro-4-nitrobenzene (3 g, 13.6 mmol, Eq: 1) and tetrakis(triphenylphosphine)palladium (0) (788 mg, 682 μmol, Eq: 0.05) in 1,2-dimethoxyethane (150 ml) was flushed with N2. K2CO3 (1.88 g, 13.6 mmol, Eq: 1), water (45 ml) and 2,4,6-trivinyl-1,3,5,2,4,6-trioxatriborinane compound with pyridine (1.65 g, 6.86 mmol, Eq: 0.503) were added at room temperature and the mixture was heated at 85° C. for 3.5 h. The mixture was evaporated. The crude product was absorbed with Isolute HM-N, dried and purified by flash chromatography on silica with heptane/ethylacetate (gradient) to afford the title compound (2.58 g, 13.4 mmol, 98.6% yield).

1H NMR (CHLOROFORM-d, 300 MHz) δ 8.41 (dd, 1H, J=2.8, 6.4 Hz), 8.13 (ddd, 1H, J=2.9, 4.4, 9.0 Hz), 7.19 (t, 1H, J=9.2 Hz), 6.87 (dd, 1H, J=11.3, 17.7 Hz), 5.99 (d, 1H, J=17.7 Hz), 5.58 (d, 1H, J=11.3 Hz)

Two intermediates were obtained in analogy

| | ESI MS | |
|---|---|---|
| Name | [M + H]⁺ | Starting material |
| tert-butyl ((1-(2-methyl- | 350.3 | 1-fluoro-2-methyl- |
| 4-nitrophenyl)piperidin- | | 4-nitrobenzene |
| 4-yl)methyl)carbamate | | |
| tert-butyl ((1-(4-nitro- | 362.3 | 1-fluoro-4-nitro- |
| 2-vinylphenyl)piperidin- | | 2-vinylbenzene |
| 4-yl)methyl)carbamate | | |

### Step 2a) tert-butyl ((1-(4-aminophenyl)piperidin-4-yl)methyl)carbamate

To a solution of tert-butyl ((1-(2-chloro-4-nitrophenyl)piperidin-4-yl)methyl)carbamate (1.7 g, 4.6 mmol, Eq: 1) in MeOH (40 ml), Pd/C (10%) (245 mg, 230 μmol, Eq: 0.05) was added under argon. The gas was changed to hydrogen and the mixture stirred for 2 hours at room temperature (MS showed the desired comp.)

The gas was changed to argon, then solution was filtered off (filter 5 um), the cat. rewashed with MeOH (3*15 ml). The clear solution was evaporated, the crude was absorbed with Isolute HM-N, dried and purified by flash chromatography over silica with heptane/ethylacetate (gradient) to afford the title compound (1.145 g, 81.6% yield) ESI MS [M+H]+: 306.3 as an off-white solid

One intermediate was obtained in analogy

| | ESI MS | |
|---|---|---|
| Name | [M + H]⁺ | Starting material |
| tert-butyl ((1-(4-amino- | 334.4 | tert-butyl ((1-(4-nitro- |
| 2-ethylphenyl)piperidin- | | 2-vinylphenyl)piperidin- |
| 4-yl)methyl)carbamate | | 4-yl)methyl)carbamate |

### Step 2b) tert-butyl ((1-(4-amino-2-chlorophenyl)piperidin-4-yl)methyl)carbamate

A solution of tert-butyl ((1-(2-chloro-4-nitrophenyl)piperidin-4-yl)methyl)carbamate (1.69 g, 4.57 mmol, Eq: 1) in ethanol (40 ml) and water (14 ml) was heated at 80° C. and supplemented with ammonium chloride (782 mg, 14.6 mmol, Eq: 3.2) and iron (3.16 g, 56.7 mmol, Eq: 12.4). After 3.5 h the hot reaction (about 60° C.) was filtered (over Dicalite) and the filter was washed with ethanol (3*30 ml). The filtrate was evaporated to dryness. The crude was absorbed with Isolute HM-N, dried and purified by flash chromatography over silica with heptane/ethylacetate (gradient) to afford the title compound (1.410 g, 4.15 mmol, 90.8% yield) ESI MS [M+H]+: 340.3 as a yellow waxy solid.

One intermediate was obtained in analogy

| | ESI MS | |
|---|---|---|
| Name | [M + H]⁺ | Starting material |
| tert-butyl ((1-(4-amino- | 320.3 | tert-butyl ((1-(2-methyl- |
| 2-methylphenyl)piperidin- | | 4-nitrophenyl)piperidin- |
| 4-yl)methyl)carbamate | | 4-yl)methyl)carbamate |

### tert-butyl ((1-(4-((3-(2,3-difluoro-4-methoxyphenyl)imidazo[1,2-a]pyrazin-8-yl)amino)phenyl)piperidin-4-yl)methyl)arbamate

A mixture of 8-chloro-3-(2,3-difluoro-4-methoxyphenyl)imidazo[1,2-a]pyrazine (80 mg, 271 μmol, Eq: 1) and tert-butyl ((1-(4-aminophenyl)piperidin-4-yl)methyl)carbamate (99.3, 325 μmol, Eq: 1.2) in acetonitrile (4 ml) and AcOH (400 μl) was shaked over night at 110° C. The solvents were removed t afford the crude product as a waxy solid ESI MS [M+H]+: 565.4 It was used directly for the next step.

Three intermediates were obtained in analogy

| | ESI MS | |
|---|---|---|
| Name | [M + H]⁺ | Starting material |
| tert-butyl ((1-(4-((3- | 579.5 | tert-butyl ((1-(4-amino- |
| (2,3-difluoro-4- | | 2-methylphenyl)piperidin- |
| methoxyphenyl)imidazo[1,2- | | 4-yl)methyl)carbamate |
| a]pyrazin-8-yl)amino)-2- | | |
| methylphenyl)piperidin-4- | | |
| yl)methyl)carbamate | | |
| tert-butyl ((1-(4-((3- | 593.5 | tert-butyl ((1-(4-amino- |
| (2,3-difluoro-4- | | 2-ethylphenyl)piperidin- |
| methoxyphenyl)imidazo[1,2- | | 4-yl)methyl)carbamate |
| a]pyrazin-8-yl)amino)-2- | | |
| ethylphenyl)piperidin-4- | | |
| yl)methyl)carbamate | | |
| tert-butyl ((1-(2-chloro-4-((3- | 599.4 | tert-butyl ((1-(4-amino- |
| (2,3-difluoro-4- | | 2-chlorophenyl)piperidin- |
| methoxyphenyl)imidazo[1,2- | | 4-yl)methyl)carbamate |
| a]pyrazin-8- | | |
| yl)amino)phenyl)piperidin- | | |
| 4-yl)methyl)carbamate | | |

To a solution of tert-butyl ((1-(4-((3-(2,3-difluoro-4-methoxyphenyl)imidazo[1,2-a]pyrazin-8-yl)amino)phenyl)piperidin-4-yl)methyl)carbamate (40 mg, 71 μmol, Eq: 1) in CH2Cl2 (3 ml) was added HCl (4 M in Dioxan) (266 ul, umol, Eq: 15). The mixture was stirred overnight at room temperature. Then the solvents were removed, 5 ml diethylether was added to the residue. The mixture was stirred for one hour, the crystals were filtered off, washed with ether and dried under vacuum to afford the title compound (33 mg, 65.9 μmol, 92.8% yield) as a white solid.

ESI MS [M+H]+: 465.4

Three examples were obtained in analogy

| | | | ESI MS | Starting |
|---|---|---|---|---|
| Example | Name | Structure | [M + H]⁺ | material |
| 113 | N-(4-(4- (aminomethyl) piperidin-1- yl)-3- methylphenyl)- 3-(2,3- difluoro-4- methoxyphenyl) imidazo [1,2- a]pyrazin-8- amine hydrochloride | 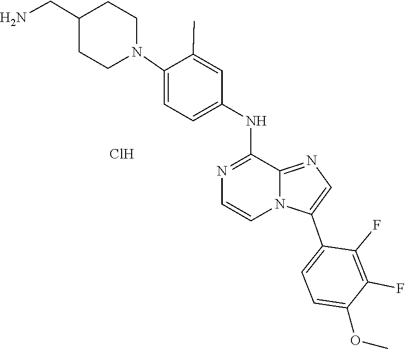 [SMILES: COc1ccc(-c2cnc3c(Nc4ccc(N5CCC(CN)CC5)c(C)c4)nccn23)c(F)c1F ; Cl] | 479.4 | tert-butyl ((4-((3- (2,3- difluoro-4- methoxyphenyl) imidazo [1,2- a]pyrazin-8- yl)amino)-2- methylphenyl) piperidin-4- yl)methyl) carbamate |
| 114 | N-(4-(4- (aminomethyl) piperidin- 1-yl)-3- ethylphenyl)- 3-(2,3- difluoro-4- methoxyphenyl) imidazo [1,2- a]pyrazin-8- amine hydrochloride | 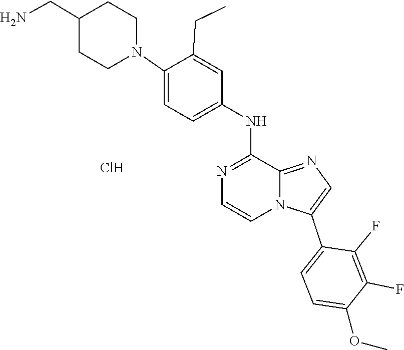 [SMILES: CCc1cc(Nc2nccn3c(-c4ccc(OC)c(F)c4F)cnc23)ccc1N1CCC(CN)CC1 ; Cl] | 493.4 | tert-butyl ((1-(4-((3- (2,3- difluoro-4- methoxyphenyl) imidazo [1,2- a]pyrazin-8- yl)amino)-2- ethylphenyl) piperidin-4- yl)methyl) carbamate |
| 115 | N-(4-(4- (aminometh yl)piperidin- 1-yl)-3- chlorophenyl)- 3-(2,3- difluoro-4- methoxyphenyl) imidazo [1,2- a]pyrazin-8- amine hydrochloride | 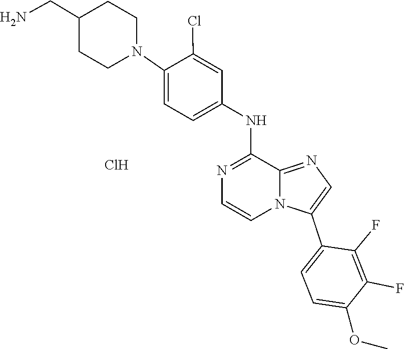 [SMILES: COc1ccc(-c2cnc3c(Nc4ccc(N5CCC(CN)CC5)c(Cl)c4)nccn23)c(F)c1F ; Cl] | 499.3 | tert-butyl (1-2- chloro-4- (3-(2,3- difluoro-4- methoxyphenyl) imidazo [1,2- a]pyrazin-8- yl)amino) phenyl) piperidin-4- yl)methyl) carbamate |

### Example 116

rac-N-(4-((((l r,4r)-4-aminocyclohexyl)methyl)sulfonyl)-3-chlorophenyl)-3-(2,3-difluoro-4-methoxyphenyl)imidazol[1,2-a]pyrazin-8-amine

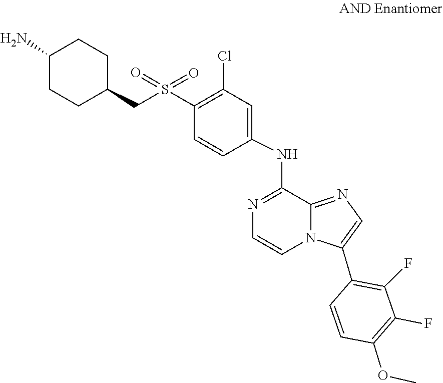 [SMILES: COc1ccc(-c2cnc3c(Nc4ccc(S(=O)(=O)C[C@H]5CC[C@H](N)CC5)c(Cl)c4)nccn23)c(F)c1F]

### Step 1) S-(2-chloro-4-nitrophenyl) benzothioate

To a mixture of 2-chloro-1-iodo-4-nitrobenzene (2.91 g, 10.3 mmol, Eq: 1), copper (I) iodide (196 mg, 1.03 mmol, Eq: 0.1), DIPEA (2.65 g, 3.59 ml, 20.5 mmol, Eq: 2) and 1,10-PHENANTHROLINE (370 mg, 2.05 mmol, Eq: 0.2) in Toluene (80 ml) under inert atmosphere and stirring at 60° C. was added a solution of benzothioic S-acid (1.45 g, 10.5 mmol, Eq: 1.02) in Toluene (20 ml). The dark mixture was stirred under reflux (bath=125° C.) for 1 h. (The mixture was absorbed with Isolute HM-N, dried and purified by flash chromatography over silica with heptane % ethyl acetate (gradient) to afford the title compound (2.54 g, 80.9% yield) as light yellow crystals. 1H NMR (300 MHz, CHLOROFORM-d) δ 8.42 (d, J=2.42 Hz, 1H), 8.19 (dd, J=2.42, 8.66 Hz, 1H), 8.00-8.07 (m, 2H), 7.85 (d, J=8.66 Hz, 1H), 7.62-7.72 (m, 1H), 7.49-7.58 (m, 2H)

### Step 2) Preparation of S-(4-amino-2-chlorophenyl) benzothioate

A solution of S-(2-chloro-4-nitrophenyl) benzothioate (2.54 g, 8.65 mmol, Eq: 1) in ethanol (60 ml) and water (20 ml) was heated at 80° C. and supplemented with ammonium chloride (1.5 g, 28 mmol, Eq: 3.24) and iron (6 g, 107 mmol, Eq: 12.4). After 20 min the hot reaction was filtered (over Dicalite) and the filter was washed with ethanol (3*30 ml). The filtrate was evaporated to dryness, resolved in ethyl acetate, absorbed with Isolute HM-N, dried and purified by flash chromatography over silica with heptane/ethylacetate (gradient) to afford the title compound (1.39 g, 60.9% yield) as light yellow crystals. ESI MS [M+H]+: 264.1

### Step 3) Preparation of S-(2-chloro-4-((3-(2,3-difluoro-4-methoxyphenyl)imidazo[1,2-a]pyrazin-8-yl)amino)phenyl) benzothioate

A mixture of S-(4-amino-2-chlorophenyl) benzothioate (357 mg, 1.35 mmol, Eq: 1) and 8-chloro-3-(2,3-difluoro-4-methoxyphenyl)imidazo[1,2-a]pyrazine (400 mg, 1.35 mmol, Eq: 1) in acetonitrile (20 ml) under Argon (sealed tube) was stirred for 15 h at 115° C. and for 3h at 130° C. The cold suspension was filtered (over Satorius 0.45 um), the solids were washed with CH3CN (3*0.5 ml) and dried under vacuum to afford the title compound (570.9 mg, 1.09 mmol, 80.7% yield) as a light grey solid. ESI MS [M+H]+: 523.1

### Step 4) Step 4) rac-tert-butyl ((1r,4r)-4-(((2-chloro-4-((3-(2,3-difluoro-4-methoxyphenyl)imidazo[1,2-a]pyrazin-8-yl)amino)phenyl)thio)methyl)cyclohexyl)carbamate

A suspension of S-(2-chloro-4-((3-(2,3-difluoro-4-methoxyphenyl)imidazo[1,2-a]pyrazin-8-yl)amino)phenyl) benzothioate (200 mg, 382 μmol, Eq: 1) in THF (6 ml) and methanol (6 ml) and Cs2CO3 (125 mg, 382 μmol, Eq: 1) was stirred for 1 h at room temperature. One third of this solution (contains 2-chloro-4-((3-(2,3-difluoro-4-methoxyphenyl)imidazo[1,2-a]pyrazin-8-yl)amino)benzenethiol (53.2 mg, 127 μmol, Eq: 1)) was transferred to a 10 ml round-bottomed flask, diluted with 2 ml MeOH and tert-butyl N-[4-(bromomethyl)cyclohexyl]carbamate (55.7 mg, 191 μmol, Eq: 1.5) and Cs2CO3 (41.4 mg, 127 μmol, Eq: 1) was added. The mixture was stirred for 16 h at room temperature. The crude was absorbed with Isolute HM-N, dried and purified by flash chromatography over silica with heptane/ethylacetate (gradient) to afford the title compound (45.4 mg, 56.7% yield) as a white solid. ESI MS [M+H]+: 630.3

### Step 5) rac-tert-butyl ((1r,4r)-4-(((2-chloro-4-((3-(2,3-difluoro-4-methoxyphenyl)imidazo[1,2-a]pyrazin-8-yl)amino)phenyl)sulfonyl)methyl)cyclohexyl)carbamate

A solution of rac-tert-butyl ((1r,4r)-4-(((2-chloro-4-((3-(2,3-difluoro-4-methoxyphenyl)imidazo[1,2-a]pyrazin-8-yl)amino)phenyl)thio)methyl) cyclohexyl)carbamate (20.2 mg, 32.1 μmol, Eq: 1) and mCPBA (22.1 mg, 83.3 μmol, Eq: 2.6) in Dichloromethane (3.44 ml) was stirred over night at room temperature. Then 2 ml of Na2SO2O3 (10% aq) was added. The mixture is stirred for 30 min, separated, the organic layer was extracted once with 3 ml Na2CO3 10% aq and once with water (3 ml), dried over MgSO4 filtered off and evaporated ot obtain the title compound (21.2 mg, 95% yield) as a light yellow solid. ESI MS [M+H]+: 662.3

### Step 6) rac-N-(4-((((1r,4r)-4-aminocyclohexyl)methyl)sulfonyl)-3-chlorophenyl)-3-(2,3-difluoro-4-methoxyphenyl)imidazo[1,2-a]pyrazin-8-amine

To a solution of rac-tert-butyl ((1r,4r)-4-(((2-chloro-4-((3-(2,3-difluoro-4-methoxyphenyl)imidazo[1,2-a]pyrazin-8-yl)amino)phenyl)sulfonyl) methyl)cyclohexyl)carbamate (21.2 mg, 32 μmol, Eq: 1) in Dichloromethane (3 ml) at 0° C. was added TFA (54.7 mg, 37 μl, 480 μmol, Eq: 15) and the mixture was stirred for 16 h at room temperature. Then the reaction was extracted with CH2Cl2 (2*15 ml) and Na2CO3 (10% aq, 2*15 ml). The organic layer was dried over MgSO4, filtered off and evaporated to afford the title compound (12.5 mg, 65% yield) as a yellow solid. ESI MS [M+H]+: 562.3

### Example 117

### Preparation of N-[4-(imidazol-1-ylmethyl)phenyl]-3-(4-methoxyphenyl)imidazo[1,2-a]pyrazin-8-amine

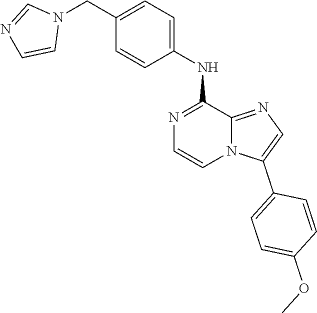

### Step 1) 1-[(2-methyl-4-nitro-phenyl)methyl]imidazole

To a mixture of imidazole (1.32 g, 19.4 mmol, 3 eq) in DMSO (15 mL) was added sodium hydrogen carbonate (1.63 g, 19.4 mmol, 3 eq) and 1-(chloromethyl)-2-methyl-4-nitro-benzene (1.2 g, 6.47 mmol, 1 eq) at 25° C. Then the mixture was stirred at 25° C. for 3 h. The mixture was poured into water (50.0 mL) and extracted with EtOAc (100.0 mL×3). The organic phase was dried and concentrated in vacuo to give the crude product as yellow oil. The crude product was purified by column (PE:EtOAC=5:1˜1:2, Rf=0.4) to give the title compound (1.2 g, 5.52 mmol, 85.45% yield) as a yellow oil ESI MS [M+H]+: 218.1

### Step 2) 4-(imidazol-1-ylmethyl)-3-methyl-aniline

To a mixture of 1-[(2-methyl-4-nitro-phenyl)methyl]imidazole (1.0 g, 4.6 mmol, 1 eq)₁-[(2-methyl-4-nitro-phenyl)methyl]imidazole (1.0 g, 4.6 mmol, 1 eq) in 2-propanol (10 mL) was added iron (1.29 g, 23.02 mmol, 5 eq) and ammonium chloride (369.37 mg, 6.91 mmol, 1.5 eq) at 25° C. Then the mixture was stirred at 80° C. for 1 h. The mixture was then filtered and concentrated in vacuo to give 4-(imidazol-1-ylmethyl)-3-methyl-aniline (800 mg, 4.27 mmol, 92.81% yield) as a yellow oil.

### Step 3) N-[4-(imidazol-1-ylmethyl)phenyl]-3-(4-methoxyphenyl)imidazo[1,2-a]pyrazin-8-amine

8-chloro-3-(4-methoxyphenyl)imidazo[1,2-a]pyrazine (30 mg, 116 μmol, Eq: 1) was combined with t-BuOH (750 μl) to give a brown solution.

4-((1H-imidazol-1-yl)methyl)aniline (30 mg, 173 μmol, Eq: 1.50), cesium carbonate (75.3 mg, 231 μmol, Eq: 2.00), t-Bu-Xphos (2.45 mg, 5.78 μmol, Eq: 0.05) and Pd2(dba)3 (1.06 mg, 1.16 μmol, Eq: 0.01) were added and the reaction mixture was stirred at 100° C. overnight. After cooling down to room temperature, 200 mg SiliaMetS Thiourea was added, then t-BuOH removed. 1 ml DMSO was added to the dry reaction mixture, and after stirring for 20 min it was filtered over dicalite.

The reaction was purified by prep HPLC to yield the title compound (16 mg, 0.116 mmol, 34.6% yield) as an off-white solid ESI MS [M+H]+: 397.1

### Example 118

### Preparation of 3-(2,3-difluoro-4-methoxy-phenyl)-N-[4-(imidazol-1-ylmethyl)-3-methyl-phenyl]imidazo[1,2-a]pyrazin-8-amine

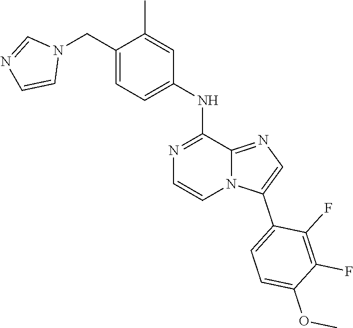 [SMILES: COc1ccc(-c2cnc3c(Nc4ccc(Cn5ccnc5)c(C)c4)nccn23)c(F)c1F]

A mixture of 4-(imidazol-1-ylmethyl)-3-methyl-aniline (69.66 mg, 0.370 mmol, 1.1 eq) and 8-chloro-3-(2,3-difluoro-4-methoxy-phenyl)imidazo[1,2-a]pyrazine (100.0 mg, 0.340 mmol, 1 eq) in toluene (1 mL) was added trifluoroacetic acid (0.1 mL, 1.3 mmol, 3.84 eq) at 25° C. and the mixture was stirred at 100° C. for 16 h.

The mixture was then concentrated in vacuo to remove the solvent. The crude product was purified by Prep-HPLC to yield the title compound (10 mg, 0.020 mmol, 6.62% yield) as a yellow solid ESI MS [M+H]+: 447.0

One example was obtained in analogy

| | | | ESI MS | Starting |
|---|---|---|---|---|
| Example | Name | Structure | [M + H]⁺ | material |
| 119 | N-[4- (imidazol-1- ylmethyl)-3- methyl- phenyl]-3- (4- methoxyphenyl) imidazo[1,2- a]pyrazin-8- amine | 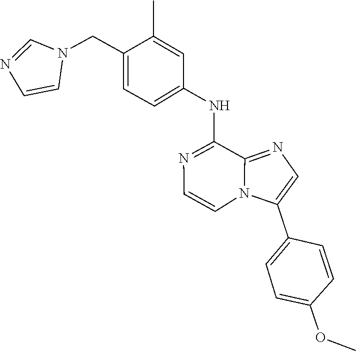 [SMILES: COc1ccc(-c2cnc3c(Nc4ccc(Cn5ccnc5)c(C)c4)nccn23)cc1] | 411.0 | 8-chloro- 3-(4- methoxyphenyl) imidazo[1,2- a]pyrazine |

### Example 120

### (4-((3-(4-methoxyphenyl)imidazo[1,2-a]pyrazin-4-yl)amino)-2-methylphenyl)methanol

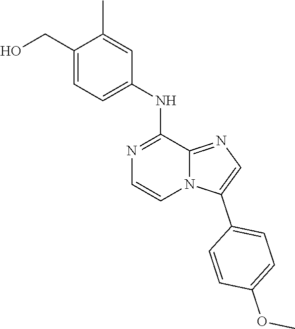 [SMILES: COc1ccc(-c2cnc3c(Nc4ccc(CO)c(C)c4)nccn23)cc1]

### Step 1) Methyl 4-((3-(4-methoxyphenyl)imidazo[1,2-a]pyrazin-8-yl)amino)-2-methylbenzoate

8-chloro-3-(4-methoxyphenyl)imidazo[1,2-a]pyrazine (50 mg, 193 μmol, Eq: 1) and methyl 4-amino-2-methylbenzoate (47.7 mg, 289 μmol, Eq: 1.50) were combined with acetonitrile (0.9 ml) and AcOH (0.1 ml). The reaction mixture was stirred at 80° C. for 4h 30. After cooling down to room temperature, acetonitrile was added to the suspension and the solid was filtered and dried under HV to afford the title compound (66.3 mg, 88.7% yield) as an off-white solid ESI MS [M+H]+: 389.2

### Step 2) (4-((3-(4-methoxyphenyl)imidazo[1,2-a]pyrazin-8-yl)amino)-2-methylphenyl)methanol

Methyl 4-((3-(4-methoxyphenyl)imidazo[1,2-a]pyrazin-8-yl)amino)-2-methylbenzoate (66 mg, 170 μmol, Eq: 1) was combined with THF (1.5 ml). After cooling down to 0° C., LiAlH₄ (12.9 mg, 340 μmol, Eq: 2.0) was added portionwise and the stirring was continued at 0° C. for 3h 30. The reaction was quenched at 0° C. by addition of a 10% NaHCO₃ solution and water. After stirring for 30 min until room temperature, an extraction was done with ethyl acetate. The crude obtained was purified by flash chromatography to afford the title compound (43.9 mg, 62.2% yield) as an off-white solid ESI MS [M+H]+: 361.2

One example was obtained in analogy

| | | | ESI MS | Starting |
|---|---|---|---|---|
| Example | Name | Structure | [M + H]⁺ | material |
| 121 | (4-((3-(4- (difluoromethoxy) phenyl) imidazo[1,2-a] pyrazin- 8-yl)amino)-2- methylphenyl) methanol | 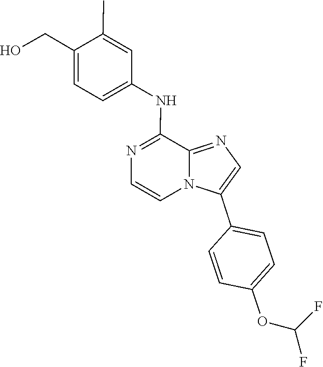 [SMILES: Cc1cc(Nc2nccn3c(-c4ccc(OC(F)F)cc4)cnc23)ccc1CO] | 425.1 | 8-chloro- 3-(4- (difluoromethoxy) phenyl)imidazo [1,2- a]pyrazine |

### Example 122

### N-(4-(2-(2-(2-aminoethoxy)ethoxy)ethoxy)-3-methylphenyl)-3-(2,3-difluoro-4-(pyridin-2-yloxy)phenyl)imidazo[1,2-a]pyrazin-8-amine

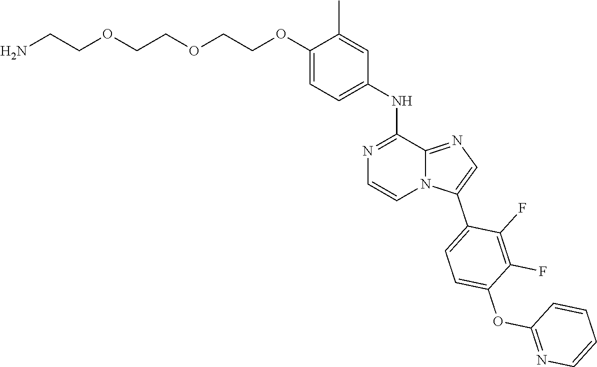 [SMILES: Cc1cc(Nc2nccn3c(-c4ccc(Oc5ccccn5)c(F)c4F)cnc23)ccc1OCCOCCOCCN]

### Step 1) 2-(2,3-difluoro-4-(4,4,5,5-tetramethyl-1,3,2-dioxaborolan-2-yl)phenoxy)pyridine and (2,3-difluoro-4-(pyridin-2-yloxy)phenyl)boronic acid

In dioxane (50 ml), a mixture of 2-(4-bromo-2,3-difluorophenoxy)pyridine (3.3 g, 10.8 mmol, Eq: 1), bis(pinacolato)diboron (3.03 g, 11.9 mmol, Eq: 1.1) and potassium acetate (2.34 g, 23.9 mmol, Eq: 2.2) was evacuated and back filled with argon. PdCl₂(DPPF)-CH₂Cl₂ adduct (793 mg, 1.08 mmol, Eq: 0.1) was added and the resulting mixture was stirred at 80° C. for 16 h and then at 100° C. for 20 h. Additional potassium acetate (1.06 g, 10.8 mmol, Eq: 1), bis(pinacolato)diboron (2.75 g, 10.8 mmol, Eq: 1) and PdCl₂(DPPF)-CH₂Cl₂ adduct (397 mg, 542 μmol, Eq: 0.05) were added. The mixture was stirred at 100° C. for 2 days. It was then filtered and concentrated to obtain a mixture of the title compounds (6.5 g, crude) as a black waxy solid which was used as is without further purification.

### Step 2) 8-chloro-3-(2,3-difluoro-4-(pyridin-2-yloxy)phenyl)imidazo[1,2-a]pyrazine

A mixture of 2-(2,3-difluoro-4-(4,4,5,5-tetramethyl-1,3,2-dioxaborolan-2-yl)phenoxy)pyridine (4.47 g, 13.4 mmol, Eq: 2.5) and (2,3-difluoro-4-(pyridin-2-yloxy)phenyl)boronic acid (3.37 g, 13.4 mmol, Eq: 2.5) and 8-chloro-3-iodoimidazo[1,2-a]pyrazine (1.5 g, 5.37 mmol, Eq: 1) were dissolved in Dioxane (50 ml) and Water (5 ml) and purged with Ar. PdCl₂(DPPF)-CH₂Cl₂ adduct (393 mg, 537 μmol, Eq: 0.1) was added and the reaction mixture was purged again. Sodium carbonate (1.71 g, 16.1 mmol, Eq: 3) was added and the reaction was heated at 90° C. overnight. The mixture was diluted with EtOAc and extracted with water. The organic layer was concentrated in vacuo onto silica gel and purified by flash chromatography to afford the title compound (860 mg, 44.7% yield) as a light yellow solid ESI MS [M+H]+: 359.2

### Step 3) 4-((3-(2,3-difluoro-4-(pyridin-2-yloxy)phenyl)imidazo[1,2-a]pyrazin-8-yl)amino)-2-methylphenol

8-chloro-3-(2,3-difluoro-4-(pyridin-2-yloxy)phenyl)imidazo[1,2-a]pyrazine (345 mg, 962 μmol, Eq: 1) was dissolved in Acetonitrile (8 ml) and 4-amino-2-methylphenol (237 mg, 1.92 mmol, Eq: 2) and acetic acid (57.8 mg, 55.1 μl, 962 μmol, Eq: 1) were added. The reaction mixture was stirred at 120° C. for 30 min in microwave reactor. A dark brown crystalline solid had formed and was isolated by filtration. It was diluted with MeOH, absorbed onto silica gel, concentrated and purified by flash chromatography to afford the title compound (184 mg, 43% yield) as a dark brown solid ESI MS [M+H]+: 446.3

### Step 4) tert-butyl (2-(2-(2-(4-((3-(2,3-difluoro-4-(pyridin-2-yloxy)phenyl)imidazo[1,2-a]pyrazin-8-yl)amino)-2-methylphenoxy)ethoxy)ethoxy)ethyl)carbamate

4-((3-(2,3-difluoro-4-(pyridin-2-yloxy)phenyl)imidazo[1,2-a]pyrazin-8-yl)amino)-2-methylphenol (20 mg, 41.3 μmol, Eq: 1), tert-butyl (2-(2-(2-bromoethoxy)ethoxy)ethyl)carbamate (15.5 mg, 49.6 μmol, Eq: 1.2) and cesium carbonate (20 mg, 62 μmol, Eq: 1.5) were dissolved in Acetone (2 ml) and the mixture was stirred at 60° C. overnight. The crude was worked up with ethyl acetate and water and then purified by flash chromatography (heptane in EtOAc) to afford the title compound (15 mg, 22.2 μmol, 53.7% yield) ESI MS [M+H]+: 677.5

### Step 5) N-(4-(2-(2-(2-aminoethoxy)ethoxy)ethoxy)-3-methylphenyl)-3-(2,3-difluoro-4-(pyridin-2-yloxy)phenyl)imidazo[1,2-a]pyrazin-8-amine

tert-butyl (2-(2-(2-(4-((3-(2,3-difluoro-4-(pyridin-2-yloxy)phenyl)imidazo[1,2-a]pyrazin-8-yl)amino)-2-methylphenoxy)ethoxy)ethoxy)ethyl)carbamate (15 mg, 22.2 μmol, Eq: 1) was dissolved in MeOH (1 ml) and 4 M HCl in dioxane (111 μl, 443 μmol, Eq: 20) was added. The reaction mixture was stirred at room temperature for 2 h. The mixture was concentrated in vacuo and adsorbed onto silica and was purified by flash chromatography (silica gel, 4 g, 0% to 100⁰/75:25:2 EtOAc/EtOH/NH₄OH in heptane) to afford the title compound (9.5 mg, 69.9% yield) ESI MS [M+H]+: 577.3

### Example 123

### [4-[[3-(2,3-difluoro-4-methoxy-phenyl)imidazo[1,2-a]pyrazin-8-yl]amino]-2-ethyl-phenyl]methyl-trimethyl-ammonium

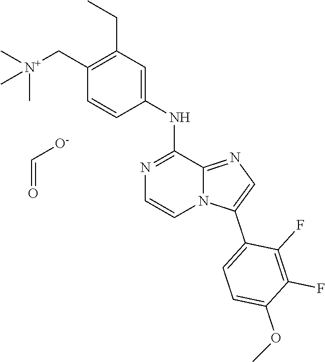

### Step 1) [4-[[3-(2,3-difluoro-4-methoxy-phenyl)imidazo[1,2-a]pyrazin-8-yl]amino]-2-ethyl-phenyl]methanol

4-((3-(2,3-difluoro-4-methoxyphenyl)imidazo[1,2-a]pyrazin-8-yl)amino)-2-ethylbenzoic acid (200 mg, 471 μmol, Eq: 1) was suspended in THF (942 μl). Borane THF complex (2.36 ml, 2.36 mmol, Eq: 5) was added dropwise. The mixture was stirred at rt for 18h and then quenched with 1 mL MeOH. The solvent was removed in vacuo and the residue was purified by flash chromatography to afford the title compound (74 mg, 0.180 mmol, 38.3%) as a light yellow powder ESI MS [M+H]+: 411.9

### Step 2) 4-[[3-(2,3-difluoro-4-methoxy-phenyl)imidazo[1,2-a]pyrazin-8-yl]amino]-2-ethyl-benzaldehyde

To a solution of (4-((3-(2,3-difluoro-4-methoxyphenyl)imidazo[1,2-a]pyrazin-8-yl)amino)-2-ethylphenyl)methanol (74 mg, 180 μmol, Eq: 1) in dry CH2Cl2 (3.61 ml) was added manganese (IV) oxide (313 mg, 3.61 mmol, Eq: 20). The mixture was stirred at rt for 1h. The mixture was filtered through a pad of celite and the cake was washed with DCM. The filtrate was concentrated in vacuo to give the crude title product (71 mg, 0.180, mmol) as a light yellow power. It was used without purification ESI MS [M+H]+: 409.6

### Step 3) [4-[[3-(2,3-difluoro-4-methoxy-phenyl)imidazo[1,2-a]pyrazin-8-yl]amino]-2-ethyl-phenyl]methyl-trimethyl-ammonium

4-((3-(2,3-difluoro-4-methoxyphenyl)imidazo[1,2-a]pyrazin-8-yl)amino)-2-ethylbenzaldehyde (70 mg, 171 μmol, Eq: 1) and dimethylamine (857 μl, 1.71 mmol, Eq: 10) was dissolved in dry MeOH (3.43 ml), the mixture was stirred at room temperature for 18h to get a clear solution. NaBH₃CN (53.9 mg, 857 μmol, Eq: 5) was then added. The mixture was stirred at rt for 24h. LC-MS indicated a full conversion. The solvent was removed in vacuo, and the residue was purified by flash chromatography. The product was dissolved in 4 mL MeCN. DIPEA (0.05 mL) and iodomethane (122 mg, 53.4 μl, 857 μmol, Eq: 5) were added. The solution was stirred at room temperature overnight. The solvent was removed in vacuo and the residue was purified by preparative HPLC to afford the title compound as a white gum ESI MS [M+]+: 452.6

Assay Procedures

Antimicrobial Susceptibility Testing:

90% Growth Inhibitory Concentration (IC90) Determination

The in vitro antimicrobial activity of the compounds was determined according to the following procedure:

The assay used a 10-points Iso-Sensitest broth medium to measure quantitatively the in vitro activity of the compounds against Acinetobacter baumannii ATCC17961 or ATCC17978.

Stock compounds in DMSO were serially twofold diluted (e.g. range from 50 to 0.097 μM final concentration) in 384 wells microtiter plates and inoculated with 49 μl the bacterial suspension in Iso-Sensitest medium to have a final cell concentration of ˜5×10⁽⁵⁾ CFU/ml in a final volume/well of 50 ul/well. Microtiter plates were incubated at 35±2° C.

Bacterial cell growth was determined with the measurement of optical density at λ=600 nm each 20 minutes over a time course of 16h. Growth inhibition was calculated during the logarithmic growth of the bacterial cells with determination of the concentration inhibiting 50% (IC50) and 90% (IC90) of the growth.

Table 1 provides the 90% growth inhibitory concentrations (IC90) in micromoles per liter of the compounds of present invention obtained against the strain Acinetobacter baumannii ATCC17978.

Table 2 provides the 90% growth inhibitory concentrations (IC90) in micromoles per liter of the compounds of present invention obtained against the strain Acinetobacter baumannii ATCC17961.

Particular compounds of the present invention exhibit an IC90 (Acinetobacter baumannii ATCC17961 or ATCC 17978)≤25 μmol/l.

More particular compounds of the present invention exhibit an IC90 (Acinetobacter baumannii ATCC 17961 or ATCC 17978)≤5 μmol/l.

Most particular compounds of the present invention exhibit an IC90 (Acinetobacter baumannii ATCC 17961 or ATCC 17978)≤1 μmol/l.

TABLE 1

| | AB ATCC |
|---|---|
| | 17978 |
| Example | IC90 (uM) |
| 1 | 0.33 |
| 2 | 0.25 |
| 4 | 0.33 |
| 5 | 0.88 |
| 7 | 0.86 |
| 8 | 0.82 |
| 9 | 0.34 |
| 10 | 0.82 |
| 11 | 0.087 |
| 12 | 0.89 |
| 13 | 0.81 |
| 14 | 0.64 |
| 16 | 0.4 |
| 17 | 0.43 |
| 18 | 0.33 |
| 19 | 0.77 |
| 20 | 0.11 |
| 34 | 0.55 |
| 35 | 0.15 |
| 36 | 0.84 |
| 38 | 0.2 |
| 40 | 0.13 |
| 41 | 0.52 |
| 42 | 0.83 |
| 43 | 0.12 |
| 44 | 0.26 |
| 45 | 0.16 |
| 46 | 0.89 |
| 47 | 0.13 |
| 49 | 0.3 |
| 50 | 0.63 |
| 51 | 0.75 |
| 52 | 0.98 |
| 53 | 0.36 |
| 54 | 0.72 |
| 55 | 0.28 |
| 56 | 0.25 |
| 57 | 0.14 |
| 58 | 0.26 |
| 59 | 0.25 |
| 60 | 0.76 |
| 61 | 0.73 |
| 65 | 0.75 |
| 75 | 0.28 |
| 76 | 0.73 |
| 77 | 0.63 |
| 78 | 0.58 |
| 79 | 0.85 |
| 80 | 0.76 |
| 81 | 0.63 |
| 82 | 0.89 |
| 83 | 0.7 |
| 84 | 0.17 |
| 85 | 0.34 |
| 86 | 0.26 |
| 87 | 0.26 |
| 88 | 0.33 |
| 89 | 0.28 |
| 90 | 0.24 |
| 91 | 0.81 |
| 92 | 0.7 |
| 93 | 0.18 |
| 95 | 0.38 |
| 117 | 0.54 |
| 119 | 0.37 |
| 120 | 0.74 |
| 121 | 0.67 |

TABLE 2

| | AB ATCC |
|---|---|
| | 17961 |
| Example | IC90 (uM) |
| 3 | 0.72 |
| 6 | 0.3 |
| 15 | 0.6 |
| 21 | 0.26 |
| 22 | 0.084 |
| 23 | 0.11 |
| 24 | 0.17 |
| 25 | 0.048 |
| 26 | 0.14 |
| 27 | 0.086 |
| 28 | 0.048 |
| 29 | 0.18 |
| 30 | 0.13 |
| 31 | 0.5 |
| 32 | 0.17 |
| 33 | 0.49 |
| 37 | 0.21 |
| 39 | 0.1 |
| 48 | 0.076 |
| 62 | 0.094 |
| 63 | 0.036 |
| 64 | 0.12 |
| 66 | 0.16 |
| 67 | 0.12 |
| 68 | 0.43 |
| 69 | 0.2 |
| 70 | 0.19 |
| 71 | 0.086 |
| 72 | 0.12 |
| 73 | 0.33 |
| 74 | 0.15 |
| 94 | 0.12 |
| 96 | 0.2 |
| 97 | 0.18 |
| 98 | 0.29 |
| 99 | 0.28 |
| 100 | 0.12 |
| 101 | 0.078 |
| 102 | 0.39 |
| 103 | 0.5 |
| 104 | 0.34 |
| 105 | 0.31 |
| 106 | 0.25 |
| 107 | 0.2 |
| 108 | 0.22 |
| 109 | 0.64 |
| 110 | 0.33 |
| 111 | 0.87 |
| 112 | 0.56 |
| 113 | 0.37 |
| 114 | 0.81 |
| 115 | 0.49 |
| 116 | 0.22 |
| 118 | 0.12 |
| 122 | 0.65 |
| 123 | 0.58 |
| 124 | 0.27 |
| 125 | 0.36 |

### Example 126

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 127

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Example 128

A compound of formula (I) can be used in a manner known per se as the active ingredient for the production of an infusion solution of the following composition:

| | |
|---|---|
| Active ingredient | 100 mg |
| Lactic acid 90% | 100 mg |
| NaOH q.s. or HCl q.s. | for adjustment to pH 4.0 |
| Sodium chloride q.s. or glucose q.s. | for adjustment of the |
| | osmolality to 290 mOsm/kg |
| Water for injection (WFI) | ad 100 ml |

### Example 129

A compound of formula (I) can be used in a manner known per se as the active ingredient for the production of an infusion solution of the following composition:

| | |
|---|---|
| Active ingredient | 100 mg |
| Hydroxypropyl-beta-cyclodextrin | 10 g |
| NaOH q.s. or HCl q.s. | for adjustment to pH 7.4 |
| Sodium chloride q.s. or glucose q.s. | for adjustment of the |
| | osmolality to 290 mOsm/kg |
| Water for injection (WFI) | ad 100 ml |

1. A compound of formula (I): 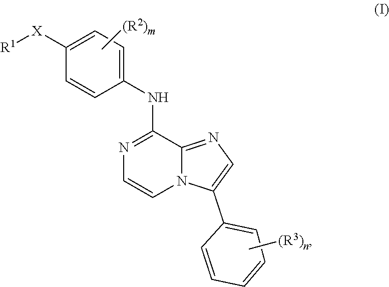
or a pharmaceutically acceptable salt thereof, wherein:
X is selected from a covalent bond, carbonyl, —(CH₂)ₚ—C(O)—NR⁴—, —NR⁴—C(O)—(CH₂)ₛ—, —(CH₂)_{q}SO₂—, —SO₂(CH₂)_{q}—, —NR⁵—S(O)₂—, —S(O)₂—NR⁵—, a group 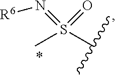
and a group 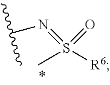
wherein
the asterisk indicates the point of attachment of R¹ to X; and
the wavy line indicates the point of attachment of X to the remainder of formula (I);
R¹ is selected from hydrogen, halogen, cyano, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, halo-C₁-C₆-alkyl, cyano-C₁-C₆-alkyl, hydroxy-C₁-C₆-alkyl, amino-C₁-C₆-alkyl, C₁-C₆-alkyl-NH—C₁-C₆-alkyl, (C₁-C₆-alkyl)₂N—C₁-C₆-alkyl, (C₁-C₆-alkyl)₃N⁺—C₁-C₆-alkyl, C₁-C₆-alkyl-NH—, (C₁-C₆-alkyl)₂N—, C₁-C₆-alkoxy, halo-C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)—NH—C₁-C₆-alkoxy, C₁-C₆-alkyl-NH—C(O)—, and a group 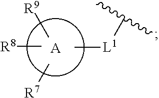
and
R² is, at each occurrence, independently selected from hydrogen, halogen, C₁-C₆-alkyl, hydroxy-C₁-C₆-alkyl and C₁-C₆-alkyl-NH—C(O)—; or
R¹ and one occurrence of R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with 1-2 substituents independently selected from halogen, cyano, hydroxy, amino, C₁-C₆-alkyl, C₁-C₆-alkoxy, halo-C₁-C₆-alkyl, halo-C₁-C₆-alkoxy and amino-C₁-C₆-alkyl-NH—;
R³ is, at each occurrence, independently selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy;
R⁴ is selected from hydrogen and C₁-C₆-alkyl;
R⁵ is selected from hydrogen and C₁-C₆-alkyl; or
R⁶ is selected from C₁-C₆-alkyl, amino-C₁-C₆-alkyl, (3- to 14-membered heterocyclyl)-C(O)—NH—C₁-C₆-alkyl; wherein said 3- to 14-membered heterocyclyl is optionally substituted with 1-2 hydroxy substituents;
R⁷, R⁸, and R⁹ are each independently selected from hydrogen, halogen, cyano, hydroxy, amino, C₁-C₆-alkyl, amino-C₁-C₆-alkyl, halo-C₁-C₆-alkyl, cyano-C₁-C₆-alkyl, hydroxy-C₁-C₆-alkyl, C₁-C₆-alkoxy, halo-C₁-C₆-alkoxy, C₁-C₆-alkyl-NH—C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl and oxo; wherein said 3- to 14-membered heterocyclyl is optionally substituted with 1-3 substituents selected from hydroxy, amino, halogen, cyano, C₁-C₆-alkyl, halo-C₁-C₆-alkyl, C₁-C₆-alkoxy, halo-C₁-C₆-alkoxy, and hydroxy-(3- to 14-membered heterocyclyl)-C(O)—;
A is selected from 3- to 14-membered heterocyclyl, 5- to 14-membered heteroaryl, C₆-C₁₄-aryl, and C₃-C₁₀-cycloalkyl;
L¹ is a covalent bond or C₁-C₆-alkyl;
m is an integer selected from 1, 2, 3 and 4;
n is an integer selected from 1, 2, 3, 4 and 5;
p is an integer selected from 0 and 1;
q is an integer selected from 0 and 1; and
r and s are both an integer selected from 1, 2, 3 and 4;
provided that when X is carbonyl, R¹ is not C₁-C₆-alkyl-NH— or (C₁-C₆-alkyl)₂N—.

2. The compound of formula (I) according to claim 1, or a pharmaceutically acceptable salt thereof, wherein the compound of formula (I) is a compound of formula (I-I): 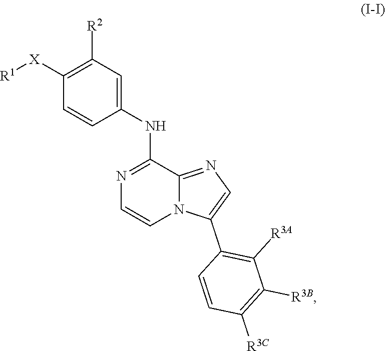
wherein:
X, R¹, and R² are as defined in claim 1; and
R^{3A}, R^{3B}, and R^{3C} are each independently defined like R³ in claim 1.

3. The compound of formula (I) according to claim 1 or 2, or a pharmaceutically acceptable salt thereof, wherein X is selected from a covalent bond, carbonyl, —(CH₂)ₚ—C(O)—NR⁴—, —(CH₂)_{q}SO₂—, —NR⁵—S(O)₂—, a group 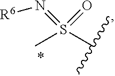
and a group 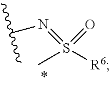
wherein
R⁴ and R⁵ are each independently selected from hydrogen and C₁-C₆-alkyl;
R⁶ is selected from C₁-C₆-alkyl, amino-C₁-C₆-alkyl, and hydroxy-(3- to 14-membered heterocyclyl)-C(O)—NH—C₁-C₆-alkyl;
the asterisk indicates the point of attachment of R¹ to X; and
the wavy line indicates the point of attachment of X to the remainder of formula (I); and
p and q are each independently 0 or 1.

4. The compound of formula (I) according to claim 1 or 2, or a pharmaceutically acceptable salt thereof, wherein X is selected from a covalent bond, carbonyl, —(CH₂)ₚ—C(O)—NR⁴—, —(CH₂)_{q}SO₂—, —NR⁵—S(O)₂—, a group 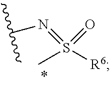
and a group 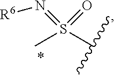
wherein
R⁴ and R⁵ are both hydrogen;
R⁶ is C₁-C₆-alkyl or hydroxy-(3- to 14-membered heterocyclyl)-C(O)—NH—C₁-C₆-alkyl;
the asterisk indicates the point of attachment of R¹ to X; and
the wavy line indicates the point of attachment of X to the remainder of formula (I);
p is 0 or 1; and
q is 0.

5. The compound of formula (I) according to claim 1 or 2, or a pharmaceutically acceptable salt thereof, wherein X is selected from a covalent bond, carbonyl, —(CH₂)ₚ—C(O)—NR⁴—, —(CH₂)_{q}SO₂—, —NR⁵—S(O)₂—, a group 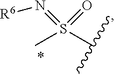
and a group 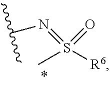
wherein
R⁴ and R⁵ are both hydrogen;
R⁶ is methyl or hydroxypyrrolidinyl-C(O)—NH—(CH₂)₃—;
the asterisk indicates the point of attachment of R¹ to X; and
the wavy line indicates the point of attachment of X to the remainder of formula (I);
p is 0 or 1; and
q is 0.

6. The compound of formula (I) according to any one of claims 1 to 5, or a pharmaceutically acceptable salt thereof, wherein
R¹ is selected from halogen, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, hydroxy-C₁-C₆-alkyl, amino-C₁-C₆-alkyl, (C₁-C₆-alkyl)₃N⁺—C₁-C₆-alkyl, (C₁-C₆-alkyl)₂N—, C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)—NH—C₁-C₆-alkoxy, and a group 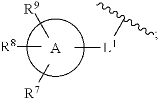
wherein
A is selected from 3- to 14-membered heterocyclyl, 5- to 14-membered heteroaryl, and C₃-C₁₀-cycloalkyl;
L¹ is a covalent bond or C₁-C₆-alkyl;
R⁷ is selected from hydrogen, C₁-C₆-alkyl, amino, C₁-C₆-alkyl, amino-C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl, and oxo; wherein said 3- to 14-membered heterocyclyl is optionally substituted with a substituent selected from hydroxy and hydroxy-(3-to 14-membered heterocyclyl)-C(O)—;
R⁸ is hydrogen or oxo;
R⁹ is hydrogen; and
r is 2 or 3.

7. The compound of formula (I) according to any one of claims 1 to 5, or a pharmaceutically acceptable salt thereof, wherein
R¹ is selected from amino, C₁-C₆-alkyl, amino-C₁-C₆-alkoxy-C₁-C₆-alkyl, amino-(CH₂CH₂O)ᵣ—, and a group 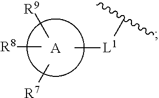
wherein
A is 3- to 14-membered heterocyclyl or C₃-C₁₀-cycloalkyl;
L¹ is a covalent bond;
R⁷ is selected from amino, amino-C₁-C₆-alkyl, (C₁-C₆-alkyl)₂N—C₁-C₆-alkyl, hydroxy-(3- to 14-membered heterocyclyl)-C(O)—, and oxo;
R⁸ is hydrogen or oxo;
R⁹ is hydrogen; and
r is 3.

8. The compound of formula (I) according to any one of claims 1 to 5, or a pharmaceutically acceptable salt thereof, wherein
R¹ is selected from amino, methyl, amino-(CH₂)₂—O—(CH₂)₂—, amino-(CH₂CH₂O)ᵣ—, and a group 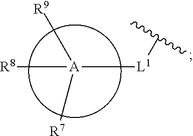
wherein
A is selected from pyrrolidinyl, imidazolidinyl, piperazinyl, piperidyl, isothiazolidinyl, and cyclohexyl;
L¹ is a covalent bond;
R⁷ is selected from amino, aminomethyl, aminobutyl, (CH₃)₂N—(CH₂)₂—, hydroxypyrrolidinyl-C(O)—, and oxo;
R⁸ is hydrogen or oxo;
R⁹ is hydrogen; and
r is 3.

9. The compound of formula (I) according to any one of claims 1 to 8, or a pharmaceutically acceptable salt thereof, wherein R² is selected from hydrogen, halogen, C₁-C₆-alkyl, hydroxy-C₁-C₆-alkyl, and C₁-C₆-alkyl-NH—C(O)—.

10. The compound of formula (I) according to any one of claims 1 to 8, or a pharmaceutically acceptable salt thereof, wherein R² is selected from hydrogen, halogen, C₁-C₆-alkyl, and C₁-C₆-alkyl-NH—C(O)—.

11. The compound of formula (I) according to any one of claims 1 to 8, or a pharmaceutically acceptable salt thereof, wherein R² is selected from hydrogen, chloro, methyl, ethyl, and CH₃—NH—C(O)—.

12. The compound of formula (I) according to any one of claims 1 to 5, or a pharmaceutically acceptable salt thereof, wherein
R¹ and one occurrence of R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with amino-C₁-C₆-alkyl-NH—.

13. The compound of formula (I) according to any one of claims 1 to 5, or a pharmaceutically acceptable salt thereof, wherein
R¹ and one occurrence of R², taken together with the atoms to which they are attached, form a C₃-C₁₀-cycloalkyl, wherein said C₃-C₁₀-cycloalkyl is substituted with amino-C₁-C₆-alkyl-NH—.

14. The compound of formula (I) according to any one of claims 1 to 5, or a pharmaceutically acceptable salt thereof, wherein
R¹ and one occurrence of R², taken together with the atoms to which they are attached, form a cyclopentene, wherein said cyclopentene is substituted with aminoethyl-NH—.

15. The compound of formula (I) according to any one of claims 1 to 14, or a pharmaceutically acceptable salt thereof, wherein R³ is selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy.

16. The compound of formula (I) according to any one of claims 1 to 14, or a pharmaceutically acceptable salt thereof, wherein R³ is selected from hydrogen, halogen, C₁-C₆-alkoxy, halo-C₁-C₆-alkoxy, and (5- to 14-membered heteroaryl)oxy.

17. The compound of formula (I) according to any one of claims 1 to 14, or a pharmaceutically acceptable salt thereof, wherein R³ is selected from hydrogen, chloro, fluoro, methoxy, difluoromethoxy, and pyridyloxy.

18. The compound of formula (I) according to any one of claims 2 to 14, or a pharmaceutically acceptable salt thereof, wherein
R^{3A} and R^{3B} are independently selected from hydrogen and halogen; and
R^{3C} is selected from halogen, C₁-C₆-alkoxy, cyano-C₁-C₆-alkoxy, halo-C₁-C₆-alkoxy, C₂-C₆-alkynyloxy, and (5- to 14-membered heteroaryl)oxy.

19. The compound of formula (I) according to any one of claims 2 to 14, or a pharmaceutically acceptable salt thereof, wherein
R^{3A} and R^{3B} are independently selected from hydrogen and halogen; and
R^{3C} is selected from C₁-C₆-alkoxy, halo-C₁-C₆-alkoxy, and (5- to 14-membered heteroaryl)oxy.

20. The compound of formula (I) according to any one of claims 2 to 14, or a pharmaceutically acceptable salt thereof, wherein
R^{3A} is selected from hydrogen and fluoro;
R^{3B} is selected from hydrogen, chloro and fluoro; and
R^{3C} is selected from methoxy, difluoromethoxy, and pyridyloxy.

21. The compound of formula (I) according to any one of claims 1 and 3 to 17, or a pharmaceutically acceptable salt thereof, wherein
m is 1; and
n is an integer selected from 1, 2, and 3.

22. The compound of formula (I) according to claim 1 or 2, or a pharmaceutically acceptable salt thereof, wherein
X is selected from a covalent bond, carbonyl, —(CH₂)ₚ—C(O)—NR⁴—, —(CH₂)_{q}SO₂—, —NR⁵—S(O)₂—, a group 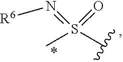
and a group 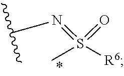
R¹ is selected from halogen, amino, hydroxy, (C₁-C₆-alkyl)₂N—C(O)—C₁-C₆-alkyl, (C₁-C₆-alkyl)₂N—C₁-C₆-alkoxy-C₁-C₆-alkyl, C₁-C₆-alkyl, hydroxy-C₁-C₆-alkyl, amino-C₁-C₆-alkyl, (C₁-C₆-alkyl)₃N⁺—C₁-C₆-alkyl, (C₁-C₆-alkyl)₂N—, C₁-C₆-alkoxy, amino-C₁-C₆-alkoxy, amino-C₁-C₆-alkoxy-C₁-C₆-alkyl, amino-(CH₂CH₂O)ᵣ—, C₁-C₆-alkyl-C(O)—NH—C₁-C₆-alkoxy, and a group 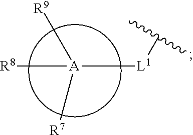
and
R² is selected from hydrogen, halogen, C₁-C₆-alkyl, hydroxy-C₁-C₆-alkyl, and C₁-C₆-alkyl-NH—C(O)—; or
R¹ and R², taken together with the atoms to which they are attached, form a 3- to 14-membered heterocyclyl or a C₃-C₁₀-cycloalkyl, wherein said 3- to 14-membered heterocyclyl or C₃-C₁₀-cycloalkyl is optionally substituted with amino-C₁-C₆-alkyl-NH—;
R³ is selected from hydrogen, halogen, C₁-C₆-alkoxy, cyano-C₁-C₆-alkoxy, halo-C₁-C₆-alkoxy, C₂-C₆-alkynyloxy, and (5- to 14-membered heteroaryl)oxy;
R⁴ and R⁵ are each independently selected from hydrogen and C₁-C₆-alkyl;
R⁶ is selected from C₁-C₆-alkyl, amino-C₁-C₆-alkyl, and hydroxy-(3- to 14-membered heterocyclyl)-C(O)—NH—C₁-C₆-alkyl;
R⁷ is selected from hydrogen, C₁-C₆-alkyl, amino, C₁-C₆-alkyl, amino-C₁-C₆-alkyl, (C₁-C₆-alkyl)₂N—C₁-C₆-alkyl, (C₁-C₆-alkyl)₂N—C₁-C₆-alkyl-C(O)—, (C₁-C₆-alkyl)₃N⁺—C₁-C₆-alkyl-C(O)—, (3- to 14-membered heterocyclyl)-C(O)—, amino-C₁-C₆-alkoxy-C₁-C₆-alkyl, and oxo; wherein said 3- to 14-membered heterocyclyl is optionally substituted with a substituent selected from hydroxy and hydroxy-(3-to 14-membered heterocyclyl)-C(O)—;
R⁸ is hydrogen or oxo;
R⁹ is hydrogen;
A is selected from 3- to 14-membered heterocyclyl, 5- to 14-membered heteroaryl, and C₃-C₁₀-cycloalkyl;
L¹ is a covalent bond or C₁-C₆-alkyl;
the asterisk indicates the point of attachment of R¹ to X;
the wavy line indicates the point of attachment of X to the remainder of formula (I);
m is 1;
n is an integer selected from 1, 2, and 3;
p and q are each independently 0 or 1; and
r is 2 or 3.

23. The compound of formula (I) according to claim 1 or 2, or a pharmaceutically acceptable salt thereof, wherein
X is selected from a covalent bond, carbonyl, —(CH₂)ₚ—C(O)—NR⁴—, —(CH₂)_{q}SO₂—, —NR⁵—S(O)₂—, a group 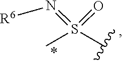
and a group 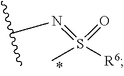
R¹ is selected from amino, C₁-C₆-alkyl, amino-C₁-C₆-alkoxy-C₁-C₆-alkyl, amino-(CH₂CH₂O)ᵣ—, and a group 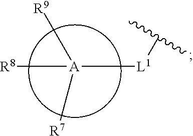
and
R² is selected from hydrogen, halogen, C₁-C₆-alkyl, and C₁-C₆-alkyl-NH—C(O)—; or
R¹ and R², taken together with the atoms to which they are attached, form a C₃-C₁₀-cycloalkyl, wherein said C₃-C₁₀-cycloalkyl is substituted with amino-C₁-C₆-alkyl-NH—;
R³ is selected from hydrogen, halogen, C₁-C₆-alkoxy, halo-C₁-C₆-alkoxy, and (5- to 14-membered heteroaryl)oxy;
R⁴ and R⁵ are both hydrogen;
R⁶ is C₁-C₆-alkyl or hydroxy-(3- to 14-membered heterocyclyl)-C(O)—NH—C₁-C₆-alkyl;
R⁷ is selected from amino, amino-C₁-C₆-alkyl, (C₁-C₆-alkyl)₂N—C₁-C₆-alkyl, hydroxy-(3- to 14-membered heterocyclyl)-C(O)—, and oxo;
R⁸ is hydrogen or oxo;
R⁹ is hydrogen;
A is 3- to 14-membered heterocyclyl or C₃-C₁₀-cycloalkyl;
L¹ is a covalent bond;
the asterisk indicates the point of attachment of R¹ to X;
the wavy line indicates the point of attachment of X to the remainder of formula (I);
m is 1;
n is an integer selected from 1, 2, and 3;
p is 0 or 1;
q is 0; and
r is 3.

24. The compound of formula (I) according to claim 1 or 2, or a pharmaceutically acceptable salt thereof, wherein
X is selected from a covalent bond, carbonyl, —(CH₂)ₚ—C(O)—NR⁴—, —(CH₂)_{q}SO₂—, —NR⁵—S(O)₂—, a group 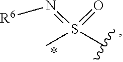
and a group 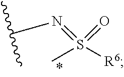
R¹ is selected from amino methyl, amino-(CH₂)₂—O—(CH₂)₂—, amino-(CH₂CH₂O)ᵣ—, and a group 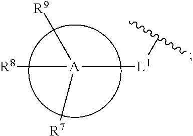
and
R² is selected from hydrogen, chloro, methyl, ethyl, and CH₃—NH—C(O)—; or
R¹ and R², taken together with the atoms to which they are attached, form a cyclopentene, wherein said cyclopentene is substituted with aminoethyl-NH—;
R³ is selected from hydrogen, chloro, fluoro, methoxy, difluoromethoxy, and pyridyloxy;
R⁴ and R⁵ are both hydrogen;
R⁶ is methyl or hydroxypyrrolidinyl-C(O)—NH—(CH₂)₃—;
R⁷ is selected from amino, aminomethyl, aminobutyl, (CH₃)₂N—(CH₂)₂—, hydroxypyrrolidinyl-C(O)—, and oxo;
R⁸ is hydrogen or oxo;
R⁹ is hydrogen;
A is selected from pyrrolidinyl, imidazolidinyl, piperazinyl, piperidyl, isothiazolidinyl, and cyclohexyl;
L¹ is a covalent bond;
the asterisk indicates the point of attachment of R¹ to X;
the wavy line indicates the point of attachment of X to the remainder of formula (I);
m is 1;
n is an integer selected from 1, 2, and 3;
p is 0 or 1;
q is 0; and
r is 3.

25. The compound of formula (I) according to claim 1 or 2, or a pharmaceutically acceptable salt thereof, wherein the compound of formula (I) is selected from:
N-[4-[[3-(3-chloro-4-methoxy-phenyl)imidazo[1,2-a]pyrazin-8-yl]amino]-2-methyl-phenyl]acetamide;
1-[4-[[3-(3-fluoro-4-methoxy-phenyl)imidazo[1,2-a]pyrazin-8-yl]amino]phenyl]pyrrolidin-2-one;
1-[2-chloro-4-[[3-[4-(difluoromethoxy)phenyl]imidazo[1,2-a]pyrazin-8-yl]amino]phenyl]pyrrolidin-2-one;
N-[4-[[3-(4-methoxyphenyl)imidazo[1,2-a]pyrazin-8-yl]amino]-2-methyl-phenyl]acetamide;
N-[4-[[3-(4-chlorophenyl)imidazo[1,2-a]pyrazin-8-yl]amino]-2-methyl-phenyl]acetamide;
N-[4-[[3-[4-(difluoromethoxy)phenyl]imidazo[1,2-a]pyrazin-8-yl]amino]-2-methyl-phenyl]acetamide;
N-[2-chloro-4-[[3-(4-methoxyphenyl)imidazo[1,2-a]pyrazin-8-yl]amino]phenyl]acetamide;
N-[4-[[3-(3-fluoro-4-methoxy-phenyl)imidazo[1,2-a]pyrazin-8-yl]amino]-2-methyl-phenyl]acetamide;
N-[4-[[3-(3-chloro-4-methoxy-phenyl)imidazo[1,2-a]pyrazin-8-yl]amino]-2-methyl-phenyl]acetamide;
4-(4-aminobutyl)-1-[4-[[3-[4-(difluoromethoxy)phenyl]imidazo[1,2-a]pyrazin-8-yl]amino]phenyl]pyrrolidin-2-one;
1-[4-[[3-[4-(difluoromethoxy)-3-fluoro-phenyl]imidazo[1,2-a]pyrazin-8-yl]amino]phenyl]pyrrolidin-2-one;
1-[4-[[3-[4-(difluoromethoxy)phenyl]imidazo[1,2-a]pyrazin-8-yl]amino]phenyl]pyrrolidin-2-one;
N-[4-[[3-(3-chloro-4-methoxy-phenyl)imidazo[1,2-a]pyrazin-8-yl]amino]-2-ethyl-phenyl]acetamide;
N-[4-[[3-(4-chloro-3-fluoro-phenyl)imidazo[1,2-a]pyrazin-8-yl]amino]-2-methyl-phenyl]acetamide;
3-amino-N-[4-[[3-(2,3-difluoro-4-methoxy-phenyl)imidazo[1,2-a]pyrazin-8-yl]amino]-2-ethyl-phenyl]propanamide;
N-[2-ethyl-4-[[3-(4-methoxyphenyl)imidazo[1,2-a]pyrazin-8-yl]amino]phenyl]acetamide;
4-(5-aminopentyl)-1-[4-[[3-[4-(difluoromethoxy)phenyl]imidazo[1,2-a]pyrazin-8-yl]amino]phenyl]pyrrolidin-2-one;
N-[2-[2-[4-[[3-(4-methoxyphenyl)imidazo[1,2-a]pyrazin-8-yl]amino]-2-methyl-anilino]-2-oxo-ethoxy]ethyl]pentanamide;
1-[4-[[3-[4-(difluoromethoxy)-3-fluoro-phenyl]imidazo[1,2-a]pyrazin-8-yl]amino]-2-methyl-phenyl]pyrrolidin-2-one;
3-[4-[[3-[4-(difluoromethoxy)phenyl]imidazo[1,2-a]pyrazin-8-yl]amino]phenyl]oxazolidin-2-one;
2-(3-aminopyrrolidin-1-yl)-N-[4-[[3-[4-(difluoromethoxy)phenyl]imidazo[1,2-a]pyrazin-8-yl]amino]-2-methyl-phenyl]acetamide;
1-[4-[[3-(4-methoxyphenyl)imidazo[1,2-a]pyrazin-8-yl]amino]-2-methyl-phenyl]pyrrolidin-2-one;
N-[4-[[3-(2-chloro-4-methoxy-phenyl)imidazo[1,2-a]pyrazin-8-yl]amino]-2-methyl-phenyl]acetamide;
1-[4-[[3-(3-chloro-4-methoxy-phenyl)imidazo[1,2-a]pyrazin-8-yl]amino]phenyl]piperidin-2-one;
N-[2-methyl-4-[[3-(2,3,4-trifluorophenyl)imidazo[1,2-a]pyrazin-8-yl]amino]phenyl]acetamide;
N-[4-[[3-(2-fluoro-4-methoxy-phenyl)imidazo[1,2-a]pyrazin-8-yl]amino]-2-methyl-phenyl]acetamide;
N-[4-[[3-[4-(difluoromethoxy)phenyl]imidazo[1,2-a]pyrazin-8-yl]amino]-2-methyl-phenyl]-2-(4-ethylpiperazin-1-yl)acetamide;
2-(2-aminoethoxy)-N-[4-[[3-(4-methoxyphenyl)imidazo[1,2-a]pyrazin-8-yl]amino]-2-methyl-phenyl]acetamide;
4-(2-aminoethoxymethyl)-1-[4-[[3-[4-(difluoromethoxy)phenyl]imidazo[1,2-a]pyrazin-8-yl]amino]phenyl]pyrrolidin-2-one;
N-[4-[[3-(3-chloro-4-methoxy-phenyl)imidazo[1,2-a]pyrazin-8-yl]amino]-2-methyl-phenyl]-2-piperazin-1-yl-acetamide;
N-[4-[[3-(4-methoxyphenyl)imidazo[1,2-a]pyrazin-8-yl]amino]-2-methyl-phenyl]-2-piperazin-1-yl-acetamide;
1-[4-[[3-(4-methoxyphenyl)imidazo[1,2-a]pyrazin-8-yl]amino]phenyl]pyrrolidin-2-one;
N-[4-[[3-[4-(cyanomethoxy)phenyl]imidazo[1,2-a]pyrazin-8-yl]amino]-2-methyl-phenyl]acetamide;
N-[4-[[3-(4-methoxyphenyl)imidazo[1,2-a]pyrazin-8-yl]amino]-2-methyl-phenyl]-2-piperazin-1-yl-acetamide;
4-[4-[[3-[4-(difluoromethoxy)phenyl]imidazo[1,2-a]pyrazin-8-yl]amino]phenyl]morpholin-3-one;
1-[4-[[3-(3-chloro-4-methoxy-phenyl)imidazo[1,2-a]pyrazin-8-yl]amino]phenyl]pyrrolidin-2-one;
N-[4-[[3-(3,4-difluorophenyl)imidazo[1,2-a]pyrazin-8-yl]amino]-2-methyl-phenyl]acetamide;
N-[4-[[3-(4-fluorophenyl)imidazo[1,2-a]pyrazin-8-yl]amino]-2-methyl-phenyl]acetamide;
1-[4-[[3-[4-(difluoromethoxy)phenyl]imidazo[1,2-a]pyrazin-8-yl]amino]phenyl]-5-methyl-pyrrolidin-2-one;
1-[4-[[3-(3-fluoro-4-methoxy-phenyl)imidazo[1,2-a]pyrazin-8-yl]amino]phenyl]pyrrolidin-2-one;
N-[4-[[3-(3-fluoro-4-methoxy-phenyl)imidazo[1,2-a]pyrazin-8-yl]amino]-2-methyl-phenyl]acetamide;
N-[4-[[3-(3-fluoro-4-methoxy-phenyl)imidazo[1,2-a]pyrazin-8-yl]amino]phenyl]acetamide;
N-[4-[[3-(3-chloro-4-methoxy-phenyl)imidazo[1,2-a]pyrazin-8-yl]amino]phenyl]acetamide;
N-[4-[[3-[3-chloro-4-(difluoromethoxy)phenyl]imidazo[1,2-a]pyrazin-8-yl]amino]phenyl]acetamide;
N-[4-[[3-(2-fluoro-4-methoxy-phenyl)imidazo[1,2-a]pyrazin-8-yl]amino]phenyl]acetamide;
N-[2-(hydroxymethyl)-4-[[3-(4-methoxyphenyl)imidazo[1,2-a]pyrazin-8-yl]amino]phenyl]acetamide;
2-(2-aminoethoxy)-N-[4-[[3-(3-chloro-4-methoxy-phenyl)imidazo[1,2-a]pyrazin-8-yl]amino]phenyl]acetamide;
N-[4-[[3-(4-methoxyphenyl)imidazo[1,2-a]pyrazin-8-yl]amino]phenyl]acetamide;
N-[4-[[3-(2,4-difluorophenyl)imidazo[1,2-a]pyrazin-8-yl]amino]-2-methyl-phenyl]acetamide;
1-[4-[[3-[4-(difluoromethoxy)phenyl]imidazo[1,2-a]pyrazin-8-yl]amino]-2-methyl-phenyl]pyrrolidin-2-one;
2-chloro-N-[4-[[3-(4-methoxyphenyl)imidazo[1,2-a]pyrazin-8-yl]amino]phenyl]acetamide;
N-[4-[[3-(4-prop-2-ynoxyphenyl)imidazo[1,2-a]pyrazin-8-yl]amino]phenyl]acetamide;
1-[4-[[3-(4-chlorophenyl)imidazo[1,2-a]pyrazin-8-yl]amino]-2-methyl-phenyl]pyrrolidin-2-one;
2-methoxy-N-[4-[[3-(4-methoxyphenyl)imidazo[1,2-a]pyrazin-8-yl]amino]phenyl]acetamide;
2-[2-(2-aminoethoxy)ethoxy]-N-[4-[[3-(3-chloro-4-methoxy-phenyl)imidazo[1,2-a]pyrazin-8-yl]amino]phenyl]acetamide;
N-[4-[[3-(3-chloro-4-methoxy-phenyl)imidazo[1,2-a]pyrazin-8-yl]amino]phenyl]-2-hydroxy-acetamide;
N-[4-[[3-[4-(difluoromethoxy)phenyl]imidazo[1,2-a]pyrazin-8-yl]amino]phenyl]acetamide;
N-[4-[[3-(4-chlorophenyl)imidazo[1,2-a]pyrazin-8-yl]amino]phenyl]acetamide;
N-[4-[[3-(3-chloro-4-methoxy-phenyl)imidazo[1,2-a]pyrazin-8-yl]amino]phenyl]-2-morpholino-acetamide;
N-[4-[[3-(3-chloro-4-methoxy-phenyl)imidazo[1,2-a]pyrazin-8-yl]amino]phenyl]-N-methyl-acetamide;
7-[[3-(3-fluoro-4-methoxy-phenyl)imidazo[1,2-a]pyrazin-8-yl]amino]-1,3,4,5-tetrahydro-1-benzazepin-2-one;
N-[4-[[3-(2,3,4-trifluorophenyl)imidazo[1,2-a]pyrazin-8-yl]amino]phenyl]acetamide;
N-[2-chloro-4-[[3-(3-chloro-4-methoxy-phenyl)imidazo[1,2-a]pyrazin-8-yl]amino]phenyl]acetamide;
N-[4-[[3-(3-fluoro-4-methoxy-phenyl)imidazo[1,2-a]pyrazin-8-yl]amino]phenyl]-2-hydroxy-acetamide;
N-[4-[[3-(3-chloro-4-methoxy-phenyl)imidazo[1,2-a]pyrazin-8-yl]amino]phenyl]-N-methyl-acetamide;
1-[4-[[3-[4-(difluoromethoxy)phenyl]imidazo[1,2-a]pyrazin-8-yl]amino]phenyl]imidazolidin-2-one;
N4-[3-(2,3-difluoro-4-methoxy-phenyl)imidazo[1,2-a]pyrazin-8-yl]-2-ethyl-benzene-1,4-diamine;
N-[4-[(4-aminocyclohexyl)methylsulfonyl]-3-chloro-phenyl]-3-(2,3-difluoro-4-methoxy-phenyl)imidazo[1,2-a]pyrazin-8-amine;
N-(3-chloro-4-methylsulfonyl-phenyl)-3-(4-methoxyphenyl)imidazo[1,2-a]pyrazin-8-amine;
3-(4-methoxyphenyl)-N-(4-methylsulfonylphenyl)imidazo[1,2-a]pyrazin-8-amine;
N-[2-(2-aminoethoxy)ethyl]-2-chloro-4-[[3-(2,3-difluoro-4-methoxy-phenyl)imidazo[1,2-a]pyrazin-8-yl]amino]benzenesulfonamide;
N-[2-(2-aminoethoxy)ethyl]-4-[[3-(2,3-difluoro-4-methoxy-phenyl)imidazo[1,2-a]pyrazin-8-yl]amino]-2-methyl-benzenesulfonamide;
2-chloro-4-[[3-(2,3-difluoro-4-methoxy-phenyl)imidazo[1,2-a]pyrazin-8-yl]amino]-N-[2-[2-(dimethylamino)ethoxy]ethyl]benzenesulfonamide;
N-[3-chloro-4-[4-[3-(dimethylamino)propyl]piperazin-1-yl]sulfonyl-phenyl]-3-(2,3-difluoro-4-methoxy-phenyl)imidazo[1,2-a]pyrazin-8-amine;
[4-[4-[[3-(2,3-difluoro-4-methoxy-phenyl)imidazo[1,2-a]pyrazin-8-yl]amino]-2-ethyl-phenyl]sulfonylpiperazin-1-yl]-[(3R)-pyrrolidin-3-yl]methanone;
[4-[4-[[3-(2,3-difluoro-4-methoxy-phenyl)imidazo[1,2-a]pyrazin-8-yl]amino]-2-ethyl-phenyl]sulfonylpiperazin-1-yl]-(4-piperidyl)methanone;
[4-[4-[[3-(2,3-difluoro-4-methoxy-phenyl)imidazo[1,2-a]pyrazin-8-yl]amino]-2-ethyl-phenyl]sulfonylpiperazin-1-yl]-[(2S,4R)-4-hydroxypyrrolidin-2-yl]methanone;
4-[[3-(2,3-difluoro-4-methoxy-phenyl)imidazo[1,2-a]pyrazin-8-yl]amino]-N-[2-[2-(dimethylamino)ethoxy]ethyl]-2-methyl-benzenesulfonamide;
3-(2,3-difluoro-4-methoxy-phenyl)-N-[4-[4-[3-(dimethylamino)propyl]piperazin-1-yl]sulfonyl-3-methyl-phenyl]imidazo[1,2-a]pyrazin-8-amine;
[4-[4-[[3-(2,3-difluoro-4-methoxy-phenyl)imidazo[1,2-a]pyrazin-8-yl]amino]-2-ethyl-phenyl]sulfonylpiperazin-1-yl]-[(2S)-pyrrolidin-2-yl]methanone;
[2-[4-[4-[[3-(2,3-difluoro-4-methoxy-phenyl)imidazo[1,2-a]pyrazin-8-yl]amino]-2-ethyl-phenyl]sulfonylpiperazin-1-yl]-2-oxo-ethyl]-trimethyl-ammonium;
1-[4-[4-[[3-(2,3-difluoro-4-methoxy-phenyl)imidazo[1,2-a]pyrazin-8-yl]amino]-2-ethyl-phenyl]sulfonylpiperazin-1-yl]-2-(dimethylamino)ethanone;
N-[2-(2-aminoethoxy)ethyl]-4-[[3-(2,3-difluoro-4-methoxy-phenyl)imidazo[1,2-a]pyrazin-8-yl]amino]benzenesulfonamide;
3-(2,3-difluoro-4-methoxy-phenyl)-N-(3-ethyl-4-piperazin-1-ylsulfonyl-phenyl)imidazo[1,2-a]pyrazin-8-amine;
3-(4-methoxyphenyl)-N-(4-morpholinosulfonylphenyl)imidazo[1,2-a]pyrazin-8-amine;
4-[[3-(4-methoxyphenyl)imidazo[1,2-a]pyrazin-8-yl]amino]-N,N-dimethyl-benzenesulfonamide;
[4-[4-[[3-(2,3-difluoro-4-methoxy-phenyl)imidazo[1,2-a]pyrazin-8-yl]amino]-2-ethyl-phenyl]sulfonylpiperazin-1-yl]-(4-hydroxy-4-piperidyl)methanone;
N-[2-(2-aminoethoxy)ethyl]-4-[[3-(4-methoxyphenyl)imidazo[1,2-a]pyrazin-8-yl]amino]-2-methyl-benzenesulfonamide;
N,N-diethyl-2-[[4-[[3-(4-methoxyphenyl)imidazo[1,2-a]pyrazin-8-yl]amino]phenyl]sulfonylamino]acetamide;
4-[[3-(3-chloro-4-methoxy-phenyl)imidazo[1,2-a]pyrazin-8-yl]amino]-N-methyl-benzenesulfonamide;
3-(4-methoxyphenyl)-N-[4-(4-methylpiperazin-1-yl)sulfonylphenyl]imidazo[1,2-a]pyrazin-8-amine;
[4-[4-[[3-(2,3-difluoro-4-methoxy-phenyl)imidazo[1,2-a]pyrazin-8-yl]amino]-2-ethyl-phenyl]sulfonylpiperazin-1-yl]-[(3R)-1-[(2S,4R)-4-hydroxypyrrolidine-2-carbonyl]pyrrolidin-3-yl]methanone;
N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine;
2-[4-[8-[4-(N,S-dimethylsulfonimidoyl)-3-methyl-anilino]imidazo[1,2-a]pyrazin-3-yl]-2,3-difluoro-phenoxy]acetonitrile;
3-(2,3-difluoro-4-methoxy-phenyl)-N-[4-(N,S-dimethylsulfonimidoyl)-3-methyl-phenyl]imidazo[1,2-a]pyrazin-8-amine;
N-[4-[S-(3-aminopropyl)-N-methyl-sulfonimidoyl]phenyl]-3-(2,3-difluoro-4-methoxy-phenyl)imidazo[1,2-a]pyrazin-8-amine;
N-[3-chloro-4-(N,S-dimethylsulfonimidoyl)phenyl]-3-(2,3-difluoro-4-methoxy-phenyl)imidazo[1,2-a]pyrazin-8-amine;
N-[4-[N-(3-aminopropyl)-S-methyl-sulfonimidoyl]-3-methyl-phenyl]-3-(2,3-difluoro-4-methoxy-phenyl)imidazo[1,2-a]pyrazin-8-amine;
N-[4-[S-(3-aminopropyl)-N-methyl-sulfonimidoyl]-3-methyl-phenyl]-3-(2,3-difluoro-4-methoxy-phenyl)imidazo[1,2-a]pyrazin-8-amine;
rac-(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
3-(2,3-difluoro-4-methoxy-phenyl)-N-[4-(N,S-dimethylsulfonimidoyl)-3-fluoro-phenyl]imidazo[1,2-a]pyrazin-8-amine;
[4-[S-[4-[[3-(2,3-difluoro-4-methoxy-phenyl)imidazo[1,2-a]pyrazin-8-yl]amino]phenyl]-N-methyl-sulfonimidoyl]-1-piperidyl]-[rac-(2S,4R)-4-hydroxypyrrolidin-2-yl]methanone;
N-[3-chloro-4-(S-ethyl-N-methyl-sulfonimidoyl)phenyl]-3-(2,3-difluoro-4-methoxy-phenyl)imidazo[1,2-a]pyrazin-8-amine;
rac-(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
(2S,4R)—N-[3-[[[4-[[3-(2,3-difluoro-4-methoxy-phenyl)imidazo[1,2-a]pyrazin-8-yl]amino]-2-methyl-phenyl]-methyl-oxo-lambda6-sulfanylidene]amino]propyl]-4-hydroxy-pyrrolidine-2-carboxamide;
N-[4-[N-(3-aminopropyl)-S-methyl-sulfonimidoyl]-3-ethyl-phenyl]-3-(2,3-difluoro-4-methoxy-phenyl)imidazo[1,2-a]pyrazin-8-amine;
3-(2,3-difluoro-4-methoxy-phenyl)-N-[4-(N,S-dimethylsulfonimidoyl)phenyl]imidazo[1,2-a]pyrazin-8-amine;
[4-[S-[4-[[3-(2,3-difluoro-4-methoxy-phenyl)imidazo[1,2-a]pyrazin-8-yl]amino]-2-methyl-phenyl]-N-methyl-sulfonimidoyl]-1-piperidyl]-[rac-(2S,4R)-4-hydroxypyrrolidin-2-yl]methanone;
N-[4-[N-(3-aminopropyl)-S-methyl-sulfonimidoyl]phenyl]-3-(2,3-difluoro-4-methoxy-phenyl)imidazo[1,2-a]pyrazin-8-amine;
rac-(2S,4R)—N-[3-[[[4-[[3-(2,3-difluoro-4-methoxy-phenyl)imidazo[1,2-a]pyrazin-8-yl]amino]phenyl]-methyl-oxo-lambda6-sulfanylidene]amino]propyl]-4-hydroxy-pyrrolidine-2-carboxamide;
3-(2,3-difluoro-4-methoxy-phenyl)-N-[4-[S-(dimethylamino)-N-methyl-sulfonimidoyl]-3-methyl-phenyl]imidazo[1,2-a]pyrazin-8-amine;
N-[4-[4-(aminomethyl)-1-piperidyl]-3-methyl-phenyl]-3-(2,3-difluoro-4-methoxy-phenyl)imidazo[1,2-a]pyrazin-8-amine;
N-[4-[4-(aminomethyl)-1-piperidyl]-3-chloro-phenyl]-3-(2,3-difluoro-4-methoxy-phenyl)imidazo[1,2-a]pyrazin-8-amine;
N-[4-[4-(aminomethyl)-1-piperidyl]phenyl]-3-(2,3-difluoro-4-methoxy-phenyl)imidazo[1,2-a]pyrazin-8-amine;
N-[4-[4-(aminomethyl)-1-piperidyl]-3-ethyl-phenyl]-3-(2,3-difluoro-4-methoxy-phenyl)imidazo[1,2-a]pyrazin-8-amine;
N1-(2-aminoethyl)-N5-[3-(2,3-difluoro-4-methoxy-phenyl)imidazo[1,2-a]pyrazin-8-yl]indane-1,5-diamine;
5-[[3-(2,3-difluoro-4-methoxy-phenyl)imidazo[1,2-a]pyrazin-8-yl]amino]indan-1-one;
2-iodo-5-[[3-(4-methoxyphenyl)imidazo[1,2-a]pyrazin-8-yl]amino]-N-methyl-benzamide;
3-(2,3-difluoro-4-methoxy-phenyl)-N-[4-[[dimethyl(oxo)-lambda6-sulfanylidene]amino]-3-methyl-phenyl]imidazo[1,2-a]pyrazin-8-amine;
3-[4-(difluoromethoxy)phenyl]-N-[4-(1,1-dioxo-1,2-thiazolidin-2-yl)phenyl]imidazo[1,2-a]pyrazin-8-amine;
N-[4-[2-[2-(2-aminoethoxy)ethoxy]ethoxy]-3-methyl-phenyl]-3-[2,3-difluoro-4-(2-pyridyloxy)phenyl]imidazo[1,2-a]pyrazin-8-amine;
2-chloro-5-[[3-(4-methoxyphenyl)imidazo[1,2-a]pyrazin-8-yl]amino]-N-methyl-benzamide;
3-(2,3-difluoro-4-methoxy-phenyl)-N-[4-(imidazol-1-ylmethyl)-3-methyl-phenyl]imidazo[1,2-a]pyrazin-8-amine;
[4-[[3-[4-(difluoromethoxy)phenyl]imidazo[1,2-a]pyrazin-8-yl]amino]-2-methyl-phenyl]methanol;
N-[4-(imidazol-1-ylmethyl)-3-methyl-phenyl]-3-(4-methoxyphenyl)imidazo[1,2-a]pyrazin-8-amine;
N-[4-(imidazol-1-ylmethyl)phenyl]-3-(4-methoxyphenyl)imidazo[1,2-a]pyrazin-8-amine;
[4-[[3-(2,3-difluoro-4-methoxy-phenyl)imidazo[1,2-a]pyrazin-8-yl]amino]-2-ethyl-phenyl]methyl-trimethyl-ammonium; and
[4-[[3-(4-methoxyphenyl)imidazo[1,2-a]pyrazin-8-yl]amino]-2-methyl-phenyl]methanol.

26. A process of manufacturing the compounds of formula (I) according to any one of claims 1 to 25, comprising:
(i) reacting a heteroaryl halide (IV), wherein R¹, R², X and m are as defined in any one of claims 1 to 25 and Y is bromo or iodo, 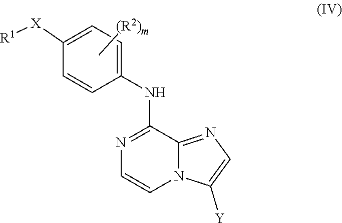
with a compound (V), wherein R³ and n are as defined in any one of claims 1 to 25 and R is hydrogen or C₁-C₆-alkyl or the two R groups, taken together with the atoms to which they are attached, form a cyclic boronic acid ester, 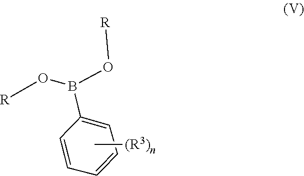
in the presence of a transition metal catalyst, to afford said compound of formula (I); or
(ii) reacting a heteroaryl chloride (VI), wherein R³ and n are as defined in any one of claims 1 to 25, 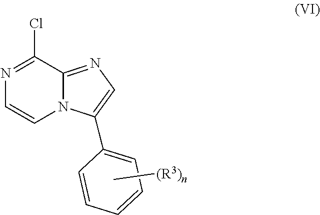
with an aniline derivative (III), wherein R¹, R², X, and m are as defined in any one of claims 1 to 25, 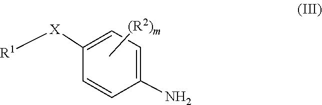
to afford said compound of formula (I); and
(iii) optionally converting said compound of formula (I) to a pharmaceutically acceptable salt thereof.

27. A compound of formula (I) according to any one of claims 1 to 25, when manufactured according to the process of claim 26.

28. A compound of formula (I) according to any one of claims 1 to 25 and 27, or a pharmaceutically acceptable salt thereof, for use as therapeutically active substance.

29. A pharmaceutical composition comprising a compound of formula (I) according to any one of claims 1 to 25 and 27, or a pharmaceutically acceptable salt thereof, and a therapeutically inert carrier.

30. A compound of formula (I) according to any of claims 1 to 25 and 27, or a pharmaceutically acceptable salt thereof, for use as antibiotic.

31. A compound of formula (I) according to any of claims 1 to 25 and 27, or a pharmaceutically acceptable salt thereof, for use in the treatment or prevention of nosocomial infections and resulting diseases.

32. A compound of formula (I) according to any of claims 1 to 25 and 27, or a pharmaceutically acceptable salt thereof, for use in the treatment or prevention of infections and resulting diseases caused by Gram-negative bacteria.

33. The compound for use according to claim 32, wherein said Gram-negative bacteria are selected from Klebsiella pneumoniae, Acinetobacter baumannii, Pseudomonas aeruginosa, Enterobacter species and E. coli.

34. The compound for use according to claim 33, wherein said Gram-negative bacteria are Acinetobacter baumannii.

35. A compound of formula (I) according to any of claims 1 to 25 and 27, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

36. A method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) according to any of claims 1 to 25 and 27, or a pharmaceutically acceptable salt thereof, to a mammal.

37. Use of a compound of formula (I) according to any of claims 1 to 25 and 27, or a pharmaceutically acceptable salt thereof, as an antibiotic.

38. Use of a compound of formula (I) according to any of claims 1 to 25 and 27, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

39. The use of a compound of formula (I) according to any of claims 1 to 25 and 27, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

40. The invention as described hereinbefore.
